(19) United States
(12) Patent Application Publication
(10) Pub. No.: US 2023/0279404 A1
(43) Pub. Date: Sep. 7, 2023

(54) ENGINEERED OXALATE DECARBOXYLASES

(71) Applicants: Codexis, Inc., Redwood City, CA (US); Societe des Produits Nestle S.A., Vevey (CH)

(72) Inventors: Chinping Chng, Menlo Park, CA (US); Nikki D. Kruse, San Carlos, CA (US); Joyce Liu, Fremont, CA (US); Adam P. Silverman, San Carlos, CA (US); Rebecca Hong Thibodeau, Honolulu, HI (US); Tricia Windgassen, Redwood City, CA (US)

(21) Appl. No.: 18/167,614

(22) Filed: Feb. 10, 2023

Related U.S. Application Data
(60) Provisional application No. 63/309,397, filed on Feb. 11, 2022.

(51) Int. Cl. C12N 15/52; C12N 9/88; A61P 3/00; C12N 15/70

(52) U.S. Cl. CPC C12N 15/52; A61P 3/00; C12N 9/88; C12N 15/70; C12Y 401/01002

(57) ABSTRACT

The present disclosure provides engineered oxalate decarboxylase (ODC) polypeptides and compositions thereof, as well as polynucleotides encoding the engineered oxalate decarboxylase polypeptides. The present disclosure also provides methods of using the engineered enzymes and compositions thereof for treating diseases or conditions associated with abnormal metabolism of oxalate.

### CROSS-REFERENCE TO RELATED APPLICATIONS

This application claims the benefit under 35 U.S.C. § 119(e) of U.S. Provisional Application No. 63/309,397, filed Feb. 11, 2022, the contents of which are incorporated by reference herein.

### REFERENCE TO A SEQUENCE LISTING, A TABLE, OR A COMPUTER PROGRAM

The Sequence Listing concurrently submitted herewith as file name CX7-220US2_ST26.xml, with a creation date of Feb. 10, 2023 and a file size of 3.11 megabytes, is part of the specification and is incorporated by reference herein.

### TECHNICAL FIELD

The present disclosure relates to engineered oxalate decarboxylase (ODC) polypeptides and compositions thereof, polynucleotides encoding the engineered oxalate decarboxylase polypeptides, and methods of using the engineered oxalate decarboxylase polypeptides and compositions thereof for therapeutic and industrial purposes.

### BACKGROUND

Oxalate is found in many foods, such as leafy greens, and is also generated in the body as a metabolic product from the digestion of amino acids and ascorbic acid. In healthy individuals, oxalate is excreted in the urine (urinary oxalate: UOx), about half of which is derived from diet and the rest of which is generated as a metabolic product (Holmes et al., Kidney Int, 2001, 59(1):270-6). UOx can be variable in healthy individuals, but a level above 40 mg/day is considered hyperoxaluria.

Hyperoxaluria is generally divided into two categories. Primary hyperoxaluria is a rare genetic condition caused by mutations in alanine:glyoxylate aminotransferase (AGT) or glycolate oxidase (GO). Secondary hyperoxaluria, sometimes referred to as enteric hyperoxaluria, is a condition in which excess oxalate is absorbed in the gastrointestinal tract, which may arise from a high oxalate diet, fat malabsorption, changes in flora of intestinal oxalate-degrading microorganisms, or genetic variations of intestinal oxalate transporters (Robijn et al., Kidney Int′l, 2011, 80(11):1146-1158). Numerous conditions that may lead to secondary hyperoxaluria include Crohn’s disease (Hueppelshaeuser et al., Pediatr Nephrol., 2012, 27(7):1103-9), bariatric surgery (Lieske et al., Semin Nephrol., 2008, 28(2):163-73.; Agrawal et al., Surg Obes Relat Dis., 2014, 10(1):88-94), short bowel syndrome (Emmett et al., Am J Kidney Dis., 2003, 41(1):230-7), cystic fibrosis (Chidekel et al., Yale J Biol Med., 1996, 69(4):317-321), and celiac disease (Ciacci et al., J Urol., 2008, 180(3):974-9).

A major effect of hyperoxaluria is urinary calcium oxalate (CaOx) formation. In normal individuals, calcium binds dietary oxalate in the colon to form CaOx, which is excreted in stools. In hyperoxaluria, dietary calcium is depleted by binding to non-absorbed fatty acids, and excess oxalate remains soluble, allowing it to passively diffuse across the colonic lumen and into circulation (Nazzal et al., Nephrol Dial Transplant., 2016, 31(3):375-82). Accumulation of circulating oxalate in the kidneys results in high levels of excretion in the urine and buildup of CaOx stones in the kidney (nephrolithiasis). Furthermore, renal calcification (nephrocalcinosis) often results and contributes to the development of chronic kidney disease (CKD) and end-stage renal disease (ESRD). Many patients with hyperoxaluria have recurrent kidney stones and experience severe kidney damage over time, which can lead to CKD, ESRD, and death. UOx levels appear to be directly correlated with the risk for increased frequency of kidney stones and CKD (Curhan et al., Kidney Int., 2008, 73(4):489-96).

Treatments for hyperoxaluria include increasing fluid intake to increase urine excretion and thereby dilute UOx and treatment with alkali citrate, which forms a complex with calcium, thereby reducing precipitation of CaOx. While patients with primary hyperoxaluria may not benefit significantly from dietary oxalate restriction, patients with secondary hyperoxaluria are advised to avoid foods high in oxalate content and/or consume a diet high in calcium or calcium supplements. Although increasing dietary calcium appears to have an impact on increasing oxalate precipitation in the colon, high-dose calcium supplementation may increase CaOx urine supersaturation if high urine output is not maintained. A different treatment approach uses bile acid sequestration agents such as organic marine hydrocolloids (OMH) and cholestyramine to reduce the adsorption of oxalate. However, the efficacy of these molecules is not clear, and there are potential issues with long-term use due to worsening of steatorrhea and malabsorption of folic acid and fat-soluble vitamins (Harper et al., Postgrad Med., 1991, 67(785):219-222).

Therapeutics in development based on recombinant technology includes an engineered bacterium (SYNB8802) that degrades oxalate in the gastrointestinal tract (Puurunen et al., J Urology, 2021, 206(Supplement 3):e378-e378), a recombinant oxalate decarboxylase from B. subtilis formulated as crystals to increase its stability and activity (Reloxaliase®; Pfau et al., Nephrology Dialysis Transplantation, 2021, 36(5):945-948), and an oxalate decarboxylase from Synechococcus elongatus that upon oral delivery degrades oxalate in the gastrointestinal tract (Quintero et al., Kidney360, 2020, 1:1284-1290). While the current enzyme-based therapies under development appear to reduce plasma oxalate in healthy subjects or patients with severe enteric hyperoxaluria, desirable are alternative robust therapeutics for treating hyperoxaluria.

### SUMMARY

The present disclosure provides engineered oxalate decarboxylase (ODC) polypeptides, biologically active fragments and compositions thereof, as well as polynucleotides encoding the engineered oxalate decarboxylase (ODC) polypeptides. In some embodiments, the oxalate decarboxylase polypeptides of the disclosure are engineered to have an improved property, including, among others, enhanced catalytic activity, reduced sensitivity to proteases, and/or increased tolerance to and activity at low pH environments. The present disclosure further provides methods for using the engineered oxalate decarboxylase polypeptides and compositions thereof for treating hyperoxaluria and other disorders characterized by elevated levels of oxalate.

In one aspect, the present disclosure provides an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 2, 4, 172, 320, 396, 616, 670, 750, or 906, or to a reference sequence corresponding to residues 1-359 of SEQ ID NO: 172, 320, or 396, wherein the amino acid sequence comprises one or more amino acid substitutions relative to the reference sequence corresponding to SEQ ID NO: 2, 4, 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2 or 4, wherein the amino acid sequence comprises one or more amino acid substitutions relative to the reference sequence corresponding to SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 172, 320, 396, 616, 670, 750, or 906, or to the reference sequence corresponding to residues 1-359 of SEQ ID NO: 172, 320, or 396, wherein the amino acid sequence comprises one or more amino acid substitutions relative to the reference sequence of SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 28-1622, or to a reference sequence corresponding to residues 1-359 of an even-numbered SEQ ID NO. of SEQ ID NOs: 28-614, wherein the amino acid sequence comprises one or more amino acid substitutions relative to the reference sequence of SEQ ID NO: 2 or 4.

In some embodiments, the amino acid sequence of the engineered oxalate decarboxylase polypeptide comprises at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution at amino acid position 31, 210, or 318, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 346, 124, 359, 174, 173, 123, 196, 304, 301, 347, 11, 284, 210, 169, 216, 195, 339, 4, 6, 180, 80, 243, 182, 7, 226, 156, 183, 227, 219, 62, 343, 16/26, 16/26/242, 16/26/183/232, 155/206/242, 16/26/339, 16/26/155/206/339, 26, 26/206/339, 31/82/210, 31/82, 31/356, 31/97/226, 31/240/270, 31/82/226, 31/240, 31, 31/210/318, or 31/210, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13, 212, 124/196/210/226, 5, 16/26/124/155/195/210/284, 196/226, 16/155/195/210/226, 16/195/210/226, 16/155/174/196, 16/195/226, 318, 16/195/196/210, 16/26/124/155/174/196/210, 195/210, 26/155/174/210, 316, 60, 16/155/174, 16/124/174/196, 16/226, 16/26/174/196/226, 17, 331, 124/195, 16/174/196, 188, 195/196/210, 174/196/210, 16/26/155/174, 16/155/195/196/226, 195/226/284, 16, 240, 16/26/124/155/195/196/226, 183/232/339/343, 183/206, 63, 173/347, 16/26/174/196, 16/284, 46, 16/124/195/196, 274, 174/196, 155/174/196, 174/196/226, 16/124/155/174/195, 16/26/155/174/196, 26/174/196/210/284, 16/195/196/284, 174, 16/124/174/195/210/226/284, 16/174/195/284, 26/174/196/210/226/284, 174/196/226/284, 124/174/196, 26/174/195/210/226, 162, 16/124/195/196/284, 206/343, 16/195/196, 16/26/174/195/196/210/226, 155/195/196/226, 18, 335, 124/155/174/195/226, 26/155/174/195/226, 195/196, 153, 155/174/195, 174/195/210/284, 174/195, 37, 124/174/195/226/284, 176, 16/155/174/195/196/226/284, 10, 169/173, 155, 33, 173/183/343/347, 104/265, or 233, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/17/196, 17/212/331, 17/26/196/212/226, 17/26/196/212/226/331, 13/17/26/196/331, 17/60/196/226, 17/46/212, 26/60/196/226/331, 17/46/196/212/274/331, 212, 17/196/226, 13/17/26/212/331, 13/17/212/226/331, 196/212/331, 13/17/196/331, 13/17/26/212/226/331, 17/60/196/212/226/331, 13/17/212, 13/17/26/331, 13/17, 13/17/26/196, 46/196/212/226, 17/60/196, 17/196/331, 13/17/46/226/331, 17/196/226/331, 17/46/196/212/331, 17/46/212/331, 17/196, 13/17/212/226, 17/212, 17/26/212, 13/17/226/331, 17/196/212/331, 17/196/212/226/331, 13/17/26/226/331, 46/60/196/226/331, 17/46/196/331, 13/17/46/196, 196/212, 13/17/26, 17/83/263, 17/83/173/227, 17/227/301, 17/83/227/263, 17/83/125, 17/83/173, 17/263/301, 17/125, 17/83/301, 17/173, 17/83/227, 17/227, 17, 17/125/227, 17/83, 128, 76, 141, 110, 79, 117, or 61, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/17/79, 13/14/17/60/79, 13/17/79/83, 13/17/79/301, 13/14/17/60/79/212, 17/60/197, 13/60/212, 13/14/17/60/79/83/301, 13/17/60/79, 17/60/301, 14/17/79, 17/60/83, 17/79, 13/17/60/79/83/301, 342, 52/190, 94/190, 190/342/351, 13/79, 13/96, 273, 126, 96, 79, 269, 40, 44, 267, 266, 160, 277, 149, 22, 234, 54, 297, 106, or 13/43, wherein the amino acid positions are relative to the reference sequence corresponding to SEQ ID NO: 2 or 4.

In some embodiments, the amino acid sequence of the engineered oxalate decarboxylase polypeptide comprises at least a substitution at amino acid position 37, 125, 162, 94, 195, 85, 103, 232, 189, 186, 155, 193, 342, 196, 63, 33, 351, 314, 187, 303, 164, 153, 346, 183, 19, 123, 350, 205,284, 199, 343, 200, 208, 169, 190, or 121, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 14/26/60/318, 14/26/60/94/162/212, 60/162/226, 60, 14/125/162/212/318, 60/125/226, 14/125/162/226, 26/60/162/226/318, 14/26/94/162/212/226, 14/60/162, 14/60/162/226, 14/94/162/318, 14/94/162, 14/60/226, 14/60/94/212, 14/60/162/212, 14/162, 14/94/212/318, 14/60, 14/26/162, 14, 14/318, 14/162/226/318, 14/125/212, 14/125/226/318, 14/26/60/162, 14/125/162/212/226, 14/94/125/162/318, 14/125/226, 277, 166, 233, 83, 5, 342, 96, 284, or 26, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 5/26/83/342, 5/26/277, 5/96/277, 5/83/277/342, 26/83/277, 5/26/83/277, 5/277/342, 5/26/166/277, 83/277/342, 83/96/277/342, 83/96/277, 26/83/277/342, 5/26/83/277/342, 5/83/277, 26/277, 83/277, 5/35/277, 5/26/83/96/277, 5/26/96/277, 5/277, 5/166/277, 26/83/96/277/342, 277, 26/277/342, 5, or 5/83/96/277/342, wherein the amino acid positions are relative to the reference sequence corresponding to SEQ ID NO: 2 or 4.

In some embodiments, the amino acid sequence of the engineered oxalate decarboxylase polypeptide comprises at least a substitution at amino acid position 26, 31, 96, 60, 318, 210, 277, 37, 16, 195, 79, 17, 13, 83, 162, or 174, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 174/318, 26/96, 60/318, 31/318, 162/174/318, 174/277/318, 26/195/318, 60/162/195, 60/162/318, 174/195/277, 16/17/174, 60/195/277, 37/277/318, 16/162/195/277, 26/79/83/162, 26/31/60/318, 16/174/195/277, 13/37/83/162, 16/17/60/195, 13/17/37/83, 31/37/162/174/318, 13/16/31/83/162, 16/60/174/195/318, 17/60/96/162/195/277, 13/26/60/83/162/174, 17/31/60/162/174/277, 16/17/26/83/96/277, 17/26/37/174/277/318, 13/16/31/60/162/174, 13/17/31/83/277/318, 13/16/17/31/83/162/318, 13/16/60/83/162/195/318, 13/16/17/31/37/83/96/162, 13/17/26/37/60/83/162/210/318, 13/16/26/31/83/96/162/174/318, 13/16/17/31/60/83/96/162/174/195/318, 13/16/26/37/60/83/162/174/195/210/318, 13/16/37/79/83/96/162/174/195/210/277, 13/17/26/31/37/60/79/83/96/162/174/210, 13/16/17/26/37/79/83/96/174/195/210/318, 13/26/31/37/60/83/96/162/174/195/210/277/318, 13/16/17/31/37/60/79/83/96/162/174/195/210/318, 13/16/17/31/60/79/83/96/162/174/195/210/277/318, 13/16/17/26/37/60/79/96/162/174/195/210/277/318, 13/26/31/37/60/79/83/96/162/174/195/210/277/318, 13/16/17/26/31/37/60/96/162/174/195/210/277/318, 13/16/17/26/31/37/79/83/96/162/174/195/210/318, 13/16/17/26/31/37/60/79/96/162/174/210/277/318, 13/17/26/31/37/60/79/83/96/162/174/195/210/277/318, 13/16/26/31/37/60/79/83/96/162/174/195/210/277/318, 16/17/26/31/37/60/79/83/96/162/174/195/210/277/318, or 13/16/17/26/31/37/60/79/83/96/162/174/195/210/277/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set of an engineered oxalate decarboxylase polypeptide set forth in Tables 4-1, 4-2, 4-3, 4-4, 4-5, 4-6, 4-7, 5-1, and 5-2, wherein the amino acid positions are relative to SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 1-359 of an even-numbered SEQ ID NO. of SEQ ID NOs: 28-614, a reference sequence corresponding to an even-numbered SEQ ID NO. of SEQ ID NOs: 28-614, or a reference sequence corresponding to an even-numbered SEQ ID NO. of SEQ ID NOs: 616-1622.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 28-1622, or to the reference sequence corresponding to residues 1-359 of an even-numbered SEQ ID NO. of SEQ ID NOs: 28-614, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to residues SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of SEQ ID NO: 172, or to the reference sequence corresponding to SEQ ID NO: 172, wherein the amino acid sequence comprises one or more amino acid substitutions relative to the reference sequence corresponding to residues 1-359 of SEQ ID NO: 172, or relative to the reference sequence corresponding SEQ ID NO: 172.

In some embodiments, the amino acid sequence of the engineered oxalate decarboxylase polypeptide comprises at least a substitution or substitution set at amino acid position(s) 13, 212, 124/196/210/226, 5, 16/26/124/155/195/210/284, 196/226, 16/155/195/210/226, 16/195/210/226, 16/155/174/196, 16/195/226, 318, 16/195/196/210, 16/26/124/155/174/196/210, 195/210, 26/155/174/210, 316, 60, 16/155/174, 16/124/174/196, 16/226, 16/26/174/196/226, 17, 331, 124/195, 16/174/196, 188, 195/196/210, 174/196/210, 16/26/155/174, 16/155/195/196/226, 195/226/284, 16, 240, 16/26/124/155/195/196/226, 183/232/339/343, 183/206, 63, 173/347, 16/26/174/196, 16/284, 46, 16/124/195/196, 274, 174/196, 155/174/196, 174/196/226, 16/124/155/174/195, 16/26/155/174/196, 26/174/196/210/284, 16/195/196/284, 174, 16/124/174/195/210/226/284, 16/174/195/284, 26/174/196/210/226/284, 174/196/226/284, 124/174/196, 26/174/195/210/226, 162, 16/124/195/196/284, 206/343, 16/195/196, 16/26/174/195/196/210/226, 155/195/196/226, 18, 335, 124/155/174/195/226, 26/155/174/195/226, 195/196, 153, 155/174/195, 174/195/210/284, 174/195, 37, 124/174/195/226/284, 176, 16/155/174/195/196/226/284, 10, 169/173, 155, 33, 173/183/343/347, 104/265, or 233, wherein the amino acid positions are relative to the reference sequence corresponding to residues 1-359 of SEQ ID NO: 172, or relative to the reference sequence corresponding SEQ ID NO: 172.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 1-359 of SEQ ID NO: 320, or to the reference sequence corresponding to SEQ ID NO: 320, wherein the amino acid sequence comprises one or more amino acid substitutions relative to the reference sequence corresponding to residues 1-359 of SEQ ID NO: 320, or relative to the reference sequence corresponding to SEQ ID NO: 320.

In some embodiments, the amino acid sequence of the engineered oxalate decarboxylase polypeptide comprises at least a substitution or substitution set at amino acid position(s) 13/17/196, 17/212/331, 17/26/196/212/226, 17/26/196/212/226/331, 13/17/26/196/331, 17/60/196/226, 17/46/212, 26/60/196/226/331, 17/46/196/212/274/331, 212, 17/196/226, 13/17/26/212/331, 13/17/212/226/331, 196/212/331, 13/17/196/331, 13/17/26/212/226/331, 17/60/196/212/226/331, 13/17/212, 13/17/26/331, 13/17, 13/17/26/196, 46/196/212/226, 17/60/196, 17/196/331, 13/17/46/226/331, 17/196/226/331, 17/46/196/212/331, 17/46/212/331, 17/196, 13/17/212/226, 17/212, 17/26/212, 13/17/226/331, 17/196/212/331, 17/196/212/226/331, 13/17/26/226/331, 46/60/196/226/331, 17/46/196/331, 13/17/46/196, 196/212, 13/17/26, 17/83/263, 17/83/173/227, 17/227/301, 17/83/227/263, 17/83/125, 17/83/173, 17/263/301, 17/125, 17/83/301, 17/173, 17/83/227, 17/227, 17, 17/125/227, 17/83, 128, 76, 141, 110, 79, 117, or 61, wherein the amino acid positions are relative to the reference sequence corresponding to residues 1-359 of SEQ ID NO: 320, or relative to the reference sequence corresponding to SEQ ID NO: 320.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to sequence of residues 1-359 of SEQ ID NO: 396, or to the reference sequence corresponding to SEQ ID NO::396, wherein the amino acid sequence comprises one or more amino acid substitutions relative to the reference sequence corresponding to residues 1-359 of SEQ ID NO: 396, or relative to the reference sequence corresponding to SEQ ID NO::396.

In some embodiments, the amino acid sequence of the engineered oxalate decarboxylase polypeptide comprises at least a substitution or substitution set at amino acid position(s) 13/17/79, 13/14/17/60/79, 13/17/79/83, 13/17/79/301, 13/14/17/60/79/212, 17/60/197, 13/60/212, 13/14/17/60/79/83/301, 13/17/60/79, 17/60/301, 14/17/79, 17/60/83, 17/79, 13/17/60/79/83/301, 342, 52/190, 94/190, 190/342/351, 13/79, 13/96, 273, 126, 96, 79, 269, 40, 44, 267, 266, 160, 277, 149, 22, 234, 54, 297, 106, or 13/43, wherein the amino acid positions are relative to the reference sequence corresponding to residues 1-359 of SEQ ID NO: 396, or relative to the reference sequence corresponding to SEQ ID NO: 396.

In some embodiments, the engineered oxalate decarboxylase polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 670, wherein the amino acid sequence comprises one or more amino acid substitutions relative to the reference sequence corresponding to SEQ ID NO: 670.

In some embodiments, the amino acid sequence of the engineered oxalate decarboxylase polypeptide comprises at least a substitution at amino acid position 37, 125, 162, 94, 195, 85, 103, 232, 189, 186, 155, 193, 342, 196, 63, 33, 351, 314, 187, 303, 164, 153, 346, 183, 19, 123, 350, 205, 284, 199, 343, 200, 208, 169, 190, or 121, or combinations thereof, wherein the amino acid positions are relative to the reference sequence corresponding to SEQ ID NO: 670.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 616, wherein the amino acid sequence comprises one or more amino acid substitutions relative to the reference sequence corresponding to SEQ ID NO: 616.

In some embodiments, the amino acid sequence of the engineered oxalate decarboxylase polypeptide comprises at least a substitution or substitution set at amino acid position(s) 14/26/60/318, 14/26/60/94/162/212, 60/162/226, 60, 14/125/162/212/318, 60/125/226, 14/125/162/226, 26/60/162/226/318, 14/26/94/162/212/226, 14/60/162, 14/60/162/226, 14/94/162/318, 14/94/162, 14/60/226, 14/60/94/212, 14/60/162/212, 14/162, 14/94/212/318, 14/60, 14/26/162, 14, 14/318, 14/162/226/318, 14/125/212, 14/125/226/318, 14/26/60/162, 14/125/162/212/226, 14/94/125/162/318, 14/125/226, 277, 166, 233, 83, 5, 342, 96, 284, or 26, wherein the amino acid positions are relative to the reference sequence corresponding SEQ ID NO: 616.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 750, wherein the amino acid sequence comprises one or more amino acid substitutions relative to the reference sequence corresponding to SEQ ID NO: 750.

In some embodiments, the amino acid sequence of the engineered oxalate decarboxylase polypeptide comprises at least a substitution or substitution set at amino acid position(s) 5/26/83/342, 5/26/277, 5/96/277, 5/83/277/342, 26/83/277, 5/26/83/277, 5/277/342, 5/26/166/277, 83/277/342, 83/96/277/342, 83/96/277, 26/83/277/342, 5/26/83/277/342, 5/83/277, 26/277, 83/277, 5/35/277, 5/26/83/96/277, 5/26/96/277, 5/277, 5/166/277, 26/83/96/277/342, 277, 26/277/342, 5, or 5/83/96/277/342, wherein the amino acid positions are relative to the reference sequence corresponding to SEQ ID NO: 750.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 906, wherein the amino acid sequence comprises one or more amino acid substitutions relative to the reference sequence corresponding to SEQ ID NO: 906.

In some embodiments, the amino acid sequence of the engineered oxalate decarboxylase polypeptide comprises at least a substitution at amino acid position 26, 31, 96, 60, 318, 210, 277, 37, 16, 195, 79, 17, 13, 83, 162, or 174, or combinations thereof, wherein the amino acid positions are relative to the reference sequence corresponding to SEQ ID NO: 906.

In some embodiments, the amino acid sequence of the engineered oxalate decarboxylase polypeptide comprises at least a substitution or substitution set at amino acid position(s) 174/318, 26/96, 60/318, 31/318, 162/174/318, 174/277/318, 26/195/318, 60/162/195, 60/162/318, 174/195/277, 16/17/174, 60/195/277, 37/277/318, 16/162/195/277, 26/79/83/162, 26/31/60/318, 16/174/195/277, 13/37/83/162, 16/17/60/195, 13/17/37/83, 31/37/162/174/318, 13/16/31/83/162, 16/60/174/195/318, 17/60/96/162/195/277, 13/26/60/83/162/174, 17/31/60/162/174/277, 16/17/26/83/96/277, 17/26/37/174/277/318, 13/16/31/60/162/174, 13/17/31/83/277/318, 13/16/17/3⅛3/162/318, 13/16/60/83/162/195/318, 13/16/17/31/37/83/96/162, 13/17/26/37/60/83/162/210/318, 13/16/26/31/83/96/162/174/318, 13/16/17/31/60/83/96/162/174/195/318, 13/16/26/37/60/83/162/174/195/210/318, 13/16/37/79/83/96/162/174/195/210/277, 13/17/26/31/37/60/79/83/96/162/174/210, 13/16/17/26/37/79/83/96/174/195/210/318, 13/26/31/37/60/83/96/162/174/195/210/277/318, 13/16/17/31/37/60/79/83/96/162/174/195/210/318, 13/16/17/31/60/79/83/96/162/174/195/210/277/318, 13/16/17/26/37/60/79/96/162/174/195/210/277/318, 13/26/31/37/60/79/83/96/162/174/195/210/277/318, 13/16/17/26/31/37/60/96/162/174/195/210/277/318, 13/16/17/26/31/37/79/83/96/162/174/195/210/318, 13/16/17/26/31/37/60/79/96/162/174/210/277/318, 13/17/26/31/37/60/79/83/96/162/174/195/210/277/318, 13/16/26/31/37/60/79/83/96/162/174/195/210/277/318, 16/17/26/31/37/60/79/83/96/162/174/195/210/277/318, or 13/16/17/26/31/37/60/79/83/96/162/174/195/210/277/318, wherein the amino acid positions are relative to the reference sequence corresponding to SEQ ID NO: 906.

In some embodiments, the amino acid sequence of the engineered oxalate decarboxylase polypeptide comprises at least a substitution or substitution set of an oxalate decarboxylase polypeptide set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises an amino acid sequence of an engineered oxalate decarboxylase polypeptide set forth in Tables 4-1, 4-2, 4-3, 4-4, 4-5, 4-6, 4-7, 5-1, and 5-2.

In some embodiments, the engineered oxalate decarboxylase polypeptide comprises an amino acid sequence comprising SEQ ID NO: 4; an amino acid sequence comprising residues 1-359 of an even-numbered SEQ ID NO. of SEQ ID NOs: 28-614; an amino acid sequence comprising an even-numbered SEQ ID NO. of SEQ ID NOs: 28-614; or an amino acid sequence comprising an even-numbered SEQ ID NO. of SEQ ID NOs: 616-1622, optionally wherein the amino acid sequence includes 1, 2, 3, 4, 5, 6, 7, 8, 9, or up to 10 amino acid substitutions.

In some embodiments, the amino acid sequence of the engineered oxalate decarboxylase polypeptide comprises residues 1-359 of SEQ ID NO: 172, 320, or 396, or comprises SEQ ID NO: 172, 320, 396, 616, 670, 750, or 906, optionally wherein the amino acid sequence includes 1, 2, 3, 4, 5, 6, 7, 8, 9, or up to 10 substitutions.

In some embodiments of the foregoing, the amino acid sequence of the engineered oxalate decarboxylase polypeptide includes 1, 2, 3, 4, or 5 substitutions.

In some embodiments, the engineered oxalate decarboxylase polypeptide of the present disclosure has oxalate decarboxylase activity and exhibits one or more improved enzyme properties compared to a reference oxalate decarboxylase having an amino acid sequence corresponding to SEQ ID NO: 2, 4, 172, 320, 396, 616, 670, 750, or 906, or an amino acid sequence corresponding to residues 1-359 of SEQ ID NO: 172, 320, or 396. In some embodiments, the improved property of the engineered oxalate decarboxylase polypeptide is selected from (a) increased activity on oxalate, (b) increased thermal stability, (c) increased stability at acidic pH, (d) increased activity at acidic pH, (e) increased activity at neutral pH, (f) increased expression, (g) increased solubility, and (h) increased resistance to proteolysis, or any combination of (a), (b), (c), (d), (e), (f), (g) and (h), compared to a reference oxalate decarboxylase having an amino acid sequence corresponding to SEQ ID NO: 2, 4, 172, 320, 396, 616, 670, 750, or 906, or an amino acid sequence corresponding to residues 1-359 of SEQ ID NO: 172, 320, or 396. Exemplary improved propertied are provided in the Examples and Tables 4-1, 4-2, 4-3, 4-4, 4-5, 4-6, 4-7, 5-1, and 5-2,

In some embodiments, the reference oxalate decarboxylase polypeptide for comparison of the improved property is the oxalate decarboxylase polypeptide having an amino acid sequence corresponding to SEQ ID NO: 2 or 4. In some embodiments the reference oxalate decarboxylase polypeptide is the wild-type oxalate decarboxylase of Gemmata sp. SH-PL17.

In some embodiments, an engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 4, 6, 8, 10, 12, 14, 16, 18, 20, 22, 24, or 26, or the reference sequence corresponding to residues 1-424 of SEQ ID NO: 6.

In some embodiments, an engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference amino acid sequence corresponding to SEQ ID NO: 4, 6, 8, 10, 12, 14, 16, 18, 20, 22, 24, or 26, or to a reference amino acid sequence corresponding to residues 1-424 of SEQ ID NO: 6, wherein the amino acid sequence comprises one or more substitutions in its amino acid sequence.

In some embodiments, the engineered oxalate decarboxylase polypeptide comprises an amino acid sequence comprising SEQ ID NO: 2, 4, 6, 8, 10, 12, 14, 16, 18, 20, 22, 24, or 26, or an amino acid comprising residues 1-424 of SEQ ID NO: 6. In some embodiments, the oxalate decarboxylase polypeptides are provided as compositions, particularly pharmaceutical compositions.

In some embodiments, the engineered oxalate decarboxylase polypeptide described herein is purified or is a purified preparation.

In another aspect, the present disclosure provides compositions comprising at least one engineered oxalate decarboxylase polypeptide provided herein. In some embodiments, the composition is a pharmaceutical composition comprising at least one engineered oxalate decarboxylase of the present disclosure, and a pharmaceutically acceptable excipient or carrier.

In a further aspect, the present disclosure provides a recombinant polynucleotide comprising a polynucleotide sequence encoding an engineered oxalate decarboxylase polypeptide described herein.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 3, a reference polynucleotide sequence corresponding to nucleotide residues 1-1077 of an odd-numbered SEQ ID NO. of SEQ ID NOs: 27-613, a reference polynucleotide sequence corresponding to an odd-numbered SEQ ID NO. of SEQ ID NOs: 27-613, or a reference polynucleotide sequence corresponding to an odd-numbered SEQ ID NO. of SEQ ID NOs: 615-1621, wherein the recombinant polynucleotide encodes an engineered oxalate decarboxylase polypeptide.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 33, 171, 319, 395, 615, 669, 749, or 905, or to a reference polynucleotide sequence corresponding to nucleotide residues 1-1077 of SEQ ID NO: 171, 319, or 395, wherein the recombinant polynucleotide encodes an engineered oxalate decarboxylase polypeptide.

In some embodiments, the recombinant polynucleotide comprises a polynucleotide sequence which is codon-optimized. In some embodiments, the polynucleotide sequence is codon optimized for expression in a bacterial cell, fungal cell, or mammalian cell.

In some embodiments, the recombinant polynucleotide comprises a polynucleotide sequence comprising SEQ ID NO: 1 or 3; a polynucleotide sequence comprising nucleotide residues 1-1077 of an odd-numbered SEQ ID NO. of SEQ ID NOs: 27-613; a polynucleotide sequence comprising an odd-numbered SEQ ID NO. of SEQ ID NOs: 27-613; or a polynucleotide sequence comprising an odd-numbered SEQ ID NO. of SEQ ID NOs: 615-1621.

In some embodiments, the recombinant polynucleotide comprises a polynucleotide sequence comprising SEQ ID NO: 3, 171, 319, 395, 615, 669, 749, or 905, or comprising nucleotide residues 1-1077 of SEQ ID NO: 171, 319, or 395.

In another aspect, the present disclosure also provides an expression vector comprising a recombinant polynucleotide encoding an engineered oxalate decarboxylase polypeptide. In some embodiments, the expression vectors further comprise at least one control sequence operably linked to the recombinant polynucleotide. In some embodiments, the control sequence comprises a promoter. In some further embodiments, the promoter is a heterologous promoter.

In a further aspect, the present disclosure also provides host cells transformed with at least one recombinant polynucleotide or an expression vector described herein. In some embodiments, the host cell comprises a recombinant polynucleotide described herein. In some embodiments, the host cell comprises an expression vector described herein. In some embodiments, the host cell is a prokaryotic cell or a eukaryotic cell.

In another aspect, the present disclosure also provides a method of producing an engineered oxalate decarboxylase polypeptide described herein, the method comprising culturing a host cell comprising a recombinant polynucleotide encoding an engineered oxalate decarboxylase polypeptide under suitable culture conditions such that the encoded engineered oxalate decarboxylase polypeptide is produced. In some embodiments, the method further comprises recovering the engineered oxalate decarboxylase polypeptide from the culture and/or host cells. In some embodiments, the methods further comprise a step of purifying the expressed engineered oxalate decarboxylase polypeptide.

In another aspect, the present disclosure provides a composition comprising an engineered oxalated decarboxylase described herein. In some embodiments, the composition is a pharmaceutical composition. In some embodiments, the pharmaceutical composition comprises an engineered oxalate decarboxylase, and a pharmaceutically acceptable carrier or excipient.

In another aspect, the engineered oxalate decarboxylase is applied to various therapeutic and industrial uses. In some embodiments, an engineered oxalate decarboxylase is used for reducing levels of oxalate in a subject. In some embodiments, a method for reducing levels of oxalate in a subject comprises administering an effective amount of an engineered oxalate decarboxylase described herein to a subject in need thereof to reduce levels of oxalate in the subject. In some embodiments, the method is used to reduce levels of oxalate in urine and/or plasma of the subject.

In some embodiments, the engineered oxalate decarboxylase is used for treating hyperoxaluria and/or preventing the symptoms of hyperoxaluria in a subject, the method comprising administering to a subject with hyperoxaluria an effective amount of an engineered oxalate decarboxylase or a composition thereof described herein. In some embodiments, the subject is treated to ameliorate one or more symptoms of hyperoxaluria. In some embodiments, the levels of oxalate in the urine and/or plasma is reduced. In some embodiments, the subject can eat a diet that is less restricted in oxalate content than diets required by subjects who are afflicted with hyperoxaluria or have pathogenically elevated levels of oxalate. In some embodiments, the subject is an infant, child, young adult, or adult.

In another aspect, the present disclosure provides use of the engineered oxalate decarboxylase, or a pharmaceutical composition thereof, for the treatment of a disorder or condition associated with elevated levels of oxalate. In some embodiments, the use of the engineered oxalate decarboxylase is for the preparation of a medicament for treating a disease or condition associated with elevated levels of oxalate. In some embodiments, the use is in the treatment of hyperoxaluria.

In another aspect, the engineered oxalate decarboxylase can be useful in industrial applications, such as for reducing formation of calcium oxalate deposits/precipitates. Deposits or precipitates of calcium oxalate are problematic in pulp and paper industries, including removal of oxalate from industrial wastewater.

### BRIEF DESCRIPTION OF THE DRAWINGS

FIG. 1 shows a graph of percent change in Uox values for individual animals in three treatment groups orally administered different doses of an engineered oxalate decarboxylase (SEQ ID NO: 906). Bars represent the % change in UOx from HOD to HOD + Treatment (HOD: high oxalate diet). Dotted line at -20% (y-axis) identifies the minimum threshold reduction value used to evaluate efficacy of treatment.

FIG. 2 shows a graph of change in UOx (mg/24 hr) by treatment group. The white bars represent groups receiving HOD, and patterned bars represent groups receiving HOD + Tx. Treatment groups received the engineered oxalate decarboxylase (SEQ ID NO: 906) dosed at 5,139 U, 10,122 U, or 41,029 U. Statistical significance between HOD and HOD + Tx was determined using a One-way ANOVA in GraphPad Prism 9.

### DETAILED DESCRIPTION

The present disclosure provides engineered oxalate decarboxylase (ODC) polypeptides and compositions thereof; polynucleotides encoding the engineered oxalate decarboxylase polypeptides; and uses of the engineered oxalate decarboxylase polypeptide for therapeutic and industrial purposes. In some embodiments, the oxalate decarboxylase polypeptides are engineered to have improved properties, including, among others, improved catalytic activity, reduced sensitivity to proteolysis, and/or increased tolerance to and stability at low pH environments.

### Abbreviations and Definitions

Unless defined otherwise, all technical and scientific terms used herein generally have the same meaning as commonly understood by one of ordinary skill in the art to which this invention pertains. It is also to be understood that the invention herein is not limited to the particular methodology, protocols, and reagents described, as these may vary, depending upon the context they are used by those of skill in the art.

Furthermore, the headings provided herein are not limitations of the various aspects or embodiments of the invention which can be had by reference to the application as a whole. Accordingly, the terms defined immediately below are more fully defined by reference to the application as a whole.

As used herein, the singular “a”, “an,” and “the” include the plural references, unless the context clearly indicates otherwise.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

The term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

As used herein, the term “comprising” and its cognates are used in their inclusive sense (i.e., equivalent to the term “including” and its corresponding cognates).

It is to be further understood that where description of embodiments use the term “comprising” and its cognates, the embodiments can also be described using language “consisting essentially of” or “consisting of.”

As used herein, the term “at least one” or “at least a” is not intended to limit the invention to any particular number of items. It is intended to encompass one, two, three, four, five, six, seven, eight, nine, ten, or more items, as desired.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“Oxalate decarboxylase polypeptide” or “oxalate decarboxylase” or “ODC” refers to a member of the enzyme class EC 4.1.1.2 or an enzyme that catalyzes the conversion of oxalate to formate and CO₂. In some embodiments, oxalate decarboxylase may additionally refer to enzymes that catalyzes the conversion of oxalate to hydrogen peroxide and CO₂.

“Protein,” “polypeptide,” and “peptide” are used interchangeably herein to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cα). For example, whereas “Ala” designates alanine without specifying the configuration about the α carbon, “D-Ala” and “L-Ala” designate D-alanine and L-alanine, respectively. When the one-letter abbreviations are used, upper case letters designate amino acids in the L-configuration about the α-carbon and lower-case letters designate amino acids in the D-configuration about the α-carbon. For example, “A” designates L-alanine and “a” designates D-alanine. When amino acid sequences are presented as a string of one-letter or three-letter abbreviations (or mixtures thereof), the sequences are presented in the amino (N) to carboxy (C) direction in accordance with common convention.

“Polynucleotide” or “nucleic acid” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for the genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid.

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” is used herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted (e.g., by the local homology algorithm of Smith and Waterman; Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA., 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (See e.g., Altschul et al., J. Mol. Biol., 1990, 215:403-410; and Altschul et al., Nucleic Acids Res., 1977, 25:3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (See e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or amino acid sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “reference sequence based on SEQ ID NO: 2 having a valine at the residue corresponding to X60” refers to a reference sequence in which the corresponding residue at position X60 in SEQ ID NO: 2 (e.g., a threonine), has been changed to valine.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered ODC, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Mutation” refers to the alteration of a nucleic acid sequence. In some embodiments, mutations result in changes to the encoded amino acid sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original amino acid sequence. In some further embodiments, the mutation comprises a deletion, such that an amino acid is deleted from the original amino acid sequence. Any number of mutations may be present in a given sequence. In some embodiments, the “substitution” comprises the deletion of an amino acid and can be denoted by “-” symbol.

“Amino acid difference” and “residue difference” refer to a difference in the amino acid residue at a position of an amino acid sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X60 as compared to SEQ ID NO: 2” refers to a difference of the amino acid residue at the polypeptide position corresponding to position 60 of SEQ ID NO: 2. Thus, if the reference polypeptide of SEQ ID NO: 2 has a threonine at position 60, then a “residue difference at position X60 as compared to SEQ ID NO: 2” refers to an amino acid substitution of any residue other than threonine at the position of the polypeptide corresponding to position 60 of SEQ ID NO: 2. In some instances herein, the specific amino acid residue difference, e.g., substitution, at a position is indicated as “XnY” where “Xn” specifies the corresponding residue and position of the reference polypeptide (as described above), and “Y” is the single letter identifier of the amino acid found in the engineered polypeptide (i.e., the different residue than in the reference polypeptide). In some instances, the original amino acid is not indicated and the amino acid difference indicated as nY (e.g., 60V). In some embodiments, the phrase “an amino acid residue nY” denotes the presence of the amino acid residue in the engineered polypeptide, which may or may not be a substitution in context of a reference sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X60Y/X60W/X60R or X60Y/W/R or 60Y/W/R). The present disclosure includes engineered amino acid sequences comprising one or more amino acid differences that include either/or both conservative and non-conservative amino acid substitutions.

“Amino acid substitution set” and “substitution set” refers to a group of amino acid substitutions within an amino acid sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the engineered polypeptides listed in any of the Tables in the Examples. In these substitution sets, the individual substitutions are separated by a semicolon (“;”; e.g., A16S;A26E or abbreviated 16S;26E) or slash (“/”; e.g., A16S/A26E, or abbreviated 16S/26E).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acid having aromatic side chains is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively. Exemplary conservative substitutions include the substitution of A, L, V, or I with other aliphatic residues (e.g., A, L, V, I) or other non-polar residues (e.g., A, L, V, I, G, M); substitution of G or M with other non-polar residues (e.g., A, L, V, I, G, M); substitution of D or E with other acidic residues (e.g., D, E); substitution of K or R with other basic residues (e.g., K, R); substitution of N, Q, S, or T with other polar residues (e.g., N, Q, S, T); substitution of H, Y, W, or F with other aromatic residues (e.g., H, Y, W, F); or substitution of C or P with other non-polar residues (e.g., C, P).

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered oxalate decarboxylase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered ODC of the present invention) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant ODC polypeptides may be present within a cell, present in the cellular medium, or prepared in various forms, such as lysates or isolated preparations. As such, in some embodiments, the recombinant ODC polypeptides provided herein are isolated polypeptides.

“Substantially pure” or “purified” polypeptide or protein refers to a composition in which the polypeptide or protein species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure ODC polypeptide composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant ODC polypeptides are substantially pure polypeptide compositions.

“Improved enzyme property” refers to an engineered ODC polypeptide that exhibits an improvement in any enzyme property as compared to a reference ODC polypeptide, such as a wild-type ODC polypeptide (e.g., wild-type ODC corresponding to SEQ ID NO: 2) or another engineered ODC polypeptide. Improved properties include but are not limited to such properties as increased enzymatic activity, increased specific activity, increased protein production, increased thermoactivity, increased thermostability, increased pH activity, increased stability, increased substrate specificity and/or affinity, increased resistance to substrate and/or end-product inhibition, increased chemical stability, improved solvent stability, increased stability to acidic pH, increased resistance to proteases, reduced aggregation, increased solubility, and reduced immunogenicity (i.e., reduced capability of inducing and/or eliciting an immune response).

“Increased enzymatic activity” and “enhanced catalytic activity” refer to an improved property of the engineered ODC polypeptides, which can be represented by an increase in specific activity (e.g., product produced/time/weight protein) and/or an increase in percent conversion of the substrate to the product (e.g., percent conversion of starting amount of substrate to product in a specified time period using a specified amount of ODC) as compared to the reference ODC enzyme (e.g., wild-type ODC and/or another engineered ODC). Exemplary methods to determine enzyme activity are provided in the Examples. Any property relating to enzyme activity may be affected, including the classical enzyme properties of Km, Vmax or kcat, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25- fold, 50-fold, 75-fold, 100-fold, 150-fold, 200-fold or more enzymatic activity than the naturally occurring ODC or another engineered ODC from which the ODC polypeptides were derived. In some specific embodiments, the engineered ODC enzyme exhibits improved enzymatic activity in the range of 1.5 to 10 fold, 1.5 to 25 fold, 1.5 to 50 fold, 1.5 to 100 fold or greater, than that of the reference ODC enzyme.

ODC enzyme activity can be measured by any standard assay known in the art (e.g., by monitoring depletion of reactants or formation of products). Exemplary methods of measuring ODC activity are provided in the Examples, such as Example 3. In some embodiments, comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells, in order to minimize variations in amount of enzyme produced by the host cells and present in the lysates.

“Increased storage stability” means that an engineered ODC polypeptide according to the invention will retain more activity compared to a reference ODC in a standard assay (e.g., as described in the Examples) after it has been produced in its storage form (e.g., solution, lyophilization, or spray-drying), and stored for a period of time ranging from a few days to multiple months at a defined temperature or temperatures (e.g., -20° C., 0° C., 4° C., 25° C., 30° C., 37° C., 45° C., 55° C., etc.).

“Conversion” refers to the enzymatic conversion (or biotransformation) of substrate(s) to the corresponding product(s). “Percent conversion” refers to the percent of the substrate that is converted to the product within a period of time under specified conditions. Thus, the “enzymatic activity” or “activity” of an ODC polypeptide can be expressed as “percent conversion” of the substrate to the product in a specific period of time.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5× Denhart’s solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tm as determined under the solution condition for a defined polynucleotide sequence. In some embodiments, a high stringency condition refers to conditions that permit hybridization of only those nucleic acid sequences that form stable hybrids in 0.018 M NaCl at 65° C. (i.e., if a hybrid is not stable in 0.018 M NaCl at 65° C., it will not be stable under high stringency conditions, as contemplated herein). High stringency conditions can be provided, for example, by hybridization in conditions equivalent to 50% formamide, 5× Denhart’s solution, 5×SSPE, 0.2% SDS at 42° C., followed by washing in 0.1×SSPE, and 0.1% SDS at 65° C. Another high stringency condition is hybridizing in conditions equivalent to hybridizing in 5X SSC containing 0.1% (w:v) SDS at 65° C. and washing in 0.1x SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism’s genome. In some embodiments, the polynucleotides encoding the ODC enzymes are codon optimized for optimal production from the host organism selected for expression.

“Control sequence” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, propeptide sequences, promoter sequences, signal peptide sequences, initiation sequences, and transcription terminators. At a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a functional relationship between two or more nucleic acid segments. By way of illustration, the term refers to a configuration in which a control sequence is appropriately placed (i.e., in a functional relationship) at a position relative to a polynucleotide of interest such that the control sequence directs or regulates the expression of the polynucleotide, and where relevant, the encoded polypeptide of interest.

“Promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Substrate” in the context of an enzymatic conversion reaction process refers to the compound or molecule acted on by the ODC polypeptide. “Product” in the context of an enzymatic conversion process refers to the compound or molecule resulting from the action of the ODC polypeptide on the substrate.

“Culturing” or “cultured” refers to the growing of a population of cells under suitable conditions using any suitable medium (e.g., liquid, gel, or solid).

“Vector” as used herein refers to a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

“Expression” as used herein includes any step involved in the production of the polypeptide including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or amino acid sequences (e.g., a promoter sequence, signal peptide, terminator sequence, etc.) that are derived from different sources and are not associated in nature.

“Host cell” and “host strain” refer to suitable hosts for expression vectors comprising DNA provided herein (e.g., a polynucleotide sequences encoding at least one ODC variant). In some embodiments, the host cells are prokaryotic or eukaryotic cells that have been transformed or transfected with vectors constructed using recombinant DNA techniques as known in the art.

“Subject” as used herein encompasses mammals such as humans, non-human primates, livestock (e.g., cows, pigs, camels, etc.), companion animals (e.g., cats, dogs, etc.), and laboratory animals (e.g., rats, mice, guinea pigs, and lagamorphs). It is intended that the term encompass females as well as males. A subject who is a “patient” means any subject that is being assessed for, treated for, or is experiencing disease.

“Infant” refers to a child in the period of the first month after birth to approximately one (1) year of age. As used herein, the term “newborn” refers to child in the period from birth to the 28th day of life. The term “premature infant” refers to an infant born after the twentieth completed week of gestation, yet before full term, generally weighing ~500 to ~2499 grams at birth.

“Child” refers to a person who has not attained the legal age for consent to treatment or research procedures. In some embodiments, the term refers to a person between the time of birth and adolescence.

“Adult” refers to a person who has attained legal age for the relevant jurisdiction (e.g., 18 years of age in the United States). In some embodiments, the term refers to any fully grown, mature organism. In some embodiments, the term “young adult” refers to a person less than 18 years of age, but who has reached sexual maturity.

“Composition” and “formulation” encompass products comprising at least one engineered ODC of the present disclosure, intended for any suitable use (e.g., pharmaceutical compositions, dietary/nutritional supplements, feed, etc.).

“Therapeutic” as used herein refers to an agent administered to a subject who shows signs or symptoms of pathology having beneficial or desirable medical effects.

“Pharmaceutical composition” as used herein refers to a composition suitable for pharmaceutical use in a mammalian subject (e.g., human) comprising a pharmaceutically effective amount of an engineered ODC polypeptide encompassed by the invention and an acceptable carrier.

“Carrier” when used in reference to a pharmaceutical composition means any of the standard pharmaceutical carrier, buffers, and excipients, such as stabilizers, preservatives, and adjuvants.

“Excipient” refers to any pharmaceutically acceptable additive, carrier, diluent, adjuvant, or other ingredient, other than the active pharmaceutical ingredient (API; e.g., the engineered ODC polypeptides of the present invention). Excipients are typically included for formulation and/or administration purposes.

“Pharmaceutically acceptable” means a material that can be administered to a subject without causing any undesirable biological effects or interacting in a deleterious manner with any of the components in which it is contained and that possesses the desired biological activity.

“Effective amount” refers to an amount sufficient to produce the desired result. The effective amount can be determined by one of general skill in the art by methods known in the art and the guidance provided herein.

“Therapeutically effective amount” when used in reference to symptoms of disease/condition refers to the amount and/or concentration of a compound (e.g., engineered ODC polypeptides) that ameliorates, attenuates, or eliminates one or more symptom of a disease/condition or prevents or delays the onset of symptom(s) (e.g., hyperoxaluria). In some embodiments, the term is use in reference to the amount of a composition that elicits the biological (e.g., medical) response by a tissue, system, or animal subject that is sought by the researcher, physician, veterinarian, or other clinician. “Therapeutically effective amount” when used in reference to a disease/condition refers to the amount and/or concentration of a composition that ameliorates, attenuates, or eliminates the disease/condition.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As such, the terms “treating,” “treat” and “treatment” encompass preventative (e.g., prophylactic), as well as palliative treatment. As is known in the art, adjustments for systemic versus localized delivery, age, body weight, general health, sex, diet, time of administration, drug interaction and the severity of the condition can be made by those skilled in the art.

### Engineered Oxalate Decarboxylase Polypeptides

In one aspect, the present disclosure provides engineered oxalate decarboxylase (ODC) polypeptides for conversion/degradation of oxalate. In some embodiments, the engineered oxalate decarboxylase polypeptides are variants of the wild-type oxalate decarboxylase of Gemmata sp. SH-PL17. In some embodiments, in the present disclosure when a particular engineered oxalate decarboxylase variant (i.e., an engineered ODC polypeptide) is referred to by reference to amino acid differences in particular amino acids residues in the sequence of a wild-type oxalate decarboxylase or reference oxalate decarboxylase, it is to be understood that variants of another oxalate decarboxylase modified in the equivalent position(s) (as determined from the optional amino acid sequence alignment between the respective amino acid sequences) are to be encompassed herein.

Furthermore, the present disclosure describes exemplary engineered ODC polypeptides having oxalate decarboxylase activity. The Examples and the exemplary engineered oxalate decarboxylase presented in the Tables (i.e., Tables 4-1, 4-2, 4-3, 4-4, 4-5, 4-6, 4-7, 5-1, and 5-2) provide sequence structural information correlating specific amino acid sequence features with the functional activity (e.g., improved property) of the engineered oxalate decarboxylase polypeptides. This structure-function correlation information is provided in the form of specific amino acid residue differences relative to the reference engineered oxalate decarboxylase polypeptide of SEQ ID NO: 2 or 4, as well as associated experimentally determined activity data for the exemplary engineered ODC polypeptides.

In some embodiments, an engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 2, 4, 172, 320, 396, 616, 670, 750, or 906, or to a reference sequence corresponding to residues 1-359 of SEQ ID NO: 172, 320, or 396, wherein the amino acid sequence comprises one or more amino acid substitutions relative to the reference sequence corresponding to SEQ ID NO: 2, 4, 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2 or 4, wherein the amino acid sequence comprises one or more amino acid substitutions relative to the reference sequence corresponding to SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 172, 320, 396, 616, 670, 750, or 906, or to the reference sequence corresponding to residues 1-359 of SEQ ID NO: 172, 320, or 396, wherein the amino acid sequence comprises one or more amino acid substitutions relative to the reference sequence of SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 28-1622, or to the reference sequence corresponding to residues 1-359 of an even-numbered SEQ ID NO. of SEQ ID NOs: 28-614, wherein the amino acid sequence comprises one or more amino acid substitutions relative to the reference sequence of SEQ ID NO: 2 or 4.

In some embodiments, the amino acid sequence of the engineered oxalate decarboxylase polypeptide comprises at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution 4C/R/S, 5C/S, 6A/S/W, 7G, 10A/Q/R, 11R, 13A/C/D/E/F/G/I/K/L/M/N/P/Q/R/S/T/V/W/Y, 14L, 16C/D/E/F/G/H/I/K/L/M/N/P/Q/R/S/T/V/W/Y, 17A/C/D/E/F/G/I/K/L/M/N/P/Q/R/S/T/V/W/Y, 18G/Q/R, 19C, 22R, 26C/D/E/F/G/H/I/K/L/M/N/P/Q/R/S/T/V/W/Y, 31A/C/D/F/G/H/I/K/L/M/N/P/Q/R/S/T/V/W/Y, 33R, 35V, 37A/C/D/E/F/G/H/I/L/M/N/P/Q/R/S/T/V/W/Y, 40S, 43C, 44S, 46R, 52T, 54L, 60A/C/D/E/F/G/H/I/K/L/M/N/P/Q/R/S/V/W/Y, 61M, 62G, 63A/S, 76S, 79C/D/E/F/G/H/I/K/L/M/N/P/Q/R/S/T/V/W/Y, 80L, 82I, 83A/C/D/E/F/G/H/I/K/L/M/N/P/Q/R/S/V/W/Y, 85E/G/R, 94N/T, 96C/D/E/F/G/H/I/K/L/M/N/P/Q/R/S/T/V/W/Y, 97T, 103Q/S/V, 104Q, 106C, 110V, 117A, 121W, 123G/Q/S, 124A/C/G/Q/R/T, 125E/F/S, 126T, 128A, 141Q, 149T, 153H/Q/S, 155L/P/R/V, 156E, 160T, 162A/C/D/E/F/H/I/K/L/M/N/P/Q/R/S/T/V/W/Y, 164R/V, 166R, 169A/G/L, 173Q/V, 174A/C/E/F/G/H/I/K/L/M/N/P/Q/R/S/T/V/W/Y, 176L, 180E, 182R, 183C/E/I/K, 186N, 187L/R/S, 188A, 189R, 190G/Q, 193A/G, 195A/C/D/E/F/G/H/I/K/L/M/N/P/Q/S/T/V/W/Y, 196F/M/R/S/V, 197E, 199G/V, 200N, 205A, 206M, 208G, 210A/C/D/E/F/G/H/I/K/L/M/N/Q/R/S/T/V/W/Y, 212A/F/G/L/S/V, 216S/W, 219V, 226S, 227E/S, 232T, 233D/H/R, 234L, 240E, 242D, 243V, 263S, 265C, 266V, 267R, 269S, 270L, 273A, 274Q/S, 277A/C/D/E/F/G/H/I/K/L/M/N/P/Q/R/S/T/V/W, 284A/R/S, 297Y, 301I/Q/T, 303T, 304D/G, 314S, 316K/V, 318A/C/D/F/G/H/I/K/L/M/N/P/Q/R/S/T/V/W/Y, 331I/L/P/V, 335R, 339G, 342A/E/P/R, 343R/W, 346G/L/Q/W, 347F/R, 350R, 351A/E/H, 356S, or 359L/W, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution P4C/R/S, T5C/S, F6A/S/W, M7G, V10A/Q/R, P11R, H13A/C/D/E/F/G/I/K/L/M/N/P/Q/R/S/T/V/W/Y, V14L, A16C/D/E/F/G/H/I/K/L/M/N/P/Q/R/S/T/V/W/Y, H17A/C/D/E/F/G/I/K/L/M/N/P/Q/R/S/T/V/W/Y, K18G/Q/R,, D19C, T22R, A26C/D/E/F/G/H/I/K/L/M/N/P/Q/R/S/T/V/W/Y, E31A/C/D/F/G/H/I/K/L/M/N/P/Q/R/S/T/V/W/Y, K33R, I35V, K37A/C/D/E/F/G/H/I/L/M/N/P/Q/R/S/T/V/W/Y, G40S, A43C, T44S, V46R, K52T, I54L, T60A/C/D/E/F/G/H/I/K/L/M/N/P/Q/R/S/V/W/Y, L61M, E62G, P63A/S, A76S, A79C/D/E/F/G/H/I/K/L/M/N/P/Q/R/S/T/V/W/Y, F80L/I, L82I, T83A/C/D/E/F/G/H/I/K/L/M/N/P/Q/R/S/V/W/Y, H85E/G/R, Q94N/T, A96C/D/E/F/G/H/I/K/L/M/N/P/Q/R/S/T/V/W/Y, A97T, G103Q/S/V, P104Q, D106C, F110V, M117A, L121W, D123G/Q/S, K124A/C/G/Q/R/T, P125E/F/S, A126T, F128A, G141Q, I149T, P153H/Q/S, A155L/P/R/V, L156E, N160T, G162A/C/D/E/F/H/I/K/L/M/N/P/Q/R/S/T/V/W/Y, P164R/V, S166R, D169A/G/L, K173Q/V, D174A/C/E/F/G/H/I/K/L/M/N/P/Q/R/S/T/V/W/Y, V176L, R180E, A182R, V183C/E/I/K, A186N, T187L/R/S, P188A, A189R, A190G/Q, Q193A/G, R195A/C/D/E/F/G/H/I/K/L/M/N/P/Q/S/T/V/W/Y, K196F/M/R/S/V, L197E, P199G/V, L200N, E205A, L206M, E208G, P210A/C/D/E/F/G/H/I/K/L/M/N/Q/R/S/T/V/W/Y, H212A/F/G/L/S/V, K216S/W, R219V, T226S, R227E/S, K232T, T233D/H/R, V234L, D240E, E242D, P243V, D263S, N265C, I266V, S267R, T269S, M270L, S273A, H274Q/S, Y277A/C/D/E/F/G/H/I/K/L/M/N/P/Q/R/S/T/V/W, K284A/R/S, S297Y, V301I/Q/T, D303T, R304D/G, T314S, N316K/V, E318A/C/D/F/G/H/I/K/L/M/N/P/Q/R/S/T/V/W/Y, A331I/L/P/V, A335R, S339G, T342A/E/P/R, S343R/W, E346G/L/Q/W, K347F/R, K350R, Q351A/E/H, A356S, or K359L/W, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution 13A/C/D/E/F/G/I/K/L/M/N/P/Q/R/S/T/V/W/Y, 14L, 16C/D/E/F/G/H/I/K/L/M/N/P/Q/R/S/T/V/W/Y, 17A/C/D/E/F/G/I/K/L/M/N/P/Q/R/S/T/V/W/Y, 26C/D/E/F/G/H/I/K/L/M/N/P/Q/R/S/T/V/W/Y, 31A/C/D/F/G/H/I/K/L/M/N/P/Q/R/S/T/V/W/Y, 37A/C/D/E/F/G/H/I/L/M/N/P/Q/R/S/T/V/W/Y, 60A/C/D/E/F/G/H/I/K/L/M/N/P/Q/R/S/V/W/Y, 79C/D/E/F/G/H/I/K/L/M/N/P/Q/R/S/T/V/W/Y, 83A/C/D/E/F/G/H/I/K/L/M/N/P/Q/R/S/V/W/Y, 96C/D/E/F/G/H/I/K/L/M/N/P/Q/R/S/T/V/W/Y, 162A/C/D/E/F/H/I/K/L/M/N/P/Q/R/S/T/V/W/Y, 174A/C/E/F/G/H/I/K/L/M/N/P/Q/R/S/T/V/W/Y, 195A/C/D/E/F/G/H/I/K/L/M/N/P/Q/S/T/V/W/Y, 196F/M/R/S/V, 210A/C/D/E/F/G/H/I/K/L/M/N/Q/R/S/T/V/W/Y, 226S, 277A/C/D/E/F/G/H/I/K/L/M/N/P/Q/R/S/T/V/W, 301I/Q/T, or 318A/C/D/F/G/H/I/K/L/M/N/P/Q/R/S/T/V/W/Y, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution 13P, 14L, 16S, 17H/G, 26E, 31K, 37G, 60V, 79G, 83W, 96L, 162K, 174K, 195L, 196V, 210A/E, 226S, 277V, 301I, or 318Q, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 31, 210, 318, or 31/210/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 31K, 210A/E, 318Q, or 31K/210A/318Q, wherein the amino acid positions are relative to the reference sequence corresponding to SEQ ID NO: 2 or 4.

In some embodiments, the amino acid sequence of the engineered oxalate decarboxylase polypeptide comprises as least a substitution or substitution set at amino acid position(s) 16, 26, 174, 195, 196, 210, 226, or 16/26/174/195/196/210/226, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 16S, 26E, 174K, 195L, 196V, 210E, 226S, or 16S/26E/174K/195L/196V/210E/226S, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13, 17, 196, or 13/17/196, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13P, 17H, 196K, or 13P/17H/196K, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 17, 60, 301, or 17/60/301, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 17G, 60V, 301I, or 17G/60V/301I, wherein the amino acid positions are relative to the reference sequence corresponding to SEQ ID NO: 2 or 4.

In some embodiments, the amino acid sequence of the engineered oxalate decarboxylase polypeptide comprises at least a substitution at amino acid position 37, wherein the amino acid position is relative to the reference sequence corresponding to SEQ ID NO: 2 or 4.

In some embodiments, the amino acid sequence of the engineered oxalate decarboxylase polypeptide comprises at least a substitution 37G, wherein the amino acid position is relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 14, 60, 162, 226, or 14/60/162/226, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 14L, 60V, 162K, 226T, or 14L/60V/162K/226T, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 83, 96, 277, or 83/96/277, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 83W, 96L, 277V, or 83W/96L/277V, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 346, 124, 359, 174, 173, 123, 196, 304, 301, 347, 11, 284, 210, 169, 216, 195, 339, 4, 6, 180, 80, 243, 182, 7, 226, 156, 183, 227, 219, 62, 343, 16/26, 16/26/242, 16/26/183/232, 155/206/242, 16/26/339, 16/26/155/206/339, 26, 26/206/339, 31/82/210, 31/82, 31/356, 31/97/226, 31/240/270, 31/82/226, 31/240, 31, 31/210/318, or 31/210, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 346G, 346L, 124G, 124T, 359L, 174K, 173V, 123Q, 196F, 304D, 301Q, 347F, 359W, 124Q, 124A, 11R, 284R, 210L, 169L, 304G, 216W, 347R, 196V, 284A, 195L, 339G, 4S, 196R, 6A, 124C, 195Y, 180E, 80L, 243V, 182R, 210E, 7G, 226S, 174A, 196M, 4C, 156E, 183E, 227S, 219V, 62G, 174G, 169G, 124R, 210V, 343R, 216S, 210R, 6W, 4R, 6S, 16S/26E, 16S/26E/242D, 16S/26E/183I/232T, 155P/206M/242D, 16S/26E/339G, 16S/26E/155P/206M/339G, 26E, 26E/206M/339G, 31K/82I/210A, 31K/82I, 31K/356S, 31K/97T/226S, 31K/240E/270L, 31K/82I/226S, 31K/240E, 31K, 31K/210A/318Q, or 31K/210A, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set E346G, E346L, K124G, K124T, K359L, D174K, K173V, D123Q, K196F, R304D, V301Q, K347F, K359W, K124Q, K124A, P11R, K284R, P210L, D169L, R304G, K216W, K347R, K196V, K284A, R195L, S339G, P4S, K196R, F6A, K124C, R195Y, R180E, F80L, P243V, A182R, P210E, M7G, T226S, D174A, K196M, P4C, L156E, V183E, R227S, R219V, E62G, D174G, D169G, K124R, P210V, S343R, K216S, P210R, F6W, P4R, F6S, A16S/A26E, A16S/A26E/E242D, A16S/A26E/V183I/K232T, A155P/L206M/E242D, A16S/A26E/S339G, A16S/A26E/A155P/L206M/S339G, A26E, A26E/L206M/S339G, E31K/L82I/P210A, E31K/L82I, E31K/A356S, E31K/A97T/T226S, E31K/D240E/M270L, E31K/L82I/T226S, E31K/D240E, E31K, E31K/P210A/E318Q, or E31K/P210A, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13, 212, 124/196/210/226, 5, 16/26/124/155/195/210/284, 196/226, 16/155/195/210/226, 16/195/210/226, 16/155/174/196, 16/195/226, 318, 16/195/196/210, 16/26/124/155/174/196/210, 195/210, 26/155/174/210, 316, 60, 16/155/174, 16/124/174/196, 16/226, 16/26/174/196/226, 17, 331, 124/195, 16/174/196, 188, 195/196/210, 174/196/210, 16/26/155/174, 16/155/195/196/226, 195/226/284, 16, 240, 16/26/124/155/195/196/226, 183/232/339/343, 183/206, 63, 173/347, 16/26/174/196, 16/284, 46, 16/124/195/196, 274, 174/196, 155/174/196, 174/196/226, 16/124/155/174/195, 16/26/155/174/196, 26/174/196/210/284, 16/195/196/284, 174, 16/124/174/195/210/226/284, 16/174/195/284, 26/174/196/210/226/284, 174/196/226/284, 124/174/196, 26/174/195/210/226, 162, 16/124/195/196/284, 206/343, 16/195/196, 16/26/174/195/196/210/226, 155/195/196/226, 18, 335, 124/155/174/195/226, 26/155/174/195/226, 195/196, 153, 155/174/195, 174/195/210/284, 174/195, 37, 124/174/195/226/284, 176, 16/155/174/195/196/226/284, 10, 169/173, 155, 33, 173/183/343/347, 104/265, or 233, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13P, 212S, 124A/196V/210E/226S, 5S, 13E, 16S/26E/124A/155P/195L/210E/284A, 196V/226S, 16S/155P/195L/210E/226S, 16S/195L/210E/226S, 16S/155P/174K/196V, 16S/195L/226S, 13L, 318E, 16S/195L/196V/210E, 16S/26E/124A/155P/174K/196V/210E, 195L/210E, 26E/155P/174K/210E, 316V, 60V, 16S/155P/174K, 16S/124A/174K/196V, 16S/226S, 16S/26E/174K/196V/226S, 17H, 331V, 124A/195L, 16S/174K/196V, 17A, 188A, 195L/196V/210E, 174K/196V/210E, 5C, 16S/26E/155P/174K, 16S/155P/195L/196V/226S, 195L/226S/284A, 17N, 16L, 240E, 16S/26E/124A/155P/195L/196V/226S, 331P, 1831/232T/339G/343R, 183I/206M, 63A, 173V/347R, 16S/26E/174K/196V, 16S/284A, 60R, 46R, 16S/124A/195L/196V, 274S, 331I, 174K/196V, 155P/174K/196V, 174K/196V/226S, 331L, 16S/124A/155P/174K/195L, 16S/26E/155P/174K/196V, 26E/174K/196V/210E/284A, 212A, 16S/195L/196V/284A, 174K, 16S/124A/174K/195L/210E/226S/284A, 16S/174K/195L/284A, 26E/174K/196V/210E/226S/284A, 212L, 174K/196V/226S/284A, 124A/174K/196V, 26E/174K/195L/210E/226S, 162A, 16S/124A/195L/196V/284A, 206M/343R, 16S/195L/196V, 16S/26E/174K/195L/196V/210E/226S, 155P/195L/196V/226S, 18Q, 335R, 124A/155P/174K/195L/226S, 17C, 26E/155P/174K/195L/226S, 195L/196V, 162T, 153H, 316K, 155P/174K/195L, 153Q, 174K/195L/210E/284A, 174K/195L, 153S, 212F, 37C, 212V, 124A/174K/195L/226S/284A, 176L, 16S/155P/174K/195L/196V/226S/284A, 10Q, 169G/173V, 155L, 18G, 60L, 10A, 18R, 33R, 212G, 10R, 13R, 173V/1831/343R/347R, 16R, 104Q/265C, 233R, or 233D, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/17/196, 17/212/331, 17/26/196/212/226, 17/26/196/212/226/331, 13/17/26/196/331, 17/60/196/226, 17/46/212, 26/60/196/226/331, 17/46/196/212/274/331, 212, 17/196/226, 13/17/26/212/331, 13/17/212/226/331, 196/212/331, 13/17/196/331, 13/17/26/212/226/331, 17/60/196/212/226/331, 13/17/212, 13/17/26/331, 13/17, 13/17/26/196, 46/196/212/226, 17/60/196, 17/196/331, 13/17/46/226/331, 17/196/226/331, 17/46/196/212/331, 17/46/212/331, 17/196, 13/17/212/226, 17/212, 17/26/212, 13/17/226/331, 17/196/212/331, 17/196/212/226/331, 13/17/26/226/331, 46/60/196/226/331, 17/46/196/331, 13/17/46/196, 196/212, 13/17/26, 17/83/263, 17/83/173/227, 17/227/301, 17/83/227/263, 17/83/125, 17/83/173, 17/263/301, 17/125, 17/83/301, 17/173, 17/83/227, 17/227, 17, 17/125/227, 17/83, 128, 76, 141, 110, 79, 117, or 61, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13P/17H/196K, 17H/212S/331V, 17H/26A/196K/212S/226T, 17H/26A/196K/212A/226T/331V, 13P/17H/26A/196K/331V, 17H/60R/196K/226T, 17H/46R/212S, 26A/60V/196K/226T/331V, 17H/46R/196K/212A/274Q/331V, 212S, 17H/196K/226T, 13P/17H/26A/212A/331V, 13P/17H/212S/226T/331V, 196K/212S/331V, 13P/17H/196K/331V, 13P/17H/26A/212A/226T/331V, 17H/60R/196K/212A/226T/331V, 13P/17H/212S, 13P/17H/26A/331V, 13P/17H, 13P/17H/26A/196K, 46R/196K/212S/226T, 17H/60R/196K, 17H/196K/331V, 13P/17H/46R/226T/331V, 17H/196K/226T/331V, 17H/60V/196K/226T, 17H/46R/196K/212S/331V, 13P/17H/212A, 17H/46R/212S/331V, 17H/196K, 13P/17H/212S/226T, 17H/212S, 17H/26A/212S, 13P/17H/226T/A331V, D17H/V196K/212S/331V, 17H/196K/212S/226T/331V, 13P/17H/26A/226T/331V, 46R/60V/196K/226T/331V, 17H/46R/196K/331V, 17H/212A/331V, 13P/17H/46R/196K, 196K/212S, 13P/17H/26A, 17G/83E/263S, 17G/83E/173Q/227E, 17G/227E/301I, 17G/83E/227E/263S, 17G/83E/125E, 17G/83E/173Q, 17G/263S/301I, 17G/125E, 17G/83E/301I, 17G/173Q, 17G/83E/227E, 17G/227E, 17G, 17G/125E/227E, 17G/83E, 17G/263S/301T, 128A, 76S, 141Q, 110V, 79S, 117A, 79G, or 61M, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/17/79, 13/17/79, 13/14/17/60/79, 13/17/79/83, 13/17/79/301, 13/14/17/60/79/212, 17/60/197, 13/60/212, 13/14/17/60/79/83/301, 13/17/60/79, 17/60/301, 14/17/79, 17/60/83, 17/79, 13/17/60/79/83/301, 342, 52/190, 94/190, 190/342/351, 13/79, 13/96, 273, 126, 96, 79, 269, 40, 44, 267, 266, 160, 277, 149, 22, 234, 54, 297, 106, or 13/43, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13H/17G/79G, 13H/14V/17G/60V/79G, 13H/17G/79G/83E, 13H/17G/79G/301I, 13H/14V/17G/60V/79G/212S, 17G/60V/197E, 13H/60V/212S, 13H/14V/17G/60V/79G/83E/301I, 13H/17G/60V/79G, 17G/60V/301I, 14V/17G/79G, 17G/60V/83E, 17G/79G, 13H/17G/60V/79G/83E/301I, 342P, 52T/190Q, 94N/190Q, 190Q/342P/351E, 13H/79G, 13H/96L, 273A, 126T, 96K, 79S, 269S, 40S, 96L, 44S, 96R, 79G, 267R, 266V, 160T, 277T, 149T, 22R, 234L, 54L, 297Y, 277A, 106C, or 13H/43C, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 37, 125, 162, 94, 195, 85, 103, 232, 189, 186, 155, 193, 342, 196, 63, 33, 351, 314, 187, 303, 164, 153, 346, 183, 19, 123, 350, 205, 284, 199, 343, 200, 208, 169, 190, or 121, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution 37G, 125F, 162K, 94T, 195W, 85R, 103S, 232T, 189R, 186N, 155R, 193A, 342A, 196V, 63S, 33R, 351H, 193G, 125S, 314S, 187R, 303T, 103V, 103Q, 155V, 164R, 153S, 37R, 346Q, 346G, 351A, 183K, 19C, 187L, 123S, 85G, 187S, 183C, 123G, 350R, 85E, 205A, 284R, 199V, 346W, 343W, 164V, 200N, 342E, 208G, 169A, 190G, 284A, 199G, 121W, or 196S, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 14/26/60/318, 14/26/60/94/162/212, 60/162/226, 60, 14/125/162/212/318, 60/125/226, 14/125/162/226, 26/60/162/226/318, 14/26/94/162/212/226, 14/60/162, 14/60/162/226, 14/94/162/318, 14/94/162, 14/60/226, 14/60/94/212, 14/60/162/212, 14/162, 14/94/212/318, 14/60, 14/26/162, 14, 14/318, 14/162/226/318, 14/125/212, 14/125/226/318, 14/26/60/162, 14/125/162/212/226, 14/94/125/162/318, 14/125/226, 277, 166, 233, 83, 5, 342, 96, 284, or 26, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 14L/26A/60V/318E, 14L/26A/60V/94T/162K/212S, 60V/162K/226T, 60V, 14L/125F/162K/212S/318E, 60V/125F/226T, 14L/125F/162K/226T, 26A/60V/162K/226T/318E, 14L/26A/94T/162K/212S/226T, 14L/60V/162K, 14L/60V/162K/226T, 14L/94T/162K/318E, 14L/94T/162K, 14L/60V/226T, 14L/60V/94T/212S, 14L/60V/162K/212S, 14L/162K, 14L/94T/212S/318E, 14L/60V, 14L/26A/162K, 14L, 14L/318E, 14L/162K/226T/318E, 14L/125F/212S, 14L/125F/226T/318E, 14L/26A/60V/162K, 14L/125F/162K/212S/226T, 14L/94T/125F/162K/318E, 14L/125F/226T, 277R, 277C, 277T, 166R, 233H, 83R, 60M, 277I, 277M, 5S, 342R, 277V, 60R, 96R, 284S, 26R, 26N, or 83W, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 5/26/83/342, 5/26/277, 5/96/277, 5/83/277/342, 26/83/277, 5/26/83/277, 5/277/342, 5/26/166/277, 83/277/342, 83/96/277/342, 83/96/277, 26/83/277/342, 5/26/83/277/342, 5/83/277, 26/277, 83/277, 5/35/277, 5/26/83/96/277, 5/26/96/277, 5/277, 5/166/277, 26/83/96/277/342, 277, 26/277/342, 5, or 5/83/96/277/342, wherein the amino acid positions are relative to the reference sequence corresponding to SEQ ID NO: 2 or 4.

In some embodiments, the amino acid sequence of the engineered oxalate decarboxylase polypeptide comprises at least substitution or substitution set 5S/26A/83W/342R, 5S/26A/277T, 5S/96L/277T, 5S/83W/277T/342R, 26A/83W/277T, 5S/26A/83W/277V, 5S/277T/342R, 5S/83W/277V/342R, 5S/26A/166R/277T, 83W/277T/342R, 83R/96L/277V/342R, 83W/96L/277T, 26A/83W/277V/342R, 5S/26A/83W/277V/342R, 5S/83R/277T/342R, 5S/26A/83W/277T/342R, 5S/83R/277V, 26A/277T, 83W/277T, 5S/35V/277T, 5S/26A/83R/96R/277V, 5S/26A/96L/277T, 5S/277T, 5S/26A/83R/277T, 5S/83R/277T, 26A/83R/277V, 5S/26A/277V, 5S/166R/277T, 26A/83W/277V, 26A/83W/96L/277V/342R, 277T, 26A/277T/342R, 83W/277V/342R, 83R/277V, 5S, 26A/277V, 83R/277T, 5S/83W/96R/277T/342R, 5S/277V, 277V, 5S/277V/342R, 83W/96L/277V, or 83W/277V, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 26, 31, 96, 60, 318, 210, 277, 37, 16, 195, 79, 17, 13, 83, 162, or 174, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution 26L, 31W, 96K, 60R, 318D, 26G, 210R, 31R, 277F, 37M, 96P, 16V, 60A, 26R, 31G, 195G, 318R, 26V, 79M, 16M, 210A, 96R, 37E, 16G, 17W, 13G, 79E, 277T, 60Q, 31L, 96G, 96T, 60G, 16W, 13T, 79R, 26Y, 26S, 195R, 17R, 13V, 277E, 37L, 210G, 60L, 13L, 83Y, 37W, 277G, 83R, 60Y, 210S, 318S, 17I, 37S, 79L, 16R, 195S, 318G, 26C, 13R, 195Y, 60E, 162V, 16C, 96V, 83M, 31M, 174V, 277L, 26M, 318M, 210N, 26A, 277R, 96F, 174G, 16Q, 277A, 13Q, 79F, 16L, 195N, 79V, 17P, 60P, 37P, 96W, 162T, 195M, 318W, 37R, 16T, 210M, 162Y, 16P, 83E, 277M, 174L, 17V, 17E, 17L, 96M, 318V, 174R, 13S, 174T, 13Y, 195A, 277W, 60M, 17A, 318A, 174H, 83S, 277I, 13E, 17K, 26K, 96A, 174D, 210V, 31S, 195C, 79K, 60W, 174S, 13I, 60H, 31E, 96E, 83K, 31Q, 16K, 174M, 37V, 79D, 318T, 195F, 26T, 174Q, 83L, 96I, 13C, 13A, 96Q, 13H, 83T, 31Y, 13W, 277P, 195T, 60S, 26Q, 210P, 174N, 83A, 162E, 37F, 37Q, 31T, 174A, 83F, 60T, 96S, 83V, 210I, 16D, 37C, 83G, 13N, 31V, 79S, 96Y, 17F, 17M, 16A, 83P, 318L, 79Q, 17S, 26W, 195D, 195W, 277S, 174E, 16E, 162F, 79Y, 79A, 17C, 17Y, 195Q, 26P, 195I, 60F, 26I, 17D, 318K, 60N, 16I, 318H, 37A, 195V, 210Y, 195E, 26H, 13D, 31P, 210W, 318P, 83C, 174P, 210T, 17T, 79T, 96H, 79W, 162L, 318E, 31A, 210C, 17N, 195K, 318Y, 17H, 13M, 60D, 277K, 16N, 79C, 13K, 60K, 37N, 60I, 162D, 162I, 16Y, 162S, 96C, 210K, 174I, 210L, 26D, 26N, 210D, 13F, 318N, 277Y, 174W, 31H, 31D, 79N, 37K, 31I, 37H, 318C, 31F, 79P, 195P, 318I, 162A, 277Q, 26F, 277C, 79H, 31C, 79I, 37I, 17Q, 31N, 277N, 37D, 277H, 277D, 96D, 174C, 162P, 318F, 83N, 60C, 83I, 162M, 210H, 195H, 174Y, 37Y, 210Q, 37T, 96N, 83H, 16H, 174F, 83D, 83Q, 162H, 162W, 162N, 16F, 162Q, 162G, 162R, 210F, or 162C, or combinations thereof, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 174/318, 26/96, 60/318, 31/318, 162/174/318, 174/277/318, 26/195/318, 60/162/195, 60/162/318, 174/195/277, 16/17/174, 60/195/277, 37/277/318, 16/162/195/277, 26/79/83/162, 26/31/60/318, 16/174/195/277, 13/37/83/162, 16/17/60/195, 13/17/37/83, 31/37/162/174/318, 13/16/31/83/162, 16/60/174/195/318, 17/60/96/162/195/277, 13/26/60/83/162/174, 17/31/60/162/174/277, 16/17/26/83/96/277, 17/26/37/174/277/318, 13/16/31/60/162/174, 13/17/31/83/277/318, 13/16/17/31/83/162/318, 13/16/60/83/162/195/318, 13/16/17/31/37/83/96/162, 13/17/26/37/60/83/162/210/318, 13/16/26/31/83/96/162/174/318, 13/16/17/31/60/83/96/162/174/195/318, 13/16/26/37/60/83/162/174/195/210/318, 13/16/37/79/83/96/162/174/195/210/277, 13/17/26/31/37/60/79/83/96/162/174/210, 13/16/17/26/37/79/83/96/174/195/210/318, 13/26/31/37/60/83/96/162/174/195/210/277/318, 13/16/17/31/37/60/79/83/96/162/174/195/210/318, 13/16/17/31/60/79/83/96/162/174/195/210/277/318, 13/16/17/26/37/60/79/96/162/174/195/210/277/318, 13/26/31/37/60/79/83/96/162/174/195/210/277/318, 13/16/17/26/31/37/60/96/162/174/195/210/277/318, 13/16/17/26/31/37/79/83/96/162/174/195/210/318, 13/16/17/26/31/37/60/79/96/162/174/210/277/318, 13/17/26/31/37/60/79/83/96/162/174/195/210/277/318, 13/16/26/31/37/60/79/83/96/162/174/195/210/277/318, 16/17/26/31/37/60/79/83/96/162/174/195/210/277/318, or 13/16/17/26/31/37/60/79/83/96/162/174/195/210/277/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 174D/318E, 26A/96A, 60T/318E, 31E/318E, 162G/174D/318E, 174D/277Y/318E, 26A/195R/318E, 60T/162G/195R, 60T/162G/318E, 174D/195R/277Y, 16A/17D/174D, 60T/195R/277Y, 37K/277Y/318E, 16A/162G/195R/277Y, 26A/79A/83T/162G, 26A/31E/60T/318E, 16A/174D/195R/277Y, 13H/37K/83T/162G, 16A/17D/60T/195R, 13H/17D/37K/83T, 31E/37K/162G/174D/318E, 13H/16A/31E/83T/162G, 16A/60T/174D/195R/318E, 17D/60T/96A/162G/195R/277Y, 13H/26A/60T/83T/162G/174D, 17D/31E/60T/162G/174D/277Y, 16A/17D/26A/83T/96A/277Y, 17D/26A/37K/174D/277Y/318E, 13H/16A/31E/60T/162G/174D, 13H/17D/31E/83T/277Y/318E, 13H/16A/17D/31E/83T/162G/318E, 13H/16A/60T/83T/162G/195R/318E, 13H/16A/17D/31E/37K/83T/96A/162G, 13H/17D/26A/37K/60T/83T/162G/210P/318E, 13H/16A/26A/37K/83T/162G/174D/195R/318E, 13H/16A/26A/31E/83T/96A/162G/174D/318E, 13H/16A/17D/31E/60T/83T/96A/162G/174D/195R/318E, 13H/16A/26A/37K/60T/83T/162G/174D/195R/210P/318E, 13H/16A/37K/79A/83T/96A/162G/174D/195R/210P/277Y, 13H/17D/26A/31E/37K/60T/79A/83T/96A/162G/174D/210P, 13H/16A/17D/26A/37K/79A/83T/96A/174D/195R/210D/318E, 13H/26A/31E/37K/60T/83T/96A/162G/174D/195R/210P/277Y/318E, 13H/16A/17D/31E/37K/60T/79A/83T/96A/162G/174D/195R/210P/318E, 13H/16A/17D/31E/60T/79A/83T/96A/162G/174D/195R/210P/277Y/318E, 13H/16A/17D/26A/37K/60T/79A/96A/162G/174D/195R/210P/277Y/318E, 13H/26A/31E/37K/60T/79A/83T/96A/162G/174D/195R/210P/277Y/318E, 13H/16A/17D/26A/31E/37K/60T/96A/162G/174D/195R/210P/277Y/318E, 13H/16A/17D/26A/31E/37K/79A/83T/96A/162G/174D/195R/210P/318E, 13H/16A/17D/26A/31E/37K/60T/79A/96A/162G/174D/210P/277Y/318E, 13H/17D/26A/31E/37K/60T/79A/83T/96A/162G/174D/195R/210P/277Y/318E, 13H/16A/26A/31E/37K/60T/79A/83T/96A/162G/174D/195R/210P/277Y/318E, 16A/17D/26A/31E/37K/60T/79A/83T/96A/162G/174D/195R/210P/277Y/318E, or 13H/16A/17D/26A/31E/37K/60T/79A/83T/96A/162G/174D/195R/210P/277H/318E, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/31/210/318, 31/210/212/318, 31/124/196/210/226/318, 5/31/210/318, 16/26/31/124/155/195/210/284/318, 31/196/210/226/318, 16/31/155/195/210/226/318, 16/31/195/210/226/318, 16/31/155/174/196/210/318, 31/210, 16/31/195/196/210/318, 16/26/31/124/155/174/196/210/318, 31/195/210/318, 26/31/155/174/210/318, 31/210/316/318, 31/60/210/318, 16/31/155/174/210/318, 16/31/124/174/196/210/318, 16/31/210/226/318, 16/26/31/174/196/210/226/318, 17/31/210/318, 31/210/318/331, 31/124/195/210/318, 16/31/174/196/210/318, 31/188/210/318, 31/195/196/210/318, 31/174/196/210/318, 16/26/31/155/174/210/318, 16/31/155/195/196/210/226/318, 31/195/210/226/284/318, 16/31/210/318, 31/210/240/318, 16/26/31/124/155/195/196/210/226/318, 31/183/210/232/318/339/343, 31/183/206/210/318, 31/63/210/318, 31/173/210/318/347, 16/26/31/174/196/210/318, 16/31/210/284/318, 31/46/210/318, 16/31/124/195/196/210/318, 31/210/274/318, 31/155/174/196/210/318, 31/174/196/210/226/318, 16/31/124/155/174/195/210/318, 16/26/31/155/174/196/210/318, 26/31/174/196/210/284/318, 16/31/195/196/210/284/318, 31/174/210/318, 16/31/124/174/195/210/226/284/318, 16/31/174/195/210/284/318, 26/31/174/196/210/226/284/318, 31/174/196/210/226/284/318, 31/124/174/196/210/318, 26/31/174/195/210/226/318, 31/162/210/318, 16/31/124/195/196/210/284/318, 31/206/210/318/343, 16/26/31/174/195/196/210/226/318, 31/155/195/196/210/226/318, 18/31/210/318, 31/210/318/335, 31/124/155/174/195/210/226/318, 26/31/155/174/195/210/226/318, 31/153/210/318, 31/155/174/195/210/318, 31/174/195/210/284/318, 31/174/195/210/318, 31/37/210/318, 31/124/174/195/210/226/284/318, 31/176/210/318, 16/31/155/174/195/196/210/226/284/318, 10/31/210/318, 31/169/173/210/318, 31/155/210/318, 31/33/210/318, 31/173/183/210/318/343/347, 31/104/210/265/318, or 31/210/233/318, wherein the amino acid positions are relative to the reference sequence corresponding to SEQ ID NO: 2 or 4.

In some embodiments, the amino acid sequence of the engineered oxalate decarboxylase polypeptide comprises a substitution or substitution set 13P/31K/210A/318Q, 31K/210A/212S/318Q, 31K/124A/196V/210E/226S/318Q, 5S/31K/210A/318Q, 13E/31K/210A/318Q, 16S/26E/31K/124A/155P/195L/210E/284A/318Q, 31K/196V/210A/226S/318Q, 16S/31K/155P/195L/210E/226S/318Q, 16S/31K/195L/210E/226S/318Q, 16S/31K/155P/174K/196V/210A/318Q, 16S/31K/195L/210A/226S/318Q, 13L/31K/210A/318Q, 31K/210A, 16S/31K/195L/196V/210E/318Q, 16S/26E/31K/124A/155P/174K/196V/210E/318Q, 31K/195L/210E/318Q, 26E/31K/155P/174K/210E/318Q, 31K/210A/316V/318Q, 31K/60V/210A/318Q, 16S/31K/155P/174K/210A/318Q, 16S/31K/124A/174K/196V/210A/318Q, 16S/31K/210A/226S/318Q, 16S/26E/31K/174K/196V/210A/226S/318Q, 17H/31K/210A/318Q, 31K/210A/318Q/331V, 31K/124A/195L/210A/318Q, 16S/31K/174K/196V/210A/318Q, 17A/31K/210A/318Q, 31K/188A/210A/318Q, 31K/195L/196V/210E/318Q, 31K/174K/196V/210E/318Q, 5C/31K/210A/318Q, 16S/26E/31K/155P/174K/210A/318Q, 16S/31K/155P/195L/196V/210A/226S/318Q, 31K/195L/210A/226S/284A/318Q, 17N/31K/210A/318Q, 16L/31K/210A/318Q, 31K/210A/240E/318Q, 16S/26E/31K/124A/155P/195L/196V/210A/226S/318Q, 31K/210A/318Q/331P, 31K/183I/210A/232T/318Q/339G/343R, 31K/183I/206M/210A/318Q, 31K/63A/210A/318Q, 31K/173V/210A/318Q/347R, 16S/26E/31K/174K/196V/210A/318Q, 16S/31K/210A/284A/318Q, 31K/60R/210A/318Q, 31K/46R/210A/318Q, 16S/31K/124A/195L/196V/210A/318Q, 31K/210A/274S/318Q, 31K/210A/318Q/331I, 31K/174K/196V/210A/318Q, 31K/155P/174K/196V/210A/318Q, 31K/174K/196V/210A/226S/318Q, 31K/210A/318Q/331L, 16S/31K/124A/155P/174K/195L/210A/318Q, 16S/26E/31K/155P/174K/196V/210A/318Q, 26E/31K/174K/196V/210E/284A/318Q, 31K/210A/212A/318Q, 16S/31K/195L/196V/210A/284A/318Q, 31K/174K/210A/318Q, 16S/31K/124A/174K/195L/210E/226S/284A/318Q, 16S/31K/174K/195L/210A/284A/318Q, 26E/31K/174K/196V/210E/226S/284A/318Q, 31K/210A/212L/318Q, 31K/174K/196V/210A/226S/284A/318Q, 31K/124A/174K/196V/210A/318Q, 26E/31K/174K/195L/210E/226S/318Q, 31K/162A/210A/318Q, 16S/31K/124A/195L/196V/210A/284A/318Q, 31K/206M/210A/318Q/343R, 16S/31K/195L/196V/210A/318Q, 16S/26E/31K/174K/195L/196V/210E/2265/318Q, 31K/155P/195L/196V/210A/226S/318Q, 18Q/31K/210A/318Q, 31K/210A/318Q/335R, 31K/124A/155P/174K/195L/210A/226S/318Q, 17C/31K/210A/318Q, 26E/31K/155P/174K/195L/210A/226S/318Q, 31K/195L/196V/210A/318Q, 31K/162T/210A/318Q, 31K/153H/210A/318Q, 31K/210A/316K/318Q, 31K/155P/174K/195L/210A/318Q, 31K/153Q/210A/318Q, 31K/174K/195L/210E/284A/318Q, 31K/174K/195L/210A/318Q, 31K/153S/210A/318Q, 31K/210A/212F/318Q, 31K/37C/210A/318Q, 31K/210A/212V/318Q, 31K/124A/174K/195L/210A/226S/284A/318Q, 31K/176L/210A/318Q, 16S/31K/155P/174K/195L/196V/210A/226S/284A/318Q, 10Q/31K/210A/318Q, 31K/169G/173V/210A/318Q, 31K/155L/210A/318Q, 18G/31K/210A/318Q, 31K/60L/210A/318Q, 10A/31K/210A/318Q, 18R/31K/210A/318Q, 31K/33R/210A/318Q, 31K/210A/212G/318Q, 10R/31K/210A/318Q, 13R/31K/210A/318Q, 31K/173V/183I/210A/318Q/343R/347R, 16R/31K/210A/318Q, 31K/104Q/210A/265C/318Q, 31K/210A/233R/318Q, or 31K/210A/233D/318Q, wherein the amino acid positions are relative to the reference sequence of SEQ ID NO: 2 or 4.

In some embodiments, the amino acid sequence of the engineered oxalate decarboxylase polypeptide comprises at least a substitution or substitution set at amino acid position(s) 13/16/17/26/31/174/195/210/226/318, 16/17/26/31/174/195/196/210/212/226/318/331, 16/17/31/174/195/210/212/318, 16/17/31/174/195/210/212/318/331, 13/16/17/31/174/195/210/226/318/331, 16/17/26/31/60/174/195/210/318, 16/17/26/31/46/174/195/196/210/212/226/318, 16/31/60/174/195/210/318/331, 16/17/26/31/46/174/195/210/212/226/274/318/331, 16/26/31/174/195/196/210/212/226/318, 16/17/26/31/174/195/210/318, 13/16/17/31/174/195/196/210/212/226/318/331, 13/16/17/26/31/174/195/196/210/212/318/331, 16/26/31/174/195/210/212/226/318/331, 13/16/17/26/31/174/195/210/226/318/331, 13/16/17/31/174/195/196/210/212/318/331, 16/17/26/31/60/174/195/210/212/318/331, 13/16/17/26/31/174/195/196/210/212/226/318, 13/16/17/31/174/195/196/210/226/318/331, 13/16/17/26/31/174/195/196/210/226/318, 13/16/17/31/174/195/210/226/318, 16/26/31/46/174/195/210/212/318, 16/17/26/31/60/174/195/210/226/318, 16/17/26/31/174/195/210/226/318/331, 13/16/17/26/31/46/174/195/196/210/318/331, 16/17/26/31/174/195/210/318/331, 16/17/26/31/46/174/195/210/212/226/318/331, 16/17/26/31/46/174/195/196/210/212/226/318/331, 16/17/26/31/174/195/210/226/318, 13/16/17/26/31/174/195/196/210/212/318, 16/17/26/31/174/195/196/210/212/226/318, 16/17/31/174/195/196/210/212/226/318, 13/16/17/26/31/174/195/196/210/318/331, 16/17/26/31/174/195/210/212/226/318/331, 16/17/26/31/174/195/210/212/318/331, 13/16/17/31/174/195/196/210/318/331, 16/26/31/46/60/174/195/210/318/331, 16/17/26/31/46/174/195/210/226/318/331, 13/16/17/26/31/46/174/195/210/226/318, 16/26/31/174/195/210/212/226/318, 13/16/17/31/174/195/196/210/226/318, 16/17/26/31/83/174/195/196/210/226/263/318, 16/17/26/31/83/173/174/195/196/210/226/227/318, 16/17/26/31/174/195/196/210/226/227/301/318, 16/17/26/31/83/174/195/196/210/226/227/263/318, 16/17/26/31/83/125/174/195/196/210/226/318, 16/17/26/31/83/173/174/195/196/210/226/318, 16/17/26/31/174/195/196/210/226/263/301/318, 16/17/26/31/125/174/195/196/210/226/318, 16/17/26/31/83/174/195/196/210/226/301/318, 16/17/26/31/173/174/195/196/210/226/318, 16/17/26/31/83/174/195/196/210/226/227/318, 16/17/26/31/174/195/196/210/226/227/318, 16/17/26/31/174/195/196/210/226/318, 16/17/26/31/125/174/195/196/210/226/227/318, 16/17/26/31/83/174/195/196/210/226/318, 16/26/31/128/174/195/196/210/226/318, 16/26/31/76/174/195/196/210/226/318, 16/26/31/141/174/195/196/210/226/318, 16/26/31/110/174/195/196/210/226/318, 16/26/31/79/174/195/196/210/226/318, 16/26/31/117/174/195/196/210/226/318, or 16/26/31/61/174/195/196/210/226/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13P/16S/17H/26E/31K/174K/195L/210E/226S/318Q, 16S/17H/26E/31K/174K/195L/196V/210E/212S/226S/318Q/331V, 16S/17H/31K/174K/195L/210E/212S/318Q, 16S/17H/31K/174K/195L/210E/212A/318Q/331V, 13P/16S/17H/31K/174K/195L/210E/226S/318Q/331V, 16S/17H/26E/31K/60R/174K/195L/210E/318Q, 16S/17H/26E/31K/46R/174K/195L/196V/210E/212S/226S/318Q, 16S/31K/60V/174K/195L/210E/318Q/331V, 16S/17H/26E/31K/46R/174K/195L/210E/212A/226S/274Q/318Q/331V, 16S/26E/31K/174K/195L/196V/210E/212S/226S/318Q, 16S/17H/26E/31K/174K/195L/210E/318Q, 13P/16S/17H/31K/174K/195L/196V/210E/212A/226S/318Q/331V, 13P/16S/17H/26E/31K/174K/195L/196V/210E/212S/318Q/331V, 16S/26E/31K/174K/195L/210E/212S/226S/318Q/331V, 13P/16S/17H/26E/31K/174K/195L/210E/226S/318Q/331V, 13P/16S/17H/31K/174K/195L/196V/210E/212A/318Q/331V, 16S/17H/26E/31K/60R/174K/195L/210E/212A/318Q/331V, 13P/16S/17H/26E/31K/174K/195L/196V/210E/212S/226S/318Q, 13P/16S/17H/31K/174K/195L/196V/210E/226S/318Q/331V, 13P/16S/17H/26E/31K/174K/195L/196V/210E/226S/318Q, 13P/16S/17H/31K/174K/195L/210E/226S/318Q, 16S/26E/31K/46R/174K/195L/210E/212S/318Q, 16S/17H/26E/31K/60R/174K/195L/210E/226S/318Q, 16S/17H/26E/31K/174K/195L/210E/226S/318Q/331V, 13P/16S/17H/26E/31K/46R/174K/195L/196V/210E/318Q/331V, 16S/17H/26E/31K/174K/195L/210E/318Q/331V, 16S/17H/26E/31K/60V/174K/195L/210E/318Q, 16S/17H/26E/31K/46R/174K/195L/210E/212S/226S/318Q/331V, 13P/16S/17H/26E/31K/174K/195L/196V/210E/212A/226S/318Q, 16S/17H/26E/31K/46R/174K/195L/196V/210E/212S/226S/318Q/331V, 16S/17H/26E/31K/174K/195L/210E/226S/318Q, 13P/16S/17H/26E/31K/174K/195L/196V/210E/212S/318Q, 16S/17H/26E/31K/174K/195L/196V/210E/212S/226S/318Q, 16S/17H/31K/174K/195L/196V/210E/212S/226S/318Q, 13P/16S/17H/26E/31K/174K/195L/196V/210E/318Q/331V, 16S/17H/26E/31K/174K/195L/210E/212S/226S/318Q/331V, 16S/17H/26E/31K/174K/195L/210E/212S/318Q/331V, 13P/16S/17H/31K/174K/195L/196V/210E/318Q/331V, 16S/26E/31K/46R/60V/174K/195L/210E/318Q/331V, 16S/17H/26E/31K/46R/174K/195L/210E/226S/318Q/331V, 16S/17H/26E/31K/174K/195L/196V/210E/212A/226S/318Q/331V, 13P/16S/17H/26E/31K/46R/174K/195L/210E/226S/318Q, 16S/26E/31K/174K/195L/210E/212S/226S/318Q, 13P/16S/17H/31K/174K/195L/196V/210E/226S/318Q, 16S/17G/26E/31K/83E/174K/195L/196V/210E/226S/263S/318Q, 16S/17G/26E/31K/83E/173Q/174K/195L/196V/210E/226S/227E/318Q, 16S/17G/26E/31K/174K/195L/196V/210E/226S/227E/3011/318Q, 16S/17G/26E/31K/83E/174K/195L/196V/210E/226S/227E/263S/318Q, 16S/17G/26E/31K/83E/125E/174K/195L/196V/210E/226S/318Q, 16S/17G/26E/31K/83E/173Q/174K/195L/196V/210E/226S/318Q, 16S/17G/26E/31K/174K/195L/196V/210E/226S/263S/301I/318Q, 16S/17G/26E/31K/125E/174K/195L/196V/210E/226S/318Q, 16S/17G/26E/31K/83E/174K/195L/196V/210E/226S/301I/318Q, 16S/17G/26E/31K/173Q/174K/195L/196V/210E/226S/318Q, 16S/17G/26E/31K/83E/174K/195L/196V/210E/226S/227E/318Q, 16S/17G/26E/31K/174K/195L/196V/210E/226S/227E/318Q, 16S/17G/26E/31K/174K/195L/196V/210E/226S/318Q, 16S/17G/26E/31K/125E/174K/195L/196V/210E/226S/227E/318Q, 16S/17G/26E/31K/83E/174K/195L/196V/210E/226S/318Q, 16S/17G/26E/31K/174K/195L/196V/210E/226S/263S/301T/318Q, 16S/26E/31K/128A/174K/195L/196V/210E/226S/318Q, 16S/26E/31K/76S/174K/195L/196V/210E/226S/318Q, 16S/26E/31K/141Q/174K/195L/196V/210E/226S/318Q, 16S/26E/31K/110V/174K/195L/196V/210E/226S/318Q, 16S/26E/31K/79S/174K/195L/196V/210E/226S/318Q, 16S/26E/31K/117A/174K/195L/196V/210E/226S/318Q, 16S/26E/31K/79G/174K/195L/196V/210E/2265/318Q, or 16S/26E/31K/61M/174K/195L/196V/210E/226S/318Q, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 16/17/26/31/79/174/195/210/226/318, 14/16/17/26/31/60/79/174/195/210/226/318, 16/17/26/31/79/83/174/195/210/226/318, 16/17/26/31/79/174/195/210/226/301/318, 14/16/17/26/31/60/79/174/195/210/212/226/318, 13/16/17/26/31/60/174/195/197/210/226/318, 16/17/26/31/60/174/195/210/212/226/318, 14/16/17/26/31/60/79/83/174/195/210/226/301/318, 16/17/26/31/60/79/174/195/210/226/318, 13/16/17/26/31/60/174/195/210/226/301/318, 13/14/16/17/26/31/79/174/195/210/226/318, 13/16/17/26/31/60/83/174/195/210/226/318, 13/16/17/26/31/79/174/195/210/226/318, 16/17/26/31/60/79/83/174/195/210/226/301/318, 13/16/17/26/31/174/195/210/226/318/342, 13/16/17/26/31/52/174/190/195/210/226/318, 13/16/17/26/31/94/174/190/195/210/226/318, 13/16/17/26/31/174/190/195/210/226/318/342/351, 16/17/26/31/96/174/195/210/226/318, 13/16/17/26/31/174/195/210/226/273/318, 13/16/17/26/31/126/174/195/210/226/318, 13/16/17/26/31/96/174/195/210/226/318, 13/16/17/26/31/174/195/210/226/269/318, 13/16/17/26/31/40/174/195/210/226/318, 13/16/17/26/31/44/174/195/210/226/318, 13/16/17/26/31/174/195/210/226/267/318, 13/16/17/26/31/174/195/210/226/266/318, 13/16/17/26/31/160/174/195/210/226/318, 13/16/17/26/31/174/195/210/226/277/318, 13/16/17/26/31/149/174/195/210/226/318, 13/16/17/22/26/31/174/195/210/226/318, 13/16/17/26/31/174/195/210/226/234/318, 13/16/17/26/31/54/174/195/210/226/318, 13/16/17/26/31/174/195/210/226/297/318, 13/16/17/26/31/106/174/195/210/226/318, or 16/17/26/31/43/174/195/210/226/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 16S/17G/26E/31K/79G/174K/195L/210E/226S/318Q, 14V/16S/17G/26E/31K/60V/79G/174K/195L/210E/226S/318Q, 16S/17G/26E/31K/79G/83E/174K/195L/210E/226S/318Q, 16S/17G/26E/31K/79G/174K/195L/210E/226S/3011/318Q, 14V/16S/17G/26E/31K/60V/79G/174K/195L/210E/212S/226S/318Q, 13P/16S/17G/26E/31K/60V/174K/195L/197E/210E/226S/318Q, 16S/17H/26E/31K/60V/174K/195L/210E/212S/226S/318Q, 14V/16S/17G/26E/31K/60V/79G/83E/174K/195L/210E/226S/301I/318Q, 16S/17G/26E/31K/60V/79G/174K/195L/210E/226S/318Q, 13P/16S/17G/26E/31K/60V/174K/195L/210E/226S/301I/318Q, 13P/14V/16S/17G/26E/31K/79G/174K/195L/210E/226S/318Q, 13P/16S/17G/26E/31K/60V/83E/174K/195L/210E/226S/318Q, 13P/16S/17G/26E/31K/79G/174K/195L/210E/226S/318Q, 16S/17G/26E/31K/60V/79G/83E/174K/195L/210E/226S/301I/318Q, 13P/16S/17H/26E/31K/174K/195L/210E/226S/318Q/342P, 13P/16S/17H/26E/31K/52T/174K/190Q/195L/210E/226S/318Q, 13P/16S/17H/26E/31K/94N/174K/190Q/195L/210E/226S/318Q, 13P/16S/17H/26E/31K/174K/190Q/195L/210E/226S/318Q/342P/351E, 16S/17H/26E/31K/79G/174K/195L/210E/226S/318Q, 16S/17H/26E/31K/96L/174K/195L/210E/226S/318Q, 13P/16S/17H/26E/31K/174K/195L/210E/226S/273A/318Q, 13P/16S/17H/26E/31K/126T/174K/195L/210E/226S/318Q, 13P/16S/17H/26E/31K/96K/174K/195L/210E/226S/318Q, 13P/16S/17H/26E/31K/79S/174K/195L/210E/226S/318Q, 13P/16S/17H/26E/31K/174K/195L/210E/226S/269S/318Q, 13P/16S/17H/26E/31K/40S/174K/195L/210E/226S/318Q, 13P/16S/17H/26E/31K/96L/174K/195L/210E/226S/318Q, 13P/16S/17H/26E/31K/44S/174K/195L/210E/226S/318Q, 13P/16S/17H/26E/31K/96R/174K/195L/210E/226S/318Q, 13P/16S/17H/26E/31K/79G/174K/195L/210E/226S/318Q, 13P/16S/17H/26E/31K/174K/195L/210E/226S/267R/318Q, 13P/16S/17H/26E/31K/174K/195L/210E/226S/266V/318Q, 13P/16S/17H/26E/31K/160T/174K/195L/210E/226S/318Q, 13P/16S/17H/26E/31K/174K/195L/210E/226S/277T/318Q, 13P/16S/17H/26E/31K/149T/174K/195L/210E/226S/318Q, 13P/16S/17H/22R/26E/31K/174K/195L/210E/226S/318Q, 13P/16S/17H/26E/31K/174K/195L/210E/226S/234L/318Q, 13P/16S/17H/26E/31K/54L/174K/195L/210E/226S/318Q, 13P/16S/17H/26E/31K/174K/195L/210E/226S/297Y/318Q, 13P/16S/17H/26E/31K/174K/195L/210E/226S/277A/318Q, 13P/16S/17H/26E/31K/106C/174K/195L/210E/226S/318Q, or 16S/17H/26E/31K/43C/174K/195L/210E/226S/318Q, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/14/16/17/26/31/37/79/174/195/210/226/318, 13/14/16/17/26/31/79/125/174/195/210/226/318, 13/14/16/17/26/31/79/162/174/195/210/226/318, 13/14/16/17/26/31/79/94/174/195/210/226/318, 13/14/16/17/26/31/79/174/195/210/226/318, 13/14/16/17/26/31/79/85/174/195/210/226/318, 13/14/16/17/26/31/79/103/174/195/210/226/318, 13/14/16/17/26/31/79/174/195/210/226/232/318, 13/14/16/17/26/31/79/174/189/195/210/226/318, 13/14/16/17/26/31/79/174/186/195/210/226/318, 13/14/16/17/26/31/79/155/174/195/210/226/318, 13/14/16/17/26/31/79/174/193/195/210/226/318, 13/14/16/17/26/31/79/174/195/210/226/318/342, 13/14/16/17/26/31/79/174/195/196/210/226/318, 13/14/16/17/26/31/63/79/174/195/210/226/318, 13/14/16/17/26/31/33/79/174/195/210/226/318, 13/14/16/17/26/31/79/174/195/210/226/318/351, 13/14/16/17/26/31/79/174/195/210/226/314/318, 13/14/16/17/26/31/79/174/187/195/210/226/318, 13/14/16/17/26/31/79/174/195/210/226/303/318, 13/14/16/17/26/31/79/164/174/195/210/226/318, 13/14/16/17/26/31/79/153/174/195/210/226/318, 13/14/16/17/26/31/79/174/195/210/226/318/346, 13/14/16/17/26/31/79/174/183/195/210/226/318, 13/14/16/17/19/26/31/79/174/195/210/226/318, 13/14/16/17/26/31/79/123/174/195/210/226/318, 13/14/16/17/26/31/79/174/195/210/226/318/350, 13/14/16/17/26/31/79/174/195/205/210/226/318, 13/14/16/17/26/31/79/174/195/210/226/284/318, 13/14/16/17/26/31/79/174/195/199/210/226/318, 13/14/16/17/26/31/79/174/195/210/226/318/343, 13/14/16/17/26/31/79/174/195/200/210/226/318, 13/14/16/17/26/31/79/174/195/208/210/226/318, 13/14/16/17/26/31/79/169/174/195/210/226/318, 13/14/16/17/26/31/79/174/190/195/210/226/318, or 13/14/16/17/26/31/79/121/174/195/210/226/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13P/14V/16S/17G/26E/31K/37G/79G/174K/195L/210E/226S/318Q, 13P/14V/16S/17G/26E/31K/79G/125F/174K/195L/210E/226S/318Q, 13P/14V/16S/17G/26E/31K/79G/162K/174K/195L/210E/226S/318Q, 13P/14V/16S/17G/26E/31K/79G/94T/174K/195L/210E/226S/318Q, 13P/14V/16S/17G/26E/31K/79G/174K/195W/210E/226S/318Q, 13P/14V/16S/17G/26E/31K/79G/85R/174K/195L/210E/226S/318Q, 13P/14V/16S/17G/26E/31K/79G/103S/174K/195L/210E/226S/318Q, 13P/14V/16S/17G/26E/31K/79G/174K/195L/210E/226S/232T/318Q, 13P/14V/16S/17G/26E/31K/79G/174K/189R/195L/210E/226S/318Q, 13P/14V/16S/17G/26E/31K/79G/174K/186N/195L/210E/226S/318Q, 13P/14V/16S/17G/26E/31K/79G/155R/174K/195L/210E/226S/318Q, 13P/14V/16S/17G/26E/31K/79G/174K/193A/195L/210E/226S/318Q, 13P/14V/16S/17G/26E/31K/79G/174K/195L/210E/226S/318Q/342A, 13P/14V/16S/17G/26E/31K/79G/174K/195L/196V/210E/226S/318Q, 13P/14V/16S/17G/26E/31K/63S/79G/174K/195L/210E/226S/318Q, 13P/14V/16S/17G/26E/31K/33R/79G/174K/195L/210E/226S/318Q), 13P/14V/16S/17G/26E/31K/79G/174K/195L/210E/226S/318Q/351H, 13P/14V/16S/17G/26E/31K/79G/174K/193G/195L/210E/226S/318Q, 13P/14V/16S/17G/26E/31K/79G/125S/174K/195L/210E/226S/318Q, 13P/14V/16S/17G/26E/31K/79G/174K/195L/210E/226S/314S/318Q, 13P/14V/16S/17G/26E/31K/79G/174K/187R/195L/210E/226S/318Q, 13P/14V/16S/17G/26E/31K/79G/174K/195L/210E/226S/303T/318Q, 13P/14V/16S/17G/26E/31K/79G/103V/174K/195L/210E/226S/318Q, 13P/14V/16S/17G/26E/31K/79G/103Q/174K/195L/210E/226S/318Q, 13P/14V/16S/17G/26E/31K/79G/155V/174K/195L/210E/226S/318Q, 13P/14V/16S/17G/26E/31K/79G/164R/174K/195L/210E/226S/318Q, 13P/14V/16S/17G/26E/31K/79G/153S/174K/195L/210E/226S/318Q, 13P/14V/16S/17G/26E/31K/79G/174K/195L/210E/226S/318Q, 13P/14V/16S/17G/26E/31K/37R/79G/174K/195L/210E/226S/318Q, 13P/14V/16S/17G/26E/31K/79G/174K/195L/210E/226S/318Q/346Q, 13P/14V/16S/17G/26E/31K/79G/174K/195L/210E/226S/318Q/346G, 13P/14V/16S/17G/26E/31K/79G/174K/195L/210E/226S/318Q/351A, 13P/14V/16S/17G/26E/31K/79G/174K/183K/195L/210E/226S/318Q, 13P/14V/16S/17G/19C/26E/31K/79G/174K/195L/210E/226S/318Q, 13P/14V/16S/17G/26E/31K/79G/174K/187L/195L/210E/226S/318Q, 13P/14V/16S/17G/26E/31K/79G/123S/174K/195L/210E/226S/318Q, 13P/14V/16S/17G/26E/31K/79G/85G/174K/195L/210E/226S/318Q, 13P/14V/16S/17G/26E/31K/79G/174K/187S/195L/210E/226S/318Q, 13P/14V/16S/17G/26E/31K/79G/174K/183C/195L/210E/226S/318Q, 13P/14V/16S/17G/26E/31K/79G/123G/174K/195L/210E/226S/318Q, 13P/14V/16S/17G/26E/31K/79G/174K/195L/210E/226S/318Q/350R, 13P/14V/16S/17G/26E/31K/79G/85E/174K/195L/210E/226S/318Q, 13P/14V/16S/17G/26E/31K/79G/174K/195L/205A/210E/226S/318Q, 13P/14V/16S/17G/26E/31K/79G/174K/195L/210E/226S/284R/318Q, 13P/14V/16S/17G/26E/31K/79G/174K/195L/199V/210E/226S/318Q, 13P/14V/16S/17G/26E/31K/79G/174K/195L/210E/226S/318Q/346W, 13P/14V/16S/17G/26E/31K/79G/174K/195L/210E/226S/318Q/343W, 13P/14V/16S/17G/26E/31K/79G/164V/174K/195L/210E/226S/318Q, 13P/14V/16S/17G/26E/31K/79G/174K/195L/200N/210E/226S/318Q, 13P/14V/16S/17G/26E/31K/79G/174K/195L/210E/226S/318Q/342E, 13P/14V/16S/17G/26E/31K/79G/174K/195L/208G/210E/226S/318Q, 13P/14V/16S/17G/26E/31K/79G/169A/174K/195L/210E/226S/318Q, 13P/14V/16S/17G/26E/31K/79G/174K/190G/195L/210E/226S/318Q, 13P/14V/16S/17G/26E/31K/79G/174K/195L/210E/226S/284A/318Q, 13P/14V/16S/17G/26E/31K/79G/174K/195L/199G/210E/226S/318Q, 13P/14V/16S/17G/26E/31K/79G/121W/174K/195L/210E/226S/318Q, or 13P/14V/16S/17G/26E/31K/79G/174K/195L/196S/210E/226S/318Q, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/16/17/31/37/60/79/174/195/210/226, 13/16/17/31/37/60/79/94/162/174/195/210/212/226/318, 13/14/16/17/26/31/37/60/79/162/174/195/210/318, 13/14/16/17/26/31/37/60/79/174/195/210/226/318, 13/16/17/26/31/37/79/125/162/174/195/210/212/226, 13/14/16/17/26/37/60/79/125/174/195/210/318, 13/16/17/26/3/1/7/79/125/162/174/195/210/318, 13/14/16/17/31/37/60/79/162/174/195/210, 13/16/17/31/37/79/94/162/174/195/210/212/318, 13/16/17/26/31/37/60/79/162/174/195/210/226/318, 13/16/17/26/31/37/60/79/162/174/195/210/318, 13/16/17/26/31/37/79/94/162/174/195/210/226, 13/16/17/26/31/37/79/94/162/174/195/210/226/318, 13/16/17/26/31/37/60/79/174/195/210/318, 13/16/17/26/31/37/60/79/94/174/195/210/212/226/318, 13/16/17/26/31/37/60/79/162/174/195/210/212/226/318, 13/16/17/26/31/37/79/162/174/195/210/226/318, 13/16/17/26/31/37/79/94/174/195/210/212/226, 13/16/17/26/31/37/60/79/174/195/210/226/318, 13/16/17/31/37/79/162/174/195/210/226/318, 13/16/17/26/31/37/79/174/195/210/226/318, 13/16/17/26/31/37/79/174/195/210/226, 13/16/17/26/31/37/79/162/174/195/210, 13/16/17/26/31/37/79/125/174/195/210/212/226/318, 13/16/17/26/31/37/79/125/174/195/210, 13/16/17/31/37/60/79/162/174/195/210/226/318, 13/16/17/26/31/37/79/125/162/174/195/210/212/318, 13/16/17/26/31/37/79/94/125/162/174/195/210/226, 13/16/17/26/31/37/79/125/174/195/210/318, 13/14/16/17/26/31/37/79/174/195/210/226/277/318, 13/14/16/17/26/31/37/79/166/174/195/210/226/318, 13/14/16/17/26/31/37/79/174/195/210/226/233/318, 13/14/16/17/26/31/37/79/83/174/195/210/226/318, 5/13/14/16/17/26/31/37/79/174/195/210/226/318, 13/14/16/17/26/31/37/79/174/195/210/226/318/342, 13/14/16/17/26/31/37/79/96/174/195/210/226/318, 13/14/16/17/26/31/37/79/174/195/210/226/284/318, or 13/14/16/17/26/31/37/79/174/195/210/226/318, wherein the amino acid positions are relative to the reference sequence of SEQ ID NO: 2 or 4.

In some embodiments, the amino acid sequence of the engineered oxalate decarboxylase polypeptide comprises at least a substitution or substitution set 13P/16S/17G/31K/37G/60V/79G/174K/195L/210E/226S, 13P/16S/17G/31K/37G/60V/79G/94T/162K/174K/195L/210E/212S/226S/318Q, 13P/14V/16S/17G/26E/31K/37G/60V/79G/162K/174K/195L/210E/318Q, 13P/14V/16S/17G/26E/31K/37G/60V/79G/174K/195L/210E/226S/318Q, 13P/16S/17G/26E/31K/37G/79G/125F/162K/174K/195L/210E/212S/226S, 13P/14V/16S/17G/26E/31K/37G/60V/79G/125F/174K/195L/210E/318Q, 13P/16S/17G/26E/31K/37G/79G/125F/162K/174K/195L/210E/318Q, 13P/14V/16S/17G/31K/37G/60V/79G/162K/174K/195L/210E, 13P/16S/17G/31K/37G/79G/94T/162K/174K/195L/210E/212S/318Q, 13P/16S/17G/26E/31K/37G/60V/79G/162K/174K/195L/210E/226S/318Q, 13P/16S/17G/26E/31K/37G/60V/79G/162K/174K/195L/210E/318Q, 13P/16S/17G/26E/31K/37G/79G/94T/162K/174K/195L/210E/226S, 13P/16S/17G/26E/31K/37G/79G/94T/162K/174K/195L/210E/226S/318Q, 13P/16S/17G/26E/31K/37G/60V/79G/174K/195L/210E/318Q, 13P/16S/17G/26E/31K/37G/60V/79G/94T/174K/195L/210E/212S/226S/318Q, 13P/16S/17G/26E/31K/37G/60V/79G/162K/174K/195L/210E/212S/226S/318Q, 13P/16S/17G/26E/31K/37G/79G/162K/174K/195L/210E/226S/318Q, 13P/16S/17G/26E/31K/37G/79G/94T/174K/195L/210E/212S/226S, 13P/16S/17G/26E/31K/37G/60V/79G/174K/195L/210E/226S/318Q, 13P/16S/17G/31K/37G/79G/162K/174K/195L/210E/226S/318Q, 13P/16S/17G/26E/31K/37G/79G/174K/195L/210E/226S/318Q, 13P/16S/17G/26E/31K/37G/79G/174K/195L/210E/226S, 13P/16S/17G/26E/31K/37G/79G/162K/174K/195L/210E, 13P/16S/17G/26E/31K/37G/79G/125F/174K/195L/210E/212S/226S/318Q, 13P/16S/17G/26E/31K/37G/79G/125F/174K/195L/210E, 13P/16S/17G/31K/37G/60V/79G/162K/174K/195L/210E/226S/318Q, 13P/16S/17G/26E/31K/37G/79G/125F/162K/174K/195L/210E/212S/318Q, 13P/16S/17G/26E/31K/37G/79G/94T/125F/162K/174K/195L/210E/226S, 13P/16S/17G/26E/31K/37G/79G/125F/174K/1951/210E/318Q, 13P/14V/16S/17G/26E/31K/37G/79G/174K/195L/210E/226S/277R/318Q, 13P/14V/16S/17G/26E/31K/37G/79G/174K/195L/210E/226S/277C/318Q, 13P/14V/16S/17G/26E/31K/37G/79G/174K/195L/210E/226S/277T/318Q, 13P/14V/16S/17G/26E/31K/37G/79G/166R/174K/195L/210E/226S/318Q, 13P/14V/16S/17G/26E/31K/37G/79G/174K/195L/210E/226S/233H/318Q, 13P/14V/16S/17G/26E/31K/37G/79G/83R/174K/195L/210E/226S/318Q, 13P/14V/16S/17G/26E/31K/37G/60M/79G/174K/195L/210E/226S/318Q, 13P/14V/16S/17G/26E/31K/37G/79G/174K/195L/210E/226S/277I/318Q, 13P/14V/16S/17G/26E/31K/37G/79G/174K/195L/210E/226S/277M/318Q, 5S/13P/14V/16S/17G/26E/31K/37G/79G/174K/195L/210E/226S/318Q, 13P/14V/16S/17G/26E/31K/37G/79G/174K/195L/210E/226S/318Q/342R, 13P/14V/16S/17G/26E/31K/37G/79G/174K/195L/210E/2265/277V/318Q, 13P/14V/16S/17G/26E/31K/37G/60R/79G/174K/195L/210E/226S/318Q, 13P/14V/16S/17G/26E/31K/37G/79G/96R/174K/195L/210E/226S/318Q, 13P/14V/16S/17G/26E/31K/37G/79G/174K/195L/210E/226S/284S/318Q, 13P/14V/16S/17G/26R/31K/37G/79G/174K/195L/210E/226S/318Q, 13P/14V/16S/17G/26N/31K/37G/79G/174K/195L/210E/226S/318Q, or 13P/14V/16S/17G/26E/31K/37G/79G/83W/174K/195L/210E/2265/318Q, wherein the amino acid positions are relative to the reference sequence of SEQ ID NO: 2 or 4.

In some embodiments, the amino acid sequence of the engineered oxalate decarboxylase polypeptide comprises at least a substitution or substitution set at amino acid position(s) 5/13/16/17/31/37/60/79/83/162/174/195/210/318/342, 5/13/16/17/31/37/60/79/162/174/195/210/277/318, 5/13/16/17/26/31/37/60/79/96/162/174/195/210/277/318, 5/13/16/17/26/31/37/60/79/83/162/174/195/210/277/318/342, 13/16/17/31/37/60/79/83/162/174/195/210/277/318, 5/13/16/17/31/37/60/79/83/162/174/195/210/277/318, 5/13/16/17/26/31/37/60/79/162/174/195/210/277/318/342, 5/13/16/17/31/37/60/79/162/166/174/195/210/277/318, 13/16/17/26/31/37/60/79/83/162/174/195/210/277/318/342, 13/16/17/26/31/37/60/79/83/96/162/174/195/210/277/318/342, 13/16/17/31/37/60/79/83/162/174/195/210/277/318/342, 5/13/16/17/31/37/60/79/83/162/174/195/210/277/318/342, 5/13/16/17/26/31/37/60/79/83/162/174/195/210/277/318, 13/16/17/31/37/60/79/162/174/195/210/277/318, 13/16/17/26/31/37/60/79/83/162/174/195/210/277/318, 5/13/16/17/26/31/35/37/60/79/162/174/195/210/277/318, 5/13/16/17/31/37/60/79/83/96/162/174/195/210/277/318, 5/13/16/17/31/37/60/79/96/162/174/195/210/277/318, 5/13/16/17/26/31/37/60/79/162/174/195/210/277/318, 5/13/16/17/26/31/37/60/79/162/166/174/195/210/277/318, 13/16/17/31/37/60/79/83/96/162/174/195/210/277/318/342, 13/16/17/26/31/37/60/79/162/174/195/210/277/318, 13/16/17/31/37/60/79/162/174/195/210/277/318/342, 5/13/16/17/26/31/37/60/79/162/174/195/210/318, 5/13/16/17/26/31/37/60/79/83/96/162/174/195/210/277/318/342, or 13/16/17/26/31/37/60/79/83/96/162/174/195/210/277/318, wherein the amino acid positions are relative to the reference sequence corresponding to SEQ ID NO: 2 or 4.

In some embodiments, the amino acid sequence of the engineered oxalate decarboxylase comprises at least a substitution or substitution set 5S/13P/16S/17G/31K/37G/60V/79G/83W/162K/174K/195L/210E/318Q/342R, 5S/13P/16S/17G/31K/37G/60V/79G/162K/174K/195L/210E/277T/318Q, 5S/13P/16S/17G/26E/31K/37G/60V/79G/96L/162K/174K/195L/210E/277T/318Q, 5S/13P/16S/17G/26E/31K/37G/60V/79G/83W/162K/174K/195L/210E/277T/318Q/342R, 13P/16S/17G/31K/37G/60V/79G/83W/162K/174K/195L/210E/277T/318Q, 5S/13P/16S/17G/31K/37G/60V/79G/83W/162K/174K/195L/210E/277V/318Q, 5S/13P/16S/17G/26E/31K/37G/60V/79G/162K/174K/195L/210E/277T/318Q/342R, 5S/13P/16S/17G/26E/31K/37G/60V/79G/83W/162K/174K/195L/210E/277V/318Q/342R, 5S/13P/16S/17G/31K/37G/60V/79G/162K/166R/174K/195L/210E/277T/318Q, 13P/16S/17G/26E/31K/37G/60V/79G/83W/162K/174K/195L/210E/277T/318Q/342R, 13P/16S/17G/26E/31K/37G/60V/79G/83R/96L/162K/174K/195L/210E/277V/318Q/342R, 13P/16S/17G/26E/31K/37G/60V/79G/83W/96L/162K/174K/195L/210E/277T/318Q, 13P/16S/17G/31K/37G/60V/79G/83W/162K/174K/195L/210E/277V/318Q/342R, 5S/13P/16S/17G/31K/37G/60V/79G/83W/162K/174K/195L/210E/277V/318Q/342R, 5S/13P/16S/17G/26E/31K/37G/60V/79G/83R/162K/174K/195L/210E/277T/318Q/342R, 5S/13P/16S/17G/31K/37G/60V/79G/83W/162K/174K/195L/210E/277T/318Q/342R, 5S/13P/16S/17G/26E/31K/37G/60V/79G/83R/162K/174K/195L/210E/277V/318Q, 13P/16S/17G/31K/37G/60V/79G/162K/174K/195L/210E/277T/318Q, 13P/16S/17G/26E/31K/37G/60V/79G/83W/162K/174K/195L/210E/277T/318Q, 5S/13P/16S/17G/26E/31K/35V/37G/60V/79G/162K/174K/195L/210E/277T/318Q, 5S/13P/16S/17G/31K/37G/60V/79G/83R/96R/162K/174K/195L/210E/277V/318Q, 5S/13P/16S/17G/31K/37G/60V/79G/96L/162K/174K/195L/210E/277T/318Q, 5S/13P/16S/17G/26E/31K/37G/60V/79G/162K/174K/195L/210E/277T/318Q, 5S/13P/16S/17G/31K/37G/60V/79G/83R/162K/174K/195L/210E/277T/318Q, 5S/13P/16S/17G/26E/31K/37G/60V/79G/83R/162K/174K/195L/210E/277T/318Q, 13P/16S/17G/31K/37G/60V/79G/83R/162K/174K/195L/210E/277V/318Q, 5S/13P/16S/17G/31K/37G/60V/79G/162K/174K/195L/210E/277V/318Q, 5S/13P/16S/17G/26E/31K/37G/60V/79G/162K/166R/174K/195L/210E/277T/318Q, 13P/16S/17G/31K/37G/60V/79G/83W/162K/174K/195L/210E/277V/318Q, 13P/16S/17G/31K/37G/60V/79G/83W/96L/162K/174K/195L/210E/277V/318Q/342R, 13P/16S/17G/26E/31K/37G/60V/79G/162K/174K/195L/210E/277T/318Q, 13P/16S/17G/31K/37G/60V/79G/162K/174K/195L/210E/277T/318Q/342R, 13P/16S/17G/26E/31K/37G/60V/79G/83W/162K/174K/195L/210E/277V/318Q/342R, 13P/16S/17G/26E/31K/37G/60V/79G/83R/162K/174K/195L/210E/277V/318Q, 5S/13P/16S/17G/26E/31K/37G/60V/79G/162K/174K/195L/210E/318Q, 13P/16S/17G/31K/37G/60V/79G/162K/174K/195L/210E/277V/318Q, 13P/16S/17G/26E/31K/37G/60V/79G/83R/162K/174K/195L/210E/277T/318Q, 5S/13P/16S/17G/26E/31K/37G/60V/79G/83W/96R/162K/174K/195L/210E/277T/318Q/342R, 5S/13P/16S/17G/26E/31K/37G/60V/79G/162K/174K/195L/210E/277V/318Q, 13P/16S/17G/26E/31K/37G/60V/79G/162K/174K/195L/210E/277V/318Q, 5S/13P/16S/17G/26E/31K/37G/60V/79G/162K/174K/195L/210E/277V/318Q/342R, 13P/16S/17G/26E/31K/37G/60V/79G/83W/96L/162K/174K/195L/210E/277V/318Q, or 13P/16S/17G/26E/31K/37G/60V/79G/83W/162K/174K/195L/210E/277V/318Q, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) 13/16/17/26/31/37/60/79/83/96/162/174/195/210/277/318, 13/16/17/26/31/37/60/79/83/96/162/174/210/277/318, 13/16/17/3⅓7/60/79/83/96/162/174/195/210/277/318, 13/16/17/26/31/37/60/79/83/162/174/195/210/277/318, 13/16/17/26/31/37/60/79/83/96/162/195/210/277/318, 13/16/17/26/37/60/79/83/96/162/174/195/210/277/318, 16/17/26/31/37/60/79/83/96/162/174/195/210/277/318, 13/16/17/26/31/37/60/79/96/162/174/195/210/277/318, 13/16/17/26/31/37/60/79/83/96/162/174/195/277/318, 13/16/17/26/31/37/79/83/96/162/174/195/210/277/318, 13/17/26/31/37/60/79/83/96/162/174/195/210/277/318, 13/16/17/26/31/37/60/83/96/162/174/195/210/277/318, 13/16/26/31/37/60/79/83/96/162/174/195/210/277/318, 13/16/17/26/31/37/60/79/83/96/162/174/195/210/277, 13/16/17/26/31/37/60/79/83/96/162/174/195/210/318, 13/16/17/26/31/60/79/83/96/162/174/195/210/277/318, or 13/16/17/26/31/37/60/79/83/96/174/195/210/277/318, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set 13P/16S/17G/26E/31K/37G/60V/79G/83W/96L/162K/174K/195L/210E/277V/318Q, 13P/16S/17G/26L/31K/37G/60V/T9G/83W/96L/162K/174K/195L/210E/277V/318Q, 13P/16S/17G/26E/31W/37G/60V/79G/83W/96L/162K/174K/195L/210E/277V/318Q, 13P/16S/17G/26E/31K/37G/60V/79G/83W/96K/162K/174K/195L/210E/277V/318Q, 13P/16S/17G/26E/31K/37G/60R/79G/83W/96L/162K/174K/195L/210E/277V/318Q, 13P/16S/17G/26E/31K/37G/60V/79G/83W/96L/162K/174K/195L/210E/277V/318D, 13P/16S/17G/26G/31K/37G/60V/79G/83W/96L/162K/174K/195L/210E/277V/318Q, 13P/16S/17G/26E/31K/37G/60V/79G/83W/96L/162K/174K/195L/210R/277V/318Q, 13P/16S/17G/26E/31R/37G/60V/79G/83W/96L/162K/174K/195L/210E/277V/318Q, 13P/16S/17G/26E/31K/37G/60V/79G/83W/96L/162K/174K/195L/210E/277F/318Q, 13P/16S/17G/26E/31K/37M/60V/79G/83W/96L/162K/174K/195L/210E/277V/318Q, 13P/16S/17G/26E/31K/37G/60V/79G/83W/96P/162K/174K/195L/210E/277V/318Q, 13P/16V/17G/26E/31K/37G/60V/79G/83W/96L/162K/174K/195L/210E/277V/318Q, 13P/16S/17G/26E/31K/37G/60A/79G/83W/96L/162K/174K/195L/210E/277V/318Q, 13P/16S/17G/26R/31K/37G/60V/79G/83W/96L/162K/174K/195L/210E/277V/318Q, 13P/16S/17G/26E/31G/37G/60V/79G/83W/96L/162K/174K/195L/210E/277V/318Q, 13P/16S/17G/26E/31K/37G/60V/79G/83W/96L/162K/174K/195G/210E/277V/318Q, 13P/16S/17G/26E/31K/37G/60V/79G/83W/96L/162K/174K/195L/210E/277V/318R, 13P/16S/17G/26V/31K/37G/60V/79G/83W/96L/162K/174K/195L/210E/277V/318Q, 13P/16S/17G/26E/31K/37G/60V/79M/83W/96L/162K/174K/195L/210E/277V/318Q, 13P/16M/17G/26E/31K/37G/60V/79G/83W/96L/162K/174K/195L/210E/277V/318Q, 13P/16S/17G/26E/31K/37G/60V/79G/83W/96L/162K/174K/195L/210A/277V/318Q, 13P/16S/17G/26E/31K/37G/60V/79G/83W/96R/162K/174K/195L/210E/277V/318Q, 13P/16S/17G/26E/31K/37E/60V/79G/83W/96L/162K/174K/195L/210E/277V/318Q, 13P/16G/17G/26E/31K/37G/60V/79G/83W/96L/162K/174K/195L/210E/277V/318Q, 13P/16S/17W/26E/31K/37G/60V/79G/83W/96L/162K/174K/195L/210E/277V/318Q, 13G/16S/17G/26E/31K/37G/60V/79G/83W/96L/162K/174K/195L/210E/277V/318Q, 13P/16S/17G/26E/31K/37G/60V/79E/83W/96L/162K/174K/195L/210E/277V/318Q, 13P/16S/17G/26E/31K/37G/60V/79G/83W/96L/162K/174K/195L/210E/277T/318Q, 13P/16S/17G/26E/31K/37G/60Q/79G/83W/96L/162K/174K/195L/210E/277V/318Q, 13P/16S/17G/26E/31L/37G/60V/79G/83W/96L/162K/174K/195L/210E/277V/318Q, 13P/16S/17G/26E/31K/37G/60V/79G/83W/96G/162K/174K/195L/210E/277V/318Q, 13P/16S/17G/26E/31K/37G/60V/79G/83W/96T/162K/174K/195L/210E/277V/318Q, 13P/16S/17G/26E/31K/37G/60G/79G/83W/96L/162K/174K/195L/210E/277V/318Q, 13P/16W/17G/26E/31K/37G/60V/79G/83W/96L/162K/174K/195L/210E/277V/318Q, 13T/16S/17G/26E/31K/37G/60V/79G/83W/96L/162K/174K/195L/210E/277V/318Q, 13P/16S/17G/26E/31K/37G/60V/79R/83W/96L/162K/174K/195L/210E/277V/318Q, 13P/16S/17G/26Y/31K/37G/60V/79G/83W/96L/162K/174K/195L/210E/277V/318Q, 13P/16S/17G/26S/31K/37G/60V/79G/83W/96L/162K/174K/195L/210E/277V/318Q, 13P/16S/17G/26E/31K/37G/60V/79G/83W/96L/162K/174K/210E/277V/318Q, 13P/16S/17R/26E/31K/37G/60V/79G/83W/96L/162K/174K/195L/210E/277V/318Q, 13V/16S/17G/26E/31K/37G/60V/79G/83W/96L/162K/174K/195L/210E/277V/318Q, 13P/16S/17G/26E/31K/37G/60V/79G/83W/96L/162K/174K/195L/210F/277F/318Q, 13P/16S/17G/26E/31K/37L/60V/79G/83W/96L/162K/174K/195L/210E/277V/318Q, 13P/16S/17G/26E/31K/37G/60V/79G/83W/96L/162K/174K/195L/210G/277V/318Q, 13P/16S/17G/26E/31K/37G/60L/79G/83W/96L/162K/174K/195L/210E/277V/318Q, 13L/16S/17G/26E/31K/37G/60V/79G/83W/96L/162K/174K/195L/210E/277V/318Q, 13P/16S/17G/26E/31K/37G/60V/79G/83Y/96L/162K/174K/195L/210E/277V/318Q, 13P/16S/17G/26E/31K/37W/60V/79G/83W/96L/162K/174K/195L/210E/277V/318Q, 13P/16S/17G/26E/31K/37G/60V/79G/83W/96L/162K/174K/195L/210E/277G/318Q, 13P/16S/17G/26E/31K/37G/60V/79G/83R/96L/162K/174K/195L/210E/277V/318Q, 13P/16S/17G/26E/31K/37G/60Y/79G/83W/96L/162K/174K/195L/210E/277V/318Q, 13P/16S/17G/26E/31K/37G/60V/79G/83W/96L/162K/174K/195L/210S/277V/318Q, 13P/16S/17G/26E/31K/37G/60V/79G/83W/96L/162K/174K/195L/210E/277V/318S, 13P/16S/17I/26E/31K/37G/60V/79G/83W/96L/162K/174K/195L/210E/277V/318Q, 13P/16S/17G/26E/31K/37S/60V/79G/83W/96L/162K/174K/195L/210E/277V/318Q, 13P/16S/17G/26E/31K/37G/60V/79L/83W/96L/162K/174K/195L/210E/277V/318Q, 13P/16R/17G/26E/31K/37G/60V/79G/83W/96L/162K/174K/195L/210E/277V/318Q, 13P/16S/17G/26E/31K/37G/60V/79G/83W/96L/162K/174K/195S/210E/277V/318Q, 13P/16S/17G/26E/31K/37G/60V/79G/83W/96L/162K/174K/195L/210E/277V/318G, 13P/16S/17G/26C/31K/37G/60V/79G/83W/96L/162K/174K/195L/210E/277V/318Q, 13R/16S/17G/26E/31K/37G/60V/79G/83W/96L/162K/174K/195L/210E/277V/318Q, 13P/16S/17G/26E/31K/37G/60V/79G/83W/96L/162K/174K/195Y/210E/277V/318Q, 13P/16S/17G/26E/31K/37G/60E/79G/83W/96L/162K/174K/195L/210E/277V/318Q, 13P/16S/17G/26E/31K/37G/60V/79G/83W/96L/162V/174K/195L/210E/277V/318Q, 13P/16C/17G/26E/31K/37G/60V/79G/83W/96L/162K/174K/195L/210E/277V/318Q, 13P/16S/17G/26E/31K/37G/60V/79G/83W/96V/162K/174K/195L/210E/277V/318Q, 13P/16S/17G/26E/31K/37G/60V/79G/83M/96L/162K/174K/195L/210E/277V/318Q, 13P/16S/17G/26E/31M/37G/60V/79G/83W/96L/162K/174K/195L/210E/277V/318Q, 13P/16S/17G/26E/31K/37G/60V/79G/83W/96L/162K/174V/195L/210E/277V/318Q, 13P/16S/17G/26E/31K/37G/60V/79G/83W/96L/162K/174K/195L/210E/277L/318Q, 13P/16S/17G/26M/31K/37G/60V/79G/83W/96L/162K/174K/195L/210E/277V/318Q, 13P/16S/17G/26E/31K/37G/60V/79G/83W/96L/162K/174K/195L/210E/277V/318M, 13P/16S/17G/26E/31K/37G/60V/79G/83W/96L/162K/174K/195L/210N/277V/318Q, 13P/16S/17G/31K/37G/60V/79G/83W/96L/162K/174K/195L/210E/277V/318Q, 13P/16S/17G/26E/31K/37G/60V/79G/83W/96L/162K/174K/195L/210E/277R/318Q, 13P/16S/17G/26E/31K/37G/60V/79G/83W/96F/162K/174K/195L/210E/277V/318Q, 13P/16S/17G/26E/31K/37G/60V/79G/83W/96L/162K/174G/195L/210E/277V/318Q, 13P/16Q/17G/26E/31K/37G/60V/79G/83W/96L/162K/174K/195L/210E/277V/318Q, 13P/16S/17G/26E/31K/37G/60V/79G/83W/96L/162K/174K/195L/210E/277A/318Q, 13Q/16S/17G/26E/31K/37G/60V/79G/83W/96L/162K/174K/195L/210E/277V/318Q, 13P/16S/17G/26E/31K/37G/60V/79F/83W/96L/162K/174K/195L/210E/277V/318Q, 13P/16L/17G/26E/31K/37G/60V/79G/83W/96L/162K/174K/195L/210E/277V/318Q, 13P/16S/17G/26E/31K/37G/60V/79G/83W/96L/162K/174K/195N/210E/277V/318Q, 13P/16S/17G/26E/31K/37G/60V/79V/83W/96L/162K/174K/195L/210E/277V/318Q, 13P/16S/17P/26E/31K/37G/60V/79G/83W/96L/162K/174K/195L/210E/277V/318Q, 13P/16S/17G/26E/31K/37G/60P/79G/83W/96L/162K/174K/195L/210E/277V/318Q, 13P/16S/17G/26E/31K/37P/60V/79G/83W/96L/162K/174K/195L/210E/277V/318Q, 13P/16S/17G/26E/31K/37G/60V/79G/83W/96W/162K/174K/195L/210E/277V/318Q, 13P/16S/17G/26E/31K/37G/60V/79G/83W/96L/162T/174K/195L/210E/277V/318Q, 13P/16S/17G/26E/31K/37G/60V/79G/83W/96L/162K/174K/195M/210E/277V/318Q, 13P/16S/17G/26E/31K/37G/60V/79G/83W/96L/162K/174K/195L/210E/277V/318W, 13P/16S/17G/26E/31K/37R/60V/79G/83W/96L/162K/174K/195L/210E/277V/318Q, 13P/16T/17G/26E/31K/37G/60V/79G/83W/96L/162K/174K/195L/210E/277V/318Q, 13P/16S/17G/26E/31K/37G/60V/79G/83W/96L/162K/174K/195L/210M/277V/318Q, 13P/16S/17G/26E/31K/37G/60V/79G/83W/96L/162Y/174K/195L/210E/277V/318Q, 13P/16P/17G/26E/31K/37G/60V/79G/83W/96L/162K/174K/195L/210E/277V/318Q, 13P/16S/17G/26E/31K/37G/60V/79G/83E/96L/162K/174K/195L/210E/277V/318Q, 13P/16S/17G/26E/31K/37G/60V/79G/83W/96L/162K/174K/195L/210E/277M/318Q, 13P/16S/17G/26E/31K/37G/60V/79G/83W/96L/162K/174L/195L/210E/277V/318Q, 13P/16S/17V/26E/31K/37G/60V/79G/83W/96L/162K/174K/195L/210E/277V/318Q, 13P/16S/17E/26E/31K/37G/60V/79G/83W/96L/162K/174K/195L/210E/277V/318Q, 13P/16S/17L/26E/31K/37G/60V/79G/83W/96L/162K/174K/195L/210E/277V/318Q, 13P/16S/17G/26E/31K/37G/60V/79G/83W/96M/162K/174K/195L/210E/277V/318Q, 13P/16S/17G/26E/31K/37G/60V/79G/83W/96L/162K/174K/195L/210E/277V/318V, 13P/16S/17G/26E/31K/37G/60V/79G/83W/96L/162K/174R/195L/210E/277V/318Q, 13S/16S/17G/26E/31K/37G/60V/79G/83W/96L/162K/174K/195L/210E/277V/318Q, 13P/16S/17G/26E/31K/37G/60V/79G/83W/96L/162K/174T/195L/210E/277V/318Q, 13Y/16S/17G/26E/31K/37G/60V/79G/83W/96L/162K/174K/195L/210E/277V/318Q, 13P/16S/17G/26E/31K/37G/60V/79G/83W/96L/162K/174K/195A/210E/277V/318Q, 13P/16S/17G/26E/31K/37G/60V/79G/83W/96L/162K/174K/195L/210E/277W/318Q, 13P/16S/17G/26E/31K/37G/60M/79G/83W/96L/162K/174K/195L/210E/277V/318Q, 13P/16S/17A/26E/31K/37G/60V/79G/83W/96L/162K/174K/195L/210E/277V/318Q, 13P/16S/17G/26E/31K/37G/60V/79G/83W/96L/162K/174K/195L/210E/277V/318A, 13P/16S/17G/26E/31K/37G/60V/79G/83W/96L/162K/174H/195L/210E/277V/318Q, 13P/16S/17G/26E/31K/37G/60V/79G/83S/96L/162K/174K/195L/210E/277V/318Q, 13P/16S/17G/26E/31K/37G/60V/79G/83W/96L/162K/174K/195L/210E/277I/318Q, 13E/16S/17G/26E/31K/37G/60V/79G/83W/96L/162K/174K/195L/210E/277V/318Q, 13P/16S/17K/26E/31K/37G/60V/79G/83W/96L/162K/174K/195L/210E/277V/318Q, 13P/16S/17G/26K/31K/37G/60V/79G/83W/96L/162K/174K/195L/210E/277V/318Q, 13P/16S/17G/26E/31K/37G/60V/79G/83W/162K/174K/195L/210E/277V/318Q, 13P/16S/17G/26E/31K/37G/60V/79G/83W/96L/162K/195L/210E/277V/318Q, 13P/16S/17G/26E/31K/37G/60V/79G/83W/96L/162K/174K/195L/210V/277V/318Q, 13P/16S/17G/26E/31S/37G/60V/79G/83W/96L/162K/174K/195L/210E/277V/318Q, 13P/16S/17G/26E/31K/37G/60V/79G/83W/96L/162K/174K/195C/210E/277V/318Q, 13P/16S/17G/26E/31K/37G/60V/79K/83W/96L/162K/174K/195L/210E/277V/318Q, 13P/16S/17G/26E/31K/37G/60W/79G/83W/96L/162K/174K/195L/210E/277V/318Q, 13P/16S/17G/26E/31K/37G/60V/79G/83W/96L/162K/174S/195L/210E/277V/318Q, 13I/16S/17G/26E/31K/37G/60V/79G/83W/96L/162K/174K/195L/210E/277V/318Q, 13P/16S/17G/26E/31K/37G/60H/79G/83W/96L/162K/174K/195L/210E/277V/318Q, 13P/16S/17G/26E/37G/60V/79G/83W/96L/162K/174K/195L/210E/277V/318Q, 13P/16S/17G/26E/31K/37G/60V/79G/83W/96E/162K/174K/195L/210E/277V/318Q, 13P/16S/17G/26E/31K/37G/60V/79G/83K/96L/162K/174K/195L/210E/277V/318Q, 13P/16S/17G/26E/31Q/37G/60V/79G/83W/96L/162K/174K/195L/210E/277V/318Q, 13P/16K/17G/26E/31K/37G/60V/79G/83W/96L/162K/174K/195L/210E/277V/318Q, 13P/16S/17G/26E/31K/37G/60V/79G/83W/96L/162K/174M/195L/210E/277V/318Q, 13P/16S/17G/26E/31K/37V/60V/79G/83W/96L/162K/174K/195L/210E/277V/318Q, 13P/16S/17G/26E/31K/37G/60V/79D/83W/96L/162K/174K/195L/210E/277V/318Q, 13P/16S/17G/26E/31K/37G/60V/79G/83W/96L/162K/174K/195L/210E/277V/318T, 13P/16S/17G/26E/31K/37G/60V/79G/83W/96L/162K/174K/195F/210E/277V/318Q, 13P/16S/17G/26T/31K/37G/60V/79G/83W/96L/162K/174K/195L/210E/277V/318Q, 13P/16S/17G/26E/31K/37G/60V/79G/83W/96L/162K/174Q/195L/210E/277V/318Q, 13P/16S/17G/26E/31K/37G/60V/79G/83L/96L/162K/174K/195L/210E/277V/318Q, 13P/16S/17G/26E/31K/37G/60V/79G/83W/96I/162K/174K/195L/210E/277V/318Q, 13C/16S/17G/26E/31K/37G/60V/79G/83W/96L/162K/174K/195L/210E/277V/318Q, 13A/16S/17G/26E/31K/37G/60V/79G/83W/96L/162K/174K/195L/210E/277V/318Q, 13P/16S/17G/26E/31K/37G/60V/79G/83W/96Q/162K/174K/195L/210E/277V/318Q, 16S/17G/26E/31K/37G/60V/79G/83W/96L/162K/174K/195L/210E/277V/318Q, 13P/16S/17G/26E/31K/37G/60V/79G/96L/162K/174K/195L/210E/277V/318Q, 13P/16S/17G/26E/31Y/37G/60V/79G/83W/96L/162K/174K/195L/210E/277V/318Q, 13W/16S/17G/26E/31K/37G/60V/79G/83W/96L/162K/174K/195L/210E/277V/318Q, 13P/16S/17G/26E/31K/37G/60V/79G/83W/96L/162K/174K/195L/210E/277P/318Q, 13P/16S/17G/26E/31K/37G/60V/79G/83W/96L/162K/174K/195T/210E/277V/318Q, 13P/16S/17G/26E/31K/37G/60S/79G/83W/96L/162K/174K/195L/210E/277V/318Q, 13P/16S/17G/26Q/31K/37G/60V/79G/83W/96L/162K/174K/195L/210E/277V/318Q, 13P/16S/17G/26E/31K/37G/60V/79G/83W/96L/162K/174K/195L/277V/318Q, 13P/16S/17G/26E/31K/37G/60V/79G/83W/96L/162K/174N/195L/210E/277V/318Q, 13P/16S/17G/26E/31K/37G/60V/79G/83A/96L/162K/174K/195L/210E/277V/318Q, 13P/16S/17G/26E/31K/37G/60V/79G/83W/96L/162E/174K/195L/210E/277V/318Q, 13P/16S/17G/26E/31K/37F/60V/79G/83W/96L/162K/174K/195L/210E/277V/318Q, 13P/16S/17G/26E/31K/37Q/60V/79G/83W/96L/162K/174K/195L/210E/277V/318Q, 13P/16S/17G/26E/31T/37G/60V/79G/83W/96L/162K/174K/195L/210E/277V/318Q, 13P/16S/17G/26E/31K/37G/60V/79G/83W/96L/162K/174A/195L/210E/277V/318Q, 13P/16S/17G/26E/31K/37G/60V/79G/83F/96L/162K/174K/195L/210E/277V/318Q, 13P/16S/17G/26E/31K/37G/79G/83W/96L/162K/174K/195L/210E/277V/318Q, 13P/16S/17G/26E/31K/37G/60V/79G/83W/96S/162K/174K/195L/210E/277V/318Q, 13P/16S/17G/26E/31K/37G/60V/79G/83V/96L/162K/174K/195L/210E/277V/318Q, 13P/16S/17G/26E/31K/37G/60V/79G/83W/96L/162K/174K/195L/210I/277V/318Q, 13P/16D/17G/26E/31K/37G/60V/79G/83W/96L/162K/174K/195L/210E/277V/318Q, 13P/16S/17G/26E/31K/37C/60V/79G/83W/96L/162K/174K/195L/210E/277V/318Q, 13P/16S/17G/26E/31K/37G/60V/79G/83G/96L/162K/174K/195L/210E/277V/318Q, 13N/16S/17G/26E/31K/37G/60V/79G/83W/96L/162K/174K/195L/210E/277V/318Q, 13P/16S/17G/26E/31V/37G/60V/79G/83W/96L/162K/174K/195L/210E/277V/318Q, 13P/16S/17G/26E/31K/37G/60V/79S/83W/96L/162K/174K/195L/210E/277V/318Q, 13P/16S/17G/26E/31K/37G/60V/79G/83W/96Y/162K/174K/195L/210E/277V/318Q, 13P/16S/17F/26E/31K/37G/60V/79G/83W/96L/162K/174K/195L/210E/277V/318Q, 13P/16S/17M/26E/31K/37G/60V/79G/83W/96L/162K/174K/195L/210E/277V/318Q, 13P/17G/26E/31K/37G/60V/79G/83W/96L/162K/174K/195L/210E/277V/318Q, 13P/16S/17G/26E/31K/37G/60V/79G/83P/96L/162K/174K/195L/210E/277V/318Q, 13P/16S/17G/26E/31K/37G/60V/79G/83W/96L/162K/174K/195L/210E/277V/318L, 13P/16S/17G/26E/31K/37G/60V/79Q/83W/96L/162K/174K/195L/210E/277V/318Q, 13P/16S/17S/26E/31K/37G/60V/79G/83W/96L/162K/174K/195L/210E/277V/318Q, 13P/16S/17G/26W/31K/37G/60V/79G/83W/96L/162K/174K/195L/210E/277V/318Q, 13P/16S/17G/26E/31K/37G/60V/79G/83W/96L/162K/174K/195D/210E/277V/318Q, 13P/16S/17G/26E/31K/37G/60V/79G/83W/96L/162K/174K/195W/210E/277V/318Q, 13P/16S/17G/26E/31K/37G/60V/79G/83W/96L/162K/174K/195L/210E/277S/318Q, 13P/16S/17G/26E/31K/37G/60V/79G/83W/96L/162K/174E/195L/210E/277V/318Q, 13P/16E/17G/26E/31K/37G/60V/79G/83W/96L/162K/174K/195L/210E/277V/318Q, 13P/16S/17G/26E/31K/37G/60V/79G/83W/96L/162F/174K/195L/210E/277V/318Q, 13P/16S/17G/26E/31K/37G/60V/79Y/83W/96L/162K/174K/195L/210E/277V/318Q, 13P/16S/17G/26E/31K/37G/60V/83W/96L/162K/174K/195L/210E/277V/318Q, 13P/16S/17C/26E/31K/37G/60V/79G/83W/96L/162K/174K/195L/210E/277V/318Q, 13P/16S/17Y/26E/31K/37G/60V/79G/83W/96L/162K/174K/195L/210E/277V/318Q, 13P/16S/17G/26E/31K/37G/60V/79G/83W/96L/162K/174K/195Q/210E/277V/318Q, 13P/16S/17G/26P/31K/37G/60V/79G/83W/96L/162K/174K/195L/210E/277V/318Q, 13P/16S/17G/26E/31K/37G/60V/79G/83W/96L/162K/174K/195I/210E/277V/318Q, 13P/16S/17G/26E/31K/37G/60F/79G/83W/96L/162K/174K/195L/210E/277V/318Q, 13P/16S/17G/26I/31K/37G/60V/79G/83W/96L/162K/174K/195L/210E/277V/318Q, 13P/16S/26E/31K/37G/60V/79G/83W/96L/162K/174K/195L/210E/277V/318Q, 13P/16S/17G/26E/31K/37G/60V/79G/83W/96L/162K/174K/195L/210E/277V/318K, 13P/16S/17G/26E/31K/37G/60N/79G/83W/96L/162K/174K/195L/210E/277V/318Q, 13P/16I/17G/26E/31K/37G/60V/79G/83W/96L/162K/174K/195L/210E/277V/318Q, 13P/16S/17G/26E/31K/37G/60V/79G/83W/96L/162K/174K/195L/210E/277V/318H, 13P/16S/17G/26E/31K/37A/60V/79G/83W/96L/162K/174K/195L/210E/277V/318Q, 13P/16S/17G/26E/31K/37G/60V/79G/83W/96L/162K/174K/195V/210E/277V/318Q, 13P/16S/17G/26E/31K/37G/60V/79G/83W/96L/162K/174K/195L/210Y/277V/318Q, 13P/16S/17G/26E/31K/37G/60V/79G/83W/96L/162K/174K/195E/210E/277V/318Q, 13P/16S/17G/26H/31K/37G/60V/79G/83W/96L/162K/174K/195L/210E/277V/318Q, 13D/16S/17G/26E/31K/37G/60V/79G/83W/96L/162K/174K/195L/210E/277V/318Q, 13P/16S/17G/26E/31P/37G/60V/79G/83W/96L/162K/174K/195L/210E/277V/318Q, 13P/16S/17G/26E/31K/37G/60V/79G/83W/96L/162K/174K/195L/210W/277V/318Q, 13P/16S/17G/26E/31K/37G/60V/79G/83W/96L/162K/174K/195L/210E/277V/318P, 13P/16S/17G/26E/31K/37G/60V/79G/83C/96L/162K/174K/195L/210E/277V/318Q, 13P/16S/17G/26E/31K/37G/60V/79G/83W/96L/162K/174P/195L/210E/277V/318Q, 13P/16S/17G/26E/31K/37G/60V/79G/83W/96L/162K/174K/195L/210T/277V/318Q, 13P/16S/17T/26E/31K/37G/60V/79G/83W/96L/162K/174K/195L/210E/277V/318Q, 13P/16S/17G/26E/31K/37G/60V/79T/83W/96L/162K/174K/195L/210E/277V/318Q, 13P/16S/17G/26E/31K/37G/60V/79G/83W/96H/162K/174K/195L/210E/277V/318Q, 13P/16S/17G/26E/31K/37G/60V/79W/83W/96L/162K/174K/195L/210E/277V/318Q, 13P/16S/17G/26E/31K/37G/60V/79G/83W/96L/162L/174K/195L/210E/277V/318Q, 13P/16S/17G/26E/31K/37G/60V/79G/83W/96L/162K/174K/195L/210E/277V, 13P/16S/17G/26E/31A/37G/60V/79G/83W/96L/162K/174K/195L/210E/277V/318Q, 13P/16S/17G/26E/31K/37G/60V/79G/83W/96L/162K/174K/195L/210C/277V/318Q, 13P/16S/17N/26E/31K/37G/60V/79G/83W/96L/162K/174K/195L/210E/277V/318Q, 13P/16S/17G/26E/31K/37G/60V/79G/83W/96L/162K/174K/195K/210E/277V/318Q, 13P/16S/17G/26E/31K/37G/60V/79G/83W/96L/162K/174K/195L/210E/277V/318Y, 13P/16S/17H/26E/31K/37G/60V/79G/83W/96L/162K/174K/195L/210E/277V/318Q, 13M/16S/17G/26E/31K/37G/60V/79G/83W/96L/162K/174K/195L/210E/277V/318Q, 13P/16S/17G/26E/31K/37G/60D/79G/83W/96L/162K/174K/195L/210E/277V/318Q, 13P/16S/17G/26E/31K/37G/60V/79G/83W/96L/162K/174K/195L/210E/277K/318Q, 13P/16N/17G/26E/31K/37G/60V/79G/83W/96L/162K/174K/195L/210E/277V/318Q, 13P/16S/17G/26E/31K/37G/60V/79C/83W/96L/162K/174K/195L/210E/277V/318Q, 13K/165/17G/26E/31K/37G/60V/79G/83W/96L/162K/174K/195L/210E/277V/318Q, 13P/16S/17G/26E/31K/37G/60K/79G/83W/96L/162K/174K/195L/210E/277V/318Q, 13P/16S/17G/26E/31K/37N/60V/79G/83W/96L/162K/174K/195L/210E/277V/318Q, 13P/16S/17G/26E/31K/37G/60I/79G/83W/96L/162K/174K/195L/210E/277V/318Q, 13P/16S/17G/26E/31K/37G/60V/79G/83W/96L/162D/174K/195L/210E/277V/318Q, 13P/16S/17G/26E/31K/37G/60V/79G/83W/96L/162I/174K/195L/210E/277V/318Q, 13P/16Y/17G/26E/31K/37G/60V/79G/83W/96L/162K/174K/195L/210E/277V/318Q, 13P/16S/17G/26E/31K/37G/60V/79G/83W/96L/162S/174K/195L/210E/277V/318Q, 13P/16S/17G/26E/31K/37G/60V/79G/83W/96C/162K/174K/195L/210E/277V/318Q, 13P/16S/17G/26E/31K/37G/60V/79G/83W/96L/162K/174K/195L/210K/277V/318Q, 13P/16S/17G/26E/31K/37G/60V/79G/83W/96L/162K/174I/195L/210E/277V/318Q, 13P/16S/17G/26E/31K/37G/60V/79G/83W/96L/162K/174K/195L/210L/277V/318Q, 13P/16S/17G/26D/31K/37G/60V/79G/83W/96L/162K/174K/195L/210E/277V/318Q, 13P/16S/17G/26N/31K/37G/60V/79G/83W/96L/162K/174K/195L/210E/277V/318Q, 13P/16S/17G/26E/31K/37G/60V/79G/83W/96L/162K/174K/195L/210D/277V/318Q, 13F/16S/17G/26E/31K/37G/60V/79G/83W/96L/162K/174K/195L/210E/277V/318Q, 13P/16S/17G/26E/31K/37G/60V/79G/83W/96L/162K/174K/195L/210E/277V/318N, 13P/16S/17G/26E/31K/37G/60V/79G/83W/96L/162K/174K/195L/210E/318Q, 13P/16S/17G/26E/31K/37G/60V/79G/83W/96L/162K/174W/195L/210E/277V/318Q, 13P/16S/17G/26E/31H/37G/60V/79G/83W/96L/162K/174K/195L/210E/277V/318Q, 13P/16S/17G/26E/31D/37G/60V/79G/83W/96L/162K/174K/195L/210E/277V/318Q, 13P/16S/17G/26E/31K/37G/60V/79N/83W/96L/162K/174K/195L/210E/277V/318Q, 13P/16S/17G/26E/31K/60V/79G/83W/96L/162K/174K/195L/210E/277V/318Q, 13P/16S/17G/26E/31I/37G/60V/79G/83W/96L/162K/174K/195L/210E/277V/318Q, 13P/16S/17G/26E/31K/37H/60V/79G/83W/96L/162K/174K/195L/210E/277V/318Q, 13P/16S/17G/26E/31K/37G/60V/79G/83W/96L/162K/174K/195L/210E/277V/318C, 13P/16S/17G/26E/31F/37G/60V/79G/83W/96L/162K/174K/195L/210E/277V/318Q, 13P/16S/17G/26E/31K/37G/60V/79P/83W/96L/162K/174K/195L/210E/277V/318Q, 13P/16S/17G/26E/31K/37G/60V/79G/83W/96L/162K/174K/195P/210E/277V/318Q, 13P/16S/17G/26E/31K/37G/60V/79G/83W/96L/162K/174K/195L/210E/277V/318I, 13P/16S/17G/26E/31K/37G/60V/79G/83W/96L/162A/174K/195L/210E/277V/318Q, 13P/16S/17G/26E/31K/37G/60V/79G/83W/96L/162K/174K/195L/210E/277Q/318Q, 13P/16S/17G/26F/31K/37G/60V/79G/83W/96L/162K/174K/195L/210E/277V/318Q, 13P/16S/17G/26E/31K/37G/60V/79G/83W/96L/162K/174K/195L/210E/277C/318Q, 13P/16S/17G/26E/31K/37G/60V/79H/83W/96L/162K/174K/195L/210E/277V/318Q, 13P/16S/17G/26E/31C/37G/60V/79G/83W/96L/162K/174K/195L/210E/277V/318Q, 13P/16S/17G/26E/31K/37G/60V/79I/83W/96L/162K/174K/195L/210E/277V/318Q, 13P/16S/17G/26E/31K/37I/60V/79G/83W/96L/162K/174K/195L/210E/277V/318Q, 13P/16S/17Q/26E/31K/37G/60V/79G/83W/96L/162K/174K/195L/210E/277V/318Q, 13P/16S/17G/26E/31N/37G/60V/79G/83W/96L/162K/174K/195L/210E/277V/318Q, 13P/16S/17G/26E/31K/37G/60V/79G/83W/96L/162K/174K/195L/210E/277N/318Q, 13P/16S/17G/26E/31K/37D/60V/79G/83W/96L/162K/174K/195L/210E/277V/318Q, 13P/16S/17G/26E/31K/37G/60V/79G/83W/96L/162K/174K/195L/210E/277H/318Q, 13P/16S/17G/26E/31K/37G/60V/79G/83W/96L/162K/174K/195L/210E/277D/318Q, 13P/16S/17G/26E/31K/37G/60V/79G/83W/96D/162K/174K/195L/210E/277V/318Q, 13P/16S/17G/26E/31K/37G/60V/79G/83W/96L/162K/174C/195L/210E/277V/318Q, 13P/16S/17G/26E/31K/37G/60V/79G/83W/96L/162P/174K/195L/210F/277V/318Q, 13P/16S/17G/26E/31K/37G/60V/79G/83W/96L/162K/174K/195L/210E/277V/318F, 13P/16S/17G/26E/31K/37G/60V/79G/83N/96L/162K/174K/195L/210E/277V/318Q, 13P/16S/17G/26E/31K/37G/60C/79G/83W/96L/162K/174K/195L/210E/277V/318Q, 13P/16S/17G/26E/31K/37G/60V/79G/83I/96L/162K/174K/195L/210E/277V/318Q, 13P/16S/17G/26E/31K/37G/60V/79G/83W/96L/162M/174K/195L/210E/277V/318Q, 13P/16S/17G/26E/31K/37G/60V/79G/83W/96L/162K/174K/195L/210H/277V/318Q, 13P/16S/17G/26E/31K/37G/60V/79G/83W/96L/162K/174K/195H/210E/277V/318Q, 13P/16S/17G/26E/31K/37G/60V/79G/83W/96L/162K/174Y/195L/210E/277V/318Q, 13P/16S/17G/26E/31K/37Y/60V/79G/83W/96L/162K/174K/195L/210E/277V/318Q, 13P/16S/17G/26E/31K/37G/60V/79G/83W/96L/162K/174K/195L/210Q/277V/318Q, 13P/16S/17G/26E/31K/37T/60V/79G/83W/96L/162K/174K/195L/210E/277V/318Q, 13P/16S/17G/26E/31K/37G/60V/79G/83W/96N/162K/174K/195L/210E/277V/318Q, 13P/16S/17G/26E/31K/37G/60V/79G/83H/96L/162K/174K/195L/210E/277V/318Q, 13P/16H/17G/26E/31K/37G/60V/79G/83W/96L/162K/174K/195L/210E/277V/318Q, 13P/16S/17G/26E/31K/37G/60V/79G/83W/96L/162K/174F/195L/210E/277V/318Q, 13P/16S/17G/26E/31K/37G/60V/79G/83D/96L/162K/174K/195L/210E/277V/318Q, 13P/16S/17G/26E/31K/37G/60V/79G/83Q/96L/162K/174K/195L/210E/277V/318Q, 13P/16S/17G/26E/31K/37G/60V/79G/83W/96L/162H/174K/195L/210E/277V/318Q, 13P/16S/17G/26E/31K/37G/60V/79G/83W/96L/162W/174K/195L/210E/277V/318Q, 13P/16S/17G/26E/31K/37G/60V/79G/83W/96L/162N/174K/195L/210E/277V/318Q, 13P/16F/17G/26E/31K/37G/60V/79G/83W/96L/162K/174K/195L/210E/277V/318Q, 13P/16S/17G/26E/31K/37G/60V/79G/83W/96L/162Q/174K/195L/210E/277V/318Q, 13P/16S/17G/26E/31K/37G/60V/79G/83W/96L/174K/195L/210E/277V/318Q, 13P/16S/17G/26E/31K/37G/60V/79G/83W/96L/162R/174K/195L/210E/277V/318Q, 13P/16S/17G/26E/31K/37G/60V/79G/83W/96L/162K/174K/195L/210F/277V/318Q, or 13P/16S/17G/26E/31K/37G/60V/79G/83W/96L/162C/174K/195L/210E/277V/318Q, wherein the amino acid positions are relative to the reference sequence of SEQ ID NO: 2 or 4.

In some embodiments, the amino acid sequence of the engineered oxalate decarboxylase polypeptide comprises at least a substitution or substitution set at amino acid position(s) 13/16/17/26/31/37/60/79/83/96/162/195/210/277, 13/16/17/31/37/60/79/83/162/174/195/210/277/318, 13/16/17/26/31/37/79/83/96/162/174/195/210/277, 13/16/17/26/37/60/79/83/96/162/174/195/210/277, 13/16/17/26/31/37/60/79/83/96/195/210/277, 13/16/17/26/31/37/60/79/83/96/162/195/210, 13/16/17/31/37/60/79/83/96/162/174/210/277, 13/16/17/26/31/37/79/83/96/174/210/277/318, 13/16/17/26/31/37/79/83/96/174/195/210/277, 13/16/17/26/31/37/60/79/83/96/162/210/318, 13/26/31/37/60/79/83/96/162/195/210/277/318, 13/16/17/26/31/37/79/83/96/162/174/210/318, 13/16/17/26/31/60/79/83/96/162/174/195/210, 13/17/26/31/37/60/79/83/96/174/210/318, 13/16/17/31/37/60/96/174/195/210/277/318, 13/16/17/37/79/83/96/162/174/195/210/277, 13/17/26/31/37/60/79/83/96/162/210/318, 16/17/26/31/60/79/96/174/195/210/277/318, 13/26/31/37/79/83/96/162/174/210/277/318, 16/26/31/60/79/96/162/174/195/210/277/318, 13/16/17/26/60/79/83/96/195/210/277, 17/26/37/60/79/96/174/195/210/277/318, 13/17/26/31/37/79/83/96/162/210/277, 13/16/26/31/37/79/83/174/210/318, 16/17/31/37/79/96/195/210/277/318, 13/16/26/37/79/83/96/195/210/318, 13/31/37/60/79/162/174/195/210/318, 13/16/31/60/79/83/96/162/195/210, 17/26/37/79/83/96/195/210/277/318, 16/26/37/60/79/96/162/174/195/210, 26/37/60/79/96/174/195/210/277, 17/26/31/37/79/96/174/210/277, 26/60/79/174/195/210/277/318, 16/31/79/96/174/195/277, 17/31/60/79/96/210/277, 17/37/60/79/195/210/277, 26/37/79/210/277, 17/31/79/96/277, 17/26/31/60/318, 16/195/277/318, 31/60/162/210/277, 16/17/79, 26/277, 26/37, 31/83, 16/17, 79/83, 60/277, 83/195, 16, 17, 13, or 277, wherein the amino acid positions are relative to the reference sequence of SEQ ID NO: 2 or 4.

In some embodiments, the amino acid sequence of the engineered oxalate decarboxylase polypeptide comprises at least a substitution or substitution set 13P/16S/17G/26E/31K/37G/60V/79G/83W/96L/162K/195L/210E/277V, 13P/16S/17G/31K/37G/60V/79G/83W/162K/174K/195L/210E/277V/318Q, 13P/16S/17G/26E/31K/37G/79G/83W/96L/162K/174K/195L/210E/277V, 13P/16S/17G/26E/37G/60V/79G/83W/96L/162K/174K/195L/210E/277V, 13P/16S/17G/26E/31K/37G/60V/79G/83W/96L/195L/210E/277V, 13P/16S/17G/26E/31K/37G/60V/79G/83W/96L/162K/195L/210E, 13P/16S/17G/31K/37G/60V/79G/83W/96L/162K/174K/210E/277V, 13P/16S/17G/26E/31K/37G/79G/83W/96L/174K/210E/277V/318Q, 13P/16S/17G/26E/31K/37G/79G/83W/96L/174K/195L/210E/277V, 13P/16S/17G/26E/31K/37G/60V/79G/83W/96L/162K/210E/318Q, 13P/26E/31K/37G/60V/79G/83W/96L/162K/195L/210E/277V/318Q, 13P/16S/17G/26E/31K/37G/79G/83W/96L/162K/174K/210E/318Q, 13P/16S/17G/26E/31K/60V/79G/83W/96L/162K/174K/195L/210E, 13P/17G/26E/31K/37G/60V/79G/83W/96L/174K/210E/318Q, 13P/16S/17G/31K/37G/60V/96L/174K/195L/210E/277V/318Q, 13P/16S/17G/37G/79G/83W/96L/162K/174K/195L/210E/277V, 13P/17G/26E/31K/37G/60V/79G/83W/96L/162K/210E/318Q, 16S/17G/26E/31K/60V/79G/96L/174K/195L/210E/277V/318Q, 13P/26E/31K/37G/79G/83W/96L/162K/174K/210E/277V/318Q, 16S/26E/31K/60V/79G/96L/162K/174K/195L/210E/277V/318Q, 13P/16S/17G/26E/60V/79G/83W/96L/195L/210E/277V, 17G/26E/37G/60V/79G/96L/174K/195L/210E/277V/318Q, 13P/17G/26E/31K/37G/79G/83W/96L/162K/210E/277V, 13P/16S/26E/31K/37G/79G/83W/174K/210E/318Q, 16S/17G/31K/37G/79G/96L/195L/210E/277V/318Q, 13P/16S/26E/37G/79G/83W/96L/195L/210E/318Q, 13P/31K/37G/60V/79G/162K/174K/195L/210E/318Q, 13P/16S/31K/60V/79G/83W/96L/162K/195L/210E, 17G/26E/37G/79G/83W/96L/195L/210E/277V/318Q, 16S/26E/37G/60V/79G/96L/162K/174K/195L/210E, 26E/37G/60V/79G/96L/174K/195L/210E/277V, 17G/26E/31K/37G/79G/96L/174K/210E/277V, 26E/60V/79G/174K/195L/210E/277V/318Q, 16S/31K/79G/96L/174K/195L/277V, 17G/31K/60V/79G/96L/210E/277V, 17G/37G/60V/79G/195L/210E/277V, 26E/37G/79G/210E/277V, 17G/31K/79G/96L/277V, 17G/26E/31K/60V/318Q, 16S/195L/277V/318Q, 31K/60V/162K/210D/277V, 16S/17G/79G, 26E/277V, 26E/37G, 31K/83W, 16S/17G, 79G/83W, 60V/277V, 83W/195L, 16S, 17G, 13P, or 277H, wherein the amino acid positions are relative to the reference sequence of SEQ ID NO: 2 or 4.

In some embodiments, the amino acid sequence of the engineered oxalate decarboxylase polypeptide comprises at least a substitution at an amino acid position set forth in Tables 4-1, 4-2, 4-3, 4-4, 4-5, 4-6, 4-7, 5-1, and 5-2, wherein the amino acid positions are relative to SEQ ID NO: 2 or 4.

In some embodiments, the amino acid sequence of the engineered oxalate decarboxylase polypeptide comprises at least a substitution set forth in Tables 4-1, 4-2, 4-3, 4-4, 4-5, 4-6, 4-7, 5-1, and 5-2, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set at amino acid position(s) set forth in Tables 4-1, 4-2, 4-3, 4-4, 4-5, 4-6, 4-7, 5-1, and 5-2, wherein the amino acid positions are relative to the reference sequence corresponding to SEQ ID NO: 2 or 4.

In some embodiments, the amino acid sequence of the engineered oxalate decarboxylase polypeptide comprises at least a substitution or substitution set of an engineered oxalate decarboxylase polypeptide set forth in any of Tables 4-1, 4-2, 4-3, 4-4, 4-5, 4-6, 4-7, 5-1, and 5-2, wherein the amino acid positions are relative to SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 172, 320, 396, 616, 670, 750, or 906, or to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of an even-numbered SEQ ID NO. of SEQ ID NOs: 28-614, a reference sequence corresponding to an even-numbered SEQ ID NO. of SEQ ID NOs: 28-614, or a reference sequence corresponding to an even-numbered SEQ ID NO. of SEQ ID NOs: 616-1622.

In some embodiments, the engineered oxalate decarboxylase polypeptide comprises an amino acid sequence having at least 770%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, it includes the proviso that the engineered oxalate decarboxylase polypeptide does not include the amino acid sequence corresponding to SEQ ID NO: 2. In some embodiments, the amino acid sequence of the engineered oxalate decarboxylase polypeptide comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to an even-numbered SEQ ID NO. of SEQ ID NOs: 28-1622, or to a reference sequence corresponding to residues 1-359 of an even-numbered SEQ ID NO. of SEQ ID NOs: 28-614, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution or amino acid residue 4C/R/S, 5C/S, 6A/S/W, 7G, 10A/Q/R, 11R, 13A/C/D/E/F/G/H/I/K/L/M/N/P/Q/R/S/T/V/W/Y, 14L/V, 16A/C/D/E/F/G/H/I/K/L/M/N/P/Q/R/S/T/V/W/Y, 17A/C/D/E/F/G/H/I/K/L/M/N/P/Q/R/S/T/V/W/Y, 18G/Q/R, 19C, 22R, 26A/C/D/E/F/G/H/I/K/L/M/N/P/Q/R/S/T/V/W/Y, 31A/C/D/E/F/G/H/I/K/L/M/N/P/Q/R/S/T/V/W/Y, 33R, 35V, 37A/C/D/E/F/G/H/I/K/L/M/N/P/Q/R/S/T/V/W/Y, 40S, 43C, 44S, 46R, 52T, 54L, 60A/C/D/E/F/G/H/I/K/L/M/N/P/Q/R/S/T/V/W/Y, 61M, 62G, 63A/S, 76S, 79A/C/D/E/F/G/H/I/K/L/M/N/P/Q/R/S/T/V/W/Y, 80L, 82I, 83A/C/D/E/F/G/H/I/K/L/M/N/P/Q/R/S/T/V/W/Y, 85E/G/R, 94N/T, 96A/C/D/E/F/G/H/I/K/L/M/N/P/Q/R/S/T/V/W/Y, 97T, 103Q/S/V, 104Q, 106C, 110V, 117A, 121W, 123G/Q/S, 124A/C/G/Q/R/T, 125E/F/S, 126T, 128A, 141Q, 149T, 153H/Q/S, 155L/P/R/V, 156E, 160T, 162A/C/D/E/F/G/H/I/K/L/M/N/P/Q/R/S/T/V/W/Y, 164R/V, 166R, 169A/G/L, 173Q/V, 174A/C/D/E/F/G/H/I/K/L/M/N/P/Q/R/S/T/V/W/Y, 176L, 180E, 182R, 183C/E/I/K, 186N, 187L/R/S, 188A, 189R, 190G/Q, 193A/G, 195A/C/D/E/F/G/H/I/K/L/M/N/P/Q/R/S/T/V/W/Y, 196F/K/M/R/S/V, 197E, 199G/V, 200N, 205A, 206M, 208G, 210A/C/D/E/F/G/H/I/K/L/M/N/P/Q/R/S/T/V/W/Y, 212A/F/G/L/S/V, 216S/W, 219V, 226S/T, 227E/S, 232T, 233D/H/R, 234L, 240E, 242D, 243V, 263S, 265C, 266V, 267R, 269S, 270L, 273A, 274Q/S, 277A/C/D/E/F/G/H/I/K/L/M/N/P/Q/R/S/T/V/W/Y, 284A/R/S, 297Y, 3011/Q/T, 303T, 304D/G, 314S, 316K/V, 318A/C/D/E/F/G/H/I/K/L/M/N/P/Q/R/S/T/V/W/Y, 331I/L/P/V, 335R, 339G, 342A/E/P/R, 343R/W, 346G/L/Q/W, 347F/R, 350R, 351A/E/H, 356S, or 359L/W, or combinations thereof, wherein the amino acid positions are relative to the reference sequence corresponding to residues SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to residues SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution or amino acid residue 13A/C/D/E/F/G/H/I/K/L/M/N/P/Q/R/S/T/V/W/Y, 14L/V, 16A/C/D/E/F/G/H/I/K/L/M/N/P/Q/R/S/T/V/W/Y, 17A/C/D/E/F/G/H/I/K/L/M/N/P/Q/R/S/T/V/W/Y, 26A/C/D/E/F/G/H/I/K/L/M/N/P/Q/R/S/T/V/W/Y, 31A/C/D/E/F/G/H/I/K/L/M/N/P/Q/R/S/T/V/W/Y, 37A/C/D/E/F/G/H/I/K/L/M/N/P/Q/R/S/T/V/W/Y, 60A/C/D/E/F/G/H/I/K/L/M/N/P/Q/R/S/T/V/W/Y, 79A/C/D/E/F/G/H/I/K/L/M/N/P/Q/R/S/T/V/W/Y, 83A/C/D/E/F/G/H/I/K/L/M/N/P/Q/R/S/T/V/W/Y, 96A/C/D/E/F/G/H/I/K/L/M/N/P/Q/R/S/T/V/W/Y, 162A/C/D/E/F/G/H/I/K/L/M/N/P/Q/R/S/T/V/W/Y, 174A/C/D/E/F/G/H/I/K/L/M/N/P/Q/R/S/T/V/W/Y, 195A/C/D/E/F/G/H/I/K/L/M/N/P/Q/R/S/T/V/W/Y, 196F/K/M/R/S/V, 210A/C/D/E/F/G/H/I/K/L/M/N/P/Q/R/S/T/V/W/Y, 226S/T, 277A/C/D/E/F/G/H/I/K/L/M/N/P/Q/R/S/T/V/W/Y, 301I/Q/T, or 318A/C/D/E/F/G/H/I/K/L/M/N/P/Q/R/S/T/V/W/Y, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution 13P, 14L, 16S, 17H/G, 26E, 31K, 37G, 60V, 79G, 83W, 96L, 162K, 174K, 195L, 196V, 210A/E, 226S, 277V, 301I, or 318Q, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitutions at amino acid position 31, 210, or 318, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution 31K, 210A/E, or 318Q, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of SEQ ID NO: 172, or to the reference sequence corresponding to SEQ ID NO: 172, wherein the amino acid sequence comprises one or more amino acid substitutions relative to the reference sequence corresponding to residues 1-359 of SEQ ID NO: 172, or relative to the reference sequence corresponding SEQ ID NO: 172.

In some embodiments, the amino acid sequence of the engineered oxalate decarboxylase polypeptide comprises at least a substitution or substitution set at amino acid position(s) 13, 212, 124/196/210/226, 5, 16/26/124/155/195/210/284, 196/226, 16/155/195/210/226, 16/195/210/226, 16/155/174/196, 16/195/226, 318, 16/195/196/210, 16/26/124/155/174/196/210, 195/210, 26/155/174/210, 316, 60, 16/155/174, 16/124/174/196, 16/226, 16/26/174/196/226, 17, 331, 124/195, 16/174/196, 188, 195/196/210, 174/196/210, 16/26/155/174, 16/155/195/196/226, 195/226/284, 16, 240, 16/26/124/155/195/196/226, 183/232/339/343, 183/206, 63, 173/347, 16/26/174/196, 16/284, 46, 16/124/195/196, 274, 174/196, 155/174/196, 174/196/226, 16/124/155/174/195, 16/26/155/174/196, 26/174/196/210/284, 16/195/196/284, 174, 16/124/174/195/210/226/284, 16/174/195/284, 26/174/196/210/226/284, 174/196/226/284, 124/174/196, 26/174/195/210/226, 162, 16/124/195/196/284, 206/343, 16/195/196, 16/26/174/195/196/210/226, 155/195/196/226, 18, 335, 124/155/174/195/226, 26/155/174/195/226, 195/196, 153, 155/174/195, 174/195/210/284, 174/195, 37, 124/174/195/226/284, 176, 16/155/174/195/196/226/284, 10, 169/173, 155, 33, 173/183/343/347, 104/265, or 233, wherein the amino acid positions are relative to the reference sequence corresponding to residues 1-359 of SEQ ID NO: 172, or relative to the reference sequence corresponding SEQ ID NO: 172.

In some embodiments, the amino acid sequence of the engineered oxalate decarboxylase polypeptide comprises at least a substitution or substitution set 13P, 212S, 124A/196V/210E/226S, 5S, 13E, 16S/26E/124A/155P/195L/210E/284A, 196V/226S, 16S/155P/195L/210E/226S, 16S/195L/210E/226S, 16S/155P/174K/196V, 16S/195L/226S, 13L, 318E, 16S/195L/196V/210E, 16S/26E/124A/155P/174K/196V/210E, 195L/210E, 26E/155P/174K/210E, 316V, 60V, 16S/155P/174K, 16S/124A/174K/196V, 16S/226S, 16S/26E/174K/196V/226S, 17H, 331V, 124A/195L, 16S/174K/196V, 17A, 188A, 195L/196V/210E, 174K/196V/210E, 5C, 16S/26E/155P/174K, 16S/155P/195L/196V/226S, 195L/226S/284A, 17N, 16L, 240E, 16S/26E/124A/155P/195L/196V/226S, 331P, 1831/232T/339G/343R, 183I/206M, 63A, 173V/347R, 16S/26E/174K/196V, 16S/284A, 60R, 46R, 16S/124A/195L/196V, 274S, 331I, 174K/196V, 155P/174K/196V, 174K/196V/226S, 331L, 16S/124A/155P/174K/195L, 16S/26E/155P/174K/196V, 26E/174K/196V/210E/284A, 212A, 16S/195L/196V/284A, 174K, 16S/124A/174K/195L/210E/226S/284A, 16S/174K/195L/284A, 26E/174K/196V/210E/226S/284A, 212L, 174K/196V/226S/284A, 124A/174K/196V, 26E/174K/195L/210E/226S, 162A, 16S/124A/195L/196V/284A, 206M/343R, 16S/195L/196V, 16S/26E/174K/195L/196V/210E/226S, 155P/195L/196V/226S, 18Q, 335R, 124A/155P/174K/195L/226S, 17C, 26E/155P/174K/195L/226S, 195L/196V, 162T, 153H, 316K, 155P/174K/195L, 153Q, 174K/195L/210E/284A, 174K/195L, 153S, 212F, 37C, 212V, 124A/174K/195L/226S/284A, 176L, 16S/155P/174K/195L/196V/226S/284A, 10Q, 169G/173V, 155L, 18G, 60L, 10A, 18R, 33R, 212G, 10R, 13R, 173V/1831/343R/347R, 16R, 104Q/265C, 233R, or 233D, wherein the amino acid positions are relative to the reference sequence corresponding to residues 1-359 of SEQ ID NO: 172, or relative to the reference sequence corresponding SEQ ID NO: 172.

In some embodiments, the amino acid sequence of the engineered oxalate decarboxylase polypeptide comprises at least a substitution or substitution set H13P, H212S, K124A/K196V/A210E/T226S, T5S, H13E, A16S/A26E/K124A/A155P/R195L/A210E/K284A, K196V/T226S, A16S/A155P/R195L/A210E/T226S, A16S/R195L/A210E/T226S, A16S/A155P/D174K/K196V, A16S/R195L/T226S, H13L, Q318E, A16S/R195L/K196V/A210E, A16S/A26E/K124A/A155P/D174K/K196V/A210E, R195L/A210E, A26E/A155P/D174K/A210E, N316V, T60V, A16S/A155P/D174K, A16S/K124A/D174K/K196V, A16S/T226S, A16S/A26E/D174K/K196V/T226S, D17H, A331V, K124A/R195L, A16S/D174K/K196V, D17A, P188A, R195L/K196V/A210E, D174K/K196V/A210E, T5C, A16S/A26E/A155P/D174K, A16S/A155P/R195L/K196V/T226S, R195L/T226S/K284A, D17N, A16L, D240E, A16S/A26E/K124A/A155P/R195L/K196V/T226S, A331P, V183I/K232T/S339G/S343R, V183I/L206M, P63A, K173V/K347R, A16S/A26E/D174K/K196V, A16S/K284A, T60R, V46R, A16S/K124A/R195L/K196V, H274S, A331I, D174K/K196V, A155P/D174K/K196V, D174K/K196V/T226S, A331L, A16S/K124A/A155P/D174K/R195L, A16S/A26E/A155P/D174K/K196V, A26E/D174K/K196V/A210E/K284A, H212A, A16S/R195L/K196V/K284A, D174K, A16S/K124A/D174K/R195L/A210E/T226S/K284A, A16S/D174K/R195L/K284A, A26E/D174K/K196V/A210E/T226S/K284A, H212L, D174K/K196V/T226S/K284A, K124A/D174K/K196V, A26E/D174K/R195L/A210E/T226S, G162A, A16S/K124A/R195L/K196V/K284A, L206M/S343R, A16S/R195L/K196V, A16S/A26E/D174K/R195L/K196V/A210E/T226S, A155P/R195L/K196V/T226S, K18Q, A335R, K124A/A155P/D174K/R195L/T226S, D17C, A26E/A155P/D174K/R195L/T226S, R195L/K196V, G162T, P153H, N316K, A155P/D174K/R195L, P153Q, D174K/R195L/A210E/K284A, D174K/R195L, P153S, H212F, K37C, H212V, K124A/D174K/R195L/T226S/K284A, V176L, A16S/A155P/D174K/R195L/K196V/T226S/K284A, V10Q, D169G/K173V, A155L, K18G, T60L, V10A, K18R, K33R, H212G, V10R, H13R, K173V/V183I/S343R/K347R, A16R, P104Q/N265C, T233R, or T233D, wherein the amino acid positions are relative to the reference sequence corresponding to residues 1-359 of SEQ ID NO: 172, or relative to the reference sequence corresponding SEQ ID NO: 172.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of SEQ ID NO: 320, or to the reference sequence corresponding to SEQ ID NO: 320, wherein the amino acid sequence comprises one or more amino acid substitutions relative to the reference sequence corresponding to residues 1-359 of SEQ ID NO: 320, or relative to the reference sequence corresponding to SEQ ID NO: 320.

In some embodiments, the amino acid sequence of the engineered oxalate decarboxylase polypeptide comprises at least a substitution or substitution set at amino acid position(s) 13/17/196, 17/212/331, 17/26/196/212/226, 17/26/196/212/226/331, 13/17/26/196/331, 17/60/196/226, 17/46/212, 26/60/196/226/331, 17/46/196/212/274/331, 212, 17/196/226, 13/17/26/212/331, 13/17/212/226/331, 196/212/331, 13/17/196/331, 13/17/26/212/226/331, 17/60/196/212/226/331, 13/17/212, 13/17/26/331, 13/17, 13/17/26/196, 46/196/212/226, 17/60/196, 17/196/331, 13/17/46/226/331, 17/196/226/331, 17/46/196/212/331, 17/46/212/331, 17/196, 13/17/212/226, 17/212, 17/26/212, 13/17/226/331, 17/196/212/331, 17/196/212/226/331, 13/17/26/226/331, 46/60/196/226/331, 17/46/196/331, 13/17/46/196, 196/212, 13/17/26, 17/83/263, 17/83/173/227, 17/227/301, 17/83/227/263, 17/83/125, 17/83/173, 17/263/301, 17/125, 17/83/301, 17/173, 17/83/227, 17/227, 17, 17/125/227, 17/83, 128, 76, 141, 110, 79, 117, or 61, wherein the amino acid positions are relative to the reference sequence corresponding to residues 1-359 of SEQ ID NO: 320, or relative to the reference sequence corresponding to SEQ ID NO: 320.

In some embodiments, the amino acid sequence of the engineered oxalate decarboxylase polypeptide comprises at least a substitution or substitution set 13P/17H/196K, 17H/212S/331V, 17H/26A/196K/212S/226T, 17H/26A/196K/212A/226T/331V, 13P/17H/26A/196K/331V, 17H/60R/196K/226T, 17H/46R/212S, 26A/60V/196K/226T/331V, 17H/46R/196K/212A/274Q/331V, 212S, 17H/196K/226T, 13P/17H/26A/212A/331V, 13P/17H/212S/226T/331V, 196K/212S/331V, 13P/17H/196K/331V, 13P/17H/26A/212A/226T/331V, 17H/60R/196K/212A/226T/331V, 13P/17H/212S, 13P/17H/26A/331V, 13P/17H, 13P/17H/26A/196K, 46R/196K/212S/226T, 17H/60R/196K, 17H/196K/331V, 13P/17H/46R/226T/331V, 17H/196K/226T/331V, 17H/60V/196K/226T, 17H/46R/196K/212S/331V, 13P/17H/212A, 17H/46R/212S/331V, 17H/196K, 13P/17H/212S/226T, 17H/212S, 17H/26A/212S, 13P/17H/226T/A331V, D17H/V196K/212S/331V, 17H/196K/212S/226T/331V, 13P/17H/26A/226T/331V, 46R/60V/196K/226T/331V, 17H/46R/196K/331V, 17H/212A/331V, 13P/17H/46R/196K, 196K/212S, 13P/17H/26A, 17G/83E/263S, 17G/83E/173Q/227E, 17G/227E/301I, 17G/83E/227E/263S, 17G/83E/125E, 17G/83E/173Q, 17G/263S/301I, 17G/125E, 17G/83E/301I, 17G/173Q, 17G/83E/227E, 17G/227E, 17G, 17G/125E/227E, 17G/83E, 17G/263S/301T, 128A, 76S, 141Q, 110V, 79S, 117A, 79G, or 61M, wherein the amino acid positions are relative to the reference sequence corresponding to residues 1-359 of SEQ ID NO: 320, or relative to the reference sequence corresponding to SEQ ID NO: 320.

In some embodiments, the amino acid sequence of the engineered oxalate decarboxylase polypeptide comprises at least a substitution or substitution set H13P/D17H/V196K, D17H/H212S/A331V, D17H/E26A/V196K/H212S/S226T, D17H/E26A/V196K/H212A/S226T/A331V, H13P/D17H/E26A/V196K/A331V, D17H/T60R/V196K/S226T, D17H/V46R/H212S, E26A/T60V/V196K/S226T/A331V, D17H/V46R/V196K/H212A/H274Q/A331V, H212S, D17H/V196K/S226T, H13P/D17H/E26A/H212A/A331V, H13P/D17H/H212S/S226T/A331V, V196K/H212S/A331V, H13P/D17H/V196K/A331V, H13P/D17H/E26A/H212A/S226T/A331V, D17H/T60R/V196K/H212A/S226T/A331V, H13P/D17H/H212S, H13P/D17H/E26A/A331V, H13P/D17H, H13P/D17H/E26A/V196K, V46R/V196K/H212S/S226T, D17H/T60R/V196K, D17H/V196K/A331V, H13P/D17H/V46R/S226T/A331V, D17H/V196K/S226T/A331V, D17H/T60V/V196K/S226T, D17H/V46R/V196K/H212S/A331V, H13P/D17H/H212A, D17H/V46R/H212S/A331V, D17H/V196K, H13P/D17H/H212S/S226T, D17H/H212S, D17H/E26A/H212S, H13P/D17H/S226T/A331V, D17H/V196K/H212S/A331V, D17H/V196K/H212S/S226T/A331V, H13P/D17H/E26A/S226T/A331V, V46R/T60V/V196K/S226T/A331V, D17H/V46R/V196K/A331V, D17H/H212A/A331V, H13P/D17H/V46R/V196K, V196K/H212S, H13P/D17H/E26A, D17G/T83E/D263S, D17G/T83E/K173Q/R227E, D17G/R227E/V301I, D17G/T83E/R227E/D263S, D17G/T83E/P125E, D17G/T83E/K173Q, D17G/D263S/V301I, D17G/P125E, D17G/T83E/V301I, D17G/K173Q, D17G/T83E/R227E, D17G/R227E, D17G, D17G/P125E/R227E, D17G/T83E, D17G/D263S/V301T, F128A, A76S, G141Q, F110V, A79S, M117A, A79G, or L61M, wherein the amino acid positions are relative to the reference sequence corresponding to residues 1-359 of SEQ ID NO: 320, or relative to the reference sequence corresponding to SEQ ID NO: 320.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of SEQ ID NO: 396, or to the reference sequence corresponding to SEQ ID NO:396, wherein the amino acid sequence comprises one or more amino acid substitutions relative to the reference sequence corresponding to residues 1-359 of SEQ ID NO: 396, or relative to the reference sequence corresponding to SEQ ID NO: 396.

In some embodiments, the amino acid sequence of the engineered oxalate decarboxylase polypeptide comprises at least a substitution or substitution set at amino acid position(s) 13/17/79, 13/14/17/60/79, 13/17/79/83, 13/17/79/301, 13/14/17/60/79/212, 17/60/197, 13/60/212, 13/14/17/60/79/83/301, 13/17/60/79, 17/60/301, 14/17/79, 17/60/83, 17/79, 13/17/60/79/83/301, 342, 52/190, 94/190, 190/342/351, 13/79, 13/96, 273, 126, 96, 79, 269, 40, 44, 267, 266, 160, 277, 149, 22, 234, 54, 297, 106, or 13/43, wherein the amino acid positions are relative to the reference sequence corresponding to residues 1-359 of SEQ ID NO: 396, or relative to the reference sequence corresponding to SEQ ID NO: 396.

In some embodiments, the amino acid sequence of the engineered oxalate decarboxylase polypeptide comprises at least a substitution or substitution set 13H/17G/79G, 13H/14V/17G/60V/79G, 13H/17G/79G/83E, 13H/17G/79G/301I, 13H/14V/17G/60V/79G/212S, 17G/60V/197E, 13H/60V/212S, 13H/14V/17G/60V/79G/83E/301I, 13H/17G/60V/79G, 17G/60V/301I, 14V/17G/79G, 17G/60V/83E, 17G/79G, 13H/17G/60V/79G/83E/301I, 342P, 52T/190Q, 94N/190Q, 190Q/342P/351E, 13H/79G, 13H/96L, 273A, 126T, 96K, 79S, 269S, 40S, 96L, 44S, 96R, 79G, 267R, 266V, 160T, 277T, 149T, 22R, 234L, 54L, 297Y, 277A, 106C, or 13H/43C, wherein the amino acid positions are relative to the reference sequence corresponding to residues 1-359 of SEQ ID NO: 396, or relative to the reference sequence corresponding to SEQ ID NO: 396.

In some embodiments, the amino acid sequence of the engineered oxalate decarboxylase polypeptide comprises at least a substitution or substitution set P13H/H17G/A79G, P13H/L14V/H17G/T60V/A79G, P13H/H17G/A79G/T83E, P13H/H17G/A79G/V301I, P13H/L14V/H17G/T60V/A79G/H212S, H17G/T60V/L197E, P13H/T60V/H212S, P13H/L14V/H17G/T60V/A79G/T83E/V301I, P13H/H17G/T60V/A79G, H17G/T60V/V301I, L14V/H17G/A79G, H17G/T60V/T83E, H17G/A79G, P13H/H17G/T60V/A79G/T83E/V301I, T342P, K52T/A190Q, Q94N/A190Q, A190Q/T342P/Q351E, P13H/A79G, P13H/A96L, S273A, A126T, A96K, A79S, T269S, G40S, A96L, T44S, A96R, A79G, S267R, I266V, N160T, Y277T, I149T, T22R, V234L, I54L, S297Y, Y277A, D106C, or P13H/A43C, wherein the amino acid positions are relative to the reference sequence corresponding to residues 1-359 of SEQ ID NO: 396, or relative to the reference sequence corresponding to SEQ ID NO: 396.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 670, wherein the amino acid sequence comprises one or more amino acid substitutions relative to the reference sequence corresponding to residues 1-359 of SEQ ID NO: 670, or the reference sequence corresponding to SEQ ID NO: 670.

In some embodiments, the amino acid sequence of the engineered oxalate decarboxylase polypeptide comprises at least a substitution at amino acid position 37, 125, 162, 94, 195, 85, 103, 232, 189, 186, 155, 193, 342, 196, 63, 33, 351, 314, 187, 303, 164, 153, 346, 183, 19, 123, 350, 205, 284, 199, 343, 200, 208, 169, 190, or 121, or combinations thereof, wherein the amino acid positions are relative to the reference sequence corresponding SEQ ID NO: 670.

In some embodiments, the amino acid sequence of the engineered oxalate decarboxylase polypeptide comprises at least a substitution 37G, 125F, 162K, 94T, 195W, 85R, 103S, 232T, 189R, 186N, 155R, 193A, 342A, 196V, 63S, 33R, 351H, 193G, 125S, 314S, 187R, 303T, 103V, 103Q, 155V, 164R, 153S, 37R, 346Q, 346G, 351A, 183K, 19C, 187L, 123S, 85G, 187S, 183C, 123G, 350R, 85E, 205A, 284R, 199V, 346W, 343W, 164V, 200N, 342E, 208G, 169A, 190G, 284A, 199G, 121W, or 196S, or combinations thereof, wherein the amino acid positions are relative to the reference sequence corresponding to SEQ ID NO: 670.

In some embodiments, the amino acid sequence of the engineered oxalate decarboxylase polypeptide comprises a substitution K37G, P125F, G162K, Q94T, L195W, H85R, G103S, K232T, A189R, A186N, A155R, Q193A, T342A, K196V, P63S, K33R, Q351H, Q193G, P125S, T314S, T187R, D303T, G103V, G103Q, A155V, P164R, P153S, K37R, E346Q, E346G, Q351A, V183K, D19C, T187L, D123S, H85G, T187S, V183C, D123G, K350R, H85E, E205A, K284R, P199V, E346W, S343W, P164V, L200N, T342E, E208G, D169A, A190G, K284A, P199G, L121W, or K196S, or combinations thereof, wherein the amino acid positions are relative to the reference sequence corresponding to SEQ ID NO: 670.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 616, wherein the amino acid sequence comprises one or more amino acid substitutions relative to the reference sequence corresponding to SEQ ID NO: 616.

In some embodiments, the amino acid sequence of the engineered oxalate decarboxylase polypeptide comprises at least a substitution or substitution set at amino acid position(s) 14/26/60/318, 14/26/60/94/162/212, 60/162/226, 60, 14/125/162/212/318, 60/125/226, 14/125/162/226, 26/60/162/226/318, 14/26/94/162/212/226, 14/60/162, 14/60/162/226, 14/94/162/318, 14/94/162, 14/60/226, 14/60/94/212, 14/60/162/212, 14/162, 14/94/212/318, 14/60, 14/26/162, 14, 14/318, 14/162/226/318, 14/125/212, 14/125/226/318, 14/26/60/162, 14/125/162/212/226, 14/94/125/162/318, 14/125/226, 277, 166, 233, 83, 5, 342, 96, 284, or 26, wherein the amino acid positions are relative to the reference sequence corresponding SEQ ID NO: 616.

In some embodiments, the amino acid sequence of the engineered oxalate decarboxylase polypeptide comprises at least a substitution or substitution set 14L/26A/60V/318E, 14L/26A/60V/94T/162K/212S, 60V/162K/226T, 60V, 14L/125F/162K/212S/318E, 60V/125F/226T, 14L/125F/162K/226T, 26A/60V/162K/226T/318E, 14L/26A/94T/162K/212S/226T, 14L/60V/162K, 14L/60V/162K/226T, 14L/94T/162K/318E, 14L/94T/162K, 14L/60V/226T, 14L/60V/94T/212S, 14L/60V/162K/212S, 14L/162K, 14L/94T/212S/318E, 14L/60V, 14L/26A/162K, 14L, 14L/318E, 14L/162K/226T/318E, 14L/125F/212S, 14L/125F/226T/318E, 14L/26A/60V/162K, 14L/125F/162K/212S/226T, 14L/94T/125F/162K/318E, 14L/125F/226T, 277R, 277C, 277T, 166R, 233H, 83R, 60M, 277I, 277M, 5S, 342R, 277V, 60R, 96R, 284S, 26R, 26N, or 83W, wherein the amino acid positions are relative to the reference sequence corresponding to SEQ ID NO: 616.

In some embodiments, the amino acid sequence of the engineered oxalate decarboxylase polypeptide comprises at least a substitution or substitution set V14L/E26A/T60V/Q318E, V14L/E26A/T60V/Q94T/G162K/H212S, T60V/G162K/S226T, T60V, V14L/P125F/G162K/H212S/Q318E, T60V/P125F/S226T, V14L/P125F/G162K/S226T, E26A/T60V/G162K/S226T/Q318E, V14L/E26A/Q94T/G162K/H212S/S226T, V14L/T60V/G162K, V14L/T60V/G162K/S226T, V14L/Q94T/G162K/Q318E, V14L/Q94T/G162K, V14L/T60V/S226T, V14L/T60V/Q94T/H212S, V14L/T60V/G162K/H212S, V14L/G162K, V14L/Q94T/H212S/Q318E, V14L/T60V, V14L/E26A/G162K, V14L, V14L/Q318E, V14L/G162K/S226T/Q318E, V14L/P125F/H212S, V14L/P125F/S226T/Q318E, V14L/E26A/T60V/G162K, V14L/P125F/G162K/H212S/S226T, V14L/Q94T/P125F/G162K/Q318E, V14L/P125F/S226T, Y277R, Y277C, Y277T, S166R, T233H, T83R, T60M, Y277I, Y277M, T5S, T342R, Y277V, T60R, A96R, K284S, E26R, E26N, or T83W, wherein the amino acid positions are relative to the reference sequence corresponding to SEQ ID NO: 616.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 750, wherein the amino acid sequence comprises one or more amino acid substitutions relative to the reference sequence corresponding to SEQ ID NO: 750.

In some embodiments, the engineered oxalate decarboxylase polypeptide comprises at least a substitution or substitution set at amino acid position(s) 5/26/83/342, 5/26/277, 5/96/277, 5/83/277/342, 26/83/277, 5/26/83/277, 5/277/342, 5/26/166/277, 83/277/342, 83/96/277/342, 83/96/277, 26/83/277/342, 5/26/83/277/342, 5/83/277, 26/277, 83/277, 5/35/277, 5/26/83/96/277, 5/26/96/277, 5/277, 5/166/277, 26/83/96/277/342, 277, 26/277/342, 5, or 5/83/96/277/342, wherein the amino acid positions are relative to the reference sequence corresponding to SEQ ID NO: 750.

In some embodiments, the amino acid sequence of the engineered oxalate decarboxylase polypeptide comprises at least a substitution or substitution set 5S/26A/83W/342R, 5S/26A/277T, 5S/96L/277T, 5S/83W/277T/342R, 26A/83W/277T, 5S/26A/83W/277V, 5S/277T/342R, 5S/83W/277V/342R, 5S/26A/166R/277T, 83W/277T/342R, 83R/96L/277V/342R, 83W/96L/277T, 26A/83W/277V/342R, 5S/26A/83W/277V/342R, 5S/83R/277T/342R, 5S/26A/83W/277T/342R, 5S/83R/277V, 26A/277T, 83W/277T, 5S/35V/277T, 5S/26A/83R/96R/277V, 5S/26A/96L/277T, 5S/277T, 5S/26A/83R/277T, 5S/83R/277T, 26A/83R/277V, 5S/26A/277V, 5S/166R/277T, 26A/83W/277V, 26A/83W/96L/277V/342R, 277T, 26A/277T/342R, 83W/277V/342R, 83R/277V, 5S, 26A/277V, 83R/277T, 5S/83W/96R/277T/342R, 5S/277V, 277V, 5S/277V/342R, 83W/96L/277V, or 83W/277V, wherein the amino acid positions are relative to the reference sequence corresponding to SEQ ID NO: 750.

In some embodiments, the amino acid sequence of the engineered oxalate decarboxylase polypeptide comprises at least a substitution or substitution set T5S/E26A/T83W/T342R, T5S/E26A/Y277T, T5S/A96L/Y277T, T5S/T83W/Y277T/T342R, E26A/T83W/Y277T, T5S/E26A/T83W/Y277V, T5S/Y277T/T342R, T5S/T83W/Y277V/T342R, T5S/E26A/S166R/Y277T, T83W/Y277T/T342R, T83R/A96L/Y277V/T342R, T83W/A96L/Y277T, E26A/T83W/Y277V/T342R, T5S/E26A/T83W/Y277V/T342R, T5S/T83R/Y277T/T342R, T5S/E26A/T83W/Y277T/T342R, T5S/T83R/Y277V, E26A/Y277T, T83W/Y277T, T5S/I35V/Y277T, T5S/E26A/T83R/A96R/Y277V, T5S/E26A/A96L/Y277T, T5S/Y277T, T5S/E26A/T83R/Y277T, T5S/T83R/Y277T, E26A/T83R/Y277V, T5S/E26A/Y277V, T5S/S166R/Y277T, E26A/T83W/Y277V, E26A/T83W/A96L/Y277V/T342R, Y277T, E26A/Y277T/T342R, T83W/Y277V/T342R, T83R/Y277V, T5S, E26A/Y277V, T83R/Y277T, T5S/T83W/A96R/Y277T/T342R, T5S/Y277V, Y277V, T5S/Y277V/T342R, T83W/A96L/Y277V, or T83W/Y277V, wherein the amino acid positions are relative to the reference sequence corresponding to SEQ ID NO: 750.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 906, wherein the amino acid sequence comprises one or more amino acid substitutions relative to the reference sequence corresponding to SEQ ID NO: 906.

In some embodiments, the amino acid sequence of the engineered oxalate decarboxylase polypeptide comprises at least a substitution at amino acid position 26, 31, 96, 60, 318, 210, 277, 37, 16, 195, 79, 17, 13, 83, 162, or 174, or combinations thereof, wherein the amino acid positions are relative to the reference sequence corresponding to SEQ ID NO: 906.

In some embodiments, the amino acid sequence of the engineered oxalate decarboxylase polypeptide comprises at least a substitution 26L, 31W, 96K, 60R, 318D, 26G, 210R, 31R, 277F, 37M, 96P, 16V, 60A, 26R, 31G, 195G, 318R, 26V, 79M, 16M, 210A, 96R, 37E, 16G, 17W, 13G, 79E, 277T, 60Q, 31L, 96G, 96T, 60G, 16W, 13T, 79R, 26Y, 26S, 195R, 17R, 13V, 277E, 37L, 210G, 60L, 13L, 83Y, 37W, 277G, 83R, 60Y, 210S, 318S, 17I, 37S, 79L, 16R, 195S, 318G, 26C, 13R, 195Y, 60E, 162V, 16C, 96V, 83M, 31M, 174V, 277L, 26M, 318M, 210N, 26A, 277R, 96F, 174G, 16Q, 277A, 13Q, 79F, 16L, 195N, 79V, 17P, 60P, 37P, 96W, 162T, 195M, 318W, 37R, 16T, 210M, 162Y, 16P, 83E, 277M, 174L, 17V, 17E, 17L, 96M, 318V, 174R, 13S, 174T, 13Y, 195A, 277W, 60M, 17A, 318A, 174H, 83S, 277I, 13E, 17K, 26K, 96A, 174D, 210V, 31S, 195C, 79K, 60W, 174S, 13I, 60H, 31E, 96E, 83K, 31Q, 16K, 174M, 37V, 79D, 318T, 195F, 26T, 174Q, 83L, 96I, 13C, 13A, 96Q, 13H, 83T, 31Y, 13W, 277P, 195T, 60S, 26Q, 210P, 174N, 83A, 162E, 37F, 37Q, 31T, 174A, 83F, 60T, 96S, 83V, 210I, 16D, 37C, 83G, 13N, 31V, 79S, 96Y, 17F, 17M, 16A, 83P, 318L, 79Q, 17S, 26W, 195D, 195W, 277S, 174E, 16E, 162F, 79Y, 79A, 17C, 17Y, 195Q, 26P, 195I, 60F, 26I, 17D, 318K, 60N, 16I, 318H, 37A, 195V, 210Y, 195E, 26H, 13D, 31P, 210W, 318P, 83C, 174P, 210T, 17T, 79T, 96H, 79W, 162L, 318E, 31A, 210C, 17N, 195K, 318Y, 17H, 13M, 60D, 277K, 16N, 79C, 13K, 60K, 37N, 60I, 162D, 162I, 16Y, 162S, 96C, 210K, 174I, 210L, 26D, 26N, 210D, 13F, 318N, 277Y, 174W, 31H, 31D, 79N, 37K, 31I, 37H, 318C, 31F, 79P, 195P, 318I, 162A, 277Q, 26F, 277C, 79H, 31C, 79I, 37I, 17Q, 31N, 277N, 37D, 277H, 277D, 96D, 174C, 162P, 318F, 83N, 60C, 83I, 162M, 210H, 195H, 174Y, 37Y, 210Q, 37T, 96N, 83H, 16H, 174F, 83D, 83Q, 162H, 162W, 162N, 16F, 162Q, 162G, 162R, 210F, or 162C, or combinations thereof, wherein the amino acid positions are relative to the reference sequence corresponding to SEQ ID NO: 906.

In some embodiments, the amino acid sequence of the engineered oxalate decarboxylase polypeptide comprises at least a substitution E26L, K31W, L96K, V60R, Q318D, E26G, E210R, K31R, V277F, G37M, L96P, S16V, V60A, E26R, K31G, L195G, Q318R, E26V, G79M, S16M, E210A, L96R, G37E, S16G, G17W, P13G, G79E, V277T, V60Q, K31L, L96G, L96T, V60G, S16W, P13T, G79R, E26Y, E26S, L195R, G17R, P13V, V277E, G37L, E210G, V60L, P13L, W83Y, G37W, V277G, W83R, V60Y, E210S, Q318S, G17I, G37S, G79L, S16R, L195S, Q318G, E26C, P13R, L195Y, V60E, K162V, S16C, L96V, W83M, K31M, K174V, V277L, E26M, Q318M, E210N, E26A, V277R, L96F, K174G, S16Q, V277A, P13Q, G79F, S16L, L195N, G79V, G17P, V60P, G37P, L96W, K162T, L195M, Q318W, G37R, S16T, E210M, K162Y, S16P, W83E, V277M, K174L, G17V, G17E, G17L, L96M, Q318V, K174R, P13S, K174T, P13Y, L195A, V277W, V60M, G17A, Q318A, K174H, W83S, V277I, P13E, G17K, E26K, L96A, K174D, E210V, K31S, L195C, G79K, V60W, K174S, P13I, V60H, K31E, L96E, W83K, K31Q, S16K, K174M, G37V, G79D, Q318T, L195F, E26T, K174Q, W83L, L96I, P13C, P13A, L96Q, P13H, W83T, K31Y, P13W, V277P, L195T, V60S, E26Q, E210P, K174N, W83A, K162E, G37F, G37Q, K31T, K174A, W83F, V60T, L96S, W83V, E210I, S16D, G37C, W83G, P13N, K31V, G79S, L96Y, G17F, G17M, S16A, W83P, Q318L, G79Q, G17S, E26W, L195D, L195W, V277S, K174E, S16E, K162F, G79Y, G79A, G17C, G17Y, L195Q, E26P, L195I, V60F, E26I, G17D, Q318K, V60N, S16I, Q318H, G37A, L195V, E210Y, L195E, E26H, P13D, K31P, E210W, Q318P, W83C, K174P, E210T, G17T, G79T, L96H, G79W, K162L, Q318E, K31A, E210C, G17N, L195K, Q318Y, G17H, P13M, V60D, V277K, S16N, G79C, P13K, V60K, G37N, V60I, K162D, K162I, S16Y, K162S, L96C, E210K, K174I, E210L, E26D, E26N, E210D, P13F, Q318N, V277Y, K174W, K31H, K31D, G79N, G37K, K31I, G37H, Q318C, K31F, G79P, L195P, Q318I, K162A, V277Q, E26F, V277C, G79H, K31C, G79I, G37I, G17Q, K31N, V277N, G37D, V277H, V277D, L96D, K174C, K162P, Q318F, W83N, V60C, W83I, K162M, E210H, L195H, K174Y, G37Y, E210Q, G37T, L96N, W83H, S16H, K174F, W83D, W83Q, K162H, K162W, K162N, S16F, K162Q, K162G, K162R, E210F, or K162C, or combinations thereof, wherein the amino acid positions are relative to the reference sequence corresponding to SEQ ID NO: 906.

In some embodiments, the amino acid sequence of the engineered oxalate decarboxylase polypeptide comprises at least a substitution or substitution set at amino acid position(s) 174/318, 26/96, 60/318, 31/318, 162/174/318, 174/277/318, 26/195/318, 60/162/195, 60/162/318, 174/195/277, 16/17/174, 60/195/277, 37/277/318, 16/162/195/277, 26/79/83/162, 26/31/60/318, 16/174/195/277, 13/37/83/162, 16/17/60/195, 13/17/37/83, 31/37/162/174/318, 13/16/31/83/162, 16/60/174/195/318, 17/60/96/162/195/277, 13/26/60/83/162/174, 17/31/60/162/174/277, 16/17/26/83/96/277, 17/26/37/174/277/318, 13/16/31/60/162/174, 13/17/31/83/277/318, 13/16/17/31/83/162/318, 13/16/60/83/162/195/318, 13/16/17/31/37/83/96/162, 13/17/26/37/60/83/162/210/318, 13/16/26/31/83/96/162/174/318, 13/16/17/31/60/83/96/162/174/195/318, 13/16/26/37/60/83/162/174/195/210/318, 13/16/37/79/83/96/162/174/195/210/277, 13/17/26/31/37/60/79/83/96/162/174/210, 13/16/17/26/37/79/83/96/174/195/210/318, 13/26/31/37/60/83/96/162/174/195/210/277/318, 13/16/17/31/37/60/79/83/96/162/174/195/210/318, 13/16/17/31/60/79/83/96/162/174/195/210/277/318, 13/16/17/26/37/60/79/96/162/174/195/210/277/318, 13/26/31/37/60/79/83/96/162/174/195/210/277/318, 13/16/17/26/31/37/60/96/162/174/195/210/277/318, 13/16/17/26/31/37/79/83/96/162/174/195/210/318, 13/16/17/26/31/37/60/79/96/162/174/210/277/318, 13/17/26/31/37/60/79/83/96/162/174/195/210/277/318, 13/16/26/31/37/60/79/83/96/162/174/195/210/277/318, 16/17/26/31/37/60/79/83/96/162/174/195/210/277/318, or 13/16/17/26/31/37/60/79/83/96/162/174/195/210/277/318, wherein the amino acid positions are relative to the reference sequence corresponding to SEQ ID NO: 906.

In some embodiments, the amino acid sequence of the engineered oxalate decarboxylase polypeptide comprises at least a substitution or substitution set 174D/318E, 26A/96A, 60T/318E, 31E/318E, 162G/174D/318E, 174D/277Y/318E, 26A/195R/318E, 60T/162G/195R, 60T/162G/318E, 174D/195R/277Y, 16A/17D/174D, 60T/195R/277Y, 37K/277Y/318E, 16A/162G/195R/277Y, 26A/79A/83T/162G, 26A/31E/60T/318E, 16A/174D/195R/277Y, 13H/37K/83T/162G, 16A/17D/60T/195R, 13H/17D/37K/83T, 31E/37K/162G/174D/318E, 13H/16A/31E/83T/162G, 16A/60T/174D/195R/318E, 17D/60T/96A/162G/195R/277Y, 13H/26A/60T/83T/162G/174D, 17D/31E/60T/162G/174D/277Y, 16A/17D/26A/83T/96A/277Y, 17D/26A/37K/174D/277Y/318E, 13H/16A/31E/60T/162G/174D, 13H/17D/31E/83T/277Y/318E, 13H/16A/17D/31E/83T/162G/318E, 13H/16A/60T/83T/162G/195R/318E, 13H/16A/17D/31E/37K/83T/96A/162G, 13H/17D/26A/37K/60T/83T/162G/210P/318E, 13H/16A/26A/37K/83T/162G/174D/195R/318E, 13H/16A/26A/31E/83T/96A/162G/174D/318E, 13H/16A/17D/31E/60T/83T/96A/162G/174D/195R/318E, 13H/16A/26A/37K/60T/83T/162G/174D/195R/210P/318E, 13H/16A/37K/79A/83T/96A/162G/174D/195R/210P/277Y, 13H/17D/26A/31E/37K/60T/79A/83T/96A/162G/174D/210P, 13H/16A/17D/26A/37K/79A/83T/96A/174D/195R/210D/318E, 13H/26A/31E/37K/60T/83T/96A/162G/174D/195R/210P/277Y/318E, 13H/16A/17D/31E/37K/60T/79A/83T/96A/162G/174D/195R/210P/318E, 13H/16A/17D/31E/60T/79A/83T/96A/162G/174D/195R/210P/277Y/318E, 13H/16A/17D/26A/37K/60T/79A/96A/162G/174D/195R/210P/277Y/318E, 13H/26A/31E/37K/60T/79A/83T/96A/162G/174D/195R/210P/277Y/318E, 13H/16A/17D/26A/31E/37K/60T/96A/162G/174D/195R/210P/277Y/318E, 13H/16A/17D/26A/31E/37K/79A/83T/96A/162G/174D/195R/210P/318E, 13H/16A/17D/26A/31E/37K/60T/79A/96A/162G/174D/210P/277Y/318E, 13H/17D/26A/31E/37K/60T/79A/83T/96A/162G/174D/195R/210P/277Y/318E, 13H/16A/26A/31E/37K/60T/79A/83T/96A/162G/174D/195R/210P/277Y/318E, 16A/17D/26A/31E/37K/60T/79A/83T/96A/162G/174D/195R/210P/277Y/318E, or 13H/16A/17D/26A/31E/37K/60T/79A/83T/96A/162G/174D/195R/210P/277H/318E, wherein the amino acid positions are relative to the reference sequence corresponding to SEQ ID NO: 906.

In some embodiments, the amino acid sequence of the engineered oxalate decarboxylase polypeptide comprises at least a substitution or substitution set K174D/Q318E, E26A/L96A, V60T/Q318E, K31E/Q318E, K162G/K174D/Q318E, K174D/V277Y/Q318E, E26A/L195R/Q318E, V60T/K162G/L195R, V60T/K162G/Q318E, K174D/L195R/V277Y, S16A/G17D/K174D, V60T/L195R/V277Y, G37K/V277Y/Q318E, S16A/K162G/L195R/V277Y, E26A/G79A/W83T/K162G, E26A/K31E/V60T/Q318E, S16A/K174D/L195R/V277Y, P13H/G37K/W83T/K162G, S16A/G17D/V60T/L195R, P13H/G17D/G37K/W83T, K31E/G37K/K162G/K174D/Q318E, P13H/S16A/K31E/W83T/K162G, S16A/V60T/K174D/L195R/Q318E, G17D/V60T/L96A/K162G/L195R/V277Y, P13H/E26A/V60T/W83T/K162G/K174D, G17D/K31E/V60T/K162G/K174D/V277Y, S16A/G17D/E26A/W83T/L96A/V277Y, G17D/E26A/G37K/K174D/V277Y/Q318E, P13H/S16A/K31E/V60T/K162G/K174D, P13H/G17D/K31E/W83T/V277Y/Q318E, P13H/S16A/G17D/K31E/W83T/K162G/Q318E, P13H/S16A/V60T/W83T/K162G/L195R/Q318E, P13H/S16A/G17D/K31E/G37K/W83T/L96A/K162G, P13H/G17D/E26A/G37K/V60T/W83T/K162G/E210P/Q318E, P13H/S16A/E26A/G37K/W83T/K162G/K174D/L195R/Q318E, P13H/S16A/E26A/K31E/W83T/L96A/K162G/K174D/Q318E, P13H/S16A/G17D/K31E/V60T/W83T/L96A/K162G/K174D/L195R/Q318E, P13H/S16A/E26A/G37K/V60T/W83T/K162G/K174D/L195R/E210P/Q318E, P13H/S16A/G37K/G79A/W83T/L96A/K162G/K174D/L195R/E210P/V277Y, P13H/G17D/E26A/K31E/G37K/V60T/G79A/W83T/L96A/K162G/K174D/E210P, P13H/S16A/G17D/E26A/G37K/G79A/W83T/L96A/K174D/L195R/E210D/Q318E, P13H/E26A/K31E/G37K/V60T/W83T/L96A/K162G/K174D/L195R/E210P/V277Y/Q318E, P13H/S16A/G17D/K31E/G37K/V60T/G7T9A/W83T/L96A/K162G/K174D/L195R/E210P/Q318E, P13H/S16A/G17D/K31E/V60T/G79A/W83T/L96A/K162G/K174D/L195R/E210P/V277Y/Q318E, P13H/S16A/G17D/E26A/G37K/V60T/G79A/L96A/K162G/K174D/L195R/E210P/V277Y/Q318E, P13H/E26A/K31E/G37K/V60T/G79A/W83T/L96A/K162G/K174D/L195R/E210P/V277Y/Q318E, P13H/S16A/G17D/E26A/K31E/G37K/V60T/L96A/K162G/K174D/L195R/E210P/V277Y/Q318E, P13H/S16A/G17D/E26A/K31E/G37K/G79A/W83T/L96A/K162G/K174D/L195R/E210P/Q318E, P13H/S16A/G17D/E26A/K31E/G37K/V60T/G79A/L96A/K162G/K174D/E210P/V277Y/Q318E, P13H/G17D/E26A/K31E/G37K/V60T/G79A/W83T/L96A/K162G/K174D/L195R/E210P/V277Y/Q3 18E, P13H/S16A/E26A/K31E/G37K/V60T/G79A/W83T/L96A/K162G/K174D/L195R/E210P/V277Y/Q3 18E, S16A/G17D/E26A/K31E/G37K/V60T/G79A/W83T/L96A/K162G/K174D/L195R/E210P/V277Y/Q3 18E, or P13H/S16A/G17D/E26A/K31E/G37K/V60T/G79AW83T/L96A/K162G/K174D/L195R/E210P/V27 7H/Q318E, wherein the amino acid positions are relative to the reference sequence corresponding to SEQ ID NO: 906.

In some embodiments, the amino acid sequence of the engineered oxalate decarboxylase polypeptide comprises at least a substitution at an amino acid position set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution set forth in any of Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution or substitution set at amino acid position(s) set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the amino acid sequence of the engineered oxalate decarboxylase polypeptide comprises at least a substitution or substitution set of an oxalate decarboxylase polypeptide set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the engineered oxalate decarboxylase polypeptide comprises the amino acid sequence of an engineered oxalate decarboxylase polypeptide set forth in Tables 4-1, 4-2, 4-3, 4-4, 4-5, 4-6, 4-7, 5-1, and 5-2.

In some embodiments, the engineered oxalate decarboxylase polypeptide comprises an amino acid sequence comprising SEQ ID NO: 4; an amino acid sequence comprising residues 1-359 of an even-numbered SEQ ID NO. of SEQ ID NOs: 28-614; an amino acid sequence comprising an even-numbered SEQ ID NO. of SEQ ID NOs: 28-614; or an amino acid sequence comprising an even-numbered SEQ ID NO. of SEQ ID NOs: 616-1622, optionally wherein the amino acid sequence includes 1, 2, 3, 4, 5, 6, 7, 8, 9, or up to 10 amino acid substitutions, insertions and/or deletions. In some embodiments, optionally the amino acid sequence of the engineered oxalate decarboxylase polypeptide includes 1, 2, 3, 4, or 5 amino acid substitutions, insertions, and/or deletions.

In some embodiments, the engineered oxalate decarboxylase polypeptide comprises an amino acid sequence corresponding to residues 1-359 of SEQ ID NO: 172, 320, or 396, or corresponding to SEQ ID NO: 172, 320, 396, 616, 670, 750, or 906, optionally wherein the amino acid sequence includes 1, 2, 3, 4, 5, 6, 7, 8, 9, or up to 10 amino acid substitutions, insertions and/or deletions. In some embodiments, optionally the amino acid sequence of the engineered oxalate decarboxylase polypeptide includes 1, 2, 3, 4, or 5 amino acid substitutions, insertions, and/or deletions.

In some embodiments of the foregoing, optionally the amino acid sequence includes 1, 2, 3, 4, 5, 6, 7, 8, 9, or up to 10 amino acid substitutions, or 1, 2, 3, 4, or 5 amino acid substitutions. In some embodiments, the substitutions comprise non-conservative substitutions. In some embodiments, the substitutions comprise conservative substitutions.

In some embodiments, the amino acid sequence of the engineered oxalate decarboxylase polypeptide comprises an even-numbered SEQ ID NO. of SEQ ID NOs: 28-174. In some embodiments, the amino acid sequence of the engineered oxalate decarboxylase polypeptide comprises an even-numbered SEQ ID NO. of SEQ ID NOs: 176-394. In some embodiments, the amino acid sequence of the engineered oxalate decarboxylase polypeptide comprises an even-numbered SEQ ID NO. of SEQ ID NOs: 396-530. In some embodiments, the amino acid sequence of the engineered oxalate decarboxylase polypeptide comprises an even-numbered SEQ ID NO. of SEQ ID NOs: 532-614. In some embodiments, the amino acid sequence of the engineered oxalate decarboxylase polypeptide comprises an even-numbered SEQ ID NO. of SEQ ID NOs: 616-728. In some embodiments, the amino acid sequence of the engineered oxalate decarboxylase polypeptide comprises an even-numbered SEQ ID NO. of SEQ ID NOs: 730-822. In some embodiments, the amino acid sequence of the engineered oxalate decarboxylase polypeptide comprises an even-numbered SEQ ID NO. of SEQ ID NOs: 824-908. In some embodiments, the amino acid sequence of the engineered oxalate decarboxylase polypeptide comprises an even-numbered SEQ ID NO. of SEQ ID NOs: 910-1516. In some embodiments, the amino acid sequence of the engineered oxalate decarboxylase polypeptide comprises an even-numbered SEQ ID NO. of SEQ ID NOs: 1518-1622.

In some embodiments, the engineered oxalate decarboxylase has oxalate decarboxylase activity. In some embodiments, the engineered oxalate decarboxylase has oxalate decarboxylase activity and exhibits one or more improved enzyme properties compared to a reference oxalate decarboxylase having a sequence corresponding to SEQ ID NO: 2, 4, 172, 320, 396, 616, 670, 750, or 906, or a reference sequence corresponding to residues 1-359 of SEQ ID NO: 172, 320, or 396.

In some embodiments, the engineered oxalate decarboxylase polypeptide has increased enzyme activity compared to the reference oxalate decarboxylase. In some embodiments, the engineered oxalate decarboxylase polypeptide has increased activity at about pH 6 or less, about pH 5.5 or less, about pH 4.5 or less, about pH 3 or less, about pH 2.6 or less, or about pH 2.4 or less, to about pH 2 compared to the reference oxalate decarboxylase.

In some embodiments, the engineered oxalate decarboxylase polypeptide has increased stability at about pH 6 or less, about pH 5.5 or less, about pH 4.5 or less, about pH 3 or less, about pH 2.6 or less, or about pH 2.4 or less, to about pH 2 compared to the reference oxalate decarboxylase polypeptide.

In some embodiments, the engineered oxalate decarboxylase polypeptide has increased thermostability compared to the reference oxalate decarboxylase.

In some embodiments, the engineered oxalate decarboxylase polypeptide has increased resistance to proteolysis at pH of about 2.2 to 3.2 relative to the reference oxalate decarboxylase polypeptide. In some embodiments, the engineered oxalate decarboxylase polypeptide described herein exhibits increased resistance to proteolysis at pH of about 2.2.

In some embodiments, the engineered oxalate decarboxylase polypeptide described herein exhibits increased resistance to proteolysis by digestive enzymes, such as pepsin or chymotrypsin, particularly pepsin, including at the pHs described in the foregoing, compared to the reference oxalate decarboxylase.

In some embodiments, the improved enzyme property of the engineered oxalate decarboxylase polypeptide is selected from (a) increased activity on oxalate, (b) increased thermal stability, (c) increased stability at acidic pH, (d) increased activity at acidic pH, (e) increased activity at neutral pH, (f) increased expression, (g) increased solubility, and (h) increased resistance to proteolysis, or any combination of (a), (b), (c), (d), (e), (f), (g) and (h), compared to a reference oxalate decarboxylase having a sequence corresponding to SEQ ID NO: 2, 4, 172, 320, 396, 616, 670, 750, or 906.

In some embodiments, the improved property of the engineered oxalate decarboxylase polypeptide is relative to the reference oxalate decarboxylase polypeptide corresponding to SEQ ID NO: 2 or 4.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference amino acid sequence corresponding to SEQ ID NO: 4, 6, 8, 10, 12, 14, 16, 18, 20, 22, 24, or 26, or to a reference amino acid sequence corresponding to residues 1-424 of SEQ ID NO: 6.

In some embodiments, the engineered oxalate decarboxylase polypeptide comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference amino acid sequence corresponding to SEQ ID NO: 4, 6, 8, 10, 12, 14, 16, 18, 20, 22, 24, or 26, or to a reference amino acid sequence corresponding to residues 1-424 of SEQ ID NO: 6, wherein the amino acid sequence comprises one or more substitutions in its amino acid sequence.

In some embodiments, the engineered oxalate decarboxylase polypeptide comprises an amino acid sequence comprising SEQ ID NO: 2, 4, 6, 8, 10, 12, 14, 16, 18, 20, 22, 24, or 26, or an amino acid comprising residues 1-424 of SEQ ID NO: 6. In some embodiments, the foregoing engineered oxalate decarboxylase are provided as compositions, particularly pharmaceutical compositions, as further described herein.

In some embodiments, the present disclosure provides fusion proteins comprising the engineered oxalate decarboxylase polypeptides described herein. In some embodiments, the engineered oxalate decarboxylase polypeptide can be fused to a variety of amino acid sequences, such as polypeptide tags that can be used for detection and/or purification. In some embodiments, the fusion protein of the engineered oxalate decarboxylase polypeptides comprises a glycine-histidine or histidine-tag (His-tag), such as the exemplary engineered oxalate decarboxylase polypeptides with the His-tag sequences disclosed herein. In some embodiments, the fusion protein of the engineered oxalate decarboxylase polypeptides comprises an epitope tag, such as c-myc or hemagglutinin (HA). In some embodiments, the fusion is to the amino (N-) terminus of an engineered oxalate decarboxylase polypeptide. In some embodiments, the fusion is to the carboxy (C-) terminus of an engineered oxalate decarboxylase polypeptide. In some embodiments, when an engineered oxalate decarboxylase polypeptide amino acid sequence herein is presented as a fusion protein, it is also to be understood that the present disclosure also encompasses the engineered oxalate decarboxylase polypeptide without the fusion polypeptide.

It is also known in the art that protein synthesis is initiated with N-terminal methionine in eukaryotes or formylmethionine in prokaryotes and mitochondria. In eukaryotes, the initiating methionine is removed by cleavage of an N-terminal signal peptide present in secreted proteins or by the action of a methionine amino peptidase (MAP). In prokaryotes, the formylmethionine can be removed by formylmethionine deformylase and the resulting methionine removed by a methionine amino peptidase. Accordingly, it is to be understood that for each and every embodiment of an engineered oxalate decarboxylase polypeptide described herein that contains an N-terminal initiating methionine or formylmethionine, the present disclosure also provides engineered oxalate decarboxylase polypeptide lacking the initiating methionine or formylmethionine. By way of example and not limitation, when the present disclosure provides an engineered oxalate decarboxylase polypeptide as an amino acid sequence comprising residues 1-359 in reference to a specified sequence, where amino acid position 1 is an initiating methionine or formylmethionine, in some embodiments, the present disclosure also provides an engineered oxalate decarboxylase polypeptide comprising residues 2 to 359 in reference to the specified sequence, which lacks the initiating methionine or formylmethionine. Similarly, when the present disclosure provides an engineered oxalate decarboxylase polypeptide comprising a sequence corresponding to a specific SEQ ID NO., in some embodiments, the present disclosure also provides an engineered oxalate decarboxylase polypeptide comprising an amino acid sequence corresponding to the specific SEQ ID NO. lacking the N-terminal initiating methionine or formylmethionine at amino acid position 1.

In some embodiments, the present disclosure provides functional or biologically active fragments of the engineered oxalate decarboxylase polypeptides described herein. In some embodiments, functional or biologically active fragments comprise at least about 90%, at least about 95%, at least about 96%, at least about 97%, at least about 98%, or at least about 99% of the activity of the engineered oxalate decarboxylase polypeptide from which it was derived (i.e., the parent engineered oxalate decarboxylase). In some embodiments, functional or biologically active fragments comprise at least about 90%, at least about 91%, at least about 92%, at least about 93%, at least about 94%, at least about 95%, at least about 96%, at least about 97%, at least about 98%, or at least about 99% of the parent sequence of the engineered oxalate decarboxylase polypeptide. In some embodiments the functional or biologically active fragment is truncated by less than 5, less than 10, less than 15, less than 10, less than 25, less than 30, less than 35, less than 40, less than 45, or less than 50 amino acids.

In some embodiments, the functional or biologically fragments comprise at least about 95%, 96%, 97%, 98%, or 99% of the activity of the engineered oxalate decarboxylase polypeptide from which it was derived (i.e., the parent engineered oxalate decarboxylase).

In some embodiments, the functional or biologically active fragment of the engineered oxalate decarboxylase polypeptide described herein includes at least a mutation or mutation set in the amino acid sequence of the engineered oxalate decarboxylase polypeptide described herein. Accordingly, in some embodiments, the functional or biologically active fragments of the engineered oxalate decarboxylase polypeptide displays the enhanced or improved property associated with the mutation or mutation set in the parent engineered oxalate decarboxylase polypeptide. In some embodiments, the functional or biologically active fragments are characterized by an improved property relative to the reference engineered oxalate polypeptide having a sequence corresponding to SEQ ID NO: 2, 4, 172, 320, 396, 616, 670, 750, or 906. In some embodiments, the functional or biologically active fragments exhibits one or more improved enzyme property of increased activity on oxalate, increased thermal stability, increased stability at acidic pH, increased activity at acidic pH, and increased resistance to proteolysis, relative to the reference oxalate decarboxylase polypeptide having a sequence corresponding to SEQ ID NO: 2, 4, 172, 320, 396, 616, 670, 750, or 906. In some embodiments, the improved property of the functional or biologically active fragment is relative to the reference engineered oxalate carboxylase of SEQ ID NO: 2 or 4.

### Polynucleotides Encoding Engineered Polypeptides. Expression Vectors and Host Cells

In another aspect, the present disclosure provides recombinant polynucleotides encoding the engineered oxalate decarboxylase polypeptides described herein. In some embodiments, the recombinant polynucleotide is operatively linked to one or more heterologous regulatory sequences that control gene expression to create a polynucleotide, e.g., an expression vector, capable of expressing the polypeptide. In some embodiments, expression constructs containing at least one heterologous polynucleotide encoding the engineered oxalate decarboxylase polypeptide(s) is introduced into appropriate host cells to express the encoded engineered oxalate decarboxylase polypeptide(s).

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered oxalate decarboxylase polypeptide. Thus, the present invention provides methods and compositions for the production of each and every possible variation of recombinant polynucleotides that encode the engineered oxalate decarboxylase polypeptides described herein by selecting combinations based on the possible codon choices, and all such variations are to be considered specifically disclosed for any polypeptide described herein, including the amino acid sequences presented in the Examples (e.g., in Tables 4-1, 4-2, 4-3, 4-4, 4-5, 4-6, 4-7, 5-1, and/or 5-2).

In some embodiments, the codons are preferably optimized for utilization by the chosen host cell for protein production. For example, preferred codons used in bacteria are typically used for expression in bacteria, preferred codons used in fungi are typically used for expression in fungi, and preferred codons used in mammals are used for expression in mammals and mammalian cells. In some embodiments, codon optimized polynucleotides encoding the engineered oxalate decarboxylase polypeptides contain preferred codons at about 40%, 50%, 60%, 70%, 80%, 90%, or greater than 90% of the codon positions in the full-length coding region.

As discussed above, it is to be understood that the present disclosure provides recombinant polynucleotides encoding each and every one of the oxalate decarboxylase polypeptides described above.

Accordingly, by way of example and not limitation, in some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 2, 4, 172, 320, 396, 616, 670, 750, or 906, or to a reference sequence corresponding to residues 1-359 of SEQ ID NO: 172, 320, or 396, wherein the amino acid sequence comprises one or more amino acid substitutions relative to the reference sequence corresponding to SEQ ID NO: 2, 4, 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2 or 4, wherein the amino acid sequence comprises one or more amino acid substitutions relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 172, 320, 396, 616, 670, 750, or 906, or to the reference sequence corresponding to residues 1-359 of SEQ ID NO: 172, 320, or 396, wherein the amino acid sequence comprises one or more amino acid substitutions relative to the reference sequence of SEQ ID NO: 2 or 4.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 28-1622, or to a reference sequence corresponding to residues 1-359 of an even-numbered SEQ ID NO. of SEQ ID NOs: 28-614, wherein the amino acid sequence comprises one or more amino acid substitutions relative to the reference sequence of SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising as least a substitution or substitution set at amino acid position(s) 31, 210, 318, or 31/210/318, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising as least a substitution or substitution set at amino acid position(s) 16, 26, 174, 195, 196, 210, 226, or 16/26/174/195/196/210/226, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 13, 17, 196, or 13/17/196, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 17, 60, 301, or 17/60/301, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution at position 37, wherein the amino acid position is relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 14, 60, 162, 226, or 14/60/162/226, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 83, 96, 277, or 83/96/277, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 346, 124, 359, 174, 173, 123, 196, 304, 301, 347, 11, 284, 210, 169, 216, 195, 339, 4, 6, 180, 80, 243, 182, 7, 226, 156, 183, 227, 219, 62, 343, 16/26, 16/26/242, 16/26/183/232, 155/206/242, 16/26/339, 16/26/155/206/339, 26, 26/206/339, 31/82/210, 31/82, 31/356, 31/97/226, 31/240/270, 31/82/226, 31/240, 31, 31/210/318, or 31/210, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution at an amino acid position set forth in Tables 4-1, 4-2, 4-3, 4-4, 4-5, 4-6, 4-7, 5-1, and 5-2, wherein the amino acid positions are relative to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution set forth in Tables 4-1, 4-2, 4-3, 4-4, 4-5, 4-6, 4-7, 5-1, and 5-2, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set at amino acid position(s) set forth in Tables 4-1, 4-2, 4-3, 4-4, 4-5, 4-6, 4-7, 5-1, and 5-2, wherein the amino acid positions are relative to the reference sequence corresponding to SEQ ID NO: 2 or 4.

In some embodiments, the recombinant polynucleotide comprises a polynucleotide sequence encoding an engineered oxalate decarboxylase polypeptide comprising an amino acid sequence comprising at least a substitution or substitution set of an engineered oxalate decarboxylase polypeptide set forth in Tables 4-1, 4-2, 4-3, 4-4, 4-5, 4-6, 4-7, 5-1, and 5-2, wherein the amino acid positions are relative to SEQ ID NO: 2 or 4.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of an even-numbered SEQ ID NO. of SEQ ID NOs: 28-614, a reference sequence corresponding to an even-numbered SEQ ID NO. of SEQ ID NOs: 28-614, or a reference sequence corresponding to an even-numbered SEQ ID NO. of SEQ ID NOs: 616-1622.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 28-1622, or to the reference sequence corresponding to residues 1-359 of an even-numbered SEQ ID NO. of SEQ ID NOs: 28-614, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence comprising at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to residues SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence comprising at least a substitution or amino acid residue 4C/R/S, 5C/S, 6A/S/W, 7G, 10A/Q/R, 11R, 13A/C/D/E/F/G/H/I/K/L/M/N/P/Q/R/S/T/V/W/Y, 14L/V, 16A/C/D/E/F/G/H/I/K/L/M/N/P/Q/R/S/T/V/W/Y, 17A/C/D/E/F/G/H/I/K/L/M/N/P/Q/R/S/T/V/W/Y, 18G/Q/R, 19C, 22R, 26A/C/D/E/F/G/H/I/K/L/M/N/P/Q/R/S/T/V/W/Y, 31A/C/D/E/F/G/H/I/K/L/M/N/P/Q/R/S/T/V/W/Y, 33R, 35V, 37A/C/D/E/F/G/H/I/K/L/M/N/P/Q/R/S/T/V/W/Y, 40S, 43C, 44S, 46R, 52T, 54L, 60A/C/D/E/F/G/H/I/K/L/M/N/P/Q/R/S/T/V/W/Y, 61M, 62G, 63A/S, 76S, 79A/C/D/E/F/G/H/I/K/L/M/N/P/Q/R/S/T/V/W/Y, 80L, 82I, 83A/C/D/E/F/G/H/I/K/L/M/N/P/Q/R/S/T/V/W/Y, 85E/G/R, 94N/T, 96A/C/D/E/F/G/H/I/K/L/M/N/P/Q/R/S/T/V/W/Y, 97T, 103Q/S/V, 104Q, 106C, 110V, 117A, 121W, 123G/Q/S, 124A/C/G/Q/R/T, 125E/F/S, 126T, 128A, 141Q, 149T, 153H/Q/S, 155L/P/R/V, 156E, 160T, 162A/C/D/E/F/G/H/I/K/L/M/N/P/Q/R/S/T/V/W/Y, 164R/V, 166R, 169A/G/L, 173Q/V, 174A/C/D/E/F/G/H/I/K/L/M/N/P/Q/R/S/T/V/W/Y, 176L, 180E, 182R, 183C/E/I/K, 186N, 187L/R/S, 188A, 189R, 190G/Q, 193A/G, 195A/C/D/E/F/G/H/I/K/L/M/N/P/Q/R/S/T/V/W/Y, 196F/K/M/R/S/V, 197E, 199G/V, 200N, 205A, 206M, 208G, 210A/C/D/E/F/G/H/I/K/L/M/N/P/Q/R/S/T/V/W/Y, 212A/F/G/L/S/V, 216S/W, 219V, 226S/T, 227E/S, 232T, 233D/H/R, 234L, 240E, 242D, 243V, 263S, 265C, 266V, 267R, 269S, 270L, 273A, 274Q/S, 277A/C/D/E/F/G/H/I/K/L/M/N/P/Q/R/S/T/V/W/Y, 284A/R/S, 297Y, 301I/Q/T, 303T, 304D/G, 314S, 316K/V, 318A/C/D/E/F/G/H/I/K/L/M/N/P/Q/R/S/T/V/W/Y, 331I/L/P/V, 335R, 339G, 342A/E/P/R, 343R/W, 346G/L/Q/W, 347F/R, 350R, 351A/E/H, 356S, or 359L/W, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence comprising at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence comprising at least a substitution at amino acid position 31, 210, or 318, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of SEQ ID NO: 172, or to the reference sequence corresponding to SEQ ID NO: 172, wherein the amino acid sequence comprises one or more amino acid substitutions relative to the reference sequence corresponding to residues 1-359 of SEQ ID NO: 172, or relative to the reference sequence corresponding SEQ ID NO: 172.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 13, 212, 124/196/210/226, 5, 16/26/124/155/195/210/284, 196/226, 16/155/195/210/226, 16/195/210/226, 16/155/174/196, 16/195/226, 318, 16/195/196/210, 16/26/124/155/174/196/210, 195/210, 26/155/174/210, 316, 60, 16/155/174, 16/124/174/196, 16/226, 16/26/174/196/226, 17, 331, 124/195, 16/174/196, 188, 195/196/210, 174/196/210, 16/26/155/174, 16/155/195/196/226, 195/226/284, 16, 240, 16/26/124/155/195/196/226, 183/232/339/343, 183/206, 63, 173/347, 16/26/174/196, 16/284, 46, 16/124/195/196, 274, 174/196, 155/174/196, 174/196/226, 16/124/155/174/195, 16/26/155/174/196, 26/174/196/210/284, 16/195/196/284, 174, 16/124/174/195/210/226/284, 16/174/195/284, 26/174/196/210/226/284, 174/196/226/284, 124/174/196, 26/174/195/210/226, 162, 16/124/195/196/284, 206/343, 16/195/196, 16/26/174/195/196/210/226, 155/195/196/226, 18, 335, 124/155/174/195/226, 26/155/174/195/226, 195/196, 153, 155/174/195, 174/195/210/284, 174/195, 37, 124/174/195/226/284, 176, 16/155/174/195/196/226/284, 10, 169/173, 155, 33, 173/183/343/347, or 104/265, 233, wherein the amino acid positions are relative to the reference sequence corresponding to residues 1-359 of SEQ ID NO: 172, or relative to the reference sequence corresponding SEQ ID NO: 172.

In some embodiments, the recombinant polynucleotide comprises a polynucleotide sequence encoding an engineered oxalate decarboxylase polypeptide comprising an amino acid sequence having at least 70%, 75%, 80%, 85%, 86%, 87%, 88%, 89%, 90%, 91%, 92%, 93%, 94%, 95%, 96%, 97%, 98%, 99%, or more sequence identity to a reference sequence corresponding to residues 1-359 of SEQ ID NO: 320, or to the reference sequence corresponding to SEQ ID NO: 320, wherein the amino acid sequence comprises one or more amino acid substitutions relative to the reference sequence corresponding to residues 1-359 of SEQ ID NO: 320, or relative to the reference sequence corresponding to SEQ ID NO: 320.

In some embodiments, the recombinant polynucleotide comprises a polynucleotide sequence encoding an engineered oxalate decarboxylase comprising an amino acid sequence comprising at least a substitution or substitution set at amino acid position(s) 13/17/196, 17/212/331, 17/26/196/212/226, 17/26/196/212/226/331, 13/17/26/196/331, 17/60/196/226, 17/46/212, 26/60/196/226/331, 17/46/196/212/274/331, 212, 17/196/226, 13/17/26/212/331, 13/17/212/226/331, 196/212/331, 13/17/196/331, 13/17/26/212/226/331, 17/60/196/212/226/331, 13/17/212, 13/17/26/331, 13/17, 13/17/26/196, 46/196/212/226, 17/60/196, 17/196/331, 13/17/46/226/331, 17/196/226/331, 17/46/196/212/331, 17/46/212/331, 17/196, 13/17/212/226, 17/212, 17/26/212, 13/17/226/331, 17/196/212/331, 17/196/212/226/331, 13/17/26/226/331, 46/60/196/226/331, 17/46/196/331, 13/17/46/196, 196/212, 13/17/26, 17/83/263, 17/83/173/227, 17/227/301, 17/83/227/263, 17/83/125, 17/83/173, 17/263/301, 17/125, 17/83/301, 17/173, 17/83/227, 17/227, 17, 17/125/227, 17/83, 128, 76, 141, 110, 79, 117, or 61, wherein the amino acid positions are relative to the reference sequence corresponding to residues 1-359 of SEQ ID NO: 320, or relative to the reference sequence corresponding to SEQ ID NO: 320.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to sequence of residues 1-359 of SEQ ID NO: 396, or to the reference sequence corresponding to SEQ ID NO: 396, wherein the amino acid sequence comprises one or more amino acid substitutions relative to the reference sequence corresponding to residues 1-359 of SEQ ID NO: 396, or relative to the reference sequence corresponding to SEQ ID NO: 396.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 13/17/79, 13/14/17/60/79, 13/17/79/83, 13/17/79/301, 13/14/17/60/79/212, 17/60/197, 13/60/212, 13/14/17/60/79/83/301, 13/17/60/79, 17/60/301, 14/17/79, 17/60/83, 17/79, 13/17/60/79/83/301, 342, 52/190, 94/190, 190/342/351, 13/79, 13/96, 273, 126, 96, 79, 269, 40, 44, 267, 266, 160, 277, 149, 22, 234, 54, 297, 106, or 13/43, wherein the amino acid positions are relative to the reference sequence corresponding to residues 1-359 of SEQ ID NO: 396, or relative to the reference sequence corresponding to SEQ ID NO: 396.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 670, wherein the amino acid sequence comprises one or more amino acid substitutions relative to the reference sequence corresponding to SEQ ID NO: 670.

In some embodiments, the recombinant polynucleotide comprises a polynucleotide sequence encoding an engineered oxalate decarboxylase polypeptide comprising an amino acid sequence comprising at least a substitution at amino acid position 37, 125, 162, 94, 195, 85, 103, 232, 189, 186, 155, 193, 342, 196, 63, 33, 351, 314, 187, 303, 164, 153, 346, 183, 19, 123, 350, 205, 284, 199, 343, 200, 208, 169, 190, or 121, or combinations thereof, wherein the amino acid positions are relative to the reference sequence corresponding to SEQ ID NO: 670.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 616, wherein the amino acid sequence comprises one or more amino acid substitutions relative to the reference sequence corresponding to SEQ ID NO: 616.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 14/26/60/318, 14/26/60/94/162/212, 60/162/226, 60, 14/125/162/212/318, 60/125/226, 14/125/162/226, 26/60/162/226/318, 14/26/94/162/212/226, 14/60/162, 14/60/162/226, 14/94/162/318, 14/94/162, 14/60/226, 14/60/94/212, 14/60/162/212, 14/162, 14/94/212/318, 14/60, 14/26/162, 14, 14/318, 14/162/226/318, 14/125/212, 14/125/226/318, 14/26/60/162, 14/125/162/212/226, 14/94/125/162/318, 14/125/226, 277, 166, 233, 83, 5, 342, 96, 284, or 26, wherein the amino acid positions are relative to the reference sequence corresponding SEQ ID NO: 616.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 750, wherein the amino acid sequence comprises one or more amino acid substitutions relative to the reference sequence corresponding to SEQ ID NO: 750.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 5/26/83/342, 5/26/277, 5/96/277, 5/83/277/342, 26/83/277, 5/26/83/277, 5/277/342, 5/26/166/277, 83/277/342, 83/96/277/342, 83/96/277, 26/83/277/342, 5/26/83/277/342, 5/83/277, 26/277, 83/277, 5/35/277, 5/26/83/96/277, 5/26/96/277, 5/277, 5/166/277, 26/83/96/277/342, 277, 26/277/342, 5, or 5/83/96/277/342, wherein the amino acid positions are relative to the reference sequence corresponding to SEQ ID NO: 750.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 906, wherein the amino acid sequence comprises one or more amino acid substitutions relative to the reference sequence corresponding to SEQ ID NO: 906.

In some embodiments, the recombinant polynucleotide comprises a polynucleotide sequence encoding an engineered oxalate decarboxylase polypeptide comprising an amino acid sequence comprising at least a substitution at amino acid position 26, 31, 96, 60, 318, 210, 277, 37, 16, 195, 79, 17, 13, 83, 162, or 174, or combinations thereof, wherein the amino acid positions are relative to the reference sequence corresponding to SEQ ID NO: 906.

In some embodiments, the recombinant polynucleotide comprises a polynucleotide sequence encoding an engineered oxalate decarboxylase polypeptide comprising an amino acid sequence comprising at least a substitution or substitution set at amino acid position(s) 174/318, 26/96, 60/318, 31/318, 162/174/318, 174/277/318, 26/195/318, 60/162/195, 60/162/318, 174/195/277, 16/17/174, 60/195/277, 37/277/318, 16/162/195/277, 26/79/83/162, 26/31/60/318, 16/174/195/277, 13/3⅞3/162, 16/17/60/195, 13/17/37/83, 31/37/162/174/318, 13/16/31/83/162, 16/60/174/195/318, 17/60/96/162/195/277, 13/26/60/83/162/174, 17/31/60/162/174/277, 16/17/26/83/96/277, 17/26/37/174/277/318, 13/16/31/60/162/174, 13/17/31/83/277/318, 13/16/17/31/83/162/318, 13/16/60/83/162/195/318, 13/16/17/31/37/83/96/162, 13/17/26/37/60/83/162/210/318, 13/16/26/31/83/96/162/174/318, 13/16/17/31/60/83/96/162/174/195/318, 13/16/26/37/60/83/162/174/195/210/318, 13/16/37/79/83/96/162/174/195/210/277, 13/17/26/31/37/60/79/83/96/162/174/210, 13/16/17/26/37/79/83/96/174/195/210/318, 13/26/31/37/60/83/96/162/174/195/210/277/318, 13/16/17/31/37/60/79/83/96/162/174/195/210/318, 13/16/17/31/60/79/83/96/162/174/195/210/277/318, 13/16/17/26/37/60/79/96/162/174/195/210/277/318, 13/26/31/37/60/79/83/96/162/174/195/210/277/318, 13/16/17/26/3⅓7/60/96/162/174/195/210/277/318, 13/16/17/26/31/37/79/83/96/162/174/195/210/318, 13/16/17/26/31/37/60/79/96/162/174/210/277/318, 13/17/26/31/37/60/79/83/96/162/174/195/210/277/318, 13/16/26/31/37/60/79/83/96/162/174/195/210/277/318, 16/17/26/31/37/60/79/83/96/162/174/195/210/277/318, or 13/16/17/26/31/37/60/79/83/96/162/174/195/210/277/318, wherein the amino acid positions are relative to the reference sequence corresponding to SEQ ID NO: 906.

In some embodiments, the recombinant polynucleotide comprises a polynucleotide sequence encoding the engineered oxalate decarboxylase polypeptide comprising an amino acid sequence comprising at least a substitution at an amino acid position set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding the engineered oxalate decarboxylase polypeptide comprising an amino acid sequence comprising at least a substitution set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding the engineered oxalate decarboxylase polypeptide comprising an amino acid sequence comprising at least a substitution or substitution set at amino acid position(s) set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding the engineered oxalate decarboxylase polypeptide comprising an amino acid sequence comprising at least a substitution or substitution set of an oxalate decarboxylase polypeptide set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

In some embodiments, the recombinant polynucleotide comprises a polynucleotide sequence encoding an engineered oxalate decarboxylase polypeptide comprising: an amino acid sequence comprising SEQ ID NO: 4; an amino acid sequence corresponding to residues 1-359 of an even-numbered SEQ ID NO. of SEQ ID NOs: 28-614; an amino acid sequence comprising an even-numbered SEQ ID NO. of SEQ ID NOs: 28-614; or an amino acid sequence comprising an even-numbered SEQ ID NO. of SEQ ID NOs: 616-1622, optionally wherein the amino acid sequence includes 1, 2, 3, 4, 5, 6, 7, 8, 9, or up to 10 substitutions. In some embodiments, the encoded engineered oxalate decarboxylase polypeptide optionally includes 1, 2, 3, 4, or 5 substitutions.

In some embodiments, the recombinant polynucleotide comprises a polynucleotide sequence encoding an engineered oxalate decarboxylase polypeptide comprising an amino acid sequence comprising residues 1-359 of SEQ ID NO: 172, 320, or 396, or comprising SEQ ID NO: 172, 320, 396, 616, 670, 750, or 906, optionally wherein the amino acid sequence includes 1, 2, 3, 4, 5, 6, 7, 8, 9, or up to 10 substitutions. In some embodiments, the encoded engineered oxalate decarboxylase polypeptide optionally includes 1, 2, 3, 4, or 5 substitutions.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to: a reference polynucleotide sequence corresponding to SEQ ID NO: 3; a reference polynucleotide sequence corresponding to nucleotide residues 1-1077 of an odd-numbered SEQ ID NO. of SEQ ID NOs: 27-613; a reference polynucleotide sequence corresponding to an odd-numbered SEQ ID NO. of SEQ ID NOs: 27-613; or a reference polynucleotide sequence corresponding to an odd-numbered SEQ ID NO. of SEQ ID NOs: 615-1621, wherein the recombinant polynucleotide encodes an engineered oxalate decarboxylase polypeptide.

In some embodiments, the recombinant polynucleotide comprising a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 33, 171, 319, 395, 615, 669, 749, or 905, or to a reference polynucleotide sequence corresponding to nucleotide residues 1-1077 of SEQ ID NO: 171, 319, or 395, wherein the recombinant polynucleotide encodes an engineered oxalate decarboxylase polypeptide.

In some embodiments, the recombinant polynucleotide comprises a polynucleotide sequence which is codon-optimized. In some embodiments, the polynucleotide sequence is codon optimized for expression in a bacterial cell, fungal cell, or mammalian cell.

In some embodiments, the recombinant polynucleotide comprises a polynucleotide sequence comprising SEQ ID NO: 1 or 3; a polynucleotide sequence comprising nucleotide residues 1-1077 of an odd-numbered SEQ ID NO. of SEQ ID NOs: 27-613; a polynucleotide sequence comprising an odd-numbered SEQ ID NO. of SEQ ID NOs: 27-613; or a polynucleotide sequence comprising an odd-numbered SEQ ID NO. of SEQ ID NOs: 615-1621.

In some embodiments, the recombinant polynucleotide hybridizes under highly stringent conditions to a reference polynucleotide sequence described herein encoding an engineered oxalate decarboxylase e.g., a recombinant polynucleotide provided in Tables 4-1, 4-2, 4-3, 4-4, 4-5, 4-6, 4-7, 5-1, and 5-2, or a reverse complement thereof. In some embodiments, the reference polynucleotide sequence corresponds to SEQ ID NO: 1 or 3; a polynucleotide sequence corresponding to nucleotide residues 1-1077 of an odd-numbered SEQ ID NO. of SEQ ID NOs: 27-613; a polynucleotide sequence corresponding to an odd-numbered SEQ ID NO. of SEQ ID NOs: 27-613; or a polynucleotide sequence corresponding to an odd-numbered SEQ ID NO. of SEQ ID NOs: 615-1621, or a reverse complement thereof, or a polynucleotide sequence encoding any of the other engineered oxalate decarboxylase provided herein. In some embodiments, the recombinant polynucleotide encodes an engineered oxalate decarboxylase and hybridizes under highly stringent conditions to a reverse complement of a reference polynucleotide sequence corresponding to SEQ ID NO: 1 or 3; a polynucleotide sequence corresponding to nucleotide residues 1-1077 of an odd-numbered SEQ ID NO. of SEQ ID NOs: 27-613; a polynucleotide sequence corresponding to an odd-numbered SEQ ID NO. of SEQ ID NOs: 27-613; or a polynucleotide sequence corresponding to an odd-numbered SEQ ID NO. of SEQ ID NOs: 615-1621.

In some embodiments, the recombinant polynucleotide hybridizes under highly stringent conditions to a reverse complement of a reference polynucleotide sequence encoding an engineered oxalate decarboxylase polypeptide, wherein the engineered oxalate decarboxylase polypeptide comprises an amino acid sequence having one or more amino acid differences relative to the reference sequence corresponding to SEQ ID NO: 2, 4, 172, 320, 396, 616, 670, 750, or 906, or relative to the reference sequence corresponding to residues 1-359 of SEQ ID NO: 172, 320, or 396, at residue positions selected from any positions as set forth in Tables 4-1, 4-2, 4-3, 4-4, 4-5, 4-6, 4-7, 5-1, and 5-2. In some embodiments, the polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to SEQ ID NO: 1 or 3; a polynucleotide sequence corresponding to nucleotide residues 1-1077 of an odd-numbered SEQ ID NO. of SEQ ID NOs: 27-613; a polynucleotide sequence corresponding to an odd-numbered SEQ ID NO. of SEQ ID NOs: 27-613; or a polynucleotide sequence corresponding to an odd-numbered SEQ ID NO. of SEQ ID NOs: 615-1621. In some additional embodiments, the polynucleotide that hybridizes under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to a polynucleotide sequence set forth in Tables 4-1, 4-2, 4-3, 4-4, 4-5, 4-6, 4-7, 5-1, and 5-2.

In some embodiments, the recombinant polynucleotide comprises a polynucleotide sequence encoding an engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference amino acid sequence corresponding to SEQ ID NO: 6, 8, 10, 12, 14, 16, 18, 20, 22, 24, or 26, or to a reference amino acid sequence corresponding to residues 1-424 of SEQ ID NO: 6.

In some embodiments, the recombinant polynucleotide comprises a polynucleotide sequence encoding the engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference amino acid sequence corresponding to SEQ ID NO: 4, 6, 8, 10, 12, 14, 16, 18, 20, 22, 24, or 26, or to a reference amino acid sequence corresponding to residues 1-424 of SEQ ID NO: 6, wherein the amino acid sequence comprises one or more substitutions in its amino acid sequence.

In some embodiments, the recombinant polynucleotide comprises a polynucleotide sequence encoding the engineered oxalate decarboxylase polypeptide comprising an amino acid sequence comprising SEQ ID NO: 4, 6, 8, 10, 12, 14, 16, 18, 20, 22, 24, or 26, or an amino acid comprising residues 1-424 of SEQ ID NO: 6.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 5, 7, 9, 11, 13, 15, 17, 19, 21, 23, or 25, or to a reference polynucleotide sequence corresponding to nucleotide residues 1-1272 of SEQ ID NO: 5, wherein the recombinant polynucleotide encodes an oxalate decarboxylase.

In some embodiments, the recombinant polynucleotide comprises a polynucleotide sequence comprising SEQ ID NO: 5, 7, 9, 11, 13, 15, 17, 19, 21, 23, or 25, or comprising nucleotide residues 1-1272 of SEQ ID NO: 5.

In some embodiments, a recombinant polynucleotide encoding any of the engineered oxalate decarboxylase polypeptides herein is manipulated in a variety of ways to facilitate expression of the engineered polypeptide. In some embodiments, the polynucleotides encoding the polypeptides are provided as expression vectors where one or more control sequences is present to regulate the expression of the polynucleotides and/or polypeptides. In some embodiments, the recombinant expression vector comprises a polynucleotide encoding an engineered oxalate decarboxylase polypeptide, and one or more expression regulating regions such as a promoter and a terminator, a replication origin, etc., depending on the type of hosts into which they are to be introduced. The techniques for modifying polynucleotides and nucleic acid sequences utilizing recombinant DNA methods are well known in the art.

In some embodiments, the control sequences include among others, promoters, leader sequences, polyadenylation sequences, propeptide sequences, signal peptide sequences, and transcription terminators. In some embodiments, suitable promoters are selected based on the host cells selection. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA, 1978, 75:3727-3731), as well as the tac promoter (See e.g., DeBoer et al., Proc. Natl Acad. Sci. USA, 1983, 80: 21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha- amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast, 1992, 8:423-488). Exemplary promoters for use in mammalian cells include, but are not limited to, those from cytomegalovirus (CMV), chicken β-actin promoter fused with the CMV enhancer, Simian vacuolating virus 40 (SV40), from Homo sapiens phosphoglycerate kinase, beta actin, elongation factor-1a or glyceraldehyde-3-phosphate dehydrogenase, or from Gallus β-actin.

In some embodiments, the control sequence is a suitable transcription terminator sequence, a sequence recognized by a host cell to terminate transcription. The terminator sequence is operably linked to the 3′ terminus of the nucleic acid sequence encoding the engineered oxalate decarboxylase polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra). Exemplary terminators for mammalian cells include, but are not limited to, those from cytomegalovirus (CMV), Simian virus 40 (SV40), from Homo sapiens growth hormone hGH, from bovine growth hormone BGH, and from human or rabbit beta globulin.

In some embodiments, the control sequence is a suitable leader sequence, 5′-cap modification, 5′ UTR, etc. In some embodiments, these regulatory sequence elements mediate binding to molecules involved in mRNA trafficking and translation, inhibit 5′-exonucleolytic degradation and confer resistance to de-capping. The leader sequence is operably linked to the 5′ terminus of the nucleic acid sequence encoding the engineered oxalate decarboxylase polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to, those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence comprises a 3′ untranslated nucleic acid region and/or polyadenylation tail nucleic acid sequence, sequences operably linked to the 3′ terminus of the protein coding nucleic acid sequence which mediate binding to proteins involved in mRNA trafficking and translation and mRNA half-life. Any polyadenylation sequence and 3′ UTR which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is also a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to, the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (See e.g., Guo and Sherman, Mol. Cell. Bio., 1995, 15:5983-5990).

In some embodiments, the control sequence is also a signal peptide (i.e., a coding region that codes for an amino acid sequence linked to the amino terminus of a polypeptide and directs the encoded polypeptide into the cell’s secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NClB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 1993, 57:109-137). In some embodiments, effective signal peptide coding regions for filamentous fungal host cells include, but are not limited to, the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to, those from the genes for Saccharomyces cerevisiae alpha-factor and Saccharomyces cerevisiae invertase. Useful signal peptides for mammalian host cells include but are not limited to those from the genes for immunoglobulin gamma (IgG). In some embodiments, the signal peptide is a signal peptide of a protein expressed in human cells.

In some embodiments, the control sequence is also a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In the embodiments herein, the recombinant expression vector may be any suitable vector (e.g., a plasmid or virus), that can be conveniently subjected to recombinant DNA procedures and bring about the expression of the engineered oxalate decarboxylase polynucleotide sequence, and/or expression of the encoded engineered oxalate decarboxylase polypeptide. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to, the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising at least one recombinant polynucleotide or an expression vector encoding at least one engineered ODC polypeptide of the present invention, the polynucleotide(s) being operatively linked to one or more control sequences for expression of the engineered ODC enzyme(s) in the host cell. Host cells suitable for use in expressing the polypeptides encoded by the expression vectors described herein are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Exemplary host cells also include various Escherichia coli strains (e.g., W3110 (ΔfhuA) and BL21).

In another aspect, the present disclosure provides a method of producing the engineered oxalate decarboxylase polypeptide, where the method comprises culturing a host cell capable of expressing a polynucleotide encoding the engineered oxalate decarboxylase polypeptide under conditions suitable for expression of the encoded polypeptide. In some embodiments, the method further comprises a step of isolating or recovering the expressed engineered oxalate decarboxylase polypeptide, e.g., from the culture and/or cells. In some embodiments, the method further comprises a step of purifying the expressed oxalate decarboxylase polypeptide. Purification of the polypeptide can use methods known in the art, including, by way of example and not limitation, precipitation and/or chromatography.

Chromatographic techniques for isolation/purification of the oxalate polypeptides include, among others, reverse phase chromatography, high-performance liquid chromatography, ionexchange chromatography, hydrophobic-interaction chromatography, size-exclusion chromatography, gel electrophoresis, and affinity chromatography. Conditions for purifying a particular enzyme depend, in part, on factors such as net charge, hydrophobicity, hydrophilicity, molecular weight, molecular shape, etc., and will be apparent to those having skill in the art. In some embodiments, affinity techniques may be used to isolate the engineered oxalate decarboxylase polypeptide. In some embodiments, the affinity chromatography can use a tag on the polypeptide, such as a his-tag. In some embodiments, the affinity chromatography can use any antibody that specifically binds an engineered oxalate decarboxylase polypeptide of interest. For the production of antibodies, various host animals, including but not limited to rabbits, mice, rats, etc., are immunized with an engineered oxalate decarboxylase polypeptide, or a fragment thereof. In some embodiments, the engineered oxalate decarboxylase polypeptide or fragment for immunization is attached to a suitable carrier, such as BSA, by means of a side chain functional group or linkers attached to a side chain functional group for immunizing an animal.

Appropriate culture media and growth conditions for host cells are well known in the art. It is contemplated that any suitable method for introducing polynucleotides for expression of the ODC polypeptides into cells will find use in the present invention. Suitable techniques include, but are not limited to electroporation, biolistic particle bombardment, liposome mediated transfection, calcium chloride transfection, and protoplast fusion.

The engineered oxalate decarboxylase polypeptides with the properties disclosed herein can be obtained by subjecting the polynucleotide encoding the naturally occurring or engineered oxalate decarboxylase polypeptide to any suitable mutagenesis and/or directed evolution methods known in the art, and/or as described herein. An exemplary directed evolution technique is mutagenesis and/or DNA shuffling (See e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. 6,537,746). Other directed evolution procedures that can be used include, among others, staggered extension process (StEP), in vitro recombination (See e.g., Zhao et al., Nat. Biotechnol., 1998, 16:258-261), mutagenic PCR (See e.g., Caldwell et al., PCR Methods Appl., 1994, 3:S136-S140), and cassette mutagenesis (See e.g., Black et al., Proc. Natl. Acad. Sci. USA, 1996, 93:3525-3529).

For example, mutagenesis and directed evolution methods can be readily applied to oxalate decarboxylase encoding polynucleotides to generate variant libraries that can be expressed, screened, and assayed. Any suitable mutagenesis and directed evolution methods find use in the present invention and are well known in the art (See e.g., U.S. Pat. Nos, 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related US and non-US counterparts; Ling et al., Anal. Biochem., 1997, 254(2):157-78; Dale et al., Meth. Mol. Biol., 57:369-74 [1996]; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. USA, 1997, 94:4504-4509; Crameri et al., Nat. Biotechnol., 1996, 14:315-319; Stemmer, Nature, 1994, 370:389-391; Stemmer, Proc. Nat. Acad. Sci. USA, 1994, 91:10747-10751; WO 95/22625; WO 97/0078; WO 97/35966; WO 98/27230; WO 00/42651; WO 01/75767; WO 2009/152336; and U.S. Pat. Appln. Publ. Nos. 2011/0082055, 2014/0005057, 2014/0214391, 2014/0221216, 2015/0133307, 2015/0134315, and 2015/0050658; all of which are incorporated herein by reference).

In some embodiments, the enzyme clones obtained following mutagenesis treatment are screened by subjecting the enzyme preparations to a defined temperature, acid pH conditions, exposure to proteases, or other assay conditions, and measuring the amount of enzyme activity after such treatments. Clones containing the polynucleotide encoding an engineered oxalate decarboxylase polypeptide of interest are then isolated, sequenced to identify the nucleotide sequence changes (if any), and used to express the enzyme in a host cell. Measuring enzyme activity from the expression libraries can be performed using any suitable method known in the art, such as described in the Examples.

For engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tet. Lett., 1981, 22:1859-69; and Matthes et al., EMBO J., 1984, 3:801-05), as it is typically practiced in automated synthetic methods. According to the phosphoramidite method, oligonucleotides are synthesized (e.g., in an automatic DNA synthesizer, purified, annealed, ligated and cloned in appropriate vectors).

Accordingly, in some embodiments, a method for preparing the engineered oxalate decarboxylase polypeptide can comprise: (a) synthesizing a polynucleotide encoding an engineered oxalate decarboxylase polypeptide comprising an amino acid sequence corresponding to any polypeptide variant as described herein, and (b) expressing the engineered oxalate decarboxylase polypeptide encoded by the polynucleotide. In some embodiments of the method, the polynucleotide encoding the polypeptide can be designed or engineered to encode a polypeptide optionally having one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

As noted above, the expressed engineered ODC polypeptide can be evaluated for any desired improved property or combination of properties (e.g., activity, stability, protease resistance, etc.) using any suitable assay known in the art, including but not limited to the assays and conditions described herein.

### Compositions of Engineered Oxalate Decarboxylase

In another aspect, the present disclosure provides compositions comprising the engineered oxalate decarboxylase polypeptides described herein for various uses, including but not limited to use as pharmaceuticals, dietary/nutritional supplements, food, feed, and fine chemical production. For example, in some embodiments, the present disclosure provides a composition comprising a food and/or feeds and at least one engineered oxalate decarboxylase polypeptide and/or at least one polynucleotide encoding at least one engineered oxalate decarboxylase polypeptide.

In some embodiments, the engineered oxalate decarboxylase polypeptide find use in any suitable edible enzyme delivery matrix. In some embodiments, the engineered oxalate decarboxylase polypeptide variants are present or formulated in an edible enzyme delivery matrix designed for rapid dispersal of the engineered oxalate decarboxylase polypeptide within the digestive tract of an animal or subject upon ingestion of the polypeptide.

In some embodiments, the engineered oxalate decarboxylase polypeptides are prepared as a pharmaceutical or dietary composition. Depending on the mode of administration, the pharmaceutical composition comprises an engineered oxalate decarboxylase polypeptide described herein, and a pharmaceutically acceptable carrier or excipient. In some embodiments, the pharmaceutical composition comprises a therapeutically effective amount of the engineered oxalate decarboxylase polypeptide. In some embodiments, the pharmaceutical or dietary composition is in the form of a solid, semi- solid, or liquid. In some embodiments, the compositions include other pharmaceutically acceptable components such as diluents, buffers, excipients, salts, emulsifiers, preservatives, stabilizers, fillers, and other ingredients. A description of pharmaceutically acceptable excipients and carriers is available in Remington’s Pharmaceutical Sciences (Mack Pub. Co., N.J. 1991) and Remington: The Science and Practice of Pharmacy, A. Adejare ed., 23 Ed., Academic Press 2020).

In some embodiments, the engineered oxalate decarboxylase polypeptides are formulated for use in oral pharmaceutical compositions. Any suitable form for use in delivering the engineered oxalate decarboxylase polypeptides find use in the present invention, including but not limited to pills, tablets, gel tabs, capsules, lozenges, dragees, powders, soft gels, sol-gels, gels, emulsions, implants, patches, sprays, ointments, liniments, creams, pastes, jellies, paints, aerosols, chewing gums, demulcents, sticks, suspensions (including but not limited to oil-based suspensions, oil-in water emulsions, etc.), slurries, syrups, controlled release formulations, suppositories, etc. In some embodiments, the engineered oxalate decarboxylase polypeptides are provided in a format suitable for injection (i.e., in an injectable formulation).

In some embodiments, compositions comprising the engineered oxalate decarboxylase polypeptides of the present invention include one or more commonly used carrier compounds, including but not limited to sugars (e.g., lactose, sucrose, mannitol, and/or sorbitol), starches (e.g., corn, wheat, rice, potato, or other plant starch), cellulose (e.g., methyl cellulose, hydroxypropylmethyl cellulose, sodium carboxy- methylcellulose), gums (e.g., arabic, tragacanth, guar, etc.), and/or proteins (e.g., gelatin, collagen, etc.). Additional components in oral formulations may include coloring and or sweetening agents (e.g., glucose, sucrose, and mannitol) and lubricating agents (e.g., magnesium stearate), as well as enteric coatings (e.g., methacrylate polymers, hydroxyl propyl methyl cellulose phthalate, and/or any other suitable enteric coating known in the art). In some embodiments, disintegrating or solubilizing agents are included (e.g., cross-linked polyvinyl pyrrolidone, agar, alginic acid or salts thereof, such as sodium alginate). In some embodiments, the engineered oxalate decarboxylase polypeptide are combined with various additional components, including but not limited to preservatives, suspending agents, thickening agents, wetting agents, alcohols, fatty acids, and/or emulsifiers, particularly in liquid formulations.

In some embodiments, the engineered oxalate decarboxylase polypeptide are combined with various additional components, including but not limited to preservatives, suspending agents, thickening agents, wetting agents, alcohols, fatty acids, and/or emulsifiers, particularly in liquid formulations.

In some embodiments, the engineered oxalate decarboxylase polypeptides are provided in biocompatible matrices such as sol- gels, including silica-based (e.g., oxysilane) sol-gels. In some embodiments, the engineered oxalate decarboxylase polypeptides are encapsulated. In some alternative embodiments, the engineered oxalate decarboxylase polypeptides are encapsulated in nanostructures (e.g., nanotubes, nanotubules, nanocapsules, or microcapsules, microspheres, liposomes, etc.).

It is not intended that the present invention be limited to any particular delivery formulation and/or means of delivery. In some embodiments, the engineered oxalate decarboxylase polypeptides be administered by any suitable means known in the art, including but not limited to parenteral, oral, topical, transdermal, intranasal, intraocular, intrathecal, via implants, etc.

In some embodiments, the engineered oxalate decarboxylase polypeptides are chemically modified by glycosylation, pegylation (i.e., modified with polyethylene glycol [PEG] or activated PEG, etc.) or other compounds (See e.g., Ikeda, Amino Acids, 2005, 29:283-287; US Pat. Nos. 7,531,341, 7,534,595, 7,560,263, and 7,553,653; U.S. Pat. Appln. Publ. Nos. 2013/0039898, 2012/0177722, etc.).

In some additional embodiments, the engineered oxalate decarboxylase polypeptides are provided in formulations comprising matrix-stabilized enzyme crystals. In some embodiments, the formulation comprises a cross-linked crystalline engineered oxalate decarboxylase polypeptide and a polymer with a reactive moiety that adheres to the enzyme crystals. The present invention also provides engineered oxalate decarboxylase polypeptides in polymers.

It is to be understood that for the compositions herein, including the pharmaceutical compositions, any of the engineered oxalate decarboxylase polypeptides described herein, including an engineered oxalate carboxylase set forth in Tables 4-1, 4-2, 4-3, 4-4, 4-5, 4-6, 4-7, 5-1, and 5-2 can be used in the compositions.

In some additional embodiments, the pharmaceutical composition comprises an oxalate decarboxylase comprising an amino acid sequence comprising SEQ ID NO: 2, 4, 6, 8, 10, 12, 14, 16, 18, 20, 22, 24, or 26, or an amino acid comprising residues 1-424 of SEQ ID NO: 6.

### Uses of the Engineered Oxalate Decarboxylases

In a further aspect, the engineered decarboxylase polypeptides are used for therapeutic, diagnostic, or industrial purposes.

In some embodiments, the engineered oxalate decarboxylase polypeptides described herein are used to reduce levels of oxalate in a subject, such as a patient afflicted with abnormally elevated or pathological levels of oxalate, such as in the urine and/or plasma. In some embodiments, a method of reducing levels of oxalate in a subject comprises administering to a subject in need thereof an effective amount of an engineered oxalate decarboxylase polypeptide described herein or a composition comprising the engineered oxalate decarboxylase, to reduce levels of oxalate in the subject. In some embodiments, an effective amount of the engineered oxalate decarboxylase is administered for reducing the levels of oxalate in urine and/or plasma of the subject.

In some embodiments, the engineered oxalate decarboxylase polypeptides are used to treat and/or prevent symptoms of hyperoxaluria. In some embodiments, a method for treating and/or preventing the symptoms of hyperoxaluria in a subject comprises administering to a subject with hyperoxaluria an effective amount of an engineered oxalate decarboxylase polypeptide described herein or a composition comprising the engineered oxalate decarboxylase. In some embodiments, the subject is a patient afflicted with primary hyperoxaluria. In some embodiments, the subject is a patient afflicted with secondary hyperoxaluria. In some embodiments, the secondary hyperoxaluria is enteric hyperoxaluria. In some embodiments, the subject is afflicted with idiopathic hyperoxaluria.

In some embodiments, an effective amount of the engineered oxalate decarboxylase is administered is a therapeutically effective amount such that the symptoms of hyperoxaluria are ameliorated. In some embodiments, an effective amount of the engineered oxalate decarboxylase is administered such that the level of oxalate in urine and/or plasma is reduced. In some embodiments, the subject treated with an engineered oxalate carboxylase is able to eat a diet that is higher in its oxalate content compared to diets required by subjects who are afflicted with hyperoxaluria.

In some embodiments of the methods above, the subject treated with an engineered oxalate decarboxylase polypeptide is an infant, child, young adult, or adult.

In some embodiments, the dosage of engineered oxalate decarboxylase polypeptide(s) administered to a patient depends upon, among others, the general condition of the patient, age, sex, weight, and the disease/condition being treated, and other factors known to those in the art. In some embodiments, the compositions are intended for single or repeat administration to a patient. Preferably, the compositions are administered repeatedly. In some embodiments, the amount of engineered oxalate decarboxylase polypeptide(s) in the composition(s) administered to a patient is sufficient to effectively treat, ameliorate and/or prevent the symptoms of the disease or condition.

In some embodiments, the engineered oxalate decarboxylase is administered at about 0.1 to about 3 g/dose. In some embodiments, the engineered oxalate decarboxylase is administered at a dose of about 0.2 to about 2.5 g, about 0.25 to about 2 g, or about 0.5 to about 1.5 g. In some embodiments, the engineered oxalate decarboxylase is administered at a dose of about 0.1, 0.2, 0.5, 1, 1.5, 2. 2.5, or 3 g.

In some embodiments, the engineered oxalated decarboxylase is administered at about 100 U/kg body weight to about 5000 U/kg body weight. In some embodiments, the engineered oxalated decarboxylase is administered at about 300 U/kg body weight to about 3000 U/kg body weight. In some embodiments, the engineered oxalated decarboxylase is administered at about, 500 U/kg to about 2000 U/kg body weight. A Unit of enzyme is provided in Example 12.

In some embodiments, the engineered oxalate decarboxylase is administered 1-5 times per day, 1-4 times per day, 1-3 times per day, or 1-2 times per day. In some embodiments, the engineered oxalate decarboxylase is administered 2-5 time per day, 2-4 times per day, or 2-4 times per day. In some embodiments, the engineered oxalate decarboxylase is administered once (1) per day, 2 times per day, 3 times per day, 4 times per day, or 5 times per day.

In some embodiments, the engineered oxalate decarboxylase is administered prior to, concurrently with, or subsequent to ingestion of food or meal. In some embodiments, the engineered oxalate decarboxylase is administered orally prior to, concurrently with, or subsequent to ingestion of food or meal. In some embodiments, the engineered oxalate decarboxylase is administered together with food or a meal.

In some embodiments, the engineered oxalate decarboxylase polypeptides are used in diagnostics, e.g., for detecting oxalate (Costello et al, J. Lab. Clin. Med., 1976, 87(5):903-908); Parkinson et al., Clin Chim Acta., 1985, 152(3):335-45). In some embodiments, a method of determining amount of oxalate in a sample comprises contacting a sample with an engineered oxalate decarboxylase described herein and determining amount of formate product. In some embodiments, the amount of formate can be determined by use of formate dehydrogenase or formyl-CoA transferase (U.S. Pat. No. 5,604,111).

In some embodiments, the engineered oxalate decarboxylase is used for industrial purposes in reducing levels of oxalate, such as in reducing formation of calcium oxalate deposits/precipitates. Deposits or precipitates of calcium oxalate are problematic in pulp and paper industries, including removal of oxalate from industrial wastewater. In some embodiments, the engineered oxalate decarboxylase is added to industrial plant filtrates containing oxalate, e.g., to prevent or reduce scaling.

The foregoing and other aspects of the invention may be better understood in connection with the following non-limiting examples. These Examples, including experiments and results achieved, are provided for illustrative purposes only and are not to be construed as limiting the present invention.

### EXAMPLES

In the experimental disclosure below, the following abbreviations apply where appropriate: ppm (parts per million); M (molar); mM (millimolar), uM and µM (micromolar); nM (nanomolar); mol (moles); gm and g (gram); mg (milligrams); ug and µg (micrograms); L and l (liter); ml and mL (milliliter); cm (centimeters); mm (millimeters); um and µm (micrometers); sec. (seconds); min(s) (minute(s)); h(s) and hr(s) (hour(s)); U (units); MW (molecular weight); rpm (rotations per minute); psi and PSI (pounds per square inch); °C (degrees Centigrade); RT and rt (room temperature); CDS (coding sequence); DNA (deoxyribonucleic acid); RNA (ribonucleic acid); AUC (area under the curve); E. coli W3110 (commonly used laboratory E. coli strain, available from the Coli Genetic Stock Center [CGSC], New Haven, CT); HTP (high throughput); HPLC (high pressure liquid chromatography); LC (liquid chromatography); MS (mass spectroscopy); LC-MS/MS (liquid chromatography with two mass spectrometers); SPE (solid phase extraction); IPTG (isopropyl β-D-1-thiogalactopyranoside); PLP (pyridoxal 5′-phosphate); BSA (bovine serum albumin); BW (body weight); FIOPC (fold improvements over positive control); FIOP (fold improvement over parent); LB (Luria broth); TB (Terrific broth).

### Example 1

### Oxalate Decarboxylase Gene Acquisition and Construction of Expression Vectors

Bacterial ODCs from different organisms in Table 1-1 were codon optimized for expression in E. coli, synthesized and cloned into the E. coli expression vector pCK110900 vector system (See e.g., U.S. Pat. Appln. Publn. 2006/0195947, which is hereby incorporated by reference herein). The plasmid construct was transformed into an E. coli strain derived from W3110. The E. coli strains containing the ODC genes were grown in 96-well format and assayed for ODC activity (40 mM oxalate, pH 4.0) as described below in Example 3. The activity obtained from these ODCs are shown in Table 1-1.

TABLE 1-1

Unchallenged activity at pH 4.0 for wild type oxalate decarboxylases

| SEQ ID NO: (nt/aa) | Source Organism | Activity at pH 4.0 Relative to SEQ ID NO: 2 |
|---|---|---|
| ½ | Gemmata sp. SH-PL17 | ++++ |
| ⅚ | Flammulina velutipes | +++ |
| ⅞ | Bacillus subtilis | + |
| 9/10 | Bacillus vallismortis | ++++ |
| 11/12 | Bacillus glycinifermentans | + |
| 13/14 | Bacillus atrophaeus | + |
| 15/16 | Bacillus megaterium | ++ |
| 17/18 | Agrobacterium tumefaciens str. C58 | ++++ |
| 19/20 | Rhizobiales bacterium | ++++ |
| 21/22 | Hyphomicrobium sp. 802 | +++ |
| 23/24 | Shinella sp. | + |
| 25/26 | Legionella maceachernii | ++ |

All activities are expressed as a change in absorbance at 340 nm (ΔA340). Levels of activity are defined as follows: “+” 0.15 to 0.50, “++” > 0.50, “+++” > 1.0,“++++”>1.5.

### Example 2

### Engineered ODC Variants Expression and Construction

Based on the results obtained from Table 1-1, the ODC from SEQ ID: 2 was subcloned with and without a C-terminal 6xHis-tag into the pJV110900 vector system (See e.g., U.S. Pat. Appln Publ. 2017/213758) to generate SEQ ID: 4 and 2 respectively. The plasmid construct was transformed into an E. coli strain derived from W3110. Directed evolution techniques generally known by those skilled in the art were used to generate libraries of gene variants from this plasmid construct (See e.g., US Pat. No. 8,383,346, WO2010/144103, and references cited herein) as well as its derivatives.

### Example 3

### High-Throughput (HTP) Growth of ODC Variants and Screening Conditions

### High-Throughput (HTP) Growth of Escherichia Coli ODC and Variants

Transformed E. coli cells were selected by plating onto LB agar plates containing 1% glucose with selection. After overnight incubation at 37° C., colonies were placed into the wells of 96-well shallow flat bottom plates (NUNC™, Thermo-Scientific) filled with 180 µl/well LB supplemented with 1% glucose and selection. The cultures were allowed to grow overnight for 18-20 hours in a shaker (200 rpm, 30° C., and 85% relative humidity; Kuhner).

Overnight growth samples (20 µL) were transferred into Costar 96-well deep plates filled with 380 µL of Terrific Broth supplemented with a selection compound. The plates were incubated for approximately 2 hours in a shaker (250 rpm, 30° C., and 85% relative humidity; Kuhner). The cells were then induced with 40 µL of 10 mM IPTG and 10 mM manganese chloride in sterile water and incubated overnight for 20-24 hours in a shaker (250 rpm, 30° C., and 85% relative humidity; Kuhner). The cells were pelleted (4000 rpm × 20 min), the supernatants were discarded, and the cells were frozen at -80° C. prior to analysis.

### Lysis of HTP Pellets

First, 400 µL of lysis buffer (1X PBS, 1 mg/ml lysozyme, and 0.5 mg/ml polymyxin B sulfate) were added to the cell pellets. The mixture was agitated for 1.5-2 hours at room temperature, and centrifuged (4000 rpm × 15 min) prior to use of the clarified lysates in the various HTP assays described herein. Analysis of these lysates by SDS-PAGE revealed the presence of an overexpressed protein bands at an apparent MW of 40-50 kDa, consistent with the expected MW of ODC. Sometimes additional dilutions of clarified lysate were performed with PBS buffer prior to challenges and analysis.

### Analysis of Clarified Lysates for ODC Activity

ODC activity was assessed using potassium oxalate as a substrate and quantifying formate produced using formate dehydrogenase (FDH). Oxalate assay solution was prepared by dissolving 4.44-44.4 mM potassium oxalate in McIlvaine buffer pH 2.0-4.0 or pH 5.5-6.5. Lysate was diluted 4-200x in 1X PBS pH 7.0-7.4. For dilutions >10x, sequential dilutions were performed, wherein the first dilution was in 1X PBS, pH 7.0-7.4 and the secondary dilutions were in water. To set up ODC activity reactions, 10 µL diluted ODC was added to 90 µL oxalate solution in a round bottom plate (Costar), except in Table 1-1, where 10uL lysate was added to 40 µL 50 mM oxalate solution. Reactions were incubated at 37° C. for 1 hour and quenched by mixing 1:1 with 1 M potassium phosphate dibasic. Formate detection assay solution was prepared by dissolving 25 mM nicotinamide adenine dinucleotide (NAD) in 125 mM potassium phosphate pH 7.5. To set up formate detection reactions, 10-25 µL quenched oxalate assay sample was added to 20 µL formate detection solution with 0-15 µL water added to bring to 45 µL total, before 5 µL 10 g/L formate dehydrogenase (FDH; Codexis) was added in a UV-Star® half area, flat bottom plate (Greiner). Reactions were incubated at 25° C. for 10 min, shaking 200 rpm, and absorbance at 340 nm was read on a Molecular Devices Spectramax plate reader. Background subtracted absorbance (Table 1-1) or absorbance relative to that of the round backbone (fold improvement over parent (FIOP)) were used for activity values.

### HTP Analysis of Clarified Lysates Pretreated With Low pH and Pepsin

The activities of variants were determined after pre-incubation at low pH in the presence of pepsin to simulate the environment of the stomach. First, clarified lysate was diluted 0x -8x in PBS buffer, then mixed 1:1 with McIlvaine buffer pH 2.2-3.2 with 0.2-1.6 mg/mL pepsin from porcine gastric mucosa (Sigma) in a round bottom plate (Costar), for a final challenge pH of 2.2-3.0 and a final pepsin concentration of 0.1-0.8 mg/mL. Samples were mixed then incubated for 1-2 hours at 37° C. Samples were then analyzed for residual ODC activity as described in Examples 3.3, except that samples were taken directly into oxalate assay solution (pH 2.4-3.0) without diluting in PBS.

### Example 4

### Screening Results of ODC Variants Based on SEQ ID NO: 4

Library variants were generated from homologs diversity and mutagenesis at surface positions based on a structural model of SEQ ID NO: 4. These variants were screened in triplicates for ODC activity at pH 3.0 with 10 mM potassium oxalate after a 1 hour simulated gastric challenge (0.2 mg/mL pepsin, pH 3.0) and unchallenged ODC activity at pH 3.0 with 4 mM and 10 mM potassium oxalate as described in Example 3. Analysis of the data relative to SEQ ID NO: 4 are listed in Table 4-1.

TABLE 4-1

Oxalate decarboxylase activity Relative to SEQ ID NO: 4

| SEQ ID NO: (nt/aa) | Amino Acid Differences (Relative to SEQ ID NO: 2 or 4) | FIOP Unchallenged Activity at pH 3.0, 10 mM oxalate Relative to SEQ ID NO: 4 | FIOP Unchallenged Activity at pH 3.0, 4 mM oxalate Relative to SEQ ID NO: 4 | FIOP Pepsin Challenge Relative to SEQ ID NO: 4 |
|---|---|---|---|---|
| ¾ | | + | + | + |
| 27/28 | E346G | | + | + |
| 29/30 | E346L | | + | ++ |
| 31/32 | K124G | + | + | + |
| 33/34 | K124T | + | + | + |
| 35/36 | K359L | + | + | ++ |
| 37/38 | D174K | + | + | ++ |
| 39/40 | K173V | + | + | ++ |
| 41/42 | D123Q | | + | + |
| 43/44 | K196F | + | + | + |
| 45/46 | R304D | | | + |
| 47/48 | V301Q | | | + |
| 49/50 | K347F | | | + |
| 51/52 | K359W | + | + | + |
| 53/54 | K124Q | + | + | ++ |
| 55/56 | K124A | + | + | ++ |
| 57/58 | P11R | | | + |
| 59/60 | K284R | + | + | + |
| 61/62 | P210L | + | + | + |
| 63/64 | D169L | + | ++ | + |
| 65/66 | R304G | + | + | + |
| 67/68 | K216W | | | + |
| 69/70 | K347R | + | + | + |
| 71/72 | K196V | + | ++ | ++ |
| 73/74 | K284A | + | + | + |
| 75/76 | R195L | + | + | ++ |
| 77/78 | S339G | + | + | ++ |
| 79/80 | P4S | | + | + |
| 81/82 | K196R | + | + | + |
| 83/84 | F6A | | | + |
| 85/86 | K124C | + | + | + |
| 87/88 | R195Y | + | + | + |
| 89/90 | R180E | + | + | + |
| 91/92 | F80L | + | + | + |
| 93/94 | P243V | | + | + |
| 95/96 | A182R | + | + | + |
| 97/98 | P210E | ++ | + | ++ |
| 99/100 | M7G | | | + |
| 101/102 | T226S | + | ++ | + |
| 103/104 | D174A | + | + | + |
| 105/106 | K196M | | + | + |
| 107/108 | P4C | ++ | ++ | ++ |
| 109/110 | L156E | | + | + |
| 111/112 | V183E | + | + | + |
| 113/114 | R227S | + | + | + |
| 115/116 | R219V | | + | + |
| 117/118 | E62G | | | + |
| 119/120 | D174G | + | + | + |
| 121/122 | D169G | + | + | + |
| 123/124 | K124R | + | + | + |
| 125/126 | P210V | + | ++ | ++ |
| 127/128 | S343R | + | + | + |
| 129/130 | K216S | + | ++ | ++ |
| 131/132 | P210R | + | + | + |
| 133/134 | F6W | | + | + |
| 135/136 | P4R | | | + |
| 137/138 | F6S | | | + |
| 139/140 | A16S/A26E | | + | ++ |
| 141/142 | A16S/A26E/E242D | | + | + |
| 143/144 | A16S/A26E/V183I/K232T | | + | ++ |
| 145/146 | A155P/L206M/E242D | | + | ++ |
| 147/148 | A16S/A26E/S339G | + | + | ++ |
| 149/150 | A16S/A26E/A155P/L206M/S339G | | + | ++ |
| 151/152 | A26E | + | + | ++ |
| 153/154 | A26E/L206M/S339G | + | + | ++ |
| 155/156 | E31K/L82I/P210A | + | + | +++ |
| 157/158 | E31K/L82I | + | + | ++ |
| 159/160 | E31K/A356S | + | + | ++ |
| 161/162 | E31K/A97T/T226S | | + | ++ |
| 163/164 | E31K/D240E/M270L | + | + | +++ |
| 165/166 | E31K/L82I/T226S | + | ++ | +++ |
| 167/168 | E31K/D240E | + | + | ++ |
| 169/170 | E31K | + | + | ++ |
| 171/172 | E31K/P210A/E318Q | + | ++ | +++ |
| 173/174 | E31K/P210A | + | + | +++ |

All activities were determined relative to the reference polypeptide of SEQ ID NO: 4. Levels of increased activity are defined as follows: “+” 0.9 to 1.1, “++” > 1.1, “+++” > 2

### Example 5

### Screening Results of ODC Variants Based on SEQ ID NO: 172

Based on the results from Table 4-1, SEQ ID NO: 172 was chosen as the backbone. Beneficial mutations identified from Table 4-1 were recombined into the backbone. The variants were assayed in triplicate for ODC activity at pH 2.6 with 4 mM potassium oxalate after a 1 hour simulated gastric challenge (0.1 mg/mL pepsin, pH 2.8) and unchallenged ODC activity at pH 2.6 with 4 mM potassium oxalate as described in Example 3. Analysis of the data relative to SEQ ID NO: 172 are listed in Table 4-2.

TABLE 4-2

Oxalate decarboxylase activity Relative to SEQ ID NO: 172

| SEQ ID NO: (nt/aa) | Amino Acid Differences (Relative to SEQ ID NO: 172) | Amino Acid Differences (Relative to SEQ ID NO: 4) | FIOP Unchallenged Activity at pH 2.6 Relative to SEQ ID NO: 172 | FIOP Pepsin Challenge Relative to SEQ ID NO: 172 |
|---|---|---|---|---|
| 175/176 | H13P | H13P/E31K/P210A/E318Q | +++ | +++ |
| 177/178 | H212S | E31K/P210A/H212S/E318Q | +++ | +++ |
| 179/180 | K124A/K196V/A210E/ T226S | E31K/K124A/K196V/P210E/T226S/E318Q | +++ | +++ |
| 181/182 | T5S | T5S/E31K/P210A/E318Q | +++ | ++ |
| 183/184 | H13E | H13E/E31K/P210A/E318Q | +++ | ++ |
| 185/186 | A16S/A26E/K124A/A155P/R195L/A210E/K284A | A16S/A26E/E31K/K124A/A155P/R195L/P210E/K284A/E318Q | +++ | +++ |
| 187/188 | K196V/T226S | E31K/K196V/P210A/T226S/E318Q | +++ | + |
| 189/190 | A16S/A155P/R195L/A210E/T226S | A16S/E31K/A155P/R195L/P210E/T226S/E318Q | +++ | +++ |
| 191/192 | A16S/R195L/A210E/T226S | A16S/E31K/R195L/P210E/T226S/E318Q | +++ | +++ |
| 193/194 | A16S/A155P/D174K/K196V | A16S/E31K/A155P/D174K/K196V/P210A/E318Q | +++ | +++ |
| 195/196 | A16S/R195L/T226S | A16S/E31K/R195L/P210A/ T226S/E318Q | +++ | +++ |
| 197/198 | H13L | H13L/E31K/P210A/E318Q | +++ | ++ |
| 199/200 | Q318E | E31K/P210A | ++ | +++ |
| 201/202 | A16S/R195L/K196V/A210E | A16S/E31K/R195L/K196V/ P210E/E318Q | ++ | +++ |
| 203/204 | A16S/A26E/K124A/A155P/D174K/K196V/A210E | A16S/A26E/E31K/K124A/A155P/D174K/K196V/P210E/E318Q | ++ | +++ |
| 205/206 | R195L/A210E | E31K/R195L/P210E/E318Q | ++ | +++ |
| 207/208 | A26E/A155P/D174K/A210E | A26E/E31K/A155P/D174K/P210E/E318Q | ++ | +++ |
| 209/210 | N316V | E31K/P210A/N316V/E318Q | ++ | +++ |
| 211/212 | T60V | E31K/T60V/P210A/E318Q | ++ | +++ |
| 213/214 | A16S/A155P/D174K | A16S/E31K/A155P/D174K/P210A/E318Q | ++ | +++ |
| 215/216 | A16S/K124A/D174K/K196V | A16S/E31K/K124A/D174K/ K196V/P210A/E318Q | ++ | +++ |
| 217/218 | A16S/T226S | A16S/E31K/P210A/T226S/E318Q | ++ | ++ |
| 219/220 | A16S/A26E/D174K/K196V/T226S | A16S/A26E/E31K/D174K/K196V/P210A/T226S/E318Q | ++ | +++ |
| 221/222 | D17H | D17H/E31K/P210A/E318Q | ++ | +++ |
| 223/224 | A331V | E31K/P210A/E318Q/A331V | ++ | +++ |
| 225/226 | K124A/R195L | E31K/K124A/R195L/P210A/E318Q | ++ | +++ |
| 227/228 | A16S/D174K/K196V | A16S/E31K/D174K/K196V/P210A/E318Q | ++ | +++ |
| 229/230 | D17A | D17A/E31K/P210A/E318Q | ++ | +++ |
| 231/232 | P188A | E31K/P188A/P210A/E318Q | ++ | ++ |
| 233/234 | R195L/K196V/A210E | E31K/R195L/K196V/P210E/E318Q | ++ | +++ |
| 235/236 | D174K/K196V/A210E | E31K/D174K/K196V/P210E/E318Q | ++ | +++ |
| 237/238 | T5C | T5C/E31K/P210A/E318Q | ++ | ++ |
| 239/240 | A16S/A26E/A155P/D174K | A16S/A26E/E31K/A155P/D174K/P210A/E318Q | ++ | +++ |
| 241/242 | A16S/A155P/R195L/K196V/T226S | A16S/E31K/A155P/R195L/K196V/P210A/T226S/E318Q | ++ | +++ |
| 243/244 | R195L/T226S/K284A | E31K/R195L/P210A/T226S/K284A/E318Q | ++ | +++ |
| 245/246 | D17N | D17N/E31K/P210A/E318Q | ++ | ++ |
| 247/248 | A16L | A16L/E31K/P210A/E318Q | ++ | +++ |
| 249/250 | D240E | E31K/P210A/D240E/E318Q | ++ | ++ |
| 251/252 | A16S/A26E/K124A/A155P/R195L/K196V/T226S | A16S/A26E/E31K/K124A/A155P/R195L/K196V/P210A/T226S/E318Q | ++ | ++ |
| 253/254 | A331P | E31K/P210A/E318Q/A331P | ++ | ++ |
| 255/256 | V183I/K232T/S339G/S343R | E31K/V183I/P210A/K232T/ E318Q/S339G/S343R | ++ | + |
| 257/258 | V183I/L206M | E31K/V183I/L206M/P210A/E318Q | ++ | ++ |
| 259/260 | P63A | E31K/P63A/P210A/E318Q | ++ | ++ |
| 261/262 | K173V/K347R | E31K/K173V/P210A/E318Q/K347R | ++ | + |
| 263/264 | A16S/A26E/D174K/K196V | A16S/A26E/E31K/D174K/K196V/P210A/E318Q | ++ | ++ |
| 265/266 | A16S/K284A | A16S/E31K/P210A/K284A/E318Q | ++ | +++ |
| 267/268 | T60R | E31K/T60R/P210A/E318Q | ++ | ++++ |
| 269/270 | V46R | E31K/V46R/P210A/E318Q | ++ | +++ |
| 271/272 | A16S/K124A/R195L/K196V | A16S/E31K/K124A/R195L/ K196V/P210A/E318Q | ++ | +++ |
| 273/274 | H274S | E31K/P210A/H274S/E318Q | ++ | +++ |
| 275/276 | A331I | E31K/P210A/E318Q/A331I | ++ | +++ |
| 277/278 | D174K/K196V | E31K/D174K/K196V/P210A/E318Q | ++ | +++ |
| 279/280 | A155P/D174K/K196V | E31K/A155P/D174K/K196V/P210A/E318Q | ++ | +++ |
| 281/282 | D174K/K196V/T226S | E31K/D174K/K196V/P210A/T226S/E318Q | ++ | +++ |
| 283/284 | A331L | E31K/P210A/E318Q/A331L | ++ | +++ |
| 285/286 | A16S/K124A/A155P/D174K/R195L | A16S/E31K/K124A/A155P/D174K/R195L/P210A/E318Q | ++ | +++ |
| 287/288 | A16S/A26E/A155P/D174K/K196V | A16S/A26E/E31K/A155P/D174K/K196V/P210A/E318Q | ++ | +++ |
| 289/290 | A26E/D174K/K196V/A210E/K284A | A26E/E31K/D174K/K196V/P210E/K284A/E318Q | ++ | +++ |
| 291/292 | H212A | E31K/P210A/H212A/E318Q | ++ | +++ |
| 293/294 | A16S/R195L/K196V/K284A | A16S/E31K/R195L/K196V/P210A/K284A/E318Q | ++ | +++ |
| 295/296 | D174K | E31K/D174K/P210A/E318Q | ++ | +++ |
| 297/298 | A16S/K124A/D174K/R195L/A210E/T226S/K284A | A16S/E31K/K124A/D174K/ R195L/P210E/T226S/K284A/E318Q | ++ | +++ |
| 299/300 | A16S/D174K/R195L/K284A | A16S/E31K/D174K/R195L/ P210A/K284A/E318Q | ++ | +++ |
| 301/302 | A26E/D174K/K196V/A210E/T226S/K284A | A26E/E31K/D174K/K196V/P210E/T226S/K284A/E318Q | ++ | +++ |
| 303/304 | H212L | E31K/P210A/H212L/E318Q | ++ | +++ |
| 305/306 | D174K/K196V/T226S/ K284A | E31K/D174K/K196V/P210A/T226S/K284A/E318Q | ++ | +++ |
| 307/308 | K124A/D174K/K196V | E31K/K124A/D174K/K196V/P210A/E318Q | ++ | +++ |
| 309/310 | A26E/D174K/R195L/A210E/T226S | A26E/E31K/D174K/R195L/P210E/T226S/E318Q | ++ | +++ |
| 311/312 | G162A | E31K/G162A/P210A/E318Q | ++ | + |
| 313/314 | A16S/K124A/R195L/K196V/K284A | A16S/E31K/K124A/R195L/K196V/P210A/K284A/E318Q | ++ | +++ |
| 315/316 | L206M/S343R | E31K/L206M/P210A/E318Q/S343R | ++ | + |
| 317/318 | A16S/R195L/K196V | A16S/E31K/R195L/K196V/P210A/E318Q | ++ | +++ |
| 319/320 | A16S/A26E/D174K/R195L/K196V/A210E/T226S | A16S/A26E/E31K/D174K/R195L/K196V/P210E/T226S/E318Q | ++ | +++ |
| 321/322 | A155P/R195L/K196V/T226S | E31K/A155P/R195L/K196V/P210A/T226S/E318Q | + | ++ |
| 323/324 | K18Q | K18Q/E31K/P210A/E318Q | + | ++ |
| 325/326 | A335R | E31K/P210A/E318Q/A335R | + | + |
| 327/328 | K124A/A155P/D174K/R195L/T226S | E31K/K124A/A155P/D174K/R195L/P210A/T226S/E318Q | + | +++ |
| 329/330 | D17C | D17C/E31K/P210A/E318Q | + | ++ |
| 331/332 | A26E/A155P/D174K/R195L/T226S | A26E/E31K/A155P/D174K/R195L/P210A/T226S/E318Q | + | +++ |
| 333/334 | R195L/K196V | E31K/R195L/K196V/P210 A/E318Q | + | ++ |
| 335/336 | G162T | E31K/G162T/P21OA/E318Q | + | ++ |
| 337/338 | P153H | E31K/P153H/P210A/E318Q | + | ++ |
| 339/340 | N316K | E31K/P210A/N316K/E318Q | + | ++ |
| 341/342 | A155P/D174K/R195L | E31K/A155P/D174K/R195L/P210A/E318Q | + | +++ |
| 343/344 | P153Q | E31K/P153Q/P210A/E318Q | + | ++ |
| 345/346 | D174K/R195L/A210E/ K284A | E31K/D174K/R195L/P210E/K284A/E318Q | + | +++ |
| 347/348 | D174K/R195L | E31K/D174K/R195L/P210A/E318Q | + | +++ |
| 349/350 | P153S | E31K/P153S/P210A/E318Q | + | ++ |
| 351/352 | H212F | E31K/P210A/H212F/E318Q | + | ++ |
| 353/354 | K37C | E31K/K37C/P210A/E318Q | + | + |
| 355/356 | H212V | E31K/P210A/H212V/E318Q | + | +++ |
| 357/358 | K124A/D174K/R195L/ T226S/K284A | E31K/K124A/D174K/R195L/P210A/T226S/K284A/E318Q | + | +++ |
| 359/360 | V176L | E31K/V176L/P210A/E318Q | + | + |
| 361/362 | A16S/A155P/D174K/R195L/K196V/T226S/K284A | A16S/E31K/A155P/D174K/R195L/K196V/P210A/T226S/K284A/E318Q | + | +++ |
| 363/364 | V10Q | V10Q/E31K/P210A/E318Q | + | ++ |
| 365/366 | D169G/K173V | E31K/D169G/K173V/P210A/E318Q | + | + |
| 367/368 | A155L | E31K/A155L/P210A/E318Q | + | + |
| 369/370 | K18G | K18G/E31K/P210A/E318Q | + | +++ |
| 371/372 | T60L | E31K/T60L/P210A/E318Q | + | ++ |
| 373/374 | V10A | V10A/E31K/P210A/E318Q | + | ++ |
| 375/376 | K18R | K18R/E31K/P210A/E318Q | + | ++ |
| 377/378 | K33R | E31K/K33R/P210A/E318Q | + | ++ |
| 379/380 | H212G | E31K/P210A/H212G/E318Q | + | + |
| 381/382 | V10R | V10R/E31K/P210A/E318Q | + | ++ |
| 383/384 | H13R | H13R/E31K/P210A/E318Q | + | + |
| 385/386 | K173V/V183I/S343R/K347R | E31K/K173V/V183I/P210A/E318Q/S343R/K347R | + | + |
| 387/388 | A16R | A16R/E31K/P210A/E318Q | + | + |
| 389/390 | P104Q/N265C | E31K/P104Q/P210A/N265C /E318Q | + | + |
| 391/392 | T233R | E31K/P210A/T233R/E318Q | + | ++ |
| 393/394 | T233D | E31K/P210A/T233D/E318Q | + | ++ |

All activities were determined relative to the reference polypeptide of SEQ ID NO: 172. Levels of increased activity are defined as follows: “+” 0.5 to 0.9, “++” > 0.9, “+++” > 1.1,“+++”> 2.

### Example 6

### Screening Results of ODC Variants Based on SEQ ID NO: 320

Based on the results from Table 4-2, SEQ ID NO: 320 was chosen as the backbone. Beneficial mutations identified from Table 4-2 and homologs diversity were recombined into the backbone. Additionally, to generate new diversity, variants with mutations at active site positions were also constructed on SEQ ID NO: 320. The variants were assayed in triplicate for ODC activity at pH 2.6 with 4 mM potassium oxalate after a 1 hour simulated gastric challenge (0.1 mg/mL pepsin, pH 2.8) and unchallenged ODC activity at pH 2.4 and 6.5 with 4 mM potassium oxalate as described in Example 3. Analysis of the data relative to SEQ ID NO: 320 are listed in Table 4-3.

TABLE 4-3

Oxalate decarboxylase activity Relative to SEQ ID NO: 320

| SEQ ID NO: (nt/aa) | Amino Acid Differences (Relative to SEQ ID NO: 320) | Amino Acid Differences (Relative to SEQ ID NO: 2 or 4) | FIOP Unchallenged Activity at pH 2.4 Relative to SEQ ID NO: 320 | FIOP Unchallenged Activity at pH 6.5 Relative to SEQ ID NO: 320 | FIOP Pepsin Challenge Relative to SEQ ID NO: 320 |
|---|---|---|---|---|---|
| 395/396 | H13P/D17H/V196K | H13P/A16S/D17H/A26E/E31K/D174K/R195L/P210E/T226S/E318Q | + | | ++ |
| 397/398 | D17H/H212S/A331V | A16S/D17H/A26E/E31K/D174K/R195L/K196V/P210E/H212S/T226S/E318Q/A331V | +++ | | +++ |
| 399/400 | D17H/E26A/V196K/H212S/S226T | A16S/D17H/E31K/D174K/R195L/P210E/H212S/E318Q | ++ | | +++ |
| 401/402 | D17H/E26A/V196K/H212A/S226T/A331V | A16S/D17H/E31K/D174K/R195L/P210E/H212A/E318Q/A331V | + | | +++ |
| 403/404 | H13P/D17H/E26A/V196K/A331V | H13P/A16S/D17H/E31K/D174K/R195L/P210E/T226S/E318Q/A331V | ++ | | ++ |
| 405/406 | D17H/T60R/V196K/S226T | A16S/D17H/A26E/E31K/T60R/D174K/R195L/P210E/E318Q | ++ | + | +++ |
| 407/408 | D17H/V46R/H212S | A16S/D17H/A26E/E31K/V46R/D174K/R19SL/K196V/P210E/H212S/T226S/E318Q | +++ | | + |
| 409/410 | E26A/T60V/V196K/S226T/A331V | A16S/E31K/T60V/D174K/R195L/P210E/E318Q/A331V | + | + | +++ |
| 411/412 | D17H/V46R/V196K/H212A/H274Q/A331V | A16S/D17H/A26E/E31K/V46R/D174K/R195L/P210E/H212A/T226S/H274Q/E318Q/A331V | + | | +++ |
| 413/414 | H212S | A16S/A26E/E31K/D174K/R195L/K196V/P210E/H212S/T226S/E318Q | ++ | | ++ |
| 415/416 | D17H/V196K/S226T | A16S/D17H/A26E/E31K/D174K/R195L/P210E/E318Q | ++ | + | +++ |
| 417/418 | H13P/D17H/E26A/H212A/A331V | H13P/A16S/D17H/E31K/D174K/R195L/K196V/P210E/H212A/T226S/E318Q/A331V | + | | +++ |
| 419/420 | H13P/D17H/H212S/S226T/A331V | H13P/A16S/D17H/A26E/E31K/D174K/R19SL/K196V/P210E/H212S/E318Q/A331V | ++ | | ++ |
| 421/422 | V196K/H212S/A331V | 16S/A26E/E31K/D174K/R195L/P210E/H212S/T226S/E318Q/A331V | ++ | + | +++ |
| 423/424 | H13P/D17H/V196K/A331V | H13P/A16S/D17H/A26E/E31K/D174K/R195L/P210E/T226S/E318Q/A331V | ++ | | +++ |
| 425/426 | H13P/D17H/E26A/H212A/S226T/A331V | H13P/A16S/D17H/E31K/D174K/R195L/K196V/P210E/H212A/E318Q/A331V | + | | ++ |
| 427/428 | D17H/T60R/V196K/H212A/S226T/A331V | A16S/D17H/A26E/E31K/T60R/D174K/R195L/P210E/H212A/E318Q/A331V | + | | +++ |
| 429/430 | H13P/D17H/H212S | H13P/A16S/D17H/A26E/E31K/D174K/R19SL/K196V/P210E/H212S/T226S/E318Q | ++ | | ++ |
| 431/432 | H13P/D17H/E26A/A331V | H13P/A16S/D17H/E31K/D174K/R195L/K196V/P210E/T226S/E318Q/A331V | ++ | | ++ |
| 433/434 | H13P/D17H | H13P/A16S/D17H/A26E/E31K/D174K/R19SL/K196V/P210E/T226S/E318Q | ++ | | ++ |
| 435/436 | H13P/D17H/E26A/V196K | H13P/A16S/D17H/E31K/D174K/R195L/P210E/T226S/E318Q | + | | ++ |
| 437/438 | V46R/V196K/H212S/S226T | A16S/A26E/E31K/V46R/D174K/R195L/P210E/H212S/E318Q | + | | ++ |
| 439/440 | D17H/T60R/V196K | A16S/D17H/A26E/E31K/T60R/D174K/R195L/P210E/T226S/E318Q | ++ | + | +++ |
| 441/442 | D17H/V196K/A331V | A16S/D17H/A26E/E31K/D174K/R195L/P210E/T226S/E318Q/A331V | ++ | + | +++ |
| 443/444 | H13P/D17H/V46R/S226T/A331V | H13P/A16S/D17H/A26E/E31K/V46R/D174K/R195L/K196V/P210E/E318Q/A331V | ++ | | ++ |
| 445/446 | D17H/V196K/S226T/A331V | A16S/D17H/A26E/E31K/D174K/R195L/P210E/E318Q/A331V | + | + | +++ |
| 447/448 | D17H/T60V/V196K/S226T | A16S/D17H/A26E/E31K/T60V/D174K/R195L/P210E/E318Q | ++ | | +++ |
| 449/450 | D17H/V46R/V196K/H212S/A331V | A16S/D17H/A26E/E31K/V46R/D174K/R195L/P210E/H212S/T226S/E318Q/A331V | ++ | | ++ |
| 451/452 | H13P/D17H/H212A | H13P/A16S/D17H/A26E/E31K/D174K/R19SL/K196V/P210E/H212A/T226S/E318Q | + | | ++ |
| 453/454 | D17H/V46R/H212S/A331V | A16S/D17H/A26E/E31K/V46R/D174K/R19SL/K196V/P210E/H212S/T226S/E318Q/A331V | ++ | | + |
| 455/456 | D17H/V196K | A16S/D17H/A26E/E31K/D174K/R195L/P210E/T226S/E318Q | ++ | + | +++ |
| 457/458 | H13P/D17H/H212S/S226T | H13P/A16S/D17H/A26E/E31K/D174K/R19SL/K196V/P210E/H212S/E318Q | ++ | | ++ |
| 459/460 | D17H/H212S | A16S/D17H/A26E/E31K/D174K/R195L/K196V/P210E/H212S/T226S/E318Q | +++ | | +++ |
| 461/462 | D17H/E26A/H212S | A16S/D17H/E31K/D174K/R195L/K196V/P210E/H212S/T226S/E318Q | +++ | | +++ |
| 463/464 | H13P/D17H/S226T/A331V | H13P/A16S/D17H/A26E/E31K/D174K/R19SL/K196V/P210E/E318Q/A331V | ++ | | +++ |
| 465/466 | D17H/V196K/H212S/A331V | A16S/D17H/A26E/E31K/D174K/R195L/P210E/H212S/T226S/E318Q/A331V | ++ | | +++ |
| 467/468 | D17H/V196K/H212S/S226T/A331V | A16S/D17H/A26E/E31K/D174K/R195L/P210E/H212S/E318Q/A331V | ++ | | +++ |
| 469/470 | H13P/D17H/E26A/S226T/A331V | H13P/A16S/D17H/E31K/D174K/R195L/K196V/P210E/E318Q/A331V | ++ | | ++ |
| 471/472 | V46R/T60V/V196K/S226T/A331V | A16S/A26E/E31K/V46R/T60V/D174K/R195L/P210E/E318Q/A331V | ++ | + | +++ |
| 473/474 | D17H/V46R/V196K/A331V | A16S/D17H/A26E/E31K/V46R/D174K/R195L/P210E/T226S/E318Q/A331V | ++ | ++ | ++ |
| 475/476 | D17H/H212A/A331V | A16S/D17H/A26E/E31K/D174K/R195L/K196V/P210E/H212A/T226S/E318Q/A331V | | | ++ |
| 477/478 | H13P/D17H/V46R/V196K | H13P/A16S/D17H/A26E/E31K/V46R/D174K/R195L/P210E/T226S/E318Q | + | | ++ |
| 479/480 | V196K/H212S | A16S/A26E/E31K/D174K/R195L/P210E/H212S/T226S/E318Q | + | | ++ |
| 481/482 | H13P/D17H/E26A | H13P/A16S/D17H/E31K/D174K/R195L/K196V/P210E/T226S/E318Q | ++ | | +++ |
| 483/484 | D17G/T83E/D263S | A16S/D17G/A26E/E31K/T83E/D174K/R19SL/K196V/P210E/T226S/D263S/E318Q | ++ | + | ++ |
| 485/486 | D17G/T83E/K173Q/R227E | A16S/D17G/A26E/E31K/T83E/K173Q/D174K/R195L/K196V/P210E/T226S/R227E/E318Q | ++ | | +++ |
| 487/488 | D17G/R227E/V301I | A16S/D17G/A26E/E31K/D174K/R195L/K196V/P210E/T226S/R227E/V301I/E318Q | ++ | | ++ |
| 489/490 | D17G/T83E/R227E/D263S | A16S/D17G/A26E/E31K/T83E/D174K/R19SL/K196V/P210E/T226S/R227E/D263S/E318Q | + | | ++ |
| 491/492 | D17G/T83E/P125E | A16S/D17G/A26E/E31K/T83E/P125E/D174K/R195L/K196V/P210E/T226S/E318Q | ++ | + | +++ |
| 493/494 | D17G/T83E/K173Q | A16S/D17G/A26E/E31K/T83E/K173Q/D174K/R195L/K196V/P210E/T226S/E318Q | +++ | ++ | +++ |
| 495/496 | D17G/D263S/V301I | A16S/D17G/A26E/E31K/D174K/R195L/K196V/P210E/T226S/D263S/V301I/E318Q | ++ | + | ++ |
| 497/498 | D17G/P125E | A16S/D17G/A26E/E31K/P125E/D174K/R195L/K196V/P210E/T226S/E318Q | ++ | + | ++ |
| 499/500 | D17G/T83E/V301I | A16S/D17G/A26E/E31K/T83E/D174K/R19SL/K196V/P210E/T226S/V301I/E318Q | +++ | ++ | +++ |
| 501/502 | D17G/K173Q | A16S/D17G/A26E/E31K/K173Q/D174K/R195L/K196V/P210E/T226S/E318Q | ++ | + | +++ |
| 503/504 | D17G/T83E/R227E | A16S/D17G/A26E/E31K/T83E/D174K/R19SL/K196V/P210E/T226S/R227E/E318Q | ++ | | +++ |
| 505/506 | D17G/R227E | A16S/D17G/A26E/E31K/D174K/R195L/K196V/P210E/T226S/R227E/E318Q | + | | ++ |
| 507/508 | D17G | A16S/D17G/A26E/E31K/D174K/R195L/K196V/P210E/T226S/E318Q | ++ | + | +++ |
| 509/510 | D17G/P125E/R227E | A16S/D17G/A26E/E31K/P125E/D174K/R195L/K196V/P210E/T226S/R227E/E318Q | + | | ++ |
| 511/512 | D17G/T83E | A16S/D17G/A26E/E31K/T83E/D174K/R19SL/K196V/P210E/T226S/E318Q | +++ | ++ | +++ |
| 513/514 | D17G/D263S/V301T | A16S/D17G/A26E/E31K/D174K/R195L/K196V/P210E/T226S/D263S/V301T/E318Q | ++ | + | ++ |
| 515/516 | F128A | A16S/A26E/E31K/F128A/D174K/R195L/K196V/P210E/T226S/E318Q | | + | |
| 517/518 | A76S | A16S/A26E/E31K/A76S/D174K/R195L/K196V/P210E/T226S/E318Q | + | + | + |
| 519/520 | G141Q | A16S/A26E/E31K/G141Q/D174K/R195L/K196V/P210E/T226S/E318Q | | + | |
| 521/522 | F110V | A16S/A26E/E31K/F110V/D174K/R195L/K196V/P210E/T226S/E318Q | | + | |
| 523/524 | A79S | A16S/A26E/E31K/A79S/D174K/R195L/K196V/P210E/T226S/E318Q | ++ | ++ | ++ |
| 525/526 | M117A | A16S/A26E/E31K/M117A/D174K/R195L/K196V/P210E/T226S/E318Q | + | ++ | |
| 527/528 | A79G | A16S/A26E/E31K/A79G/D174K/R195L/K196V/P210E/T226S/E318Q | ++ | ++ | +++ |
| 529/530 | L61M | A16S/A26E/E31K/L61M/D174K/R195L/K196V/P210E/T226S/E318Q | + | + | |

All activities were determined relative to the reference polypeptide of SEQ ID NO: 320. Levels of increased activity are defined as follows: “+” 0.9 to 1.1, “++” > 1.1, “+++” > 1.5

### Example 7

### Screening Results of ODC Variants Based on SEQ ID NO: 396

Based on the results from Table 4-3, SEQ ID NO: 396 was chosen as the backbone. Beneficial mutations identified from Table 4-1 and Table 4-3, and diversity from homologs were recombined into the backbone. Additionally, to generate new diversity, variants with mutagenesis at additional positions were also constructed on SEQ ID NO: 396. The variants were assayed in triplicate for ODC activity at pH 2.4 with 4 mM potassium oxalate after a 1 hour simulated gastric challenge (0.4 mg/mL pepsin, pH 2.6) and unchallenged ODC activity at pH 2.4 and 5.5 with 4 mM potassium oxalate as described in Example 3. Analysis of the data relative to SEQ ID NO: 396 are listed in Table 4-4.

TABLE 4-4

Oxalate decarboxylase activity Relative to SEQ ID NO: 396

| SEQ ID NO: (nt/aa) | Amino Acid Differences (Relative to SEQ ID NO: 396) | Amino Acid Differences (Relative to SEQ ID NO: 4) | FIOP Unchallenged Activity at pH 2.4 Relative to SEQ ID NO: 396 | FIOP Unchallenged Activity at pH 5.5 Relative to SEQ ID NO: 396 | FIOP Pepsin Challenge Relative to SEQ ID NO: 396 |
|---|---|---|---|---|---|
| 531/532 | P13H/H17G/A79G | A16S/D17G/A26E/E31K/A79G/D174K/R195L/P210E/T226S/E318Q | ++++ | ++++ | +++ |
| 533/534 | P13H/L14V/H17G/T60V/A79G | L14V/A16S/D17G/A26E/E31K/T60V/A79G/D174K/R195L/P210E/T226S/E318Q | +++ | ++++ | +++ |
| 535/536 | P13H/H17G/A79G/T83E | A16S/D17G/A26E/E31K/A79G/T83E/D174K/R195L/P210E/T226S/E318Q | ++++ | ++++ | +++ |
| 537/538 | P13H/H17G/A79G/V301I | A16S/D17G/A26E/E31K/A79G/D174K/R195L/P210E/T226S/V3011/E318Q | ++++ | ++++ | +++ |
| 539/540 | P13H/L14V/H17G/T60V/A79G/H212S | L14V/A16S/D17G/A26E/E31K/T60V/A79G/D174K/R195L/P210E/H212S/T226S/E318Q | +++ | +++ | +++ |
| 541/542 | H17G/T60V/L197E | H13P/A16S/D17G/A26E/E31K/T60V/D174K/R195L/L197E/P210E/T226S/E318Q | +++ | +++ | + |
| 543/544 | P13H/T60V/H212S | A16S/D17H/A26E/E31K/T60V/D174K/R195L/P210E/H212S/T226S/E318Q | +++ | +++ | +++ |
| 545/546 | P13H/L14V/H17G/T60V/A79G/T83E/V301I | L14V/A16S/D17G/A26E/E31K/T60V/A79G/T83E/D174K/R195L/P210E/T226S/V301I/E318Q | +++ | ++++ | +++ |
| 547/548 | P13H/H17G/T60V/A79G | A16S/D17G/A26E/E31K/T60V/A79G/D174K/R195L/P210E/T226S/E318Q | ++++ | ++++ | +++ |
| 549/550 | H17G/T60V/V301I | H13P/A16S/D17G/A26E/E31K/T60V/D174K/R195L/P210E/T226S/V301I/E318Q | +++ | +++ | +++ |
| 551/552 | L14V/H17G/A79G | H13P/L14V/A16S/D17G/A26E/E31K/A79G/D174K/R195L/P210E/T226S/E318Q | +++ | +++ | +++ |
| 553/554 | H17G/T60V/T83E | H13P/A16S/D17G/A26E/E31K/T60V/T83E/D174K/R195L/P210E/T226S/E318Q | +++ | +++ | +++ |
| 555/556 | H17G/A79G | H13P/A16S/D17G/A26E/E31K/A79G/D174K/R195L/P210E/T226S/E318Q | +++ | +++ | +++ |
| 557/558 | P13H/H17G/T60V/A79G/T83E/V301I | A16S/D17G/A26E/E31K/T60V/A79G/T83E/D174K/R195L/P210E/T226S/V301I/E318Q | ++++ | ++++ | +++ |
| 559/560 | T342P | H13P/A16S/D17H/A26E/E31K/D174K/R195L/P210E/T226S/E318Q/T342P | ++ | ++ | +++ |
| 561/562 | K52T/A190Q | H13P/A16S/D17H/A26E/E31K/K52T/D174K/A190Q/R195L/P210E/T226S/E318Q | + | ++ | + |
| 563/564 | Q94N/A190Q | H13P/A16S/D17H/A26E/E31K/Q94N/D174K/A190Q/R195L/P210E/T226S/E318Q | ++ | ++ | + |
| 565/566 | A190Q/T342P/Q351E | H13P/A16S/D17H/A26E/E31K/D174K/A190Q/R195L/P210E/T226S/E318Q/T342P/Q351E | ++ | ++ | + |
| 567/568 | P13H/A79G | A16S/D17H/A26E/E31K/A79G/D174K/R195L/P210E/T226S/E318Q | +++ | ++++ | +++ |
| 569/570 | P13H/A96L | A16S/D17H/A26E/E31K/A96L/D174K/R195L/P210E/T226S/E318Q | +++ | +++ | +++ |
| 571/572 | S273A | H13P/A16S/D17H/A26E/E31K/D174K/R195L/P210E/T226S/S273A/E318Q | +++ | ++ | ++ |
| 573/574 | A126T | H13P/A16S/D17H/A26E/E31K/A126T/D174K/R195L/P210E/T226S/E318Q | + | ++ | ++ |
| 575/576 | A96K | H13P/A16S/D17Ή/A26E/E31K/A96K/D174K/R195L/P210E/T226S/E318Q | ++ | ++ | ++ |
| 577/578 | A79S | HI3P/A16S/D17H/A26E/E31K/A79S/D174K/R195L/P210E/T226S/E318Q | +++ | +++ | +++ |
| 579/580 | T269S | H13P/A16S/D17H/A26E/E31K/D174K/R195L/P210E/T226S/T269S/E318Q | ++ | ++ | ++ |
| 581/582 | G40S | H13P/A16S/D17H/A26E/E31K/G40S/D174K/R195L/P210E/T226S/E318Q | ++ | + | +++ |
| 583/584 | A96L | H13P/A16S/D17H/A26E/E31K/A96L/D174K/R195L/P210E/T226S/E318Q | +++ | ++ | +++ |
| 585/586 | T44S | H13P/A16S/D17H/A26E/E31K/T44S/D174K/R195L/P210E/T226S/E318Q | + | ++ | + |
| 587/588 | A96R | H13P/A16S/D17H/A26E/E31K/A96R/D174K/R195L/P210E/T226S/E318Q | +++ | +++ | +++ |
| 589/590 | A79G | H13P/A16S/D17H/A26E/E31K/A79G/D174K/R195L/P210E/T226S/E318Q | ++ | ++ | ++ |
| 591/592 | S267R | H13P/A16S/D17H/A26E/E31K/D174K/R195L/P210E/T226S/S267R/E318Q | ++ | ++ | ++ |
| 593/594 | 1266V | H13P/A16S/D17Ή/A26E/E31K/D174K/R195L/P210E/T226S/I266V/E318Q | ++ | ++ | ++ |
| 595/596 | N160T | H13P/A16S/D17Ή/A26E/E31K/N160T/D174K/R195L/P210E/T226S/E318Q | + | + | + |
| 597/598 | Y277T | H13P/A16S/D17Ή/A26E/E31K/D174K/R195L/P210E/T226S/Y277T/E318Q | ++ | ++ | + |
| 599/600 | I149T | H13P/A16S/D17H/A26E/E31K/I149T/D174K/R195L/P210E/T226S/E318Q | ++ | ++ | + |
| 601/602 | T22R | H13P/A16S/D17H/T22R/A26E/E31K/D174K/R195L/P210E/T226S/E318Q | ++ | ++ | ++ |
| 603/604 | V234L | H13P/A16S/D17H/A26E/E31K/D174K/R195L/P210E/T226S/V234L/E318Q | +++ | ++ | ++ |
| 605/606 | I54L | H13P/A16S/D17H/A26E/E31K/I54L/D174K/R195L/P210E/T226S/E318Q | ++ | ++ | ++ |
| 607/608 | S297Y | H13P/A16S/D17H/A26E/E31K/D174K/R195L/P210E/T226S/S297Y/E318Q | ++ | ++ | +++ |
| 609/610 | Y277A | H13P/A16S/D17H/A26E/E31K/D174K/R195L/P210E/T226S/Y277A/E318Q | +++ | ++ | ++ |
| 611/612 | D106C | H13P/A16S/D17H/A26E/E31K/D106C/D174K/R195L/P210E/T226S/E318Q | ++ | ++ | ++ |
| 613/614 | P13H/A43C | A16S/D17H/A26E/E31K/A43C/D174K/R195L/P210E/T226S/E318Q | +++ | +++ | +++ |

All activities were determined relative to the reference polypeptide of SEQ ID NO: 396. Levels of increased activity are defined as follows: “+” 0.5 to 0.9, “++” > 0.9, “+++” > 1.1,“++++”> 2

### Example 8

### Screening Results of ODC Variants Based on SEQ ID NO: 670

Based on the results from Table 4-4, SEQ ID NO: 552 was selected, codon optimized, and 6xHis-tag removed to generate SEQ ID NO: 670, which was used as the next backbone. To generate new diversity, variants with mutagenesis at different positions were constructed into the backbone. The variants were assayed in triplicate for ODC activity at pH 2.5 with 4 mM potassium oxalate after a 1 hour simulated gastric challenge (0.8 mg/mL pepsin, pH 2.6) and unchallenged ODC activity at pH 2.5 and 5.5 with 4 mM potassium oxalate as described in Example 3. Analysis of the data relative to SEQ ID NO: 670 are listed in Table 4-5.

TABLE 4-5

Oxalate decarboxylase activity Relative to SEQ ID NO: 670

| SEQ ID NO: (nt/aa) | Amino Acid Differences (Relative to SEQ ID NO: 670) | Amino Acid Differences (Relative to SEQ ID NO: 2) | FIOP Unchallenged Activity at pH 2.5 Relative to SEQ ID NO: 670 | FIOP Unchallenged Activity at pH 5.5 Relative to SEQ ID NO: 670 | FIOP Pepsin Challenge Relative to SEQ ID NO: 670 |
|---|---|---|---|---|---|
| 615/616 | K37G | H13P/L14V/A16S/D17G/A26E/E31K/K37G/A79G/D174K/R195L/P210E/T226S/E318Q | ++ | ++ | +++ |
| 617/618 | P125F | H13P/L14V/A16S/D17G/A26E/E31K/A79G/P125F/D174K/R195L/P210E/T226S/E318Q | + | + | +++ |
| 619/620 | G162K | H13P/L14V/A16S/D17G/A26E/E31K/A79G/G162K/D174K/R195L/P210E/T226S/E318Q | ++ | ++ | +++ |
| 621/622 | Q94T | H13P/L14V/A16S/D17G/A26E/E31K/A79G/Q94T/D174K/R195L/P210E/T226S/E318Q | ++ | ++ | +++ |
| 623/624 | L195W | H13P/L14V/A16S/D17G/A26E/E31K/A79G/D174K/R195W/P210E/T226S/E318Q | ++ | ++ | +++ |
| 625/626 | H85R | H13P/L14V/A16S/D17G/A26E/E31K/A79G/H85R/D174K/R195L/P210E/T226S/E318Q | ++ | ++ | +++ |
| 627/628 | G103S | H13P/L14V/A16S/D17G/A26E/E31K/A79G/G103S/D174K/R195L/P210E/T226S/E318Q | ++ | ++ | +++ |
| 629/630 | K232T | H13P/L14V/A16S/D17G/A26E/E31K/A79G/D174K/R195L/P210E/T226S/K232T/E318Q | ++ | ++ | +++ |
| 631/632 | A189R | H13P/L14V/A16S/D17G/A26E/E31K/A79G/D174K/A189R/R195L/P210E/T226S/E318Q | ++ | ++ | +++ |
| 633/634 | A186N | H13P/L14V/A16S/D17G/A26E/E31K/A79G/D174K/A186N/R195L/P210E/T226S/E318Q | ++ | ++ | +++ |
| 635/636 | A155R | H13P/L14V/A16S/D17G/A26E/E31K/A79G/A155R/D174K/R195L/P210E/T226S/E318Q | ++ | ++ | +++ |
| 637/638 | Q193A | H13P/L14V/A16S/D17G/A26E/E31K/A79G/D174K/Q193A/R195L/P210E/T226S/E318Q | ++ | ++ | +++ |
| 639/640 | T342A | H13P/L14V/A16S/D17G/A26E/E31K/A79G/D174K/R195L/P210E/T226S/E318Q/T342A | ++ | ++ | +++ |
| 641/642 | K196V | H13P/L14V/A16S/D17G/A26E/E31K/A79G/D174K/R195L/K196V/P210E/T226S/E318Q | ++ | + | +++ |
| 643/644 | P63S | H13P/L14V/A16S/D17G/A26E/E31K/P63S/A79G/D174K/R195L/P210E/T226S/E318Q | + | ++ | +++ |
| 645/646 | K33R | H13P/L14V/A16S/D17G/A26E/E31K/K33R/A79G/D174K/R195L/P210E/T226S/E318Q | + | + | ++ |
| 647/648 | Q351H | H13P/L14V/A16S/D17G/A26E/E31K/A79G/D174K/R195L/P210E/T226S/E318Q/Q351H | ++ | ++ | ++ |
| 649/650 | Q193G | H13P/L14V/A16S/D17G/A26E/E31K/A79G/D174K/Q193G/R195L/P210E/T226S/E318Q | ++ | ++ | ++ |
| 651/652 | P125S | H13P/L14V/A16S/D17G/A26E/E31K/A79G/P125S/D174K/R195L/P210E/T226S/E318Q | ++ | ++ | ++ |
| 653/654 | T314S | H13P/L14V/A16S/D17G/A26E/E31K/A79G/D174K/R195L/P210E/T226S/T314S/E318Q | ++ | ++ | ++ |
| 655/656 | T187R | H13P/L14V/A16S/D17G/A26E/E31K/A79G/D174K/T187R/R195L/P210E/T226S/E318Q | +++ | ++ | ++ |
| 657/658 | D303T | H13P/L14V/A16S/D17G/A26E/E31K/A79G/D174K/R195L/P210E/T226S/D303T/E318Q | ++ | ++ | ++ |
| 659/660 | G103V | H13P/L14V/A16S/D17G/A26E/E31K/A79G/G103V/D174K/R195L/P210E/T226S/E318Q | ++ | ++ | ++ |
| 661/662 | G103Q | H13P/L14V/A16S/D17G/A26E/E31K/A79G/G103Q/D174K/R195L/P210E/T226S/E318Q | ++ | ++ | ++ |
| 663/664 | A155V | H13P/L14V/A16S/D17G/A26E/E31K/A79G/A155V/D174K/R195L/P210E/T226S/E318Q | + | + | ++ |
| 665/666 | P164R | H13P/L14V/A16S/D17G/A26E/E31K/A79G/P164R/D174K/R195L/P210E/T226S/E318Q | + | ++ | ++ |
| 667/668 | P153S | H13P/L14V/A16S/D17G/A26E/E31K/A79G/P153S/D174K/R195L/P210E/T226S/E318Q | + | ++ | ++ |
| 669/670 | | H13P/L14V/A16S/D17G/A26E/E31K/A79G/D174K/R195L/P210E/T226S/E318Q | ++ | ++ | ++ |
| 671/672 | K37R | H13P/L14V/A16S/D17G/A26E/E31K/K37R/A79G/D174K/R195L/P210E/T226S/E318Q | ++ | ++ | ++ |
| 673/674 | E346Q | H13P/L14V/A16S/D17G/A26E/E31K/A79G/D174K/R195L/P210E/T226S/E318Q/E346Q | ++ | ++ | ++ |
| 675/676 | E346G | H13P/L14V/A16S/D17G/A26E/E31K/A79G/D174K/R195L/P210E/T226S/E318Q/E346G | ++ | ++ | ++ |
| 677/678 | Q351A | H13P/L14V/A16S/D17G/A26E/E31K/A79G/D174K/R195L/P210E/T226S/E318Q/Q351A | ++ | ++ | ++ |
| 679/680 | V183K | H13P/L14V/A16S/D17G/A26E/E31K/A79G/D174K/V183K/R195L/P210E/T226S/E318Q | ++ | ++ | ++ |
| 681/682 | D19C | H13P/L14V/A16S/D17G/D19C/A26E/E31K/A79G/D174K/R195L/P210E/T226S/E318Q | + | ++ | ++ |
| 683/684 | T187L | H13P/L14V/A16S/D17G/A26E/E31K/A79G/D174K/T187L/R195L/P210E/T226S/E318Q | ++ | + | ++ |
| 685/686 | D123S | H13P/L14V/A16S/D17G/A26E/E31K/A79G/D123S/D174K/R195L/P210E/T226S/E318Q | ++ | + | ++ |
| 687/688 | H85G | H13P/L14V/A16S/D17G/A26E/E31K/A79G/H85G/D174K/R195L/P210E/T226S/E318Q | ++ | ++ | ++ |
| 689/690 | T187S | H13P/L14V/A16S/D17G/A26E/E31K/A79G/D174K/T187S/R195L/P210E/T226S/E318Q | ++ | ++ | ++ |
| 691/692 | V183C | H13P/L14V/A16S/D17G/A26E/E31K/A79G/D174K/V183C/R195L/P210E/T226S/E318Q | + | ++ | ++ |
| 693/694 | D123G | H13P/L14V/A16S/D17G/A26E/E31K/A79G/D123G/D174K/R195L/P210E/T226S/E318Q | ++ | ++ | ++ |
| 695/696 | K350R | H13P/L14V/A16S/D17G/A26E/E31K/A79G/D174K/R195L/P210E/T226S/E318Q/K350R | ++ | ++ | ++ |
| 697/698 | H85E | H13P/L14V/A16S/D17G/A26E/E31K/A79G/H85E/D174K/R195L/P210E/T226S/E318Q | ++ | ++ | ++ |
| 699/700 | E205A | H13P/L14V/A16S/D17G/A26E/E31K/A79G/D174K/R195L/E205A/P210E/T226S/E318Q | +++ | ++ | + |
| 701/702 | K284R | H13P/L14V/A16S/D17G/A26E/E31K/A79G/D174K/R195L/P210E/T226S/K284R/E318Q | ++ | ++ | + |
| 703/704 | P199V | H13P/L14V/A16S/D17G/A26E/E31K/A79G/D174K/R195L/P199V/P210E/T226S/E318Q | + | ++ | + |
| 705/706 | E346W | H13P/L14V/A16S/D17G/A26E/E31K/A79G/D174K/R195L/P210E/T226S/E318Q/E346W | + | ++ | + |
| 707/708 | S343W | H13P/L14V/A16S/D17G/A26E/E31K/A79G/D174K/R195L/P210E/T226S/E318Q/S343W | + | ++ | + |
| 709/710 | P164V | H13P/L14V/A16S/D17G/A26E/E31K/A79G/P164V/D174K/R195L/P210E/T226S/E318Q | + | ++ | + |
| 711/712 | L200N | H13P/L14V/A16S/D17G/A26E/E31K/A79G/D174K/R195L/L200N/P210E/T226S/E318Q | + | ++ | + |
| 713/714 | T342E | H13P/L14V/A16S/D17G/A26E/E31K/A79G/D174K/R195L/P210E/T226S/E318Q/T342E | ++ | ++ | + |
| 715/716 | E208G | H13P/L14V/A16S/D1G/A26E/E31K/A79G/D174K/R195L/E208G/P210E/T226S/E318Q | ++ | ++ | + |
| 717/718 | D169A | H13P/L14V/A16S/D17G/A26E/E31K/A79G/D169A/D174K/R195L/P210E/T226S/E318Q | + | ++ | + |
| 719/720 | A190G | H13P/L14V/A16S/D17G/A26E/E31K/A79G/D174K/A190G/R195L/P210E/T226S/E318Q | + | ++ | + |
| 721/722 | K284A | H13P/L14V/A16S/D17G/A26E/E31K/A79G/D174K/R195L/P210E/T226S/K284A/E318Q | ++ | ++ | + |
| 723/724 | P199G | H13P/L14V/A16S/D1G/A26E/E31K/A79G/D174K/R195L/P199G/P210E/T226S/E318Q | + | ++ | + |
| 725/726 | L121W | H13P/L14V/A16S/D17G/A26E/E31K/A79G/L121W/D174K/R195L/P210E/T226S/E318Q | ++ | ++ | + |
| 727/728 | K196S | H13P/L14V/A16S/D17G/A26E/E31K/A79G/D174K/R195L/K196S/P210E/T226S/E318Q | + | + | + |

All activities were determined relative to the reference polypeptide of SEQ ID NO: 670. Levels of increased activity are defined as follows: “+” 0.5 to 0.9, “++” > 0.9, “+++” > 1.1,“++++”> 2

### Example 9

### Screening Results of ODC Variants Based on SEQ ID NO: 616

Based on the results from Table 4-5, SEQ ID NO: 616 was chosen as the backbone. To generate new diversity, beneficial mutations from Table 4-3 and Table 4-5 variants were recombined into the backbone, along with reversion mutations of neutral and deleterious mutations from Tables 4-1, 4-2, and 4-4. Additionally, to generate new diversity, variants with mutagenesis at different positions were constructed into the backbone. The variants were assayed in triplicate for ODC activity at pH 2.5 with 4 mM potassium oxalate after a 1-2 hour simulated gastric challenge (0.8 mg/mL pepsin, pH 2.5 or pH 2.4) and unchallenged ODC activity at pH 2.5 and 5.5 with 4 mM potassium oxalate as described in Example 3. Analysis of the data relative to SEQ ID NO: 616 are listed in Table 4-6.

TABLE 4-6

Oxalate decarboxylase activity Relative to SEQ ID NO: 616

| SEQ ID NO: (nt/aa) | Amino Acid Differences (Relative to SEQ ID NO: 616) | Amino Acid Differences (Relative to SEQ ID NO: 2) | FIOP Unchallenged Activity at pH 2.5 Relative to SEQ ID NO: 616 | FIOP Unchallenged Activity at pH 5.5 Relative to SEQ ID NO: 616 | FIOP Pepsin Challenge (1 hr) Relative to SEQ ID NO: 616 | FIOP Pepsin Challenge (2 hrs) Relative to SEQ ID NO: 616 |
|---|---|---|---|---|---|---|
| 729/730 | V14L/E26A/T60V/Q318E | H13P/A16S/D17G/E31K/K37G/T60V/A79G/D174K/R195L/P210E/T226S | + | + | ++ | ++++ |
| 731/732 | V14L/E26A/T60V/Q94T/G162K/H212S | H13P/A16S/D17G/E31K/K37G/T60V/A79G/Q94T/G162K/D174K/R195L/P210E/H212S/T226S/E318Q | + | | ++ | ++++ |
| 733/734 | T60V/G162K/S226T | H13P/L14V/A16S/D17G/A26E/E31K/K37G/T60V/A79G/G162K/D174K/R195L/P210E/E318Q | + | + | ++ | +++ |
| 735/736 | T60V | H13P/L14V/A16S/D17G/A26E/E31K/K37G/T60V/A79G/D174K/R195L/P210E/T226S/E318Q | + | + | ++ | ++ |
| 737/738 | V14L/P125F/G162K/H212S/Q318E | H13P/A16S/D17G/A26E/E31K/K37G/A79G/P125F/G162K/D174K/R195L/P210E/H212S/T226S | | | ++ | +++ |
| 739/740 | T60V/P125F/S226T | H13P/L14V/A16S/D17G/A26E/E31K/K37G/T60V/A79G/P125F/D174K/R195L/P210E/E318Q | | | ++ | ++ |
| 741/742 | V14L/P125F/G162K/S226T | H13P/A16S/D17G/A26E/E31K/K37G/A79G/P125F/G162K/D174K/R195L/P210E/E318Q | | + | ++ | +++ |
| 743/744 | E26A/T60V/G162K/S226T/Q318E | H13P/L14V/A16S/D17G/E31K/K37G/T60V/A79G/G162K/D174K/R195L/P210E | + | + | ++ | +++ |
| 745/746 | V14L/E26A/Q94T/G162K/H212S/S226T | H13P/A16S/D17G/E31K/K37G/A79G/Q94T/G162K/D174K/R195L/P210E/H212S/E318Q | + | | ++ | +++ |
| 747/748 | V14L/T60V/G162K | H13P/A16S/D17G/A26E/E31K/K37G/T60V/A79G/G162K/D174K/R195L/P210E/T226S/E318Q | + | + | ++ | ++++ |
| 749/750 | V14L/T60V/G162K/S226T | H13P/A16S/D17G/A26E/E31K/K37G/T60V/A79G/G162K/D174K/R195L/P210E/E318Q | + | + | ++ | ++++ |
| 751/752 | V14L/Q94T/G162K/Q318E | H13P/A16S/D17G/A26E/E31K/K37G/A79G/Q94T/G162K/D174K/R195L/P210E/T226S | | + | ++ | +++ |
| 753/754 | V14L/Q94T/G162K | H13P/A16S/D17G/A26E/E31K/K37G/A79G/Q94T/G162K/D174K/R195L/P210E/T226S/E318Q | + | + | ++ | |
| 755/756 | V14L/T60V/S226T | H13P/A16S/D17G/A26E/E31K/K37G/T60V/A79G/D174K/R195L/P210E/E318Q | + | + | ++ | |
| 757/758 | V14L/T60V/Q94T/H212S | H13P/A16S/D17G/A26E/E31K/K37G/T60V/A79G/Q94T/D174K/R195L/P210E/H212S/T226S/E318Q | + | | ++ | ++++ |
| 759/760 | V14L/T60V/G162K/H212S | H13P/A16S/D17G/A26E/E31K/K37G/T60V/A79G/G162K/D174K/R195L/P210E/H212S/T226S/E318Q | + | | ++ | ++++ |
| 761/762 | V14L/G162K | H13P/A16S/D17G/A26E/E31K/K37G/A79G/G162K/D174K/R195L/P210E/T226S/E318Q | + | + | ++ | +++ |
| 763/764 | V14L/Q94T/H212S/Q318E | H13P/A16S/D17G/A26E/E31K/K37G/A79G/Q94T/D174K/R195L/P210E/H212S/T226S | | | ++ | |
| 765/766 | V14L/T60V | H13P/A16S/D17G/A26E/E31K/K37G/T60V/A79G/D174K/R195L/P210E/T226S/E318Q | + | + | ++ | ++++ |
| 767/768 | V14L/E26A/G162K | H13P/A16S/D17G/E31K/K37G/A79G/G162K/D174K/R195L/P210E/T226S/E318Q | + | + | ++ | ++++ |
| 769/770 | V14L | H13P/A16S/D17G/A26E/E31K/K37G/A79G/D174K/R195L/P210E/T226S/E318Q | + | + | ++ | +++ |
| 771/772 | V14L/Q318E | H13P/A16S/D17G/A26E/E31K/K37G/A79G/D174K/R195L/P210E/T226S | | | ++ | ++ |
| 773/774 | V14L/G162K/S226T/Q318E | H13P/A16S/D17G/A26E/E31K/K37G/A79G/G162K/D174K/R195L/P210E | | | ++ | |
| 775/776 | V14L/P125F/H212S | H13P/A16S/D17G/A26E/E31K/K37G/A79G/P125F/D174K/R195L/P210E/H212S/T226S/E318Q | | | ++ | ++ |
| 777/778 | V14L/P125F/S226T/Q318E | H13P/A16S/D17G/A26E/E31K/K37G/A79G/P125F/D174K/R195L/P210E | | | ++ | |
| 779/780 | V14L/E26A/T60V/G162K | H13P/A16S/D17G/E31K/K37G/T60V/A79G/G162K/D174K/R195L/P210E/T226S/E318Q | + | + | ++ | +++ |
| 781/782 | V14L/P125F/G162K/H212S/S226T | H13P/A16S/D17G/A26E/E31K/K37G/A79G/P125F/G162K/D174K/R195L/P210E/H212S/E318Q | | | ++ | ++ |
| 783/784 | V14L/Q94T/P125F/G162K/Q318E | H13P/A16S/D17G/A26E/E31K/K37G/A79G/Q94T/P125F/G162K/D174K/R195L/P210E/T226S | + | + | ++ | +++ |
| 785/786 | V14L/P125F/S226T | H13P/A16S/D17G/A26E/E31K/K37G/A79G/P125F/D174K/R195L/P210E/E318Q | | | ++ | ++ |
| 787/788 | Y277R | H13P/L14V/A16S/D17G/A26E/E31K/K37G/A79G/D174K/R195L/P210E/T226S/Y277R/E318Q | | + | ++ | |
| 789/790 | Y277C | H13P/L14V/A16S/D17G/A26E/E31K/K37G/A79G/D174K/R195L/P210E/T226S/Y277C/E318Q | + | + | ++ | ++ |
| 791/792 | Y277T | H13P/L14V/A16S/D17G/A26E/E31K/K37G/A79G/D174K/R195L/P210E/T226S/Y277T/E318Q | + | + | ++ | ++ |
| 793/794 | S166R | H13P/L14V/A16S/D17G/A26E/E31K/K37G/A79G/S166R/D174K/R195L/P210E/T226S/E318Q | + | + | ++ | ++ |
| 795/796 | T233H | H13P/L14V/A16S/D17G/A26E/E31K/K37G/A79G/D174K/R195L/P210E/T226S/T233H/E318Q | | | ++ | + |
| 797/798 | T83R | H13P/L14V/A16S/D17G/A26E/E31K/K37G/A79G/T83R/D174K/R195L/P210E/T226S/E318Q | + | + | ++ | + |
| 799/800 | T60M | H13P/L14V/A16S/D17G/A26E/E31K/K37G/T60M/A79G/D174K/R195L/P210E/T226S/E318Q | + | + | ++ | ++ |
| 801/802 | Y277I | H13P/L14V/A16S/D17G/A26E/E31K/K37G/A79G/D174K/R195L/P210E/T226S/Y277I/E318Q | + | + | ++ | ++ |
| 803/804 | Y277M | H13P/L14V/A16S/D17G/A26E/E31K/K37G/A79G/D174K/R195L/P210E/T226S/Y277M/E318Q | + | + | ++ | + |
| 805/806 | T5S | TSS/H13P/L14V/A16S/D17G/A26E/E31K/K37G/A79G/D174K/R195L/P210E/T226S/E318Q | + | + | ++ | ++ |
| 807/808 | T342R | H13P/L14V/A16S/D17G/A26E/E31K/K37G/A79G/D174K/R195L/P210E/T226S/E318Q/T342R | + | + | ++ | + |
| 809/810 | Y277V | H13P/L14V/A16S/D17G/A26E/E31K/K37G/A79G/D174K/R195L/P210E/T226S/Y277V/E318Q | + | + | ++ | ++ |
| 811/812 | T60R | H13P/L14V/A16S/D17G/A26E/E31K/K37G/T60R/A79G/D174K/R195L/P210E/T226S/E31Q | | | ++ | ++ |
| 813/814 | A96R | H13P/L14V/A16S/D17G/A26E/E31K/K37G/A79G/A96R/D174K/R195L/P210E/T226S/E31Q | ++ | + | ++ | ++ |
| 815/816 | K284S | H13P/L14V/A16S/D17G/A26E/E31K/K37G/A79G/D174K/R195L/P210E/T226S/K284S/E318Q | + | + | ++ | + |
| 817/818 | E26R | H13P/L14V/A16S/D17G/A26R/E31K/K37G/A79G/D174K/R195L/P210E/T226S/E318Q | + | + | ++ | ++ |
| 819/820 | E26N | H13P/L14V/A16S/D17G/A26N/E31K/K37G/A79G/D174K/R195L/P210E/T226S/E318Q | + | + | ++ | |
| 821/822 | T83W | H13P/L14V/A16S/D17G/A26E/E31K/K37G/A79G/T83W/D174K/R195L/P210E/T226S/E318Q | | | ++ | |

All activities were determined relative to the reference polypeptide of SEQ ID NO: 616. Levels of increased activity are defined as follows: “+” 0.9 to 1.1, “++” > 1.1, “+++” > 2.0,“++++”>3.0

### Example 10

### Screening Results of ODC Variants Based on SEQ ID NO: 750

Based on the results from Table 4-6, SEQ ID NO: 750 was chosen as the backbone. Beneficial mutations identified from Table 7 were recombined into the backbone, along with reversions of mutations from Table 4-6. The variants were assayed for ODC activity at pH 2.4 with 4 mM potassium oxalate after a 1 hour simulated gastric challenge (0.8 mg/mL pepsin, pH 2.2), and unchallenged ODC activity at pH 2.4 and 5.5 with 4 mM potassium oxalate as described in Example 3. Analysis of the data relative to SEQ ID NO: 750 are listed in Table 4-7.

TABLE 4-7

Oxalate decarboxylase activity Relative to SEQ ID NO: 750

| SEQ ID NO: (nt/aa) | Amino Acid Differences (Relative to SEQ ID NO: 750) | Amino Acid Differences (Relative to SEQ ID NO: 2) | FIOP Unchallenged Activity at pH 2.5 Relative to SEQ ID NO: 750 | FIOP Unchallenged Activity at pH 5.5 Relative to SEQ ID NO: 750 | FIOP Pepsin Challenge Relative to SEQ ID NO: 750 |
|---|---|---|---|---|---|
| 823/824 | T5S/E26A/T83W/T342R | T5S/H13P/A16S/D17G/E31K/K37G/T60V/A79G/T83W/G162K/D174K/R195L/P210E/E318Q/T342R | + | | +++ |
| 825/826 | T5S/E26A/Y277T | T5S/H13P/A16S/D17G/E31K/K37G/T60V/A79G/G162K/D174K/R195L/P210E/Y277T/E318Q | | | ++++ |
| 827/828 | T5S/A96L/Y277T | T5S/H13P/A16S/D17G/A26E/E31K/K37G/T60V/A79G/A96L/G162K/D174K/R195L/P210E/Y277T/E318Q | ++ | + | ++++ |
| 829/830 | T5S/T83W/Y277T/T342R | T5S/H13P/A16S/D17G/A26E/E31K/K37G/T60V/A79G/T83W/G162K/D174K/R195L/P210E/Y277T/E318Q/T342R | + | | +++ |
| 831/832 | E26A/T83W/Y277T | H13P/A16S/D17G/E31K/K37G/T60V/A79G/T83W/G162K/D174K/R195L/P210E/Y277T/E318Q | + | | ++++ |
| 833/834 | T5S/E26A/T83W/Y277V | T5S/H13P/A16S/D17G/E31K/K37G/T60V/A79G/T83W/G162K/D174K/R195L/P210E/Y277V/E318Q | + | | ++++ |
| 835/836 | T5S/Y277T/T342R | T5S/H13P/A16S/D17G/A26E/E31K/K37G/T60V/A79G/G162K/D174K/R195L/P210E/Y277T/E318Q/T342R | + | | ++++ |
| 837/838 | T5S/T83W/Y277V/T342R | T5S/H13P/A16S/D17G/A26E/E31K/K37G/T60V/A79G/T83W/G162K/D174K/R195L/P210E/Y277V/E318Q/T342R | ++ | | +++ |
| 839/840 | T5S/E26A/S166R/Y277T | T5S/H13P/A16S/D17G/E31K/K37G/T60V/A79G/G162K/S166R/D174K/R195L/P210E/Y277T/E318Q | ++ | | +++ |
| 841/842 | T83W/Y277T/T342R | H13P/A16S/D17G/A26E/E31K/K37G/T60V/A79G/T83W/G162K/D174K/R195L/P210E/Y277T/E318Q/T342R | ++ | | ++++ |
| 843/844 | T83R/A96L/Y277V/T342R | H13P/A16S/D17G/A26E/E31K/K37G/T60V/A79G/T83R/A96L/G162K/D174K/R195L/P210E/Y277V/E318Q/T342R | ++ | + | +++ |
| 845/846 | T83W/A96L/Y277T | H13P/A16S/D17G/A26E/E31K/K37G/T60V/A79G/T83W/A96L/G162K/D174K/R195L/P210E/Y277T/E318Q | ++ | | ++++ |
| 847/848 | E26A/T83W/Y277V/T342R | H13P/A16S/D17G/E31K/K37G/T60V/A79G/T83W/G162K/D174K/R195L/P210E/Y277V/E318Q/T342R | ++ | | ++++ |
| 849/850 | T5S/E26A/T83W/Y277V/T342R | T5S/H13P/A16S/D17G/E31K/K37G/T60V/A79G/T83W/G162K/D174K/R195L/P210E/Y277V/E318Q/T342R | ++ | | +++ |
| 851/852 | T5S/T83R/Y277T/T342R | T5S/H13P/A16S/D17G/A26E/E31K/K37G/T60V/A79G/T83R/G162K/D174K/R195L/P210E/Y277T/E318Q/T342R | ++ | | +++ |
| 853/854 | T5S/E26A/T83W/Y277T/T342R | T5S/H13P/A16S/D17G/E31K/K37G/T60V/A79G/T83W/G162K/D174K/R195L/P210E/Y277T/E318Q/T342R | ++ | | +++ |
| 855/856 | T5S/T83R/Y277V | T5S/H13P/A16S/D17G/A26E/E31K/K37G/T60V/A79G/T83R/G162K/D174K/R195L/P210E/Y277V/E318Q | + | + | +++ |
| 857/858 | E26A/Y277T | H13P/A16S/D17G/E31K/K37G/T60V/A79G/G162K/D174K/R195L/P210E/Y277T/E318Q | + | | +++ |
| 859/860 | T83W/Y277T | H13P/A16S/D17G/A26E/E31K/K37G/T60V/A79G/T83W/G162K/D174K/R195L/P210E/Y277T/E318Q | + | | +++ |
| 861/862 | T5S/I35V/Y277T | T5S/H13P/A16S/D17G/A26E/E31K/I35V/K37G/T60V/A79G/G162K/D174K/R195L/P210E/Y277T/E318Q | | | +++ |
| 863/864 | T5S/E26A/T83R/A96R/Y277V | T5S/H13P/A16S/D17G/E31K/K37G/T60V/A79G/T83R/A96R/G162K/D174K/R195L/P210E/Y277V/E318Q | ++ | + | +++ |
| 865/866 | T5S/E26A/A96L/Y277T | T5S/H13P/A16S/D17G/E31K/K37G/T60V/A79G/A96L/G162K/D174K/R195L/P210E/Y277T/E318Q | ++ | + | +++ |
| 867/868 | T5S/Y277T | T5S/H13P/A16S/D17G/A26E/E31K/K37G/T60V/A79G/G162K/D174K/R195L/P210E/Y277T/E318Q | + | + | +++ |
| 869/870 | T5S/E26A/T83R/Y277T | T5S/H13P/A16S/D17G/E31K/K37G/T60V/A79G/T83R/G162K/D174K/R195L/P210E/Y277T/E318Q | ++ | + | +++ |
| 871/872 | T5S/T83R/Y277T | T5S/H13P/A16S/D17G/A26E/E31K/K37G/T60V/A79G/T83R/G162K/D174K/R195L/P210E/Y277T/E318Q | + | | ++++ |
| 873/874 | E26A/T83R/Y277V | H13P/A16S/D17G/E31K/K37G/T60V/A79G/T83R/G162K/D174K/R195L/P210E/Y277V/E318Q | ++ | + | +++ |
| 875/876 | T5S/E26A/Y277V | T5S/H13P/A16S/D17G/E31K/K37G/T60V/A79G/G162K/D174K/R195L/P210E/Y277V/E318Q | + | | +++ |
| 877/878 | T5S/S166R/Y277T | T5S/H13P/A16S/D17G/A26E/E31K/K37G/T60V/A79G/G162K/S166R/D174K/R195L/P210E/Y277T/E318Q | ++ | + | +++ |
| 879/880 | E26A/T83W/Y277V | H13P/A16S/D17G/E31K/K37G/T60V/A79G/T83W/G162K/D174K/R195L/P210E/Y277V/E318Q | + | + | +++ |
| 881/882 | E26A/T83W/A96L/Y277V/T342R | H13P/A16S/D17G/E31K/K37G/T60V/A79G/T83W/A96L/G162K/D174K/R195L/P210E/Y277V/E318Q/T342R | ++ | + | ++ |
| 883/884 | Y277T | H13P/A16S/D17G/A26E/E31K/K37G/T60V/A79G/G162K/D174K/R195L/P210E/Y277T/E318Q | + | + | ++++ |
| 885/886 | E26A/Y277T/T342R | H13P/A16S/D17G/E31K/K37G/T60V/A79G/G162K/D174K/R195L/P210E/Y277T/E318Q/T342R | ++ | + | +++ |
| 887/888 | T83W/Y277V/T342R | H13P/A16S/D17G/A26E/E31K/K37G/T60V/A79G/T83W/G162K/D174K/R195L/P210E/Y277V/E318Q/T342R | ++ | | +++ |
| 889/890 | T83R/Y277V | H13P/A16S/D17G/A26E/E31K/K37G/T60V/A79G/T83R/G162K/D174K/R195L/P210E/Y277V/E318Q | + | + | ++++ |
| 891/892 | T5S | T5S/H13P/A16S/D17G/A26E/E31K/K37G/T60V/A79G/G162K/D174K/R195L/P210E/E318Q | + | + | +++ |
| 893/894 | E26A/Y277V | H13P/A16S/D17G/E31K/K37G/T60V/A79G/G162K/D174K/R195L/P210E/Y277V/E318Q | + | | ++++ |
| 895/896 | T83R/Y277T | H13P/A16S/D17G/A26E/E31K/K37G/T60V/A79G/T83R/G162K/D174K/R195L/P210E/Y277T/E318Q | + | + | ++++ |
| 897/898 | T5S/T83W/A96R/Y277T/T342R | T5S/H13P/A16S/D17G/A26E/E31K/K37G/T60V/A79G/T83W/A96R/G162K/D174K/R195L/P210E/Y277T/E318Q/T342R | ++ | | +++ |
| 899/900 | T5S/Y277V | T5S/H13P/A16S/D17G/A26E/E31K/K37G/T60V/A79G/G162K/D174K/R195L/P210E/Y277V/E318Q | + | | ++++ |
| 901/902 | Y277V | H13P/A16S/D17G/A26E/E31K/K37G/T60V/A79G/G162K/D174K/R195L/P210E/Y277V/E318Q | + | + | ++++ |
| 903/904 | T5S/Y277V/T342R | T5S/H13P/A16S/D17G/A26E/E31K/K37G/T60V/A79G/G162K/D174K/R195L/P210E/Y277V/E318Q/T342R | ++ | + | ++++ |
| 905/906 | T83W/A96L/Y277V | H13P/A16S/D17G/A26E/E31K/K37G/T60V/A79G/T83W/A96L/G162K/D174K/R195L/P210E/Y277V/E318Q | ++ | | ++++ |
| 907/908 | T83W/Y277V | H13P/A16S/D17G/A26E/E31K/K37G/T60V/A79G/T83W/G162K/D174K/R195L/P210E/Y277V/E318Q | + | | ++++ |

All activities were determined relative to the reference polypeptide of SEQ ID NO: 750. Levels of increased activity are defined as follows: “+” 0.9 to 1.1, “++” > 1.1, “+++” > 2.0,“++++”>3.0

### Example 11

### Screening Results of ODC Variants Based on SEQ ID NO: 906

Single point mutations were constructed on the ODC polypeptide of SEQ ID NO: 906 through site saturation mutagenesis at the 16 positions that differ from the ODC polypeptide of SEQ ID NO: 2. Additionally, reversion mutations at these 16 positions were recombined into SEQ ID NO: 906. The variants were assayed for ODC activity at pH 2.8 with 4 mM potassium oxalate after a 1 hour simulated gastric challenge (0.3 mg/mL pepsin, pH 3.2), and at pH 2.5 with 4 mM potassium oxalate after a 1 hour simulated gastric challenge (0.8 mg/mL pepsin, pH 2.8) as described in Example 3. Unchallenged ODC activity was assayed at pH 2.8 and 5.5 with 4 mM potassium oxalate as described in Example 3. Analysis of the activity data in reference to both ODC polypeptides of SEQ ID NO: 906 and SEQ ID NO: 2, are listed in Tables 5-1 and 5-2.

TABLE 5-1

Oxalate decarboxylase activity Relative to SEQ ID NO: 4 or SEQ ID NO: 906

| SEQ ID NO: (nt/aa) | Amino Acid Differences (Relative to SEQ ID NO: 906) | Amino Acid Differences (Relative to SEQ ID NO: 2) | FIOP Unchallenged Activity pH 2.8 relative to SEQ ID NO: 2 | FIOP Unchallenged Activity pH 4.5 relative to SEQ ID NO: 2 | FIOP 0.3 g/L Pepsin Challenge relative to SEQ ID NO: 2 | FIOP Unchallenged Activity pH 2.8 relative to SEQ ID NO: 906 | FIOP Unchallenged Activity pH 4.5 relative to SEQ ID NO: 906 | FIOP 0.8 g/L Pepsin Challenge relative to SEQ ID NO: 906 |
|---|---|---|---|---|---|---|---|---|
| ½ | P13H/S16A/G17D/E26A/K31E/G37K/V60T/G79A/W83T/L96A/K162G/K174D/L195R/E210P/V277Y/Q318E | | + | + | + | | + | |
| 905/906 | | H13P/A16S/D17G/A26E/E31K/K37G/T60V/A79G/T83W/A96L/G162K/D174K/R195L/P210E/Y277V/E318Q | ++ | + | ++++ | + | + | + |
| 909/910 | E26L | H13P/A16S/D17G/A26L/E31K/K37G/T60V/A79G/T83W/A96L/G162K/D174K/R195L/P210E/Y277V/E318Q | | | + | | | |
| 911/912 | K31W | H13P/A16S/D17G/A26E/E31W/K37G/T60V/A79G/T83W/A96L/G162K/D174K/R195L/P210E/Y277V/E318Q | + | | ++++ | | | |
| 913/914 | L96K | H13P/A16S/D17G/A26E/E31K/K37G/T60V/A79G/T83W/A96K/G162K/D174K/R195L/P210E/Y277V/E318Q | ++ | + | ++++ | + | + | |
| 915/916 | V60R | H13P/A16S/D17G/A26E/E31K/K37G/T60R/A79G/T83W/A96L/G162K/D174K/R195L/P210E/Y277V/E318Q | ++ | | ++++ | | + | + |
| 917/918 | Q318D | H13P/A16S/D17G/A26E/E31K/K37G/T60V/A79G/T83W/A96L/G162K/D174K/R195L/P210E/Y277V/E318D | ++ | | ++++ | | | |
| 919/920 | E26G | H13P/A16S/D17G/A26G/E31K/K37G/T60V/A79G/T83W/A96L/G162K/D174K/R195L/P210E/Y277V/E318Q | ++ | | ++++ | | | |
| 921/922 | E210R | H13P/A16S/D17G/A26E/E31K/K37G/T60V/A79G/T83W/A96L/G162K/D174K/R195L/P210R/Y277V/E318Q | ++ | | ++++ | | | |
| 923/924 | K31R | H13P/A16S/D17G/A26E/E31R/K37G/T60V/A79G/T83W/A96L/G162K/D174K/R195L/P210E/Y277V/E318Q | ++ | | ++++ | + | | |
| 925/926 | V277F | H13P/A16S/D17G/A26E/E31K/K37G/T60V/A79G/T83W/A96L/G162K/D174K/R195L/P210E/Y277F/E318Q | ++ | + | ++++ | + | + | |
| 927/928 | G37M | H13P/A16S/D17G/A26E/E31K/K37M/T60V/A79G/T83W/A96L/G162K/D174K/R195L/P210E/Y277V/E318Q | ++ | | ++++ | | | |
| 929/930 | L96P | H13P/A16S/D17G/A26E/E31K/K37G/T60V/A79G/T83W/A96P/G162K/D174K/R195L/P210E/Y277V/E318Q | + | | ++++ | | | |
| 931/932 | S16V | H13P/A16V/D17G/A26E/E31K/K37G/T60V/A79G/T83W/A96L/G162K/D174K/R195L/P210E/Y277V/E318Q | ++ | + | ++++ | + | + | |
| 933/934 | V60A | H13P/A16S/D17G/A26E/E31K/K37G/T60A/A79G/T83W/A96L/G162K/D174K/R195L/P210E/Y277V/E318Q | ++ | | ++++ | | | |
| 935/936 | E26R | H13P/A16S/D17G/A26R/E31K/K37G/T60V/A79G/T83W/A96L/G162K/D174K/R195L/P210E/Y277V/E318Q | ++ | | +++ | | | |
| 937/938 | K31G | H13P/A16S/D17G/A26E/E31G/K37G/T60V/A79G/T83W/A96L/G162K/D174K/R195L/P210E/Y277V/E318Q | ++ | | ++++ | | | |
| 939/940 | L195G | H13P/A16S/D17G/A26E/E31K/K37G/T60V/A79G/T83W/A96L/G162K/D174K/R195G/P210E/Y277V/E318Q | ++ | | ++++ | | | |
| 941/942 | Q318R | H13P/A16S/D17G/A26E/E31K/K37G/T60V/A79G/T83W/A96L/G162K/D174K/R195L/P210E/Y277V/E318R | ++ | | ++++ | | | |
| 943/944 | E26V | H13P/A16S/D17G/A26V/E31K/K37G/T60V/A79G/T83W/A96L/G162K/D174K/R195L/P210E/Y277V/E318Q | | | | | | |
| 945/946 | G79M | H13P/A16S/D17G/A26E/E31K/K37G/T60V/A79M/T883W/A96L/G162K/D174K/R195L/P210E/Y277V/E318Q | | | | | | |
| 947/948 | S16M | H13P/A16M/D17G/A26E/E31K/K37G/T60V/A79G/T83W/A96L/G162K/D174K/R195L/P210E/Y277V/E318Q | ++ | + | ++++ | + | + | |
| 949/950 | E210A | H13P/A16S/D17G/A26E/E31K/K37G/T60V/A79G/T83W/A96L/G162K/D174K/R195L/P210A/Y277V/E318Q | ++ | + | +++ | + | + | |
| 951/952 | L96R | H13P/A16S/D17G/A26E/E31K/K37G/T60V/A79G/T83W/A96R/G162K/D174K/R195L/P210E/Y277V/E318Q | ++ | + | ++++ | + | ++ | ++ |
| 953/954 | G37E | H13P/A16S/D17G/A26E/E31K/K37E/T60V/A79G/T83W/A96L/G162K/D174K/R195L/P210E/Y277V/E318Q | ++ | | +++ | | | |
| 955/956 | S16G | H13P/A16G/D17G/A26E/E31K/K37G/T60V/A79G/T83W/A96L/G162K/D174K/R195L/P210E/Y277V/E318Q | ++ | + | ++++ | + | + | ++ |
| 957/958 | G17W | H13P/A16S/D17W/A26E/E31K/K37G/T60V/A79G/T83W/A96L/G162K/D174K/R195L/P210E/Y277V/E318Q | + | | ++++ | | | |
| 959/960 | P13G | H13G/A16S/D17G/A26E/E31K/K37G/T60V/A79G/T83W/A96L/G162K/D174K/R195L/P210E/Y277V/E318Q | ++ | | ++++ | | | |
| 961/962 | G79E | H13P/A16S/D17G/A26E/E31K/K37G/T60V/A79E/T83W/A96L/G162K/D174K/R195L/P210E/Y277V/E318Q | | | | | | |
| 963/964 | V277T | H13P/A16S/D17G/A26E/E31K/K37G/T60V/A79G/T83W/A96L/G162K/D174K/R195L/P210E/Y277T/E318Q | +++ | + | ++++ | + | + | ++ |
| 965/966 | V60Q | H13P/A16S/D17G/A26E/E31K/K37G/T60Q/A79G/T83W/A96L/G162K/D174K/R195L/P210E/Y277V/E318Q | +++ | + | ++++ | ++ | + | + |
| 967/968 | K31L | H13P/A16S/D17G/A26E/E31L/K37G/T60V/A79G/T83W/A96L/G162K/D174K/R195L/P210E/Y277V/E318Q | ++ | | ++++ | | | |
| 969/970 | L96G | H13P/A16S/D17G/A26E/E31K/K37G/T60V/A79G/T83W/A96G/G162K/D174K/R195L/P210E/Y277V/E318Q | ++ | | ++++ | | | |
| 971/972 | L96T | H13P/A16S/D17G/A26E/E31K/K37G/T60V/A79G/T83W/A96T/G162K/D174K/R195L/P210E/Y277V/E318Q | ++ | + | ++++ | + | ++ | ++ |
| 973/974 | V60G | H13P/A16S/D17G/A26E/E31K/K37G/T60G/A79G/T83W/A96L/G162K/D174K/R195L/P210E/Y277V/E318Q | ++ | | ++++ | | | |
| 975/976 | S16W | H13P/A16W/D17G/A26E/E31K/K37G/T60V/A79G/T83W/A96L/G162K/D174K/R195L/P210E/Y277V/E318Q | ++ | | ++++ | | | |
| 977/978 | P13T | H13T/A16S/D17G/A26E/E31K/K37G/T60V/A79G/T83W/A96L/G162K/D174K/R195L/P210E/Y277V/E318Q | ++ | | ++++ | | | |
| 979/980 | G79R | H13P/A16S/D17G/A26E/E31K/K37G/T60V/A79R/T83W/A96L/G162K/D174K/R195L/P210E/Y277V/E318Q | | | | | | |
| 981/982 | E26Y | H13P/A16S/D17G/A26E/E31K/K37G/T60V/A79G/T83W/A96L/G162K/D174K/R195L/P210E/Y277V/E318Q | | | | | | |
| 983/984 | E26S | H13P/A16S/D17G/A26S/E31K/K37G/T60V/A79G/T83W/A96L/G162K/D174K/R195L/P210E/Y277V/E318Q | +++ | + | ++++ | + | + | |
| 985/986 | L195R | H13P/A16S/D17G/A26E/E31K/K37G/T60V/A79G/T83W/A96L/G162K/D174K/P210E/Y277V/E318Q | +++ | + | ++++ | + | + | |
| 987/988 | G17R | H13P/A16S/D17R/A26E/E31K/K37G/T60V/A79G/T83W/A96L/G162K/D174K/R195L/P210E/Y277V/E318Q | ++ | | +++ | | | |
| 989/990 | P13V | H13V/A16S/D17G/A26E/E31K/K37G/T60V/A79G/T83W/A96L/G162K/D174K/R195L/P210E/Y277V/E318Q | ++ | | ++++ | | | |
| 991/992 | V277E | H13P/A16S/D17G/A26E/E31K/K37G/T60V/A79G/T83W/A96L/G162K/D174K/R195L/P210E/Y277E/E318Q | ++ | | ++++ | | | |
| 993/994 | G37L | H13P/A16S/D17G/A26E/E31K/K37L/T60V/A79G/T83W/A96L/G162K/D174K/R195L/P210E/Y277V/E318Q | ++ | | ++++ | | | |
| 995/996 | E210G | H13P/A16S/D17G/A26E/E31K/K37G/T60V/A79G/T83W/A96L/G162K/D174K/R195L/P210G/Y277V/E318Q | ++ | | ++++ | | | |
| 997/998 | V60L | H13P/A16S/D17G/A26E/E31K/K37G/T60L/A79G/T83W/A96L/G162K/D174K/R195L/P210E/Y277V/E318Q | ++ | | ++++ | | | |
| 999/1000 | P13L | H13L/A16S/D17G/A26E/E31K/K37G/T60V/A79G/T83W/A96L/G162K/D174K/R195L/P210E/Y277V/E318Q | ++ | | ++++ | | | |
| 1001/1002 | W83Y | H13P/A16S/D17G/A26E/E31K/K37G/T60V/A79G/T83Y/A96L/G162K/D174K/R195L/P210E/Y277V/E318Q | +++ | + | ++++ | ++ | ++ | ++ |
| 1003/1004 | G37W | H13P/A16S/D17G/A26E/E31K/K37W/T60V/A79G/T83W/A96L/G162K/D174K/R195L/P210E/Y277V/E318Q | | | +++ | | | |
| 1005/1006 | V277G | H13P/A16S/D17G/A26E/E31K/K37G/T60V/A79G/T83W/A96L/G162K/D174K/R195L/P210E/Y277G/E318Q | + | | ++++ | | | |
| 1007/1008 | W83R | H13P/A16S/D17G/A26E/E31K/K37G/T60V/A79G/T83R/A96L/G162K/D174K/R195L/P210E/Y277V/E318Q | +++ | + | ++++ | ++ | + | + |
| 1009/1010 | V60Y | H13P/A16S/D17G/A26E/E31K/K37G/T60Y/A79G/T83W/A96L/G162K/D174K/R195L/P210E/Y277V/E318Q | ++ | | ++++ | | | |
| 1011/1012 | E210S | H13P/A16S/D17G/A26E/E31K/K37G/T60V/A79G/T83W/A96L/G162K/D174K/R195L/P210S/Y277V/E318Q | ++ | | ++++ | | + | |
| 1013/1014 | Q318S | H13P/A16S/D17G/A26E/E31K/K37G/T60V/A79G/T83W/A96L/G162K/D174K/R195L/P210E/Y277V/E318S | ++ | | ++++ | | | |
| 1015/1016 | G17I | H13P/A16S/D17I/A26E/E31K/K37G/T60V/A79G/T83W/A96L/G162K/D174K/R195L/P210E/Y277V/E318Q | ++ | | ++++ | | | |
| 1017/1018 | G37S | H13P/A16S/D17G/A26E/E31K/K37S/T60V/A79G/T83W/A96L/G162K/D174K/R195L/P210E/Y277V/E318Q | ++ | | ++++ | | | |
| 1019/1020 | G79L | H13P/A16S/D17G/A26E/E31K/K37G/T60V/A79L/T83W/A96L/G162K/D174K/R195L/P210E/Y277V/E318Q | | | | | | |
| 1021/1022 | S16R | H13P/A16R/D17G/A26E/E31K/K37G/T60V/A79G/T83W/A96L/G162K/D174K/R195L/P210E/Y277V/E318Q | ++ | | ++++ | | | |
| 1023/1024 | L195S | H13P/A16S/D17G/A26E/E31K/K37G/T60V/A79G/T83W/A96L/G162K/D174K/R195S/P210E/Y277V/E318Q | ++ | + | ++++ | | + | |
| 1025/1026 | Q318G | H13P/A16S/D17G/A26E/E31K/K37G/T60V/A79G/T83W/A96L/G162K/D174K/R195L/P210E/Y277V/E318G | + | | ++++ | | | |
| 1027/1028 | E26C | H13P/A16S/D17G/A26C/E31K/K37G/T60V/A79G/T83W/A96L/G162K/D174K/R195L/P210E/Y277V/E318Q | +++ | + | ++++ | + | + | |
| 1029/1030 | P13R | H13R/A16S/D17G/A26E/E31K/K37G/T60V/A79G/T83W/A96L/G162K/D174K/R195L/P210E/Y277V/E318Q | ++ | | ++++ | | | |
| 1031/1032 | L195Y | H13P/A16S/D17G/A26E/E31K/K37G/T60V/A79G/T83W/A96L/G162K/D174K/R195Y/P210E/Y277V/E318Q | ++ | | ++++ | | | |
| 1033/1034 | V60E | H13P/A16S/D17G/A26E/E31K/K37G/T60E/A79G/T83W/A96L/G162K/D174K/R195L/P210E/Y277V/E318Q | +++ | + | ++++ | ++ | + | |
| 1035/1036 | K162V | H13P/A16S/D17G/A26E/E31K/K37G/T60V/A79G/T83W/A96L/G162V/D174K/R195L/P210E/Y277V/E318Q | +++ | + | ++++ | + | + | |
| 1037/1038 | S16C | H13P/A16C/D17G/A26E/E31K/K37G/T60V/A79G/T83W/A96L/G162K/D174K/R195L/P210E/Y277V/E318Q | ++ | | ++++ | | | |
| 1039/1040 | L96V | H13P/A16S/D17G/A26E/E31K/K37G/T60V/A79G/T83W/A96V/G162K/D174K/R195L/P210E/Y277V/E318Q | ++ | | ++++ | | + | |
| 1041/1042 | W83M | H13P/A16S/D17G/A26E/E31K/K37G/T60V/A79G/T83M/A96L/G162K/D174K/R195L/P210E/Y277V/E318Q | +++ | + | ++++ | ++ | + | ++ |
| 1043/1044 | K31M | H13P/A16S/D17G/A26E/E31M/K37G/T60V/A79G/T83W/A96L/G162K/D174K/R195L/P210E/Y277V/E318Q | ++ | | ++++ | + | + | |
| 1045/1046 | K174V | H13P/A16S/D17G/A26E/E31K/K37G/T60V/A79G/T83W/A96L/G162K/D174V/R195L/P210E/Y277V/E318Q | ++ | | ++++ | | | |
| 1047/1048 | V277L | H13P/A16S/D17G/A26E/E31K/K37G/T60V/A79G/T83W/A96L/G162K/D174K/R195L/P210E/Y277L/E318Q | ++ | | ++++ | | | |
| 1049/1050 | E26M | H13P/A16S/D17G/A26M/E31K/K37G/T60V/A79G/T83W/A96L/G162K/D174K/R195L/P210E/Y277V/E318Q | ++ | | +++ | | | |
| 1051/1052 | Q318M | H13P/A16S/D17G/A26E/E31K/K37G/T60V/A79G/T83W/A96L/G162K/D174K/R195L/P210E/Y277V/E318M | ++ | | ++++ | | | |
| 1053/1054 | E210N | H13P/A16S/D17G/A26E/E31K/K37G/T60V/A79G/T83W/A96L/G162K/D174K/R195L/P210N/Y277V/E318Q | ++ | | +++ | | | |
| 1055/1056 | E26A | H13P/A16S/D17G/E31K/K37G/T60V/A79G/T83W/A96L/G162K/D174K/R195L/P210E/Y277V/E318Q | +++ | + | ++++ | + | + | + |
| 1057/1058 | V277R | H13P/A16S/D17G/A26E/E31K/K37G/T60V/A79G/T83W/A96L/G162K/D174K/R195L/P210E/Y277R/E318Q | ++ | | ++++ | | | |
| 1059/1060 | L96F | H13P/A16S/D17G/A26E/E31K/K37G/T60V/A79G/T83W/A96F/G162K/D174K/R195L/P210E/Y277V/E318Q | +++ | + | ++++ | ++ | + | + |
| 1061/1062 | K174G | H13P/A16S/D17G/A26E/E31K/K37G/T60V/A79G/T83W/A96L/G162K/D174G/R195L/P210E/Y277V/E318Q | +++ | + | ++++ | + | + | ++ |
| 1063/1064 | S16Q | H13P/A16Q/D17G/A26E/E31K/K37G/T60V/A79G/T83W/A96L/G162K/D174K/R195L/P210E/Y277V/E318Q | ++ | + | ++++ | + | + | + |
| 1065/1066 | V277A | H13P/A16S/D17G/A26E/E31K/K37G/T60V/A79G/T83W/A96L/G162K/D174K/R195L/P210E/Y277A/E318Q | ++ | | ++++ | | | |
| 1067/1068 | P13Q | H13Q/A16S/D17G/A26E/E31K/K37G/T60V/A79G/T83W/A96L/G162K/D174K/R195L/P210E/Y277V/E318Q | ++ | | ++++ | | | |
| 1069/1070 | G79F | H13P/A16S/D17G/A26E/E31K/K37G/T60V/A79F/T83W/A96L/G162K/D174K/R195L/P210E/Y277V/E318Q | | | | | | |
| 1071/1072 | S16L | H13P/A16L/D17G/A26E/E31K/K37G/T60V/A79G/T83W/A96L/G162K/D174K/R195L/P210E/Y277V/E318Q | ++ | | ++++ | | | |
| 1073/1074 | L195N | H13P/A16S/D17G/A26E/E31K/K37G/T60V/A79G/T83W/A96L/G162K/D174K/R195N/P210E/Y277V/E318Q | ++ | ++ | ++++ | + | ++ | + |
| 1075/1076 | G79V | H13P/A16S/D17G/A26E/E31K/K37G/T60V/A79V/T83W/A96L/G162K/D174K/R195L/P210E/Y277V/E318Q | | | | | | |
| 1077/1078 | G17P | H13P/A16S/D17P/A26E/E31K/K37G/T60V/A79G/T83W/A96L/G162K/D174K/R195L/P210E/Y277V/E318Q | + | | ++++ | | | |
| 1079/1080 | V60P | H13P/A16S/D17G/A26E/E31K/K37G/T60P/A79G/T83W/A96L/G162K/D174K/R195L/P210E/Y277V/E318Q | | | | | | |
| 1081/1082 | G37P | H13P/A16S/D17G/A26E/E31K/K37P/T60V/A79G/T83W/A96L/G162K/D174K/R195L/P210E/Y277V/E318Q | | | ++ | | | |
| 1083/1084 | L96W | H13P/A16S/D17G/A26E/E31K/K37G/T60V/A79G/T83W/A96W/G162K/D174K/R195L/P210E/Y277V/E318Q | ++ | + | ++++ | + | + | + |
| 1085/1086 | K162T | H13P/A16S/D17G/A26E/E31K/K37G/T60V/A79G/T83W/A96L/G162T/D174K/R195L/P210E/Y277V/E318Q | +++ | + | ++++ | + | + | ++ |
| 1087/1088 | L195M | H13P/A16S/D17G/A26E/E31K/K37G/T60V/A79G/T83W/A96L/G162K/D174K/R195M/P210E/Y277V/E318Q | ++ | + | ++++ | + | ++ | |
| 1089/1090 | Q318W | H13P/A16S/D17G/A26E/E31K/K37G/T60V/A79G/T83W/A96L/G162K/D174K/R195L/P210E/Y277V/E318W | + | | ++++ | | | |
| 1091/1092 | G37R | H13P/A16S/D17G/A26E/E31K/K37R/T60V/A79G/T83W/A96L/G162K/D174K/R195L/P210E/Y277V/E318Q | ++ | | +++ | | | |
| 1093/1094 | S16T | H13P/A16T/D17G/A26E/E31K/K37G/T60V/A79G/T83W/A96L/G162K/D174K/R195L/P210E/Y277V/E318Q | ++ | + | ++++ | + | + | |
| 1095/1096 | E210M | H13P/A16S/D17G/A26E/E31K/K37G/T60V/A79G/T83W/A96L/G162K/D174K/R195L/P210M/Y277V/E318Q | ++ | | +++ | | | |
| 1097/1098 | K162Y | H13P/A16S/D17G/A26E/E31K/K37G/T60V/A79G/T83W/A96L/G162Y/D174K/R195L/P210E/Y277V/E318Q | ++ | | ++++ | + | | + |
| 1099/1100 | S16P | H13P/A16P/D17G/A26E/E31K/K37G/T60V/A79G/T83W/A96L/G162K/D174K/R195L/P210E/Y277V/E318Q | + | | ++++ | | | |
| 1101/1102 | W83E | H13P/A16S/D17G/A26E/E31K/K37G/T60V/A79G/T83E/A96L/G162K/D174K/R195L/P210E/Y277V/E318Q | ++ | + | ++++ | + | + | |
| 1103/1104 | V277M | H13P/A16S/D17G/A26E/E31K/K37G/T60V/A79G/T83W/A96L/G162K/D174K/R195L/P210E/Y277M/E318Q | +++ | + | ++++ | + | + | |
| 1105/1106 | K174L | H13P/A16S/D17G/A26E/E31K/K37G/T60V/A79G/T83W/A96L/G162K/D174L/R195L/P210E/Y277V/E318Q | ++ | | ++++ | | | |
| 1107/1108 | G17V | H13P/A16S/D17V/A26E/E31K/K37G/T60V/A79G/T83W/A96L/G162K/D174K/R195L/P210E/Y277V/E318Q | ++ | | +++ | | | |
| 1109/1110 | G17E | H13P/A16S/D17E/A26E/E31K/K37G/T60V/A79G/T83W/A96L/G162K/D174K/R195L/P210E/Y277V/E318Q | ++ | + | +++ | + | + | |
| 1111/1112 | G17L | H13P/A16S/D17L/A26E/E31K/K37G/T60V/A79G/T83W/A96L/G162K/D174K/R195L/P210E/Y277V/E318Q | ++ | | ++++ | | | |
| 1113/1114 | L96M | H13P/A16S/D17G/A26E/E31K/K37G/T60V/A79G/T83W/A96M/G162K/D174K/R195L/P210E/Y277V/E318Q | ++ | + | ++++ | + | + | + |
| 1115/1116 | Q318V | H13P/A16S/D17G/A26E/E31K/K37G/T60V/A79G/T83W/A96L/G162K/D174K/R195L/P210E/Y277V/E318V | ++ | | ++++ | | | |
| 1117/1118 | K174R | H13P/A16S/D17G/A26E/E31K/K37G/T60V/A79G/T83W/A96L/G162K/D174R/R195L/P210E/Y277V/E318Q | +++ | + | ++++ | ++ | ++ | |
| 1119/1120 | P13S | H13S/A16S/D17G/A26E/E31K/K37G/T60V/A79G/T83W/A96L/G162K/D174K/R195L/P210E/Y277V/E318Q | ++ | | ++++ | + | | |
| 1121/1122 | K174T | H13P/A16S/D17G/A26E/E31K/K37G/T60V/A79G/T83W/A96L/G162K/D174T/R195L/P210E/Y277V/E318Q | +++ | + | ++++ | + | + | + |
| 1123/1124 | P13Y | H13Y/A16S/D17G/A26E/E31K/K37G/T60V/A79G/T83W/A96L/G162K/D174K/R195L/P210E/Y277V/E318Q | + | | ++++ | | | |
| 1125/1126 | L195A | H13P/A16S/D17G/A26E/E31K/K37G/T60V/A79G/T83W/A96L/G162K/D174K/R195A/P210E/Y277V/E318Q | ++ | + | ++++ | | ++ | |
| 1127/1128 | V277W | H13P/A16S/D17G/A26E/E31K/K37G/T60V/A79G/T83W/A96L/G162K/D174K/R195L/P210E/Y277W/E318Q | ++ | + | ++++ | + | + | |
| 1129/1130 | V60M | H13P/A16S/D17G/A26E/E31K/K37G/T60M/A79G/T83W/A96L/G162K/D174K/R195L/P210E/Y277V/E318Q | ++ | | ++++ | + | + | |
| 1131/1132 | G17A | H13P/A16S/D17A/A26E/E31K/K37G/T60V/A79G/T83W/A96L/G162K/D174K/R195L/P210E/Y277V/E318Q | ++ | | ++++ | | + | |
| 1133/1134 | Q318A | H13P/A16S/D17G/A26E/E31K/K37G/T60V/A79G/T83W/A96L/G162K/D174K/R195L/P210E/Y277V/E318A | ++ | | ++++ | | | |
| 1135/1136 | K174H | H13P/A16S/D17G/A26E/E31K/K37G/T60V/A79G/T83W/A96L/G162K/D174H/R195L/P210E/Y277V/E318Q | ++ | + | ++++ | + | + | + |
| 1137/1138 | W83S | H13P/A16S/D17G/A26E/E31K/K37G/T60V/A79G/T83S/A96L/G162K/D174K/R195L/P210E/Y277V/E318Q | +++ | + | ++++ | ++ | ++ | |
| 1139/1140 | V277I | H13P/A16S/D17G/A26E/E31K/K37G/T60V/A79G/T83W/A96L/G162K/D174K/R195L/P210E/Y277I/E318Q | +++ | | ++++ | + | + | |
| 1141/1142 | P13E | H13E/A16S/D17G/A26E/E31K/K37G/T60V/A79G/T83W/A96L/G162K/D174K/R195L/P210E/Y277V/E318Q | ++ | | ++++ | | | |
| 1143/1144 | G17K | H13P/A16S/D17K/A26E/E31K/K37G/T60V/A79G/T83W/A96L/G162K/D174K/R195L/P210E/Y277V/E318Q | ++ | | ++++ | | | |
| 1145/1146 | E26K | H13P/A16S/D17G/A26K/E31K/K37G/T60V/A79G/T83W/A96L/G162K/D174K/R195L/P210E/Y277V/E318Q | ++ | | ++++ | | | |
| 1147/1148 | L96A | H13P/A16S/D17G/A26E/E31K/K37G/T60V/A79G/T83W/G162K/D174K/R195L/P210E/Y277V/E318Q | ++ | + | ++++ | | + | + |
| 1149/1150 | K174D | H13P/A16S/D17G/A26E/E31K/K37G/T60V/A79G/T83W/A96L/G162K/R195L/P210E/Y277V/E318Q | +++ | + | ++++ | + | + | ++ |
| 1151/1152 | E210V | H13P/A16S/D17G/A26E/E31K/K37G/T60V/A79G/T83W/A96L/G162K/D174K/R195L/P210V/Y277V/E318Q | ++ | | +++ | | | |
| 1153/1154 | K31S | H13P/A16S/D17G/A26E/E31S/K37G/T60V/A79G/T83W/A96L/G162K/D174K/R195L/P210E/Y277V/E318Q | ++ | | ++++ | | | |
| 1155/1156 | L195C | H13P/A16S/D17G/A26E/E31K/K37G/T60V/A79G/T83W/A96L/G162K/D174K/R195C/P210E/Y277V/E318Q | ++ | + | ++++ | | + | |
| 1157/1158 | G79K | H13P/A16S/D17G/A26E/E31K/K37G/T60V/A79K/T83W/A96L/G162K/D174K/R195L/P210E/Y277V/E318Q | | | | | | |
| 1159/1160 | V60W | H13P/A16S/D17G/A26E/E31K/K37G/T60W/A79G/T83W/A96L/G162K/D174K/R195L/P210E/Y277V/E318Q | ++ | | ++++ | | | |
| 1161/1162 | K174S | H13P/A16S/D17G/A26E/E31K/K37G/T60V/A79G/T83W/A96L/G162K/D174S/R195L/P210E/Y277V/E318Q | +++ | + | ++++ | + | + | ++ |
| 1163/1164 | P13I | H13I/A16S/D17G/A26E/E31K/K37G/T60V/A79G/T83W/A96L/G162K/D174K/R195L/P210E/Y277V/E318Q | ++ | | ++++ | | | |
| 1165/1166 | V60H | H13P/A16S/D17G/A26E/E31K/K37G/T60H/A79G/T83W/A96L/G162K/D174K/R195L/P210E/Y277V/E318Q | ++ | + | ++++ | + | + | |
| 1167/1168 | K31E | H13P/A16S/D17G/A26E/K37G/T60V/A79G/T83W/A96L/G162K/D174K/R195L/P210E/Y277V/E318Q | +++ | ++ | ++++ | ++ | ++ | |
| 1169/1170 | L96E | H13P/A16S/D17G/A26E/E31K/K37G/T60V/A79G/T83W/A96E/G162K/D174K/R195L/P210E/Y277V/E318Q | ++ | + | ++++ | + | + | ++ |
| 1171/1172 | W83K | H13P/A16S/D17G/A26E/E31K/K37G/T60V/A79G/T83K/A96L/G162K/D174K/R195L/P210E/Y277V/E318Q | +++ | + | ++++ | ++ | ++ | + |
| 1173/1174 | K31Q | H13P/A16S/D17G/A26E/E31Q/K37G/T60V/A79G/T83W/A96L/G162K/D174K/R195L/P210E/Y277V/E318Q | +++ | | ++++ | + | + | |
| 1175/1176 | S16K | H13P/A16K/D17G/A26E/E31K/K37G/T60V/A79G/T83W/A96L/G162K/D174K/R195L/P210E/Y277V/E318Q | ++ | | ++++ | | + | |
| 1177/1178 | K174M | H13P/A16S/D17G/A26E/E31K/K37G/T60V/A79G/T83W/A96L/G162K/D174M/R195L/P210E/Y277V/E318Q | ++ | | ++++ | | | |
| 1179/1180 | G37V | H13P/A16S/D17G/A26E/E31K/K37V/T60V/A79G/T83W/A96L/G162K/D174K/R195L/P210E/Y277V/E318Q | | | +++ | | | |
| 1181/1182 | G79D | H13P/A16S/D17G/A26E/E31K/K37G/T60V/A79D/T83W/A96L/G162K/D174K/R195L/P210E/Y277V/E318Q | | | | | | |
| 1183/1184 | Q318T | H13P/A16S/D17G/A26E/E31K/K37G/T60V/A79G/T83W/A96L/G162K/D174K/R195L/P210E/Y277V/E318T | ++ | | ++++ | | | |
| 1185/1186 | L195F | H13P/A16S/D17G/A26E/E31K/K37G/T60V/A79G/T83W/A96L/G162K/D174K/R195F/P210E/Y277V/E318Q | +++ | + | ++++ | + | + | |
| 1187/1188 | E26T | H13P/A16S/D17G/A26T/E31K/K37G/T60V/A79G/T83W/A96L/G162K/D174K/R195L/P210E/Y277V/E318Q | + | | ++++ | | | |
| 1189/1190 | K174Q | H13P/A16S/D17G/A26E/E31K/K37G/T60V/A79G/T83W/A96L/G162K/D174Q/R195L/P210E/Y277V/E318Q | +++ | + | +++ | + | + | ++ |
| 1191/1192 | W83L | H13P/A16S/D17G/A26E/E31K/K37G/T60V/A79G/T83L/A96L/G162K/D174K/R195L/P210E/Y277V/E318Q | ++ | + | ++++ | + | + | + |
| 1193/1194 | L96I | H13P/A16S/D17G/A26E/E31K/K37G/T60V/A79G/T83W/A96I/G162K/D174K/R195L/P210E/Y277V/E318Q | ++ | | ++++ | | + | |
| 1195/1196 | P13C | H13C/A16S/D17G/A26E/E31K/K37G/T60V/A79G/T83W/A96L/G162K/D174K/R195L/P210E/Y277V/E318Q | ++ | | ++++ | | | |
| 1197/1198 | P13A | H13A/A16S/D17G/A26E/E31K/K37G/T60V/A79G/T83W/A96L/G162K/D174K/R195L/P210E/Y277V/E318Q | ++ | + | ++++ | + | + | |
| 1199/1200 | L96Q | H13P/A16S/D17G/A26E/E31K/K37G/T60V/A79G/T83W/A96Q/G162K/D174K/R195L/P210E/Y277V/E318Q | ++ | | ++++ | + | + | ++ |
| 1201/1202 | P13H | A16S/D17G/A26E/E31K/K37G/T60V/A79G/T83W/A96L/G162K/D174K/R195L/P210E/Y277V/E318Q | ++ | | ++++ | | | |
| 1203/1204 | W83T | H13P/A16S/D17G/A26E/E31K/K37G/T60V/A79G/A96L/G162K/D174K/R195L/P210E/Y277V/E318Q | +++ | ++ | ++++ | ++ | ++ | + |
| 1205/1206 | K31Y | H13P/A16S/D17G/A26E/E31Y/K37G/T60V/A79G/T83W/A96L/G162K/D174K/R195L/P210E/Y277V/E318Q | ++ | | ++++ | | | |
| 1207/1208 | P13W | H13W/A16S/D17G/A26E/E31K/K37G/T60V/A79G/T83W/A96L/G162K/D174K/R195L/P210E/Y277V/E318Q | | | +++ | | | |
| 1209/1210 | V277P | H13P/A16S/D17G/A26E/E31K/K37G/T60V/A79G/T83W/A96L/G162K/D174K/R195L/P210E/Y277P/E318Q | | | ++ | | | |
| 1211/1212 | L195T | H13P/A16S/D17G/A26E/E31K/K37G/T60V/A79G/T83W/A96L/G162K/D174K/R195T/P210E/Y277V/E318Q | ++ | + | ++++ | | ++ | |
| 1213/1214 | V60S | H13P/A16S/D17G/A26E/E31K/K37G/T60S/A79G/T83W/A96L/G162K/D174K/R195L/P210E/Y277V/E318Q | ++ | | ++++ | | | |
| 1215/1216 | E26Q | H13P/A16S/D17G/A26Q/E31K/K37G/T60V/A79G/T83W/A96L/G162K/D174K/R195L/P210E/Y277V/E318Q | +++ | + | +++ | ++ | ++ | + |
| 1217/1218 | E210P | H13P/A16S/D17G/A26E/E31K/K37G/T60V/A79G/T83W/A96L/G162K/D174K/R195L/Y277V/E318Q | ++ | + | +++ | | + | |
| 1219/1220 | K174N | H13P/A16S/D17G/A26E/E31K/K37G/T60V/A79G/T83W/A96L/G162K/D174N/R195L/P210E/Y277V/E318Q | | | | | | |
| 1221/1222 | W83A | H13P/A16S/D17G/A26E/E31K/K37G/T60V/A79G/T83A/A96L/G162K/D174K/R195L/P210E/Y277V/E318Q | +++ | + | ++++ | ++ | ++ | + |
| 1223/1224 | K162E | H13P/A16S/D17G/A26E/E31K/K37G/T60V/A79G/T83W/A96L/G162E/D174K/R195L/P210E/Y277V/E318Q | +++ | + | +++ | ++ | + | ++ |
| 1225/1226 | G37F | H13P/A16S/D17G/A26E/E31K/K37F/T60V/A79G/T83W/A96L/G162K/D174K/R195L/P210E/Y277V/E318Q | + | | +++ | | | |
| 1227/1228 | G37Q | H13P/A16S/D17G/A26E/E31K/K37Q/T60V/A79G/T83W/A96L/G162K/D174K/R195L/P210E/Y277V/E318Q | ++ | | +++ | | | |
| 1229/1230 | K31T | H13P/A16S/D17G/A26E/E31T/K37G/T60V/A79G/T83W/A96L/G162K/D174K/R195L/P210E/Y277V/E318Q | ++ | + | ++++ | + | + | |
| 1231/1232 | K174A | H13P/A16S/D17G/A26E/E31K/K37G/T60V/A79G/T83W/A96L/G162K/D174A/R195L/P210E/Y277V/E318Q | ++ | | ++++ | + | + | + |
| 1233/1234 | W83F | H13P/A16S/D17G/A26E/E31K/K37G/T60V/A79G/T83F/A96L/G162K/D174K/R195L/P210E/Y277V/E318Q | +++ | + | ++++ | ++ | ++ | ++ |
| 1235/1236 | V60T | H13P/A16S/D17G/A26E/E31K/K37G/A79G/T83W/A96L/G162K/D174K/R195L/P210E/Y277V/E318Q | +++ | + | ++++ | ++ | + | |
| 1237/1238 | L96S | H13P/A16S/D17G/A26E/E31K/K37G/T60V/A79G/T83W/A96S/G162K/D174K/R195L/P210E/Y277V/E318Q | ++ | + | ++++ | + | ++ | + |
| 1239/1240 | W83V | H13P/A16S/D17G/A26E/E31K/K37G/T60V/A79G/T83V/A96L/G162K/D174K/R195L/P210E/Y277V/E318Q | +++ | ++ | ++++ | ++ | ++ | ++ |
| 1241/1242 | E210I | H13P/A16S/D17G/A26E/E31K/K37G/T60V/A79G/T83W/A96L/G162K/D174K/R195L/P210I/Y277V/E318Q | ++ | | ++++ | | | |
| 1243/1244 | S16D | H13P/A16D/D17G/A26E/E31K/K37G/T60V/A79G/T83W/A96L/G162K/D174K/R195L/P210E/Y277V/E318Q | ++ | | ++++ | | | |
| 1245/1246 | G37C | H13P/A16S/D17G/A26E/E31K/K37C/T60V/A79G/T83W/A96L/G162K/D174K/R195L/P210E/Y277V/E318Q | ++ | | +++ | | | |
| 1247/1248 | W83G | H13P/A16S/D17G/A26E/E31K/K37G/T60V/A79G/T83G/A96L/G162K/D174K/R195L/P210E/Y277V/E318Q | +++ | + | ++++ | ++ | ++ | |
| 1249/1250 | P13N | H13N/A16S/D17G/A26E/E31K/K37G/T60V/A79G/T83W/A96L/G162K/D174K/R195L/P210E/Y277V/E318Q | ++ | | ++++ | | | |
| 1251/1252 | K31V | H13P/A16S/D17G/A26E/E31V/K37G/T60V/A79G/T83W/A96L/G162K/D174K/R195L/P210E/Y277V/E318Q | +++ | | ++++ | + | + | |
| 1253/1254 | G79S | H13P/A16S/D17G/A26E/E31K/K37G/T60V/A79S/T83W/A96L/G162K/D174K/R195L/P210E/Y277V/E318Q | ++ | | ++++ | | | |
| 1255/1256 | L96Y | H13P/A16S/D17G/A26E/E31K/K37G/T60V/A79G/T83W/A96Y/G162K/D174K/R195L/P210E/Y277V/E318Q | ++ | + | ++++ | + | + | + |
| 1257/1258 | G17F | H13P/A16S/D17F/A26E/E31K/K37G/T60V/A79G/T83W/A96L/G162K/D174K/R195L/P210E/Y277V/E318Q | ++ | | ++++ | | | |
| 1259/1260 | G17M | H13P/A16S/D17M/A26E/E31K/K37G/T60V/A79G/T83W/A96L/G162K/D174K/R195L/P210E/Y277V/E318Q | ++ | + | ++++ | + | + | |
| 1261/1262 | S16A | H13P/D17G/A26E/E31K/K37G/T60V/A79G/T83W/A96L/G162K/D174K/R195L/P210E/Y277V/E318Q | +++ | + | ++++ | ++ | + | |
| 1263/1264 | W83P | H13P/A16S/D17G/A26E/E31K/K37G/T60V/A79G/T83P/A96L/G162K/D174K/R195L/P210E/Y277V/E318Q | | | + | | | |
| 1265/1266 | Q318L | H13P/A16S/D17G/A26E/E31K/K37G/T60V/A79G/T83W/A96L/G162K/D174K/R195L/P210E/Y277V/E318L | ++ | | ++++ | | | |
| 1267/1268 | G79Q | H13P/A16S/D17G/A26E/E31K/K37G/T60V/A79Q/T83W/A96L/G162K/D174K/R195L/P210E/Y277V/E318Q | | | | | | |
| 1269/1270 | G17S | H13P/A16S/D17S/A26E/E31K/K37G/T60V/A79G/T83W/A96L/G162K/D174K/R195L/P210E/Y277V/E318Q | ++ | + | ++++ | + | + | |
| 1271/1272 | E26W | H13P/A16S/D17G/A26W/E31K/K37G/T60V/A79G/T83W/A96L/G162K/D174K/R195L/P210E/Y277V/E318Q | | | | | | |
| 1273/1274 | L195D | H13P/A16S/D17G/A26E/E31K/K37G/T60V/A79G/T83W/A96L/G162K/D174K/R195D/P210E/Y277V/E318Q | ++ | + | ++++ | | + | |
| 1275/1276 | L195W | H13P/A16S/D17G/A26E/E31K/K37G/T60V/A79G/T83W/A96L/G162K/D174K/R195W/P210E/Y277V/E318Q | ++ | | ++++ | | | |
| 1277/1278 | V277S | H13P/A16S/D17G/A26E/E31K/K37G/T60V/A79G/T83W/A96L/G162K/D174K/R195L/P210E/Y277S/E318Q | ++ | | ++++ | + | + | |
| 1279/1280 | K174E | H13P/A16S/D17G/A26E/E31K/K37G/T60V/A79G/T83W/A96L/G162K/D174E/R195L/P210E/Y277V/E318Q | +++ | ++ | ++++ | ++ | ++ | ++ |
| 1281/1282 | S16E | H13P/A16E/D17G/A26E/E31K/K37G/T60V/A79G/T83W/A96L/G162K/D174K/R195L/P210E/Y277V/E318Q | ++ | + | ++++ | | + | |
| 1283/1284 | K162F | H13P/A16S/D17G/A26E/E31K/K37G/T60V/A79G/T83W/A96L/G162F/D174K/R195L/P210E/Y277V/E318Q | | | | | | |
| 1285/1286 | G79Y | H13P/A16S/D17G/A26E/E31K/K37G/T60V/A79Y/T83W/A96L/G162K/D174K/R195L/P210E/Y277V/E318Q | | | | | | |
| 1287/1288 | G79A | H13P/A16S/D17G/A26E/E31K/K37G/T60V/T83W/A96L/G162K/D174K/R195L/P210E/Y277V/E318Q | ++ | | ++++ | | | |
| 1289/1290 | G17C | H13P/A16S/D17C/A26E/E31K/K37G/T60V/A79G/T83W/A96L/G162K/D174K/R195L/P210E/Y277V/E318Q | ++ | | +++ | | | |
| 1291/1292 | G17Y | H13P/A16S/D17Y/A26E/E31K/K37G/T60V/A79G/T83W/A96L/G162K/D174K/R195L/P210E/Y277V/E318Q | ++ | | +++ | | | |
| 1293/1294 | L195Q | H13P/A16S/D17G/A26E/E31K/K37G/T60V/A79G/T83W/A96L/G162K/D174K/R195Q/P210E/Y277V/E318Q | + | | ++++ | | | |
| 1295/1296 | E26P | H13P/A16S/D17G/A26P/E31K/K37G/T60V/A79G/T83W/A96L/G162K/D174K/R195L/P210E/Y277V/E318Q | ++ | + | ++++ | + | + | + |
| 1297/1298 | L1951 | H13P/A16S/D17G/A26E/E31K/K37G/T60V/A79G/T83W/A96L/G162K/D174K/R195I/P210E/Y277V/E318Q | ++ | + | ++++ | | + | + |
| 1299/1300 | V60F | H13P/A16S/D17G/A26E/E31K/K37G/T60F/A79G/T83W/A96L/G162K/D174K/R195L/P210E/Y277V/E318Q | ++ | | ++++ | | | |
| 1301/1302 | E26I | H13P/A16S/D17G/A26I/E31K/K37G/T60V/A79G/T83W/A96L/G162K/D174K/R195L/P210E/Y277V/E318Q | | | | | | |
| 1303/1304 | G17D | H13P/A16S/A26E/E31K/K37G/T60V/A79G/T83W/A96L/G162K/D174K/R195L/P210E/Y277V/E318Q | ++ | + | ++++ | + | + | |
| 1305/1306 | Q318K | H13P/A16S/D17G/A26E/E31K/K37G/T60V/A79G/T83W/A96L/G162K/D174K/R195L/P210E/Y277V/E318K | ++ | | ++++ | | | |
| 1307/1308 | V60N | H13P/A16S/D17G/A26E/E31K/K37G/T60N/A79G/T83W/A96L/G162K/D174K/R195L/P210E/Y277V/E318Q | ++ | | ++++ | + | + | |
| 1309/1310 | S161 | H13P/A16I/D17G/A26E/E31K/K37G/T60V/A79G/T83W/A96L/G162K/D174K/R195L/P210E/Y277V/E318Q | ++ | | ++++ | | | |
| 1311/1312 | Q318H | H13P/A16S/D17G/A26E/E31K/K37G/T60V/A79G/T83W/A96L/G162K/D174K/R195L/P210E/Y277V/E318H | ++ | | ++++ | | | |
| 1313/1314 | G37A | H13P/A16S/D17G/A26E/E31K/K37A/T60V/A79G/T83W/A96L/G162K/D174K/R195L/P210E/Y277V/E318Q | ++ | | +++ | | | |
| 1315/1316 | L195V | H13P/A16S/D17G/A26E/E31K/K37G/T60V/A79G/T83W/A96L/G162K/D174K/R195V/P210E/Y277V/E318Q | ++ | + | ++++ | + | ++ | ++ |
| 1317/1318 | E210Y | H13P/A16S/D17G/A26E/E31K/K37G/T60V/A79G/T83W/A96L/G162K/D174K/R195L/P210Y/Y277V/E318Q | ++ | | +++ | | | |
| 1319/1320 | L195E | H13P/A16S/D17G/A26E/E31K/K37G/T60V/A79G/T83W/A96L/G162K/D174K/R195E/P210E/Y277V/E318Q | ++ | | ++++ | | + | |
| 1321/1322 | E26H | H13P/A16S/D17G/A26H/E31K/K37G/T60V/A79G/T83W/A96L/G162K/D174K/R195L/P210E/Y277V/E318Q | + | | ++++ | | | |
| 1323/1324 | P13D | H13D/A16S/D17G/A26E/E31K/K37G/T60V/A79G/T83W/A96L/G162K/D174K/R195L/P210E/Y277V/E318Q | ++ | | ++++ | | + | |
| 1325/1326 | K31P | H13P/A16S/D17G/A26E/E31P/K37G/T60V/A79G/T83W/A96L/G162K/D174K/R195L/P210E/Y277V/E318Q | ++ | + | ++++ | + | + | |
| 1327/1328 | E210W | H13P/A16S/D17G/A26E/E31K/K37G/T60V/A79G/T83W/A96L/G162K/D174K/R195L/P210W/Y277V/E318Q | + | | ++++ | | | |
| 1329/1330 | Q318P | H13P/A16S/D17G/A26E/E31K/K37G/T60V/A79G/T83W/A96L/G162K/D174K/R195L/P210E/Y277V/E318P | | | | | | |
| 1331/1332 | W83C | H13P/A16S/D17G/A26E/E31K/K37G/T60V/A79G/T83C/A96L/G162K/D174K/R195L/P210E/Y277V/E318Q | +++ | + | ++++ | ++ | ++ | + |
| 1333/1334 | K174P | H13P/A16S/D17G/A26E/E31K/K37G/T60V/A79G/T83W/A96L/G162K/D174P/R195L/P210E/Y277V/E318Q | ++ | | ++++ | | + | |
| 1335/1336 | E210T | H13P/A16S/D17G/A26E/E31K/K37G/T60V/A79G/T83W/A96L/G162K/D174K/R195L/P210T/Y277V/E318Q | ++ | | ++++ | | | |
| 1337/1338 | G17T | H13P/A16S/D17T/A26E/E31K/K37G/T60V/A79G/T83W/A96L/G162K/D174K/R195L/P210E/Y277V/E318Q | ++ | | ++++ | | | |
| 1339/1340 | G79T | H13P/A16S/D17G/A26E/E31K/K37G/T60V/A79T/T83W/A96L/G162K/D174K/R195L/P210E/Y277V/E318Q | | | ++ | | | |
| 1341/1342 | L96H | H13P/A16S/D17G/A26E/E31K/K37G/T60V/A79G/T83W/A96H/G162K/D174K/R195L/P210E/Y277V/E318Q | +++ | + | ++++ | + | ++ | + |
| 1343/1344 | G79W | H13P/A16S/D17G/A26E/E31K/K37G/T60V/A79W/T83W/A96L/G162K/D174K/R195L/P210E/Y277V/E318Q | | | | | | |
| 1345/1346 | K162L | H13P/A16S/D17G/A26E/E31K/K37G/T60V/A79G/T83W/A96L/G162L/D174K/R195L/P210E/Y277V/E318Q | +++ | + | ++++ | + | + | ++ |
| 1347/1348 | Q318E | H13P/A16S/D17G/A26E/E31K/K37G/T60V/A79G/T83W/A96L/G162K/D174K/R195L/P210E/Y277V | ++ | + | ++++ | + | + | ++ |
| 1349/1350 | K31A | H13P/A16S/D17G/A26E/E31A/K37G/T60V/A79G/T83W/A96L/G162K/D174K/R195L/P210E/Y277V/E318Q | ++ | + | ++++ | + | + | |
| 1351/1352 | E210C | H13P/A16S/D17G/A26E/E31K/K37G/T60V/A79G/T83W/A96L/G162K/D174K/R195L/P210C/Y277V/E318Q | ++ | | ++++ | | | |
| 1353/1354 | G17N | H13P/A16S/D17N/A26E/E31K/K37G/T60V/A79G/T83W/A96L/G162K/D174K/R195L/P210E/Y277V/E318Q | ++ | + | ++++ | | + | |
| 1355/1356 | L195K | H13P/A16S/D17G/A26E/E31K/K37G/T60V/A79G/T83W/A96L/G162K/D174K/R195K/P210E/Y277V/E318Q | ++ | | ++++ | | | |
| 1357/1358 | Q318Y | H13P/A16S/D17G/A26E/E31K/K37G/T60V/A79G/T83W/A96L/G162K/D174K/R195L/P210E/Y277V/E318Y | + | | ++++ | | | |
| 1359/1360 | G17H | H13P/A16S/D17H/A26E/E31K/K37G/T60V/A79G/T83W/A96L/G162K/D174K/R195L/P210E/Y277V/E318Q | ++ | + | +++ | + | + | |
| 1361/1362 | P13M | H13M/A16S/D17G/A26E/E31K/K37G/T60V/A79G/T83W/A96L/G162K/D174K/R195L/P210E/Y277V/E318Q | ++ | | ++++ | | | |
| 1363/1364 | V60D | H13P/A16S/D17G/A26E/E31K/K37G/T60D/A79G/T83W/A96L/G162K/D174K/R195L/P210E/Y277V/E318Q | ++ | | ++++ | | | |
| 1365/1366 | V277K | H13P/A16S/D17G/A26E/E31K/K37G/T60V/A79G/T83W/A96L/G162K/D174K/R195L/P210E/Y277K/E318Q | ++ | | ++++ | | | |
| 1367/1368 | S16N | H13P/A16N/D17G/A26E/E31K/K37G/T60V/A79G/T83W/A96L/G162K/D174K/R195L/P210E/Y277V/E318Q | ++ | | ++++ | | | |
| 1369/1370 | G79C | H13P/A16S/D17G/A26E/E31K/K37G/T60V/A79C/T83W/A96L/G162K/D174K/R195L/P210E/Y277V/E318Q | | | +++ | | | |
| 1371/1372 | P13K | H13K/A16S/D17G/A26E/E31K/K37G/T60V/A79G/T83W/A96L/G162K/D174K/R195L/P210E/Y277V/E318Q | ++ | | ++++ | | | |
| 1373/1374 | V60K | H13P/A16S/D17G/A26E/E31K/K37G/T60K/A79G/T83W/A96L/G162K/D174K/R195L/P210E/Y277V/E318Q | +++ | + | ++++ | ++ | + | + |
| 1375/1376 | G37N | H13P/A16S/D17G/A26E/E31K/K37N/T60V/A79G/T83W/A96L/G162K/D174K/R195L/P210E/Y277V/E318Q | ++ | | ++++ | | + | |
| 1377/1378 | V60I | H13P/A16S/D17G/A26E/E31K/K37G/T60I/A79G/T83W/A96L/G162K/D174K/R195L/P210E/Y277V/E318Q | ++ | + | ++++ | + | ++ | |
| 1379/1380 | K162D | H13P/A16S/D17G/A26E/E31K/K37G/T60V/A79G/T83W/A96L/G162D/D174K/R195L/P210E/Y277V/E318Q | +++ | + | ++++ | ++ | ++ | + |
| 1381/1382 | K162I | H13P/A16S/D17G/A26E/E31K/K37G/T60V/A79G/T83W/A96L/G162I/D174K/R195L/P210E/Y277V/E318Q | ++ | + | ++++ | + | + | |
| 1383/1384 | S16Y | H13P/A16Y/D17G/A26E/E31K/K37G/T60V/A79G/T83W/A96L/G162K/D174K/R195L/P210E/Y277V/E318Q | ++ | | ++++ | | | |
| 1385/1386 | K162S | H13P/A16S/D17G/A26E/E31K/K37G/T60V/A79G/T83W/A96L/G162S/D174K/R195L/P210E/Y277V/E318Q | ++ | | ++++ | + | + | ++ |
| 1387/1388 | L96C | H13P/A16S/D17G/A26E/E31K/K37G/T60V/A79G/T83W/A96C/G162K/D174K/R195L/P210E/Y277V/E318Q | +++ | ++ | ++++ | + | ++ | ++ |
| 1389/1390 | E210K | H13P/A16S/D17G/A26E/E31K/K37G/T60V/A79G/T83W/A96L/G162K/D174K/R195L/P210K/Y277V/E318Q | ++ | | +++ | | | |
| 1391/1392 | K174I | H13P/A16S/D17G/A26E/E31K/K37G/T60V/A79G/T83W/A96L/G162K/D174I/R195L/P210E/Y277V/E318Q | ++ | | ++++ | | | |
| 1393/1394 | E210L | H13P/A16S/D17G/A26E/E31K/K37G/T60V/A79G/T83W/A96L/G162K/D174K/R195L/P210L/Y277V/E318Q | ++ | | ++++ | | | |
| 1395/1396 | E26D | H13P/A16S/D17G/A26D/E31K/K37G/T60V/A79G/T83W/A96L/G162K/D174K/R195L/P210E/Y277V/E318Q | + | | ++++ | | | |
| 1397/1398 | E26N | H13P/A16S/D17G/A26N/E31K/K37G/T60V/A79G/T83W/A96L/G162K/D174K/R195L/P210E/Y277V/E318Q | ++ | | ++++ | | | |
| 1399/1400 | E210D | H13P/A16S/D17G/A26E/E31K/K37G/T60V/A79G/T83W/A96L/G162K/D174K/R195L/P210D/Y277V/E318Q | ++ | | ++++ | | | |
| 1401/1402 | P13F | H13F/A16S/D17G/A26E/E31K/K37G/T60V/A79G/T83W/A96L/G162K/D174K/R195L/P210E/Y277V/E318Q | ++ | | +++ | | | |
| 1403/1404 | Q318N | H13P/A16S/D17G/A26E/E31K/K37G/T60V/A79G/T83W/A96L/G162K/D174K/R195L/P210E/Y277V/E318N | ++ | | ++++ | | | |
| 1405/1406 | V277Y | H13P/A16S/D17G/A26E/E31K/K37G/T60V/A79G/T83W/A96L/G162K/D174K/R195L/P210E/E318Q | ++ | + | ++++ | + | + | |
| 1407/1408 | K174W | H13P/A16S/D17G/A26E/E31K/K37G/T60V/A79G/T83W/A96L/G162K/D174W/R195L/P210E/Y277V/E318Q | ++ | | ++++ | | | |
| 1409/1410 | K31H | H13P/A16S/D17G/A26E/E31H/K37G/T60V/A79G/T83W/A96L/G162K/D174K/R195L/P210E/Y277V/E318Q | ++ | + | ++++ | + | + | |
| 1411/1412 | K31D | H13P/A16S/D17G/A26E/E31D/K37G/T60V/A79G/T83W/A96L/G162K/D174K/R195L/P210E/Y277V/E318Q | ++ | + | ++++ | + | + | |
| 1413/1414 | G79N | H13P/A16S/D17G/A26E/E31K/K37G/T60V/A79N/T83W/A96L/G162K/D174K/R195L/P210E/Y277V/E318Q | | | | | | |
| 1415/1416 | G37K | H13P/A16S/D17G/A26E/E31K/T60V/A79G/T83W/A96L/G162K/D174K/R195L/P210E/Y277V/E318Q | ++ | | ++++ | | | |
| 1417/1418 | K31I | H13P/A16S/D17G/A26E/E311/K37G/T60V/A79G/T83W/A96L/G162K/D174K/R195L/P210E/Y277V/E318Q | ++ | | ++++ | | | |
| 1419/1420 | G37H | H13P/A16S/D17G/A26E/E31K/K37H/T60V/A79G/T83W/A96L/G162K/D174K/R195L/P210E/Y277V/E318Q | ++ | | +++ | | | |
| 1421/1422 | Q318C | H13P/A16S/D17G/A26E/E31K/K37G/T60V/A79G/T83W/A96L/G162K/D174K/R195L/P210E/Y277V/E318C | ++ | | ++++ | | | |
| 1423/1424 | K31F | H13P/A16S/D17G/A26E/E31F/K37G/T60V/A79G/T83W/A96L/G162K/D174K/R195L/P210E/Y277V/E318Q | ++ | | ++++ | | | |
| 1425/1426 | G79P | H13P/A16S/D17G/A26E/E31K/K37G/T60V/A79P/T83W/A96L/G162K/D174K/R195L/P210E/Y277V/E318Q | | | | | | |
| 1427/1428 | L195P | H13P/A16S/D17G/A26E/E31K/K37G/T60V/A79G/T83W/A96L/G162K/D174K/R195P/P210E/Y277V/E318Q | ++ | | ++++ | | | |
| 1429/1430 | Q318I | H13P/A16S/D17G/A26E/E31K/K37G/T60V/A79G/T83W/A96L/G162K/D174K/R195L/P210E/Y277V/E318I | ++ | | ++++ | | | |
| 1431/1432 | K162A | H13P/A16S/D17G/A26E/E31K/K37G/T60V/A79G/T83W/A96L/G162A/D174K/R195L/P210E/Y277V/E318Q | +++ | + | ++++ | + | + | ++ |
| 1433/1434 | V277Q | H13P/A16S/D17G/A26E/E31K/K37G/T60V/A79G/T83W/A96L/G162K/D174K/R195L/P210EIY277Q/E318Q | ++ | | ++++ | + | + | |
| 1435/1436 | E26F | H13P/A16S/D17G/A26F/E31K/K37G/T60V/A79G/T83W/A96L/G162K/D174K/R195L/P210E/Y277V/E318Q | | | | | | |
| 1437/1438 | V277C | H13P/A16S/D17G/A26E/E31K/K37G/T60V/A79G/T83W/A96L/G162K/D174K/R195L/P210EIY277C/E318Q | ++ | | ++++ | | + | |
| 1439/1440 | G79H | H13P/A16S/D17G/A26E/E31K/K37G/T60V/A79H/T83W/A96L/G162K/D174K/R195L/P210E/Y277V/E318Q | | | | | | |
| 1441/1442 | K31C | H13P/A16S/D17G/A26E/E31C/K37G/T60V/A79G/T83W/A96L/G162K/D174K/R195L/P210E/Y277V/E318Q | ++ | | ++++ | | | |
| 1443/1444 | G79I | H13P/A16S/D17G/A26E/E31K/K37G/T60V/A79I/T83W/A96L/G162K/D174K/R195L/P210E/Y277V/E318Q | | | | | | |
| 1445/1446 | G37I | H13P/A16S/D17G/A26E/E31K/K37I/T60V/A79G/T83W/A96L/G162K/D174K/R195L/P210E/Y277V/E318Q | | | +++ | | | |
| 1447/1448 | G17Q | H13P/A16S/D17Q/A26E/E31K/K37G/T60V/A79G/T83W/A96L/G162K/D174K/R195L/P210E/Y277V/E318Q | +++ | + | ++++ | ++ | ++ | |
| 1449/1450 | K31N | H13P/A16S/D17G/A26E/E31N/K37G/T60V/A79G/T83W/A96L/G162K/D174K/R195L/P210E/Y277V/E318Q | ++ | | ++++ | | + | |
| 1451/1452 | V277N | H13P/A16S/D17G/A26E/E31K/K37G/T60V/A79G/T83W/A96L/G162K/D174K/R195L/P210E/Y277N/E318Q | ++ | | ++++ | | | |
| 1453/1454 | G37D | H13P/A16S/D17G/A26E/E31K/K37D/T60V/A79G/T83W/A96L/G162K/D174K/R195L/P210E/Y277V/E318Q | ++ | | ++++ | | | |
| 1455/1456 | V277H | H13P/A16S/D17G/A26E/E31K/K37G/T60V/A79G/T83W/A96L/G162K/D174K/R195L/P210E/Y277H/E318Q | ++ | | ++++ | + | + | |
| 1457/1458 | V277D | H13P/A16S/D17G/A26E/E31K/K37G/T60V/A79G/T83W/A96L/G162K/D174K/R195L/P210E/Y277D/E318Q | + | | ++++ | | | |
| 1459/1460 | L96D | H13P/A16S/D17G/A26E/E31K/K37G/T60V/A79G/T83W/A96D/G162K/D174K/R195L/P210E/Y277V/E318Q | ++ | + | ++++ | + | + | ++ |
| 1461/1462 | K174C | H13P/A16S/D17G/A26E/E31K/K37G/T60V/A79G/T83W/A96L/G162K/D174C/R195L/P210E/Y277V/E318Q | ++ | | ++++ | | | |
| 1463/1464 | K162P | H13P/A16S/D17G/A26E/E31K/K37G/T60V/A79G/T83W/A96L/G162P/D174K/R195L/P210E/Y277V/E318Q | ++ | | ++++ | | + | |
| 1465/1466 | Q318F | H13P/A16S/D17G/A26E/E31K/K37G/T60V/A79G/T83W/A96L/G162K/D174K/R195L/P210E/Y277V/E318F | + | | ++++ | | | |
| 1467/1468 | W83N | H13P/A16S/D17G/A26E/E31K/K37G/T60V/A79G/T83N/A96L/G162K/D174K/R195L/P210E/Y277V/E318Q | +++ | + | ++++ | + | + | + |
| 1469/1470 | V60C | H13P/A16S/D17G/A26E/E31K/K37G/T60C/A79G/T83W/A96L/G162K/D174K/R195L/P210E/Y277V/E318Q | ++ | | ++++ | + | | |
| 1471/1472 | W83I | H13P/A16S/D17G/A26E/E31K/K37G/T60V/A79G/T83I/A96L/G162K/D174K/R195L/P210E/Y277V/E318Q | ++ | + | ++++ | + | + | + |
| 1473/1474 | K162M | H13P/A16S/D17G/A26E/E31K/K37G/T60V/A79G/T83W/A96L/G162M/D174K/R195L/P210E/Y277V/E318Q | +++ | | ++++ | + | + | ++ |
| 1475/1476 | E210H | H13P/A16S/D17G/A26E/E31K/K37G/T60V/A79G/T83W/A96L/G162K/D174K/R195L/P210H/Y277V/E318Q | ++ | | ++++ | | | |
| 1477/1478 | L195H | H13P/A16S/D17G/A26E/E31K/K37G/T60V/A79G/T83W/A96L/G162K/D174K/R195H/P210E/Y277V/E318Q | ++ | ++ | ++++ | + | ++ | |
| 1479/1480 | K174Y | H13P/A16S/D17G/A26E/E31K/K37G/T60V/A79G/T83W/A96L/G162K/D174Y/R195L/P210E/Y277V/E318Q | ++ | | ++++ | | | |
| 1481/1482 | G37Y | H13P/A16S/D17G/A26E/E31K/K37Y/T60V/A79G/T83W/A96L/G162K/D174K/R195L/P210E/Y277V/E318Q | + | | +++ | | | |
| 1483/1484 | E210Q | H13P/A16S/D17G/A26E/E31K/K37G/T60V/A79G/T83W/A96L/G162K/D174K/R195L/P210Q/Y277V/E318Q | ++ | + | ++++ | + | + | ++ |
| 1485/1486 | G37T | H13P/A16S/D17G/A26E/E31K/K37T/T60V/A79G/T83W/A96L/G162K/D174K/R195L/P210E/Y277V/E318Q | ++ | | +++ | | | |
| 1487/1488 | L96N | H13P/A16S/D17G/A26E/E31K/K37G/T60V/A79G/T83W/A96N/G162K/D174K/R195L/P210E/Y277V/E318Q | ++ | | ++++ | | | + |
| 1489/1490 | W83H | H13P/A16S/D17G/A26E/E31K/K37G/T60V/A79G/T83H/A96L/G162K/D174K/R195L/P210E/Y277V/E318Q | ++ | + | ++++ | + | + | + |
| 1491/1492 | S16H | H13P/A16H/D17G/A26E/E31K/K37G/T60V/A79G/T83W/A96L/G162K/D174K/R195L/P210E/Y277V/E318Q | ++ | | ++++ | | | |
| 1493/1494 | K174F | H13P/A16S/D17G/A26E/E31K/K37G/T60V/A79G/T83W/A96L/G162K/D174F/R195L/P210E/Y277V/E318Q | ++ | | ++++ | | | |
| 1495/1496 | W83D | H13P/A16S/D17G/A26E/E31K/K37G/T60V/A79G/T83D/A96L/G162K/D174K/R195L/P210E/Y277V/E318Q | ++ | + | ++++ | + | + | |
| 1497/1498 | W83Q | H13P/A16S/D17G/A26E/E31K/K37G/T60V/A79G/T83Q/A96L/G162K/D174K/R195L/P210E/Y277V/E318Q | +++ | + | ++++ | + | + | + |
| 1499/1500 | K162H | H13P/A16S/D17G/A26E/E31K/K37G/T60V/A79G/T83W/A96L/G162H/D174K/R195L/P210E/Y277V/E318Q | +++ | + | ++++ | ++ | + | + |
| 1501/1502 | K162W | H13P/A16S/D17G/A26E/E31K/K37G/T60V/A79G/T83W/A96L/G162W/D174K/R195L/P210E/Y277V/E318Q | ++ | | +++ | | | |
| 1503/1504 | K162N | H13P/A16S/D17G/A26E/E31K/K37G/T60V/A79G/T83W/A96L/G162N/D174K/R195L/P210E/Y277V/E318Q | ++ | + | ++++ | + | + | ++ |
| 1505/1506 | S16F | H13P/A16F/D17G/A26E/E31K/K37G/T60V/A79G/T83W/A96L/G162K/D174K/R195L/P210E/Y277V/E318Q | + | | ++++ | | | |
| 1507/1508 | K162Q | H13P/A16S/D17G/A26E/E31K/K37G/T60V/A79G/T83W/A96L/G162Q/D174K/R195L/P210E/Y277V/E318Q | +++ | + | ++++ | + | + | ++ |
| 1509/1510 | K162G | H13P/A16S/D17G/A26E/E31K/K37G/T60V/A79G/T83W/A96L/D174K/R195L/P210E/Y277V/E318Q | ++ | | ++++ | + | + | + |
| 1511/1512 | K162R | H13P/A16S/D17G/A26E/E31K/K37G/T60V/A79G/T83W/A96L/G162R/D174K/R195L/P210E/Y277V/E318Q | +++ | + | ++++ | + | + | + |
| 1513/1514 | E210F | H13P/A16S/D17G/A26E/E31K/K37G/T60V/A79G/T83W/A96L/G162K/D174K/R195L/P210F/Y277V/E318Q | + | | ++++ | | | |
| 1515/1516 | K162C | H13P/A16S/D17G/A26E/E31K/K37G/T60V/A79G/T83W/A96L/G162C/D174K/R195L/P210E/Y277V/E318Q | +++ | + | ++++ | ++ | + | ++ |

Activities were determined relative to either the reference polypeptide of SEQ ID NO: 4 or SEQ ID NO: 906. Levels of increased activity are defined as follows: “+” 0.9 to 1.1, “++” > 1.1, “+++” > 2.0,“++++”>3.5

TABLE 5-2

Oxalate decarboxylase activity Relative to SEQ ID NO: 4 or SEQ ID NO: 906

| SEQ ID NO: (nt/aa) | Amino Acid Differences (Relative to SEQ ID NO: 906) | Amino Acid Differences (Relative to SEQ ID NO: 2) | FIOP Unchallenged Activity at pH 2.8 relative to SEQ ID NO: 2 | FIOP Unchallenged Activity at pH 4.5 relative to SEQ ID NO: 2 | FIOP 0.3 g/L Pepsin Challenge relative to SEQ ID NO: 2 | FIOP Unchallenged Activity at pH 4.5 relative to SEQ ID NO: 906 | FIOP Unchallenged Activity at pH 2.8 relative to SEQ ID NO: 906 | FIOP 0.8 g/L Pepsin Challenge relative to SEQ ID NO: 906 |
|---|---|---|---|---|---|---|---|---|
| 905/906 | | H13P/A16S/D17G/A26E/E31K/K37G/T60V/A79G/T83W/A96L/G162K/D 174K/R195L/P210E/Y277V/E318Q | ++ | | +++ | + | + | + |
| ½ | P13H/S16A/G17D/E26A/K31E/G37K/V60T/G79A/W83T/L96A/K162G/K174 D/L195R/E210P/V277Y/Q318E | | + | + | + | | ++ | |
| 1517/1518 | K174D/Q318E | H13P/A16S/D17G/A26E/E31K/K37G/T60V/A79G/T83W/A96L/G162K/R 195L/P210E/Y277V | +++ | ++ | ++++ | ++ | ++ | ++ |
| 1519/1520 | E26A/L96A | H13P/A16S/D17G/E31K/K37G/T60V/A79G/T83W/G162K/D174K/R195L/P210E/Y277V/E318Q | ++ | + | +++ | | ++ | + |
| 1521/1522 | V60T/Q318E | H13P/A16S/D17G/A26E/E31K/K37G/A79G/T83W/A96L/G162K/D174K/R195L/P210E/Y277V | +++ | + | +++ | ++ | ++ | |
| 1523/1524 | K31E/Q318E | H13P/A16S/D17G/A26E/K37G/T60V/A79G/T83W/A96L/G162K/D174K/R195L/P210E/Y277V | +++ | + | +++ | ++ | ++ | |
| 1525/1526 | K162G/K174D/Q318E | H13P/A16S/D17G/A26E/E31K/K37G/T60V/A79G/T83W/A96L/R195L/P 210E/Y277V | +++ | + | +++ | ++ | ++ | ++ |
| 1527/1528 | K174D/V277Y/Q318E | H13P/A16S/D17G/A26E/E31K/K37G/T60V/A79G/T83W/A96L/G162K/R 195L/P210E | +++ | + | +++ | ++ | ++ | ++ |
| 1529/1530 | E26A/L195R/Q318 E | H13P/A16S/D17G/E31K/K37G/T60V/A79G/T83W/A96L/G162K/D174K/P210E/Y277V | ++ | | +++ | + | + | + |
| 1531/1532 | V60T/K162G/L195 R | H13P/A16S/D17G/A26E/E31K/K37G/A79G/T83W/A96L/D174K/P210E/Y277V/E318Q | +++ | + | +++ | ++ | ++ | |
| 1533/1534 | V60T/K162G/Q318E | H13P/A16S/D17G/A26E/E31K/K37G/A79G/T83W/A96L1D174K/R195L1 P210E/Y277V | +++ | + | +++ | ++ | + | |
| 1535/1536 | K174D/L195R/V277Y | H13P/A16S/D17G/A26E/E31K/K37G/T60V/A79G/T83W/A96L/G162K/P 210E/E318Q | ++ | + | +++ | ++ | ++ | |
| 1537/1538 | S16A/G17D/K174D | H13P/A26E/E31K/K37G/T60V/A79G/T83W/A96L/G162K/R195L/P210E/Y277V/E318Q | ++ | + | ++++ | + | + | |
| 1539/1540 | V60T/L195R/V277Y | H13P/A16S/D17G/A26E/E31K/K37G/A79G/T83W/A96L/G162K/D174K/P210E/E318Q | ++ | + | +++ | + | + | |
| 1541/1542 | G37K/V277Y/Q318E | H13P/A16S/D17G/A26E/E31K/T60V/A79G/T83W/A96L/G162K/D174K/R195L/P210E | ++ | + | ++++ | | + | |
| 1543/1544 | S16A/K162G/L195 R/V277Y | H13P/D17G/A26E/E31K/K37G/T60V/A79G/T83W/A96L/D174K/P210E/E318Q | ++ | | +++ | + | + | |
| 1545/1546 | E26A/G79A/W83T/K162G | H13P/A16S/D17G/E31K/K37G/T60V/A96L/D174K/R195L/P210E/Y277V/E318Q | ++ | | ++++ | | | |
| 1547/1548 | E26A/K31E/V60T/Q318E | H13P/A16S/D17G/K37G/A79G/T83W/A96L/G162K/D174K/R195L/P210 E/Y277V | +++ | ++ | ++++ | ++ | ++ | |
| 1549/1550 | S16A/K174D/L195 R/V277Y | H13P/D17G/A26E/E31K/K37G/T60V/A79G/T83W/A96L/G162K/P210E/E318Q | ++ | + | ++++ | + | + | |
| 1551/1552 | P13H/G37K/W83T/K162G | A16S/D17G/A26E/E31K/T60V/A79G/A96L/D174K/R195L/P210E/Y277V/E318Q | ++ | + | ++++ | + | + | |
| 1553/1554 | S16A/G17D/V60T/L195R | H13P/A26E/E31K/K37G/A79G/T83W/A96L/G162K/D174K/P210E/Y277V/E318Q | +++ | + | +++ | ++ | + | |
| 1555/1556 | P13H/G17D/G37K/W83T | A16S/A26E/E31K/T60V/A79G/A96L/G162K/D174K/R195L/P210E/Y277 V/E318Q | ++ | + | +++ | | + | |
| 1557/1558 | K31E/G37K/K162G/K174D/Q318E | H13P/A16S/D17G/A26E/T60V/A79G/T83W/A96L/R195L/P210E/Y277V | +++ | + | +++ | ++ | ++ | |
| 1559/1560 | P13H/S16A/K31E/W83T/K162G | D17G/A26E/K37G/T60V/A79G/A96L/D174K/R195L/P210E/Y277V/E318 Q | +++ | ++ | ++++ | + | ++ | |
| 1561/1562 | S16A/V60T/K174D/L195R/Q318E | H13P/D17G/A26E/E31K/K37G/A79G/T83W/A96L/G162K/P210E/Y277V | +++ | + | +++ | + | + | |
| 1563/1564 | G17D/V60T/L96A/K162G/L195R/V27 7Y | H13P/A16S/A26E/E31K/K37G/A79G/T83W/D174K/P210E/E318Q | +++ | + | +++ | + | ++ | |
| 1565/1566 | P13H/E26A/V60T/W83T/K162G/K174D | A16S/D17G/E31K/K37G/A79G/A96L/R195L/P210E/Y277V/E318Q | +++ | ++ | +++ | ++ | ++ | |
| 1567/1568 | G17D/K31E/V60T/K162G/K174D/V277Y | H13P/A16S/A26E/K37G/A79G/T83W/A96L/R195L/P210E/E318Q | +++ | ++ | +++ | ++ | ++ | |
| 1569/1570 | S16A/G17D/E26A/W83T/L96A/V277Y | H13P/E31K/K37G/T60V/A79G/G162K/D174K/R195L/P210E/E318Q | ++ | ++ | ++++ | | ++ | |
| 1571/1572 | G17D/E26A/G37K/K174D/V277Y/Q318E | H13P/A16S/E31K/T60V/A79G/T83W/A96L/G162K/R195L/P210E | ++ | + | ++++ | | ++ | |
| 1573/1574 | P13H/S16A/K31E/V60T/K162G/K174 D | D17G/A26E/K37G/A79G/T83W/A96L/R195L/P210E/Y277V/E318Q | ++ | + | ++++ | + | ++ | |
| 1575/1576 | P13H/G17D/K31E/W83T/V277Y/Q318E | A16S/A26E/K37G/T60V/AT9G/A96L/G162K/D174K/R195L/P210E | ++ | + | +++ | | ++ | |
| 1577/1578 | P13H/S16A/G17D/K31E/W83T/K162 G/Q318E | A26E/K37G/T60V/A79G/A96L/D174K/R195L/P210E/Y277V | ++ | ++ | ++++ | + | ++ | |
| 1579/1580 | P13H/S16A/V60T/W83T/K162G/L195R/Q318E | D17G/A26E/E31K/K37G/A79G/A96L/D174K/P210E/Y277V | ++ | ++ | ++++ | | ++ | |
| 1581/1582 | P13H/S16A/G17D/K31E/G37K/W83T/L96A/K162G | A26E/T60V/A79G/D174K/R195L/P210E/Y277V/E318Q | ++ | + | +++ | | ++ | |
| 1583/1584 | P13H/G17D/E26A/G37K/V60T/W83T/K162G/E210P/Q318E | A16S/E31K/A79G/A96L/D174K/R195L/Y277V | ++ | | ++++ | | + | ++++ |
| 1585/1586 | P13H/S16A/E26A/G37K/W83T/K162 G/K174D/L195R/Q 318E | D17G/E31K/T60V/A79G/A96L/P210E/Y277V | +++ | + | +++ | ++ | ++ | |
| 1587/1588 | P13H/S16A/E26A/K31E/W83T/L96A/K162G/K174D/Q31 8E | D17G/K37G/T60V/A79G/R195L/P210E/Y277V | ++ | ++ | +++ | + | ++ | |
| 1589/1590 | P13H/S16A/G17D/K31E/V60T/W83T/L96A/K162G/K174 D/L195R/Q318E | A26E/K37G/A79G/P210E/Y277V | +++ | ++ | +++ | + | ++ | |
| 1591/1592 | P13H/S16A/E26A/ G37K/V60T/W83T/K162G/K174D/L19 5R/E210P/Q318E | D17G/E31K/A79G/A96L/Y277V | ++ | + | ++++ | + | ++ | |
| 1593/1594 | P13H/S16A/G37K/G79A/W83T/L96A/K162G/K174D/L19 5R/E210P/V277Y | D17G/A26E/E31K/T60V/E318Q | ++ | | ++++ | | + | |
| 1595/1596 | P13H/G17D/E26A/K31E/G37K/V60T/G79A/W83T/L96A/K162G/K174D/E210P | A16S/R195L/Y277V/E318Q | ++ | | ++++ | | + | |
| 1597/1598 | P13H/S16A/G17D/E26A/G37K/G79A/W83T/L96A/K174 D/L195R/E210D/Q 318E | E31K/T60V/G162K/P210D/Y277V | ++ | | ++++ | | + | |
| 1599/1600 | P13H/E26A/K31E/G37K/V60T/W83T/L96A/K162G/K174 D/L195R/E210P/V277Y/Q318E | A16S/D17G/A79G | ++ | + | ++++ | | ++ | |
| 1601/1602 | P13H/S16A/G17D/K31E/G37K/V60T/G79A/W83T/L96A/K162G/K174D/L19 5R/E210P/Q318E | A26E/Y277V | ++ | + | +++ | | ++ | |
| 1603/1604 | P13H/S16A/G17D/K31E/V60T/G79A/W83T/L96A/K162 G/K174D/L195R/E 210P/V277Y/Q318 E | A26E/K37G | + | + | +++ | | ++ | |
| 1605/1606 | P13H/S16A/G17D/E26A/G37K/V60T/G79A/L96A/K162G/K174D/L195R/E210P/V277Y/Q318E | E31K/T83W | | | ++ | | | |
| 1607/1608 | P13H/E26A/K31E/G37K/V60T/G79A/W83T/L96A/K162 G/K174D/L195R/E 210P/V277Y/Q318 E | A16S/D17G | ++ | | +++ | | + | |
| 1609/1610 | P13H/S16A/G17D/E26A/K31E/G37K/V60T/L96A/K162G /K174D/L195R/E210P/V277Y/Q318E | A79G/T83W | + | + | ++ | | ++ | |
| 1611/1612 | P13H/S16A/G17D/E26A/K31E/G37K/G79A/W83T/L96A/K162G/K174D/L19 5R/E210P/Q318E | T60V/Y277V | ++ | + | +++ | | ++ | |
| 1613/1614 | P13H/S16A/G17D/E26A/K31E/G37K/V60T/G79A/L96A/K162G/K174D/E210P/V277Y/Q318E | T83W/R195L | | | ++ | | | |
| 1615/1616 | P13H/G17D/E26A/K31E/G37K/V60T/G79A/W83T/L96A/K162G/K174D/L19 5R/E210P/V277Y/Q318E | A16S | ++ | + | +++ | | + | |
| 1617/1618 | P13H/S16A/E26A/ K31E/G37K/V60T/ G79A/W83T/L96A/ K162G/K174D/L19 5R/E210P/V277Y/Q318E | D17G | ++ | + | +++ | | ++ | |
| 1619/1620 | S16A/G17D/E26A/ K31E/G37K/V60T/ G79A/W83T/L96A/ K162G/K174D/L19 5R/E210P/V277Y/Q318E | H13P | ++ | | +++ | | | |
| 1621/1622 | P13H/S16A/G17D/ E26A/K31E/G37K/ V60T/G79A/W83T/ L96A/K162G/K174 D/L195R/E210P/V277H/Q318E | Y277H | ++ | ++ | +++ | | ++ | |

Activities were determined relative to either the reference polypeptide of SEQ ID NO: 4 or SEQ ID NO: 906. Levels of increased activity are defined as follows: “+” 0.9 to 1.1, “++” > 1.1, “+++” > 2.0,“++++”>3.5

### Example 12

### In Vivo Dose Response in an Enteric Hyperoxaluria Mini Pig Model With Oral Treatment

### Preparation of Engineered Oxalate Decarboxylase

The expression construct for SEQ ID NO: 905 was fermented at high cell density in a fed-batch format. The fermentation process consisted of an inoculum preparation phase and three main fermentation phases: batch, fed-batch growth, and fed-batch expression. Cells were harvested by bucket centrifugation, and the pellets were stored at -80° C. until further use.

Cell pellets were thawed, resuspended, and lysed by a homogenizer. The whole cell lysate was clarified by flocculation followed by centrifugation. The clarified lysate was concentrated and underwent an acid precipitation step to remove whole cell proteins. Upon centrifugation, the target enzyme in the clarified lysate was purified by cation exchange chromatography using a Tosoh GigaCap S 650 M column. The column eluate was concentrated, and buffer exchanged into processing buffer (25 mM Tris, 150 mM NaCl, pH 8.0) by tangential flow filtration against a 100 kDa PES membrane (Repligen). The buffer exchanged protein was then batch sterile filtered through a 0.2 µm PES filter (Sartorius Sartopore 2 XLG) and lyophilized to obtain a dried yellow powder.

### In Vivo Studies

A validated model of enteric hyperoxaluria (HyOx) was produced in naive Gottingen minipigs (n=36 females, 15 kg average body weight, aged 7-8 months) and carried forward into in vivo studies. The study objective was to evaluate the ability of an orally administered engineered oxalate decarboxylase (OxDC) enzyme (SEQ ID NO: 906) to break down dietary oxalate in the GI tract, primarily the stomach, thereby lowering urine oxalate levels (UOx).

On days 0-6, all animals were fed a low oxalate diet (LOD: Research Diets Product D22071205 (0 g sodium oxalate/ kg feed)) to establish a baseline. Animals were then switched to a high oxalate diet during days 7-20 (HOD: Research Diets Product D22071206 (1.52 g sodium oxalate/ kg feed)) to simulate a disease state. On days 14-20, treatment (SEQ ID NO: 906) was admixed into HOD immediately prior to feeding. Animals were fed a set meal ration twice daily (AM and PM), approximately 8 hours apart, throughout the study. Animals were dosed with either 5,139 Units, 10,122 Units or 41,029 Units per meal, dependent upon group assignment. One unit of the engineered oxalate decarboxylase (SEQ ID NO: 906) is defined as the amount of engineered oxalate decarboxylase polypeptide which catalyzes the substrate and generates 1.0 µmol of formate per minute at 25° C. The unit of specific activity of SEQ ID NO: 906 is U/mg which is equivalent to µmol/min/mg.

Urine was collected for a total of 24 hours on each collection day, (days 5, 6, 12, 13, 19 and 20) for a total of 6 collections per animal. During urine collection, animals were single housed, and all urine excreted in a 24 hour period was collected into individual vessels. Urine volume was recorded, and aliquots were frozen at -70° C. for later UOx analysis using the oxalate analysis kit from Trinity BioSciences (591D-1KT).

Urine samples were prepared according to the kit directions (Trinity Biosciences, 591D-1KT), employing a minor modification to scale volumes for a plate reader in place of a cuvette. Data analysis was carried out using GraphPad Prism 9 (GraphPad Software, San Diego, CA). Individual UOx values (normalized as mg oxalate/24 hours) as well as percent change in UOx were examined.

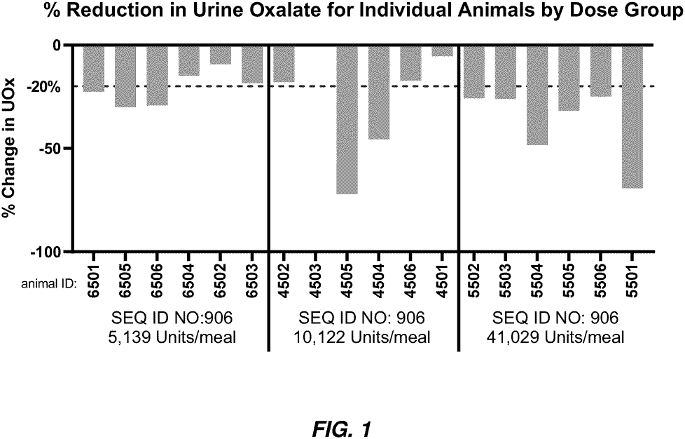

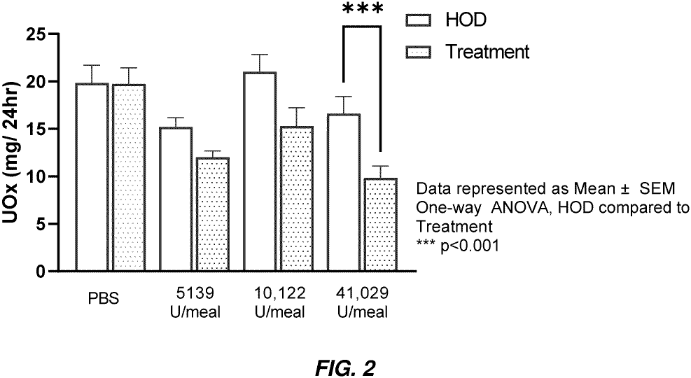

To evaluate efficacy of SEQ ID NO: 906 on urine oxalate, a minimum threshold reduction of 20% in UOx from HOD to HOD + Treatment was set. This threshold value is a relevant endpoint for models of Hyperoxaluria because this reduction is believed to reduce the number of future kidney stone occurrences. The response to treatment appears to be dose dependent, as 6 of 6 individual animals responded with a 20% or greater reduction in UOx in the high dose group (FIG. 1). A reduction in UOx with treatment was observed in all groups, however this reduction was statistically significant in the highest dose group only (FIG. 2). In groups that received vehicle (PBS) during the treatment phase, there was no difference between HOD and HOD + Treatment.

What is claimed is:

1. An engineered oxalate decarboxylase polypeptide comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 2, 4, 172, 320, 396, 616, 670, 750, or 906, or to a reference sequence corresponding to residues 1-359 of SEQ ID NO: 172, 320, or 396, wherein the amino acid sequence comprises one or more amino acid substitutions relative to the reference sequence corresponding to SEQ ID NO: 2, 4, 172, 320, 396, 616, 670, 750, or 906, or relative to the reference sequence corresponding to residues 1-359 of SEQ ID NO: 172, 320, or 396.

2. The engineered oxalate decarboxylase polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 2 or 4, wherein the amino acid sequence comprises one or more amino acid substitutions relative to the reference sequence corresponding to SEQ ID NO: 2 or 4.

3. The engineered oxalate decarboxylase polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 172, 320, 396, 616, 670, 750, or 906, or to the reference sequence corresponding to residues 1-359 of SEQ ID NO: 172, 320, or 396, wherein the amino acid sequence comprises one or more amino acid substitutions relative to the reference sequence of SEQ ID NO: 2 or 4.

4. The engineered oxalate decarboxylase polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 28-1622, or to a reference sequence corresponding to residues 1-359 of an even-numbered SEQ ID NO. of SEQ ID NOs: 28-614, wherein the amino acid sequence comprises one or more amino acid substitutions relative to the reference sequence of SEQ ID NO: 2 or 4.

5. The engineered oxalate decarboxylase polypeptide of any one of claims 1-4, wherein the amino acid sequence of the engineered oxalate decarboxylase polypeptide comprises at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to SEQ ID NO: 2 or 4.

6. The engineered oxalate decarboxylase polypeptide of any one of claims 1-5, wherein the amino acid sequence of the engineered oxalate decarboxylase polypeptide comprises at least a substitution 4C/R/S, 5C/S, 6A/S/W, 7G, 10A/Q/R, 11R, 13A/C/D/E/F/G/I/K/L/M/N/P/Q/R/S/T/V/W/Y, 14L, 16C/D/E/F/G/H/I/K/L/M/N/P/Q/R/S/T/V/W/Y, 17A/C/D/E/F/G/I/K/L/M/N/P/Q/R/S/T/V/W/Y, 18G/Q/R, 19C, 22R, 26C/D/E/F/G/H/I/K/L/M/N/P/Q/R/S/T/V/W/Y, 31A/C/D/F/G/H/I/K/L/M/N/P/Q/R/S/T/V/W/Y, 33R, 35V, 37A/C/D/E/F/G/H/I/L/M/N/P/Q/R/S/T/V/W/Y, 40S, 43C, 44S, 46R, 52T, 54L, 60A/C/D/E/F/G/H/I/K/L/M/N/P/Q/R/S/V/W/Y, 61M, 62G, 63A/S, 76S, 79C/D/E/F/G/H/I/K/L/M/N/P/Q/R/S/T/V/W/Y, 80L, 82I, 83A/C/D/E/F/G/H/I/K/L/M/N/P/Q/R/S/V/W/Y, 85E/G/R, 94N/T, 96C/D/E/F/G/H/I/K/L/M/N/P/Q/R/S/T/V/W/Y, 97T, 103Q/S/V, 104Q, 106C, 110V, 117A, 121W, 123G/Q/S, 124A/C/G/Q/R/T, 125E/F/S, 126T, 128A, 141Q, 149T, 153H/Q/S, 155L/P/R/V, 156E, 160T, 162A/C/D/E/F/H/I/K/L/M/N/P/Q/R/S/T/V/W/Y, 164R/V, 166R, 169A/G/L, 173Q/V, 174A/C/E/F/G/H/I/K/L/M/N/P/Q/R/S/T/V/W/Y, 176L, 180E, 182R, 183C/E/I/K, 186N, 187L/R/S, 188A, 189R, 190G/Q, 193A/G, 195A/C/D/E/F/G/H/I/K/L/M/N/P/Q/S/T/V/W/Y, 196F/M/R/S/V, 197E, 199G/V, 200N, 205A, 206M, 208G, 210A/C/D/E/F/G/H/I/K/L/M/N/Q/R/S/T/V/W/Y, 212A/F/G/L/S/V, 216S/W, 219V, 226S, 227E/S, 232T, 233D/H/R, 234L, 240E, 242D, 243V, 263S, 265C, 266V, 267R, 269S, 270L, 273A, 274Q/S, 277A/C/D/E/F/G/H/I/K/L/M/N/P/Q/R/S/T/V/W, 284A/R/S, 297Y, 301I/Q/T, 303T, 304D/G, 314S, 316K/V, 318A/C/D/F/G/H/I/K/L/M/N/P/Q/R/S/T/V/W/Y, 331I/L/P/V, 335R, 339G, 342A/E/P/R, 343R/W, 346G/L/Q/W, 347F/R, 350R, 351A/E/H, 356S, or 359L/W, or combinations thereof, wherein the amino acid positions are relative to the reference sequence corresponding to SEQ ID NO: 2 or 4.

7. The engineered oxalate decarboxylase polypeptide of any one of claims 1-5, wherein the amino acid sequence of the engineered oxalate decarboxylase polypeptide comprises at least a substitution at amino acid position 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 2 or 4.

8. The engineered oxalate decarboxylase polypeptide of claim 7, wherein the amino acid sequence of the engineered oxalate decarboxylase polypeptide comprises at least a substitution 13A/C/D/E/F/G/I/K/L/M/N/P/Q/R/S/T/V/W/Y, 14L, 16C/D/E/F/G/H/I/K/L/M/N/P/Q/R/S/T/V/W/Y, 17A/C/D/E/F/G/I/K/L/M/N/P/Q/R/S/T/V/W/Y, 26C/D/E/F/G/H/I/K/L/M/N/P/Q/R/S/T/V/W/Y, 31A/C/D/F/G/H/I/K/L/M/N/P/Q/R/S/T/V/W/Y, 37A/C/D/E/F/G/H/I/L/M/N/P/Q/R/S/T/V/W/Y, 60A/C/D/E/F/G/H/I/K/L/M/N/P/Q/R/S/V/W/Y, 79C/D/E/F/G/H/I/K/L/M/N/P/Q/R/S/T/V/W/Y, 83A/C/D/E/F/G/H/I/K/L/M/N/P/Q/R/S/V/W/Y, 96C/D/E/F/G/H/I/K/L/M/N/P/Q/R/S/T/V/W/Y, 162A/C/D/E/F/H/I/K/L/M/N/P/Q/R/S/T/V/W/Y, 174A/C/E/F/G/H/I/K/L/M/N/P/Q/R/S/T/V/W/Y, 195A/C/D/E/F/G/H/I/K/L/M/N/P/Q/S/T/V/W/Y, 196F/M/R/S/V, 210A/C/D/E/F/G/H/I/K/L/M/N/Q/R/S/T/V/W/Y, 226S, 277A/C/D/E/F/G/H/I/K/L/M/N/P/Q/R/S/T/V/W, 301I/Q/T, or 318A/C/D/F/G/H/I/K/L/M/N/P/Q/R/S/T/V/W/Y, or combinations thereof, wherein the amino acid positions are relative to the reference sequence corresponding to SEQ ID NO: 2 or 4.

9. The engineered oxalate decarboxylase polypeptide of claim 7, wherein the amino acid sequence of the engineered oxalate decarboxylase polypeptide comprises at least a substitution 13P, 14L, 16S, 17H/G, 26E, 31K, 37G, 60V, 79G, 83W, 96L, 162K, 174K, 195L, 196V, 210A/E, 226S, 277V, 301I, or 318Q, or combinations thereof, wherein the amino acid positions are relative to the reference sequence corresponding to SEQ ID NO: 2 or 4.

10. The engineered oxalate decarboxylase polypeptide of any one of claims 1-5, wherein the amino acid sequence comprises at least a substitution or substitution set at amino acid position(s) 346, 124, 359, 174, 173, 123, 196, 304, 301, 347, 11, 284, 210, 169, 216, 195, 339, 4, 6, 180, 80, 243, 182, 7, 226, 156, 183, 227, 219, 62, 343, 16/26, 16/26/242, 16/26/183/232, 155/206/242, 16/26/339, 16/26/155/206/339, 26, 26/206/339, 31/82/210, 31/82, 31/356, 31/97/226, 31/240/270, 31/82/226, 31/240, 31, 31/210/318, or 31/210, wherein the amino acid positions are relative to the reference sequence corresponding to SEQ ID NO: 2 or 4.

11. The engineered oxalate decarboxylase polypeptide of claim 1, wherein the amino acid sequence of the engineered oxalate decarboxylase polypeptide comprises at least a substitution or substitution set of an engineered oxalate decarboxylase polypeptide set forth in Tables 4-1, 4-2, 4-3, 4-4, 4-5, 4-6, 4-7, 5-1, and 5-2, wherein the amino acid positions are relative to SEQ ID NO: 2 or 4.

12. The engineered oxalate decarboxylase polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396.

13. The engineered oxalate decarboxylase polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of an even-numbered SEQ ID NO. of SEQ ID NOs: 28-614, a reference sequence corresponding to an even-numbered SEQ ID NO. of SEQ ID NOs: 28-614, or a reference sequence corresponding to an even-numbered SEQ ID NO. of SEQ ID NOs: 616-1622.

14. The engineered oxalate decarboxylase polypeptide of claim 1, comprising an amino sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to an even-numbered SEQ ID NO. of SEQ ID NOs: 28-1622, or to the reference sequence corresponding to residues 1-359 of an even-numbered SEQ ID NO. of SEQ ID NOs: 28-614, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

15. The engineered oxalate decarboxylase polypeptide of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 172, 320, 396, 616, 670, 750, or 906, or to a reference sequence corresponding to residues 1-359 of SEQ ID NO: 172, 320, or 396, wherein the amino acid sequence comprises one or more substitutions relative to the reference sequence corresponding to SEQ ID NO: 172, 320, 396, 616, 670, 750, or 906, or relative to the reference sequence corresponding to residues 1-359 of SEQ ID NO: 172, 320, or 396.

16. The engineered oxalate decarboxylase polypeptide of claim 14 or 15, wherein the amino acid sequence of the engineered oxalate decarboxylase polypeptide comprises at least a substitution at amino acid position 4, 5, 6, 7, 10, 11, 13, 14, 16, 17, 18, 19, 22, 26, 31, 33, 35, 37, 40, 43, 44, 46, 52, 54, 60, 61, 62, 63, 76, 79, 80, 82, 83, 85, 94, 96, 97, 103, 104, 106, 110, 117, 121, 123, 124, 125, 126, 128, 141, 149, 153, 155, 156, 160, 162, 164, 166, 169, 173, 174, 176, 180, 182, 183, 186, 187, 188, 189, 190, 193, 195, 196, 197, 199, 200, 205, 206, 208, 210, 212, 216, 219, 226, 227, 232, 233, 234, 240, 242, 243, 263, 265, 266, 267, 269, 270, 273, 274, 277, 284, 297, 301, 303, 304, 314, 316, 318, 331, 335, 339, 342, 343, 346, 347, 350, 351, 356, or 359, or combinations thereof, wherein the amino acid positions are relative to the reference sequence corresponding to residues SEQ ID NO: 172, 320, 396, 616, 670, 750, or 906, or relative to the reference sequence corresponding to residues 1-359 of SEQ ID NO: 172, 320, or 396.

17. The engineered oxalate decarboxylase polypeptide of any one of claims 14-16, wherein the amino acid sequence of the engineered oxalate decarboxylase polypeptide comprises at least a substitution or amino acid residue 4C/R/S, 5C/S, 6A/S/W, 7G, 10A/Q/R, 11R, 13A/C/D/E/F/G/H/I/K/L/M/N/P/Q/R/S/T/V/W/Y, 14L/V, 16A/C/D/E/F/G/H/I/K/L/M/N/P/Q/R/S/T/V/W/Y, 17A/C/D/E/F/G/H/I/K/L/M/N/P/Q/R/S/T/V/W/Y, 18G/Q/R, 19C, 22R, 26A/C/D/E/F/G/H/I/K/L/M/N/P/Q/R/S/T/V/W/Y, 31A/C/D/E/F/G/H/I/K/L/M/N/P/Q/R/S/T/V/W/Y, 33R, 35V, 37A/C/D/E/F/G/H/I/K/L/M/N/P/Q/R/S/T/V/W/Y, 40S, 43C, 44S, 46R, 52T, 54L, 60A/C/D/E/F/G/H/I/K/L/M/N/P/Q/R/S/T/V/W/Y, 61M, 62G, 63A/S, 76S, 79A/C/D/E/F/G/H/I/K/L/M/N/P/Q/R/S/T/V/W/Y, 80L, 82I, 83A/C/D/E/F/G/H/I/K/L/M/N/P/Q/R/S/T/V/W/Y, 85E/G/R, 94N/T, 96A/C/D/E/F/G/H/I/K/L/M/N/P/Q/R/S/T/V/W/Y, 97T, 103Q/S/V, 104Q, 106C, 110V, 117A, 121W, 123G/Q/S, 124A/C/G/Q/R/T, 125E/F/S, 126T, 128A, 141Q, 149T, 153H/Q/S, 155L/P/R/V, 156E, 160T, 162A/C/D/E/F/G/H/I/K/L/M/N/P/Q/R/S/T/V/W/Y, 164R/V, 166R, 169A/G/L, 173Q/V, 174A/C/D/E/F/G/H/I/K/L/M/N/P/Q/R/S/T/V/W/Y, 176L, 180E, 182R, 183C/E/I/K, 186N, 187L/R/S, 188A, 189R, 190G/Q, 193A/G, 195A/C/D/E/F/G/H/I/K/L/M/N/P/Q/R/S/T/V/W/Y, 196F/K/M/R/S/V, 197E, 199G/V, 200N, 205A, 206M, 208G, 210A/C/D/E/F/G/H/I/K/L/M/N/P/Q/R/S/T/V/W/Y, 212A/F/G/L/S/V, 216S/W, 219V, 226S/T, 227E/S, 232T, 233D/H/R, 234L, 240E, 242D, 243V, 263S, 265C, 266V, 267R, 269S, 270L, 273A, 274Q/S, 277A/C/D/E/F/G/H/I/K/L/M/N/P/Q/R/S/T/V/W/Y, 284A/R/S, 297Y, 301I/Q/T, 303T, 304D/G, 314S, 316K/V, 318A/C/D/E/F/G/H/I/K/L/M/N/P/Q/R/S/T/V/W/Y, 331I/L/P/V, 335R, 339G, 342A/E/P/R, 343R/W, 346G/L/Q/W, 347F/R, 350R, 351A/E/H, 356S, or 359L/W, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

18. The engineered oxalate decarboxylase polypeptide of any one of claims 14-17, wherein the amino acid sequence of the engineered oxalate decarboxylase polypeptide comprises at least one or more substitutions at amino acid positions: 13, 14, 16, 17, 26, 31, 37, 60, 79, 83, 96, 162, 174, 195, 196, 210, 226, 277, 301, or 318, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

19. The engineered oxalate decarboxylase polypeptide of claim 18, wherein the amino acid sequence of the engineered oxalate decarboxylase polypeptide comprises at least a substitution or amino acid residue 13A/C/D/E/F/G/H/I/K/L/M/N/P/Q/R/S/T/V/W/Y, 14L/V, 16A/C/D/E/F/G/H/I/K/L/M/N/P/Q/R/S/T/V/W/Y, 17A/C/D/E/F/G/H/I/K/L/M/N/P/Q/R/S/T/V/W/Y, 26A/C/D/E/F/G/H/I/K/L/M/N/P/Q/R/S/T/V/W/Y, 31A/C/D/E/F/G/H/I/K/L/M/N/P/Q/R/S/T/V/W/Y, 37A/C/D/E/F/G/H/I/K/L/M/N/P/Q/R/S/T/V/W/Y, 60A/C/D/E/F/G/H/I/K/L/M/N/P/Q/R/S/T/V/W/Y, 79A/C/D/E/F/G/H/I/K/L/M/N/P/Q/R/S/T/V/W/Y, 83A/C/D/E/F/G/H/I/K/L/M/N/P/Q/R/S/T/V/W/Y, 96A/C/D/E/F/G/H/I/K/L/M/N/P/Q/R/S/T/V/W/Y, 162A/C/D/E/F/G/H/I/K/L/M/N/P/Q/R/S/T/V/W/Y, 174A/C/D/E/F/G/H/I/K/L/M/N/P/Q/R/S/T/V/W/Y, 195A/C/D/E/F/G/H/I/K/L/M/N/P/Q/R/S/T/V/W/Y, 196F/K/M/R/S/V, 210A/C/D/E/F/G/H/I/K/L/M/N/P/Q/R/S/T/V/W/Y, 226S/T, 277A/C/D/E/F/G/H/I/K/L/M/N/P/Q/R/S/T/V/W/Y, 301I/Q/T, or 318A/C/D/E/F/G/H/I/K/L/M/N/P/Q/R/S/T/V/W/Y, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

20. The engineered oxalate decarboxylase polypeptide of claim 18, wherein the amino acid sequence of the engineered oxalate decarboxylase polypeptide comprises at least a substitution 13P, 14L, 16S, 17H/G, 26E, 31K, 37G, 60V, 79G, 83W, 96L, 162K, 174K, 195L, 196V, 210A/E, 226S, 277V, 301I, or 318Q, or combinations thereof, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

21. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 1-359 of SEQ ID NO: 172, or to the reference sequence corresponding to SEQ ID NO: 172, wherein the amino acid sequence comprises one or more amino acid substitutions relative to the reference sequence corresponding to residues 1-359 of SEQ ID NO: 172, or relative to the reference sequence corresponding SEQ ID NO: 172.

22. The engineered oxalate decarboxylase polypeptide of claim 21, wherein the amino acid sequence of the engineered oxalate decarboxylase polypeptide comprises at least a substitution or substitution set at amino acid position(s) 13, 212, 124/196/210/226, 5, 16/26/124/155/195/210/284, 196/226, 16/155/195/210/226, 16/195/210/226, 16/155/174/196, 16/195/226, 318, 16/195/196/210, 16/26/124/155/174/196/210, 195/210, 26/155/174/210, 316, 60, 16/155/174, 16/124/174/196, 16/226, 16/26/174/196/226, 17, 331, 124/195, 16/174/196, 188, 195/196/210, 174/196/210, 16/26/155/174, 16/155/195/196/226, 195/226/284, 16, 240, 16/26/124/155/195/196/226, 183/232/339/343, 183/206, 63, 173/347, 16/26/174/196, 16/284, 46, 16/124/195/196, 274, 174/196, 155/174/196, 174/196/226, 16/124/155/174/195, 16/26/155/174/196, 26/174/196/210/284, 16/195/196/284, 174, 16/124/174/195/210/226/284, 16/174/195/284, 26/174/196/210/226/284, 174/196/226/284, 124/174/196, 26/174/195/210/226, 162, 16/124/195/196/284, 206/343, 16/195/196, 16/26/174/195/196/210/226, 155/195/196/226, 18, 335, 124/155/174/195/226, 26/155/174/195/226, 195/196, 153, 155/174/195, 174/195/210/284, 174/195, 37, 124/174/195/226/284, 176, 16/155/174/195/196/226/284, 10, 169/173, 155, 33, 173/183/343/347, 104/265, or 233, wherein the amino acid positions are relative to the reference sequence corresponding to residues 1-359 of SEQ ID NO: 172, or relative to the reference sequence corresponding SEQ ID NO: 172.

23. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 1-359 of SEQ ID NO: 320, or to the reference sequence corresponding to SEQ ID NO: 320, wherein the amino acid sequence comprises one or more amino acid substitutions relative to the reference sequence corresponding to residues 1-359 of SEQ ID NO: 320, or relative to the reference sequence corresponding to SEQ ID NO: 320.

24. The engineered oxalate decarboxylase polypeptide of claim 23, wherein the amino acid sequence of the engineered oxalate decarboxylase polypeptide comprises at least a substitution or substitution set at amino acid position(s) 13/17/196, 17/212/331, 17/26/196/212/226, 17/26/196/212/226/331, 13/17/26/196/331, 17/60/196/226, 17/46/212, 26/60/196/226/331, 17/46/196/212/274/331, 212, 17/196/226, 13/17/26/212/331, 13/17/212/226/331, 196/212/331, 13/17/196/331, 13/17/26/212/226/331, 17/60/196/212/226/331, 13/17/212, 13/17/26/331, 13/17, 13/17/26/196, 46/196/212/226, 17/60/196, 17/196/331, 13/17/46/226/331, 17/196/226/331, 17/46/196/212/331, 17/46/212/331, 17/196, 13/17/212/226, 17/212, 17/26/212, 13/17/226/331, 17/196/212/331, 17/196/212/226/331, 13/17/26/226/331, 46/60/196/226/331, 17/46/196/331, 13/17/46/196, 196/212, 13/17/26, 17/83/263, 17/83/173/227, 17/227/301, 17/83/227/263, 17/83/125, 17/83/173, 17/263/301, 17/125, 17/83/301, 17/173, 17/83/227, 17/227, 17, 17/125/227, 17/83, 128, 76, 141, 110, 79, 117, or 61, wherein the amino acid positions are relative to the reference sequence corresponding to residues 1-359 of SEQ ID NO: 320, or relative to the reference sequence corresponding to SEQ ID NO: 320.

25. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to sequence of residues 1-359 of SEQ ID NO: 396, or to the reference sequence corresponding to SEQ ID NO: 396, wherein the amino acid sequence comprises one or more amino acid substitutions relative to the reference sequence corresponding to residues 1-359 of SEQ ID NO: 396, or relative to the reference sequence corresponding to SEQ ID NO: 396.

26. The engineered oxalate decarboxylase polypeptide of claim 25, wherein the amino acid sequence of the engineered oxalate decarboxylase polypeptide comprises at least a substitution or substitution set at amino acid position(s) 13/17/79, 13/14/17/60/79, 13/17/79/83, 13/17/79/301, 13/14/17/60/79/212, 17/60/197, 13/60/212, 13/14/17/60/79/83/301, 13/17/60/79, 17/60/301, 14/17/79, 17/60/83, 17/79, 13/17/60/79/83/301, 342, 52/190, 94/190, 190/342/351, 13/79, 13/96, 273, 126, 96, 79, 269, 40, 44, 267, 266, 160, 277, 149, 22, 234, 54, 297, 106, or 13/43, wherein the amino acid positions are relative to the reference sequence corresponding to residues 1-359 of SEQ ID NO: 396, or relative to the reference sequence corresponding to SEQ ID NO: 396.

27. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 670, wherein the amino acid sequence comprises one or more amino acid substitutions relative to the reference sequence corresponding to SEQ ID NO: 670.

28. The engineered oxalate decarboxylase polypeptide of claim 27, wherein the amino acid sequence of the engineered oxalate decarboxylase polypeptide comprises at least a substitution at amino acid position 37, 125, 162, 94, 195, 85, 103, 232, 189, 186, 155, 193, 342, 196, 63, 33, 351, 314, 187, 303, 164, 153, 346, 183, 19, 123, 350, 205, 284, 199, 343, 200, 208, 169, 190, or 121, or combinations thereof, wherein the amino acid positions are relative to the reference sequence corresponding to SEQ ID NO: 670.

29. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 616, wherein the amino acid sequence comprises one or more amino acid substitutions relative to the reference sequence corresponding to SEQ ID NO: 616.

30. The engineered oxalate decarboxylase polypeptide of claim 29, wherein the amino acid sequence of the engineered oxalate decarboxylase polypeptide comprises at least a substitution or substitution set at amino acid position(s) 14/26/60/318, 14/26/60/94/162/212, 60/162/226, 60, 14/125/162/212/318, 60/125/226, 14/125/162/226, 26/60/162/226/318, 14/26/94/162/212/226, 14/60/162, 14/60/162/226, 14/94/162/318, 14/94/162, 14/60/226, 14/60/94/212, 14/60/162/212, 14/162, 14/94/212/318, 14/60, 14/26/162, 14, 14/318, 14/162/226/318, 14/125/212, 14/125/226/318, 14/26/60/162, 14/125/162/212/226, 14/94/125/162/318, 14/125/226, 277, 166, 233, 83, 5, 342, 96, 284, or 26, wherein the amino acid positions are relative to the reference sequence corresponding SEQ ID NO: 616.

31. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to SEQ ID NO: 750, wherein the amino acid sequence comprises one or more amino acid substitutions relative to the reference sequence corresponding to SEQ ID NO: 750.

32. The engineered oxalate decarboxylase polypeptide of claim 31, wherein the amino acid sequence of the engineered oxalate decarboxylase polypeptide comprises at least a substitution or substitution set at amino acid position(s) 5/26/83/342, 5/26/277, 5/96/277, 5/83/277/342, 26/83/277, 5/26/83/277, 5/277/342, 5/26/166/277, 83/277/342, 83/96/277/342, 83/96/277, 26/83/277/342, 5/26/83/277/342, 5/83/277, 26/277, 83/277, 5/35/277, 5/26/83/96/277, 5/26/96/277, 5/277, 5/166/277, 26/83/96/277/342, 277, 26/277/342, 5, or 5/83/96/277/342, wherein the amino acid positions are relative to the reference sequence corresponding to SEQ ID NO: 750.

33. The engineered oxalate decarboxylase polypeptide of claim 15, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to SEQ ID NO: 906, wherein the amino acid sequence comprises one or more amino acid substitutions relative to the reference sequence corresponding to SEQ ID NO: 906.

34. The engineered oxalate decarboxylase polypeptide of claim 33, wherein the amino acid sequence of the engineered oxalate decarboxylase polypeptide comprises at least a substitution at amino acid position 26, 31, 96, 60, 318, 210, 277, 37, 16, 195, 79, 17, 13, 83, 162, or 174, or combinations thereof, wherein the amino acid positions are relative to the reference sequence corresponding to SEQ ID NO: 906.

35. The engineered oxalate decarboxylase polypeptide of claim 33, wherein the amino acid sequence of the engineered oxalate decarboxylase polypeptide comprises at least a substitution or substitution set at amino acid position(s) 174/318, 26/96, 60/318, 31/318, 162/174/318, 174/277/318, 26/195/318, 60/162/195, 60/162/318, 174/195/277, 16/17/174, 60/195/277, 37/277/318, 16/162/195/277, 26/79/83/162, 26/31/60/318, 16/174/195/277, 13/37/83/162, 16/17/60/195, 13/17/37/83, 31/37/162/174/318, 13/16/31/83/162, 16/60/174/195/318, 17/60/96/162/195/277, 13/26/60/83/162/174, 17/31/60/162/174/277, 16/17/26/83/96/277, 17/26/37/174/277/318, 13/16/31/60/162/174, 13/17/31/83/277/318, 13/16/17/31/83/162/318, 13/16/60/83/162/195/318, 13/16/17/31/37/83/96/162, 13/17/26/37/60/83/162/210/318, 13/16/26/31/83/96/162/174/318, 13/16/17/31/60/83/96/162/174/195/318, 13/16/26/37/60/83/162/174/195/210/318, 13/16/37/79/83/96/162/174/195/210/277, 13/17/26/31/37/60/79/83/96/162/174/210, 13/16/17/26/37/79/83/96/174/195/210/318, 13/26/31/37/60/83/96/162/174/195/210/277/318, 13/16/17/31/37/60/79/83/96/162/174/195/210/318, 13/16/17/31/60/79/83/96/162/174/195/210/277/318, 13/16/17/26/37/60/79/96/162/174/195/210/277/318, 13/26/31/37/60/79/83/96/162/174/195/210/277/318, 13/16/17/26/31/37/60/96/162/174/195/210/277/318, 13/16/17/26/31/37/79/83/96/162/174/195/210/318, 13/16/17/26/31/37/60/79/96/162/174/210/277/318, 13/17/26/31/37/60/79/83/96/162/174/195/210/277/318, 13/16/26/31/37/60/79/83/96/162/174/195/210/277/318, 16/17/26/31/37/60/79/83/96/162/174/195/210/277/318, or 13/16/17/26/31/37/60/79/83/96/162/174/195/210/277/318, wherein the amino acid positions are relative to the reference sequence corresponding to SEQ ID NO: 906.

36. The engineered oxalate decarboxylase polypeptide of claim 1, wherein the amino acid sequence of the engineered oxalate decarboxylase polypeptide comprises at least a substitution or substitution set of an oxalate decarboxylase polypeptide set forth in Tables 4-1, 4-2, 4-3, 4-4, 4-5, 4-6, 4-7, 5-1, and 5-2, wherein the amino acid positions are relative to the reference sequence corresponding to SEQ ID NO: 172, 320, 396, 616, 670, 750, or 906, or relative to the reference sequence corresponding to residues 1-359 of SEQ ID NO: 172, 320, or 396.

37. The engineered oxalate decarboxylase polypeptide of claim 1, comprising an amino acid sequence comprising SEQ ID NO: 4, an amino acid sequence comprising residues 1-359 of an even-numbered sequence of SEQ ID NOS: 28-614, an amino acid comprising an even-numbered SEQ ID NO. of SEQ ID NOs: 28-614, or an amino acid sequence comprising an even-numbered SEQ ID NO. of SEQ ID NOs: 616-1622, optionally wherein the amino acid sequence includes 1, 2, 3, 4, 5, 6, 7, 8, 9, or up to 10 substitutions.

38. The engineered oxalate decarboxylase polypeptide of claim 1, wherein the amino acid sequence of the engineered oxalate decarboxylase polypeptide comprises residues 1-359 of SEQ ID NO: 172, 320, or 396, or comprises SEQ ID NO: 172, 320, 396, 616, 670, 750, or 906, optionally wherein the amino acid sequence includes 1, 2, 3, 4, 5, 6, 7, 8, 9, or up to 10 substitutions.

39. The engineered oxalate decarboxylase polypeptide of claim 37 or 38, wherein the amino acid sequence includes 1, 2, 3, 4, or 5 substitutions.

40. The engineered oxalate decarboxylase polypeptide of any one of claims 1-39, wherein the engineered oxalate decarboxylase polypeptide has oxalate decarboxylase activity and exhibits one or more improved enzyme properties compared to a reference oxalate decarboxylase having a sequence corresponding to SEQ ID NO: 2, 4, 172, 320, 396, 616, 670, 750, or 906, or a reference sequence corresponding to residues 1-359 of SEQ ID NO: 172, 320, or 396.

41. The engineered oxalate decarboxylase polypeptide of claim 40, wherein the improved property is increased activity at about pH 6 or less, about pH 5.5 or less, about pH 4.5 or less, about pH 3 or less, about pH 2.6 or less, or about pH 2.4 or less, to about pH 2 compared to the reference oxalate decarboxylase.

42. The engineered oxalate decarboxylase polypeptide of claim 40, wherein the improved property is increased thermostability compared to the reference oxalate decarboxylase.

43. The engineered oxalate decarboxylase polypeptide of claim 40, wherein the improved property is increased stability at about pH 6 or less, about pH 5.5 or less, about pH 4.5 or less, about pH 3 or less, about pH 2.6 or less, or about pH 2.4 or less, to about pH 2 compared to the reference oxalate decarboxylase.

44. The engineered oxalate decarboxylase polypeptide of claim 40, wherein the improved property is increased resistance to proteolysis by pepsin or chymotrypsin at pH of about 2.2 to 3.2, compared to the reference oxalate decarboxylase.

45. The engineered oxalate decarboxylase polypeptide of claim 40, wherein the improved enzyme property of the engineered oxalate decarboxylase polypeptide is selected from (a) increased activity on oxalate, (b) increased thermal stability, (c) increased stability at acidic pH, (d) increased activity at acidic pH, (e) increased activity at neutral pH, (f) increased expression, (g) increased solubility, and (h) increased resistance to proteolysis, or any combination of (a), (b), (c), (d), (e), (f), (g) and (h), compared to a reference oxalate decarboxylase having an amino acid sequence corresponding to SEQ ID NO: 2, 4, 172, 320, 396, 616, 670, 750, or 906, or an amino acid sequence corresponding to residues 1-359 of SEQ ID NO: 172, 320, or 396.

46. The engineered oxalate decarboxylase polypeptide of any one of claims 40-45, wherein the reference oxalate decarboxylase has the amino acid sequence corresponding to SEQ ID NO: 2 or 4.

47. The engineered oxalate decarboxylase polypeptide of any of claims 1-46, wherein the engineered oxalate decarboxylase polypeptide is purified.

48. A pharmaceutical composition comprising at least one engineered oxalate decarboxylase polypeptide of any of claims 1-47, and a pharmaceutically acceptable excipient or carrier.

49. The pharmaceutical composition of claim 48, wherein the composition is suitable for oral administration to a human patient.

50. A recombinant polynucleotide comprising a polynucleotide sequence encoding at least an engineered oxalate decarboxylase polypeptide of any of claims 1-46.

51. The recombinant polynucleotide of claim 50, comprising a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 3, a reference polynucleotide sequence corresponding to nucleotide residues 1-1077 of an odd-numbered SEQ ID NO. of SEQ ID NOs: 27-613, a reference polynucleotide sequence corresponding to an odd-numbered SEQ ID NO. of SEQ ID NOs: 27-613, or reference polynucleotide sequence corresponding to an odd-numbered SEQ ID NO. of SEQ ID NOs: 615-1621, wherein the recombinant polynucleotide encodes an engineered oxalate decarboxylase polypeptide.

52. The recombinant polynucleotide of claim 50, wherein the polynucleotide sequence comprises at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to SEQ ID NO: 33, 171, 319, 395, 615, 669, 749, or 905, or to a reference polynucleotide sequence corresponding to nucleotide residues 1 to 1077 of SEQ ID NO: 171, 319, or 395, wherein the recombinant polynucleotide encodes an engineered oxalate decarboxylase polypeptide.

53. The recombinant polynucleotide of any of claims 50-52, wherein the polynucleotide sequence is codon-optimized.

54. The recombinant polynucleotide of claim 50, comprising a polynucleotide sequence comprising SEQ ID NO: 1 or 3; a polynucleotide sequence comprising nucleotide residues 1-1077 of an odd-numbered SEQ ID NO. of SEQ ID NOs: 27-613; a polynucleotide sequence comprising an odd-numbered SEQ ID NO. of SEQ ID NOs: 27-613; or a polynucleotide sequence comprising an odd-numbered SEQ ID NO. of SEQ ID NOs: 615-1621.

55. The recombinant polynucleotide of claim 50, comprising the polynucleotide sequence comprising SEQ ID NO: 3, 171, 319, 395, 615, 669, 749, or 905, or comprising nucleotide residues 1-1077 of SEQ ID NO: 171, 319, or 395.

56. An expression vector comprising at least one recombinant polynucleotide of any one of claims 50-55.

57. The expression vector of claim 56, wherein the recombinant polynucleotide is operably linked to a control sequence.

58. The expression vector of claim 57, wherein the control sequence comprises a promoter.

59. A host cell comprising at least one recombinant polynucleotide of any one of claims 50-55, or the expression vector of any one of claims 56-58.

60. The host cell of claim 59, wherein the host cell is a prokaryotic cell or eukaryotic cell.

61. The host cell of claim 60, wherein the host cell is a bacterial cell.

62. A method of producing an engineered oxalate decarboxylase polypeptide comprising culturing a host cell of any one of claims 59-61, under suitable culture conditions such that the engineered oxalate decarboxylase polypeptide is produced.

63. The method of claim 62, further comprising recovering the engineered oxalate decarboxylase polypeptide from the culture and/or host cells.

64. The method of claim 62 or 63, further comprising a step of purifying the engineered oxalate decarboxylase polypeptide.

65. A method of reducing levels of oxalate in a subject, comprising administering to a subject in need thereof an effective amount of an engineered oxalate decarboxylase polypeptide of any one of claims 1-47 or a composition of claim 48 or 49 to reduce levels of oxalate in the subject.

66. The method of claim 65, wherein reducing the levels of oxalate is in urine and/or plasma levels of oxalate.

67. A method for treating and/or preventing the symptoms of hyperoxaluria in a subject, comprising administering to a subject with hyperoxaluria an effective amount of an engineered oxalate decarboxylase polypeptide of any of claims 1-47 or a composition of claim 48 or 49.

68. The method of claim 67, wherein the hyperoxaluria is secondary, enteric, or idiopathic hyperoxaluria.

69. The method of claims 67 or 68, wherein the symptoms of hyperoxaluria are ameliorated.

70. The method of claim 69, wherein the levels of oxalate in urine and/or plasma is reduced.

71. The method of any of claims 67-70, wherein the subject is able to eat a diet that is higher in its oxalate content as compared to diets required by subjects who are afflicted with hyperoxaluria.

72. Use of the engineered oxalate decarboxylase of any one of claims 1-47 or the composition of claim 48 or 49 for treating a disease or condition associated with elevated levels of oxalate.

73. Use of the engineered oxalate decarboxylase of any one of claims 1-47 in the preparation of a medicament for treating a disease or condition associated with elevated levels of oxalate.

74. The use of claim 72 or 73, wherein the disease or condition associated with elevated levels of oxalate is hyperoxaluria.
